SEQUENCE LISTING (353 sequences)

SEQ ID NO: 1 - protein, 288 aa, Homo sapiens
  REGION 1..288: note = CD80(B7-1)
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - protein, 329 aa, Homo sapiens
  REGION 1..329: note = CD86(B7-2)
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 3 - protein, 290 aa, Homo sapiens
  REGION 1..290: note = CD274 (PD-L1, B7-H1)
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 273 aa, Homo sapiens
  REGION 1..273: note = PDCD1LG2(PD-L2, CD273)
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 5 - protein, 302 aa, Homo sapiens
  REGION 1..302: note = ICOSLG(B7RP1, CD275, ICOSL, B7-H2)
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 6 - protein, 534 aa, Homo sapiens
  REGION 1..534: note = CD276(B7-H3)
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 7 - protein, 282 aa, Homo sapiens
  REGION 1..282: note = VTCN1(B7-H4)
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 8 - protein, 220 aa, Homo sapiens
  REGION 1..220: note = CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 9 - protein, 223 aa, Homo sapiens
  REGION 1..223: note = CTLA4
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 10 - protein, 288 aa, Homo sapiens
  REGION 1..288: note = PDCD1(PD-1)
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 11 - protein, 199 aa, Homo sapiens
  REGION 1..199: note = ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 12 - protein, 289 aa, Homo sapiens
  REGION 1..289: note = BTLA(CD272)
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 13 - protein, 458 aa, Homo sapiens
  REGION 1..458: note = CD4
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 14 - protein, 235 aa, Homo sapiens
  REGION 1..235: note = CD8A(CD8-alpha)
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 15 - protein, 210 aa, Homo sapiens
  REGION 1..210: note = CD8B(CD8-beta)
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 16 - protein, 525 aa, Homo sapiens
  REGION 1..525: note = LAG3
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 17 - protein, 301 aa, Homo sapiens
  REGION 1..301: note = HAVCR2(TIM-3)
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 18 - protein, 526 aa, Homo sapiens
  REGION 1..526: note = CEACAM1
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 19 - protein, 244 aa, Homo sapiens
  REGION 1..244: note = TIGIT
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 20 - protein, 417 aa, Homo sapiens
  REGION 1..417: note = PVR(CD155)
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 21 - protein, 538 aa, Homo sapiens
  REGION 1..538: note = PVRL2(CD112)
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 22 - protein, 336 aa, Homo sapiens
  REGION 1..336: note = CD226
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 23 - protein, 351 aa, Homo sapiens
  REGION 1..351: note = CD2
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 24 - protein, 180 aa, Homo sapiens
  REGION 1..180: note = CD160
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQA

SEQ ID NO: 25 - protein, 278 aa, Homo sapiens
  REGION 1..278: note = CD200
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 26 - protein, 325 aa, Homo sapiens [sequence omitted]

SEQ ID NO: 27 - protein, 201 aa, Homo sapiens
  REGION 1..201: note = NC R3 (NKp30)
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 28 - protein, 208 aa, Homo sapiens [sequence omitted]

SEQ ID NO: 29 - protein, 224 aa, Homo sapiens
  REGION 1..224: note = CD86 ECD (24-247)
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 30 - protein, 220 aa, Homo sapiens
  REGION 1..220: note = CD274 (PD-L1, B7-H1) ECD
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 31 - protein, 201 aa, Homo sapiens
  REGION 1..201: note = PDCD1LG2(PD-L2, CD273) ECD
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 32 - protein, 238 aa, Homo sapiens [sequence omitted]

SEQ ID NO: 33 - protein, 438 aa, Homo sapiens
  REGION 1..438: note = CD276(B7-H3) ECD
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 34 - protein, 235 aa, Homo sapiens
  REGION 1..235: note = VTCN1(B7-H4) ECD
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 35 - protein, 134 aa, Homo sapiens
  REGION 1..134: note = CD28 ECD
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 36 - protein, 126 aa, Homo sapiens [sequence omitted]

SEQ ID NO: 37 - protein, 150 aa, Homo sapiens [sequence omitted]

SEQ ID NO: 38 - protein, 120 aa, Homo sapiens
  REGION 1..120: note = ICOS ECD
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 39 - protein, 127 aa, Homo sapiens
  REGION 1..127: note = BTLA(CD272) ECD
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 40 - protein, 371 aa, Homo sapiens
  REGION 1..371: note = CD4 ECD
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQP

SEQ ID NO: 41 - protein, 161 aa, Homo sapiens
  REGION 1..161: note = CD8A(CD8-alpha) ECD
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 42 - protein, 149 aa, Homo sapiens
  REGION 1..149: note = CD8B(CD8-beta) ECD
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 43 - protein, 422 aa, Homo sapiens
  REGION 1..422: note = LAG3 ECD
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 44 - protein, 181 aa, Homo sapiens
  REGION 1..181: note = HAVCR2(TIM-3) ECD
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 45 - protein, 394 aa, Homo sapiens
  REGION 1..394: note = CEACAM1 ECD
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPG

SEQ ID NO: 46 - protein, 120 aa, Homo sapiens
  REGION 1..120: note = TIGIT ECD
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 47 - protein, 323 aa, Homo sapiens
  REGION 1..323: note = PVR(CD155) ECD
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 48 - protein, 329 aa, Homo sapiens
  REGION 1..329: note = PVRL2(CD112) ECD
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 49 - protein, 236 aa, Homo sapiens [sequence omitted]

SEQ ID NO: 50 - protein, 185 aa, Homo sapiens
  REGION 1..185: note = CD2 ECD
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 51 - protein, 133 aa, Homo sapiens
  REGION 1..133: note = CD160 ECD
INITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLS

SEQ ID NO: 52 - protein, 202 aa, Homo sapiens
  REGION 1..202: note = CD200 ECD
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKG

SEQ ID NO: 53 - protein, 215 aa, Homo sapiens
  REGION 1..215: note = CD200R1(CD200R) ECD
MDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKL

SEQ ID NO: 54 - protein, 117 aa, Homo sapiens
  REGION 1..117: note = NC R3 (NKp30) ECD
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 55 - protein, 254 aa, Homo sapiens
  REGION 1..254: note = Mature CD80(B7-1)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 56 - protein, 306 aa, Homo sapiens
  REGION 1..306: note = Mature CD86(B7-2)
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 57 - protein, 272 aa, Homo sapiens
  REGION 1..272: note = Mature CD274 (PD-L1, B7-H1)
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 58 - protein, 254 aa, Homo sapiens
  REGION 1..254: note = Mature PDCD1LG2(PD-L2, CD273)
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 59 - protein, 284 aa, Homo sapiens
  REGION 1..284: note = Mature ICOSLG(B7RP1, CD275, ICOSL, B7-H2)
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 60 - protein, 506 aa, Homo sapiens
  REGION 1..506: note = Mature CD276(B7-H3)
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 61 - protein, 258 aa, Homo sapiens
  REGION 1..258: note = Mature VTCN1(B7-H4)
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 62 - protein, 202 aa, Homo sapiens
  REGION 1..202: note = Mature CD28
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 63 - protein, 188 aa, Homo sapiens
  REGION 1..188: note = Mature CTLA4
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 64 - protein, 268 aa, Homo sapiens
  REGION 1..268: note = Mature PDCD1(PD-1)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 65 - protein, 179 aa, Homo sapiens
  REGION 1..179: note = Mature ICOS
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 66 - protein, 259 aa, Homo sapiens
  REGION 1..259: note = Mature BTLA(CD272)
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 67 - protein, 433 aa, Homo sapiens
  REGION 1..433: note = Mature CD4
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 68 - protein, 214 aa, Homo sapiens
  REGION 1..214: note = Mature CD8A(CD8-alpha)
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 69 - protein, 189 aa, Homo sapiens
  REGION 1..189: note = Mature CD8B(CD8-beta)
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 70 - protein, 497 aa, Homo sapiens
  REGION 1..497: note = Mature LAG3
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 71 - protein, 280 aa, Homo sapiens
  REGION 1..280: note = Mature HAVCR2(TIM-3)
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 72 - protein, 492 aa, Homo sapiens
  REGION 1..492: note = Mature CEACAM1
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 73 - protein, 222 aa, Homo sapiens
  REGION 1..222: note = Mature TIGIT
MTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 74 - protein, 397 aa, Homo sapiens
  REGION 1..397: note = Mature PVR(CD155)
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 75 - protein, 507 aa, Homo sapiens
  REGION 1..507: note = Mature PVRL2(CD112)
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 76 - protein, 318 aa, Homo sapiens
  REGION 1..318: note = Mature CD226
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 77 - protein, 327 aa, Homo sapiens
  REGION 1..327: note = Mature CD2
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 78 - protein, 154 aa, Homo sapiens
  REGION 1..154: note = Mature CD160
INITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQA

SEQ ID NO: 79 - protein, 248 aa, Homo sapiens
  REGION 1..248: note = Mature CD200
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 80 - protein, 297 aa, Homo sapiens
  REGION 1..297: note = Mature CD200R1(CD200R)
MDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 81 - protein, 183 aa, Homo sapiens
  REGION 1..183: note = Mature NC R3 (NKp30)
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 82 - protein, 414 aa, Homo sapiens
  REGION 1..414: note = VSIG8
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 83 - protein, 393 aa, Homo sapiens
  REGION 1..393: note = Mature VSIG8
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 84 - protein, 242 aa, Homo sapiens
  REGION 1..242: note = VSIG8 ECD
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIG

SEQ ID NO: 85 - protein, 224 aa, synthetic construct
  REGION 1..224: note = CD86 ECD v1
APLKIQAYFNETADLPCQFANSQNLSLSELVVFWQDQENLVLNEVYLGKEKFDSVHSKYMGRTSFDSDSWALRLHNLQIKDKGLYQCIIYHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 86 - protein, 224 aa, synthetic construct
  REGION 1..224: note = CD86 ECD v2
APLKIQAYFNETADLPCQFANSQNLSLSELVVFWQDQENLVLNEVYLGKEKFGSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIVLEDPQPPPDHIP

SEQ ID NO: 87 - protein, 224 aa, synthetic construct
  REGION 1..224: note = CD86 ECD v3
  VARIANT 215: note = Xaa is any amino acid
  VARIANT 217: note = Xaa is any amino acid
  VARIANT 218: note = Xaa is any amino acid
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEXPXXPPDHIP

SEQ ID NO: 88 - protein, 224 aa, synthetic construct
  REGION 1..224: note = CD86 ECD v4
APLKIQAYFNETADLPCQFANSQNQSLSELVVFWQDQENLVLKEVYLGKEKFDSVHSKYMGRTSFDSDSWTLRLHNLQNKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSTSLSVSFPDVTSNMTIFCILDTDKTRLLSSPFSIELEDPQPPPDHIP

SEQ ID NO: 89 - protein, 224 aa, synthetic construct
  REGION 1..224: note = CD86 ECD v5
APLKIQAYFNETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLPVSFPDVTSNMTIFCIMETDKTRLLTSPFSIELEDPQPPPDHIP

SEQ ID NO: 90 - protein, 224 aa, synthetic construct
  REGION 1..224: note = CD86 ECD v6
APLKIQAYFNETADLPCQFANSQNLSLSELVVFWQDQENLVLNEVYLGKEKFDSVHSKYMGRTSFDSDSWTLRLHNLQIKDKGLYRCIILHKTPTGMIRIHQMNSELSVLANFSQPEIVPISNITENVYINMTCSSIHGYPEPKKMSDLLRTKNSTIEYDGVMQKSQDNVTELYDVSISLPVSFPDVTSNMTIFCILETDKTRLLSSPFSIELEDHQPPPDHIP

SEQ ID NO: 91 - protein, 224 aa, synthetic construct
  REGION 1..224: note = CD86 ECD v7
  VARIANT 201: note = Xaa is any amino acid
APLKIQAYFNETADLPCQFANSQNLSLSELVVIWQDQENLVLNEVYLGKEKFDSVHSKYMGRTSFDSDSWTLRLHNLQIKDKGLYQCIIYHKKPTGMIRIHQMNSELSVLANFSQPEIVPISNITENAYINLTCSSIHGYAEPKKMSVLLRTKNSTIGYDGVMQKSQDNVTELYDVSISLPVSFPDVTSNMTIFCILETDXTRLLSSPFSIELEDPQPPPDHIP

SEQ ID NO: 92 - protein, 224 aa, synthetic construct
  REGION 1..224: note = CD86 ECD v8
APLKIQAYFNETADLPCQFANSQNLSLSELVVFWQDQEDLVLNEVYLGKEKFDSVHSKYMGRTSFDSDSWTLRLHNLQIRDKGLYRCFILHKTPTGMIRIHQMNSELSVLANFSQPEIVPISDITENVYINLTCSSIHGYPEPKKMSVLLRTKDSTIEYDGVMQKSQDNVTELYDVSISLSVSFPDVTSNMTIFCILETDKTRLLSSPFSIELEDPQPPPDHIP

SEQ ID NO: 93 - protein, 224 aa, synthetic construct
  REGION 1..224: note = CD86 ECD v9
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLANFSQPEIVPISNITENVYINLACSSIHGYPEPKKMSVLLRTKNSTIEYDGVMQKSQDNVTELYDVSISLPVSFPDVTSNMTIFCILETDKTRLLSSPFSIELEVPQPPPDHIP

SEQ ID NO: 94 - protein, 224 aa, synthetic construct
  REGION 1..224: note = CD86 ECD v10
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 95 - protein, 224 aa, synthetic construct
  REGION 1..224: note = CD86 ECD v11
APLKIQAYFNETADLPCQFANSQNLSLSELVVFWQDQENMVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISSSVPFPDVTSNMTIFCILETDKTRLLSSPFSIELEDPQPPPDHIP

SEQ ID NO: 96 - protein, 224 aa, synthetic construct
  REGION 1..224: note = CD86 ECD v12
APLKIQAYFNETADLPCKFANSQNLSLSELVVIWQDQENSVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 97 - protein, 224 aa, synthetic construct
  REGION 1..224: note = CD86 ECD v13
APLKIQAYFNETADLPCQFANSQNLSLSELVVFWQDQENLVLNEVYLGKEKFDSVHSKYMGRTSFDSDSWTLRLHNLQIKDKGLYKCIILHKKPTGMIRIHQMNSELSVLANFSQPEIVPISNITENVYINLTCSSTHGYPEPKKMSVLLRTKNSTIEYDGVMQKSQDNVTELYDVSISLPVSFPDVTSNMTIFCILETDKTRLLSSPFSIELEDPQPPPDHIP

SEQ ID NO: 98 - protein, 224 aa, synthetic construct
  REGION 1..224: note = CD86 ECD v14
APLKIQAYFNETADLPCQFANSQNLSLSELVVFWQDQENLVLNEVYLGKEKFDSVHSKYMGRTSFDSDSWTLRLHNPQIKDKGLYQCIIYHKKPTGMIRIHQMNSELSVLANFSQPEIVPISNITENVYINLTCSSIHGYPEPKKMSVLLRTRNSTIEYDGVMQKSQDNDTELYDVSISLPVSFPDVTSNMTIFCILETDKTRLLSSPFSIELEDPQPPPDHIP

SEQ ID NO: 99 - protein, 224 aa, synthetic construct
  REGION 1..224: note = CD86 ECD v15
APLKIQAYFNETADLPCQFANSQNLSLGELVVIWQDQENLVLNEVYLGKEKLDSVHSKYMGRTSFDSDSWTLRLHNLQIKDKGLYQCIILHKKPTGMIRIHHMNSELSVLANFSQPEIVPISNITENVYINLTCSSIHGYPEPKKMSVLLRTKNSTIEYDGVMQKSQDNVTELYDVSTSLSVSFPDVTSNMTIFCILETDKTRLLSSPFSIELEDPQPPPDHIP

SEQ ID NO: 100 - protein, 224 aa, synthetic construct
  REGION 1..224: note = CD86 ECD v16
  VARIANT 212: note = Xaa is any amino acid
  VARIANT 221: note = Xaa is any amino acid
APLKIQAYFNETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPEKMSVLLRTKNSTIEYDGVMQKSQDNVTELYDVSISSSVSFPDVTSNMTIFCILETDKTRLLSSPFSIXLEDPQPPPXHIP

SEQ ID NO: 101 - protein, 224 aa, synthetic construct
  REGION 1..224: note = CD86 ECD v17
  VARIANT 221: note = Xaa is any amino acid
APLKIQAYFNETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTENSTIEYDGVMQKSQDNVTGLYDVSISLSVSFPDVTSNMNIFCILETDKTRLLSSPFSIELEDPQPPPXHIP

SEQ ID NO: 102 - protein, 224 aa, synthetic construct
  REGION 1..224: note = CD86 ECD v18
  VARIANT 215: note = Xaa is any amino acid
APLKIQAYFNETADLPCQFANSQNLSLSELVVIWQDQENLVLNEVYLGKEKFDSVHSKYMGRTSFDSDSWTLRLHNLQIKDKGLYRCIIYHKKPTGMIRIHQMNSELSVLANFSQPEIVPISNITENVYINLTCSSIHGYPEPKKMSVLLRTKNSTIEYDGVMQKSQDNVTELYEVSISLSVSFPDVTSNMTIFCVLDTDKTRLLSSPFSIELEXPQPPPDHIP

SEQ ID NO: 103 - protein, 224 aa, synthetic construct
  REGION 1..224: note = CD86 ECD v19
  VARIANT 212: note = Xaa is any amino acid
  VARIANT 215: note = Xaa is any amino acid
APLKIQAYFNETADLPCQFANSQNLSLSELVVFWQDQENLVLNEIYLGKEKFDSVHSKYMGRTSFDSNSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVPFPDVTSNMTIFCILETDKTRLSSSPFSIXLEXPQPPPDHIP

SEQ ID NO: 104 - protein, 224 aa, synthetic construct
  REGION 1..224: note = CD86 ECD v20
APLKIQAYFNETADLPCQFANSQNQSLSELVVFWQDQVNLVLNEVYLGKEKFDSVHSKYMGRTSFDSDSWTLRLHNLQIKDKGLYQCIIHHKKPTGMIRIHQMNSELSVLANFGQPEIVPISNITENVYINLTCSSIHGYPEHKKMSVLLRTKNSTIEYDGVMQKSQDNVTELYDVSISLSVSFPDVTSNMTIFCILETDKTRLLSSPFSIELEDPQPPPDHIP

SEQ ID NO: 105 - protein, 224 aa, synthetic construct
  REGION 1..224: note = CD86 ECD v21
APLKIQAYFNETADLPCQFANSQNQSLSELVVFWQDQENLVLNEVYLGKEKFDSVHSKYMGRTSFDSDSWTLRLHNLQIKDKGLYQCIIYHKKPTGMIRIHQMNSELSVLANFSQPEIVPISNITENVYINLTCSSIHGYPEPKKMSVLLRTKNSTIEYDGVMQKSQDNVTELYDVSISSSVSFPDVTSNMTIFCILETDKTRLLSSPFSIELEDPQPPPDHIP

SEQ ID NO: 106 - protein, 224 aa, synthetic construct
  REGION 1..224: note = CD86 ECD v22
APLKIQAYFNETADLPCQFANSQNQSLSELVVFWQDQENLVLNEVYLGKEKFDSVHSKYMGRTSFDSDSWTLRLHNLQIKDKGLYQCIIYHKKPTGMIRIHQMNSELSVLANFSQPEIVPISNITENVNINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 107 - protein, 224 aa, synthetic construct
  REGION 1..224: note = CD86 ECD v23
APLKIQAYFNETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTVFCILETDKTRLLSSPFSIELEDPQPPPDHIP

SEQ ID NO: 108 - protein, 223 aa, synthetic construct
  REGION 1..223: note = CD86 ECD v24
APLKIQAYFNETADLPCQFANSQNQSLSELVVFWQDQENLVLNEVYLGKEKFDSVHSKYMGRTSFDSDSWTLRLHNLQIEDKGLYQCIIYHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDTF

SEQ ID NO: 109 - protein, 224 aa, synthetic construct
  REGION 1..224: note = CD86 ECD v25
APLKIQAYFNETADLPCQFANSQNQSLSELVVFWQDQENLVLNEVYLGKEKFDSVHSKYMGRTSFDSDSWTLRLHNLQIMDKGLYQCTIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 110 - protein, 224 aa, synthetic construct
  REGION 1..224: note = CD86 ECD v26
APLKIQAYFNETADLPCQFANSQNQSLSELVVFWQDQENLVLNEVYLGKEKFDSVHSKYMGRTSFDSDSWTLRLHNLQIKDKGLYQCIIHHIKPTGMIRIHQMNSELSVLANSSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 111 - protein, 224 aa, synthetic construct
  REGION 1..224: note = CD86 ECD v27
APLKIQAYFNETADLPCQFANSQNQSLSELVVFWQDQENLVLNEVYLGKEKFDSVHSKYKGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 112 - protein, 224 aa, synthetic construct
  REGION 1..224: note = CD86 ECD v28
APLKIQAYFNETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 113 - protein, 224 aa, synthetic construct
  REGION 1..224: note = CD86 ECD v29
APLKIQAYFNETADLPCQFANSQNLSLSELVVIWQDQENLVLNEVYLGKEKFDSVHSKYMGRTSFDSDSWTLRLHNLQIKDKGLYRCIILHKTPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 114 - protein, 224 aa, synthetic construct
  REGION 1..224: note = CD86 ECD v30
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 115 - protein, 224 aa, synthetic construct
  REGION 1..224: note = CD86 ECD v31
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFSDVTSNMTIFCILETDKTRLLSSPFSIELEDPQPPPDHIP

SEQ ID NO: 116 - protein, 223 aa, synthetic construct
  REGION 1..223: note = CD86 ECD v32
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFSDVTSNMTIFCILETDKTRLLSSPFSIELEDPQPPPDHI

SEQ ID NO: 117 - protein, 224 aa, synthetic construct
  REGION 1..224: note = CD86 ECD v33
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSIRLSVSFPDVTSNMTIFCILETDKTRLLSSPFSIELEDPQPPPDHIP

SEQ ID NO: 118 - protein, 224 aa, synthetic construct
  REGION 1..224: note = CD86 ECD v34
APLKIQAYFNETADLPCQFANSQNLSLSELVVFWQDQENLVLNEVYLGKEKFDSVHSKYMGRTSFDSDSWTLRLHNLQIKDKGLYQCIIYHKKPTGMIRIHQMNSELSVLANFSQPEIVPISNITENVYINLTCSSIHGYPEPKKMSVLLRTKNSTIEYDGVMQKSQDNVTELYDVSISLPVSFPDVTSNMTVFCILETDKTRLLSSPFSIELEDPQPPPDHIP

SEQ ID NO: 119 - protein, 224 aa, synthetic construct
  REGION 1..224: note = CD86 ECD v35
APLKIQAYFNETADLPCQFANSQNLSLSELVVFWQDQENLVLNEVYLGKEKFDSVHSKYMGRTSFDSDSWTLRLHNLQIKDTGLYQCIILHKKPTGMIRIHQMNSELSVLANFSQPEIVPISNITENVYINLTCSSIHGYPEPKKMSVLLRSKNSTIEYDGVMQKSQDNVTELYDVSISLSVSFPDVTSNMTIFCILETDKTRLLSPPFSIELEDPQPPPDHIP

SEQ ID NO: 120 - protein, 224 aa, synthetic construct
  REGION 1..224: note = CD86 ECD v36
APLKIQAYFNETADLPCQFANSQNLSLSELVVFWQDQENLVLNEVYLGKEKFDSVHSKYMGRTSFDSDSWTLRLHNLQIKDKGLYRCIILHKTPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 121 - protein, 224 aa, synthetic construct
  REGION 1..224: note = CD86 ECD v37
APLKIQAYFNETADLPCQFANSQNQSLGELVVFWQDQENLVLNEVYLGKEKFDSVHSKYMGRTSFDSDSWTLRLHNLQIKDKGLYQCIIYHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 122 - protein, 99 aa, synthetic construct
  REGION 1..99: note = CD86(B7-2) IgV WT
NETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELS

SEQ ID NO: 123 - protein, 111 aa, synthetic construct
  REGION 1..111: note = CD86(B7-2) IgV (24-134)
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLA

SEQ ID NO: 124 - protein, 111 aa, synthetic construct
  REGION 1..111: note = CD86 IgV v44
APLKIQAYFNETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLA

SEQ ID NO: 125 - protein, 111 aa, synthetic construct
  REGION 1..111: note = CD86 IgV v45
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLA

SEQ ID NO: 126 - protein, 111 aa, synthetic construct
  REGION 1..111: note = CD86 IgV v46
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMNSELSVLA

SEQ ID NO: 127 - protein, 111 aa, synthetic construct
  REGION 1..111: note = CD86 IgV v47
APLKIQAYFNETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELSVLA

SEQ ID NO: 128 - protein, 111 aa, synthetic construct
  REGION 1..111: note = CD86 IgV v48
APLKIQAYFNETADLPCQFANSQNQSLSELVVFWQDQENLVLNEVYLGKEKFDSVHSKYKGRTSFDSDSWTLRLHNLQIKDKGLYQCIILHKKPTGMIRIHQMNSELSVLA

SEQ ID NO: 129 - protein, 111 aa, synthetic construct
  REGION 1..111: note = CD86 IgV v49
APLKIQAYFNETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELSVLA

SEQ ID NO: 130 - protein, 111 aa, synthetic construct
  REGION 1..111: note = CD86 IgV v50
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLA

SEQ ID NO: 131 - protein, 224 aa, synthetic construct; identical to SEQ ID NO: 114
  REGION 1..224: note = CD86 IgC WT v1

SEQ ID NO: 132 - protein, 224 aa, synthetic construct
  REGION 1..224: note = CD86 IgC WT v2
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 133 - protein, 224 aa, synthetic construct
  REGION 1..224: note = CD86 IgC WT v3
APLKIQAYFNETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 134 - protein, 224 aa, synthetic construct; identical to SEQ ID NO: 114
  REGION 1..224: note = CD86 WT IgC v4

SEQ ID NO: 135 - protein, 355 aa, synthetic construct
  REGION 1..355: note = CD86 NKP30 Stack v1
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLANFSQPEIVPISNITENVYINLACSSIHGYPEPKKMSVLLRTKNSTIEYDGVMQKSQDNVTELYDVSISLPVSFPDVTSNMTIFCILETDKTRLLSSPFSIELEVPQPPPDHIPGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 136 - protein, 355 aa, synthetic construct
  REGION 1..355: note = CD86 NKP30 Stack v2
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 137 - protein, 355 aa, synthetic construct
  REGION 1..355: note = CD86 NKP30 Stack v3
APLKIQAYFNETADLPCQFANSQNQSLSELVVFWQDQENLVLNEVYLGKEKFDSVHSKYKGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 138 - protein, 355 aa, synthetic construct
  REGION 1..355: note = CD86 NKP30 Stack v4
APLKIQAYFNETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTVFCILETDKTRLLSSPFSIELEDPQPPPDHIPGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 139 - protein, 355 aa, synthetic construct
  REGION 1..355: note = CD86 NKP30 Stack v5
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSIRLSVSFPDVTSNMTIFCILETDKTRLLSSPFSIELEDPQPPPDHIPGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 140 - protein, 239 aa, synthetic construct
  REGION 1..239: note = CD86 NKP30 Stack v6
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSGGGGSGGGGSGGGGSLWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 141 - protein, 111 aa, synthetic construct
  REGION 1..111: note = CD86 IgV v51
APLKIQAYFNETADLPCQFANSQNLSLSELVVFWQDQENLVLNEVYLGKEKFDSVHSKYMGRTSFDSDSWALRLHNLQIKDKGLYQCIIYHKKPTGMIRIHQMNSELSVLA

SEQ ID NO: 142 - protein, 111 aa, synthetic construct
  REGION 1..111: note = CD86 IgV v52
APLKIQAYFNETADLPCQFANSQNLSLSELVVFWQDQENLVLNEVYLGKEKFGSVHSKYMGRTSFDSDSWTLRLHNLQIKDKGLYQCIIHHKKPTGMIRIHQMNSELSVLA

SEQ ID NO: 143 - protein, 111 aa, synthetic construct; identical to SEQ ID NO: 130
  REGION 1..111: note = CD86 IgV v53

SEQ ID NO: 144 - protein, 111 aa, synthetic construct
  REGION 1..111: note = CD86 IgV v54
APLKIQAYFNETADLPCQFANSQNQSLSELVVFWQDQENLVLKEVYLGKEKFDSVHSKYMGRTSFDSDSWTLRLHNLQNKDKGLYQCIILHKKPTGMIRIHQMNSELSVLA

SEQ ID NO: 145 - protein, 111 aa, synthetic construct; identical to SEQ ID NO: 124
  REGION 1..111: note = CD86 IgV v55

SEQ ID NO: 146 - protein, 111 aa, synthetic construct
  REGION 1..111: note = CD86 IgV v56
APLKIQAYFNETADLPCQFANSQNLSLSELVVFWQDQENLVLNEVYLGKEKFDSVHSKYMGRTSFDSDSWTLRLHNLQIKDKGLYRCIILHKTPTGMIRIHQMNSELSVLA

SEQ ID NO: 147 - protein, 111 aa, synthetic construct
  REGION 1..111: note = CD86 IgV v57
APLKIQAYFNETADLPCQFANSQNLSLSELVVIWQDQENLVLNEVYLGKEKFDSVHSKYMGRTSFDSDSWTLRLHNLQIKDKGLYQCIIYHKKPTGMIRIHQMNSELSVLA

SEQ ID NO: 148 - protein, 111 aa, synthetic construct
  REGION 1..111: note = CD86 IgV v58
APLKIQAYFNETADLPCQFANSQNLSLSELVVFWQDQEDLVLNEVYLGKEKFDSVHSKYMGRTSFDSDSWTLRLHNLQIRDKGLYRCFILHKTPTGMIRIHQMNSELSVLA

SEQ ID NO: 149 - protein, 111 aa, synthetic construct; identical to SEQ ID NO: 125
  REGION 1..111: note = CD86 IgV v59

SEQ ID NO: 150 - protein, 111 aa, synthetic construct
  REGION 1..111: note = CD86 IgV v60
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 151 - protein, 111 aa, synthetic construct
  REGION 1..111: note = CD86 IgV v61
APLKIQAYFNETADLPCQFANSQNLSLSELVVFWQDQENMVLNEVYLGKEKFDSVHSKYMGRTSFDSDSWTLRLHNLQIKDKGLYQCIILHKKPTGMIRIHQMNSELSVLA

SEQ ID NO: 152 - protein, 111 aa, synthetic construct
  REGION 1..111: note = CD86 IgV v62
APLKIQAYFNETADLPCKFANSQNLSLSELVVIWQDQENSVLNEVYLGKEKFDSVHSKYMGRTSFDSDSWTLRLHNLQIKDKGLYQCIILHKKPTGMIRIHQMNSELSVLA

SEQ ID NO: 153 - protein, 111 aa, synthetic construct
  REGION 1..111: note = CD86 IgV v63
APLKIQAYFNETADLPCQFANSQNLSLSELVVFWQDQENLVLNEVYLGKEKFDSVHSKYMGRTSFDSDSWTLRLHNLQIKDKGLYKCIILHKKPTGMIRIHQMNSELSVLA

SEQ ID NO: 154 - protein, 111 aa, synthetic construct
  REGION 1..111: note = CD86 IgV v64
APLKIQAYFNETADLPCQFANSQNLSLSELVVFWQDQENLVLNEVYLGKEKFDSVHSKYMGRTSFDSDSWTLRLHNPQIKDKGLYQCIIYHKKPTGMIRIHQMNSELSVLA

SEQ ID NO: 155 - protein, 111 aa, synthetic construct
  REGION 1..111: note = CD86 IgV v65
APLKIQAYFNETADLPCQFANSQNLSLGELVVIWQDQENLVLNEVYLGKEKLDSVHSKYMGRTSFDSDSWTLRLHNLQIKDKGLYQCIILHKKPTGMIRIHHMNSELSVLA

SEQ ID NO: 156 - protein, 111 aa, synthetic construct; identical to SEQ ID NO: 129
  REGION 1..111: note = CD86 IgV v66

SEQ ID NO: 157 - protein, 111 aa, synthetic construct; identical to SEQ ID NO: 129
  REGION 1..111: note = CD86 IgV v67

SEQ ID NO: 158 - protein, 111 aa, synthetic construct
  REGION 1..111: note = CD86 IgV v68
APLKIQAYFNETADLPCQFANSQNLSLSELVVIWQDQENLVLNEVYLGKEKFDSVHSKYMGRTSFDSDSWTLRLHNLQIKDKGLYRCIIYHKKPTGMIRIHQMNSELSVLA

SEQ ID NO: 159 - protein, 111 aa, synthetic construct
  REGION 1..111: note = CD86 IgV v69
APLKIQAYFNETADLPCQFANSQNLSLSELVVFWQDQENLVLNEIYLGKEKFDSVHSKYMGRTSFDSNSWTLRLHNLQIKDKGLYQCIILHKKPTGMIRIHQMNSELSVLA

SEQ ID NO: 160 - protein, 111 aa, synthetic construct
  REGION 1..111: note = CD86 IgV v70
APLKIQAYFNETADLPCQFANSQNQSLSELVVFWQDQVNLVLNEVYLGKEKFDSVHSKYMGRTSFDSDSWTLRLHNLQIKDKGLYQCIIHHKKPTGMIRIHQMNSELSVLA

SEQ ID NO: 161 - protein, 111 aa, synthetic construct
  REGION 1..111: note = CD86 IgV v71
APLKIQAYFNETADLPCQFANSQNQSLSELVVFWQDQENLVLNEVYLGKEKFDSVHSKYMGRTSFDSDSWTLRLHNLQIKDKGLYQCIIYHKKPTGMIRIHQMNSELSVLA

SEQ ID NO: 162 - protein, 111 aa, synthetic construct; identical to SEQ ID NO: 161
  REGION 1..111: note = CD86 IgV v72

SEQ ID NO: 163 - protein, 111 aa, synthetic construct; identical to SEQ ID NO: 127
  REGION 1..111: note = CD86 IgV v73

SEQ ID NO: 164 - protein, 111 aa, synthetic construct
  REGION 1..111: note = CD86 IgV v74
APLKIQAYFNETADLPCQFANSQNQSLSELVVFWQDQENLVLNEVYLGKEKFDSVHSKYMGRTSFDSDSWTLRLHNLQIEDKGLYQCIIYHKKPTGMIRIHQMNSELSVLA

SEQ ID NO: 165 - protein, 111 aa, synthetic construct
  REGION 1..111: note = CD86 IgV v75
APLKIQAYFNETADLPCQFANSQNQSLSELVVFWQDQENLVLNEVYLGKEKFDSVHSKYMGRTSFDSDSWTLRLHNLQIMDKGLYQCTIHHKKPTGMIRIHQMNSELSVLA

SEQ ID NO: 166 - protein, 111 aa, synthetic construct
  REGION 1..111: note = CD86 IgV v76
APLKIQAYFNETADLPCQFANSQNQSLSELVVFWQDQENLVLNEVYLGKEKFDSVHSKYMGRTSFDSDSWTLRLHNLQIKDKGLYQCIIHHIKPTGMIRIHQMNSELSVLA

SEQ ID NO: 167 - protein, 111 aa, synthetic construct; identical to SEQ ID NO: 128
  REGION 1..111: note = CD86 IgV v77

SEQ ID NO: 168 - protein, 111 aa, synthetic construct; identical to SEQ ID NO: 129
  REGION 1..111: note = CD86 IgV v78

SEQ ID NO: 169 - protein, 111 aa, synthetic construct
  REGION 1..111: note = CD86 IgV v79
APLKIQAYFNETADLPCQFANSQNLSLSELVVIWQDQENLVLNEVYLGKEKFDSVHSKYMGRTSFDSDSWTLRLHNLQIKDKGLYRCIILHKTPTGMIRIHQMNSELSVLA

SEQ ID NO: 170 - protein, 111 aa, synthetic construct; identical to SEQ ID NO: 130
  REGION 1..111: note = CD86 IgV v80

SEQ ID NO: 171 - protein, 111 aa, synthetic construct; identical to SEQ ID NO: 130
  REGION 1..111: note = CD86 IgV v81

SEQ ID NO: 172 - protein, 111 aa, synthetic construct; identical to SEQ ID NO: 130
  REGION 1..111: note = CD86 IgV v82

SEQ ID NO: 173 - protein, 111 aa, synthetic construct; identical to SEQ ID NO: 130
  REGION 1..111: note = CD86 IgV v83

SEQ ID NO: 174 - protein, 111 aa, synthetic construct
  REGION 1..111: note = CD86 IgV v84
APLKIQAYFNETADLPCQFANSQNLSLSELVVFWQDQENLVLNEVYLGKEKFDSVHSKYMGRTSFDSDSWTLRLHNLQIKDKGLYQCIIYHKKPTGMIRIHQMNSELSVLA

SEQ ID NO: 175 - protein, 111 aa, synthetic construct
  REGION 1..111: note = CD86 IgV v85
APLKIQAYFNETADLPCQFANSQNLSLSELVVFWQDQENLVLNEVYLGKEKFDSVHSKYMGRTSFDSDSWTLRLHNLQIKDTGLYQCIILHKKPTGMIRIHQMNSELSVLA

SEQ ID NO: 176 - protein, 111 aa, synthetic construct; identical to SEQ ID NO: 146
  REGION 1..111: note = CD86 IgV v86

SEQ ID NO: 177 - protein, 111 aa, synthetic construct
  REGION 1..111: note = CD86 IgV v87
APLKIQAYFNETADLPCQFANSQNQSLGELVVFWQDQENLVLNEVYLGKEKFDSVHSKYMGRTSFDSDSWTLRLHNLQIKDKGLYQCIIYHKKPTGMIRIHQMNSELSVLA

SEQ ID NO: 178 - protein, 99 aa, synthetic construct
  REGION 1..99: note = CD86 IgV WT v1
NETADLPCQFANSQNLSLSELVVFWQDQENLVLNEVYLGKEKFDSVHSKYMGRTSFDSDSWALRLHNLQIKDKGLYQCIIYHKKPTGMIRIHQMNSELS

SEQ ID NO: 179 - protein, 99 aa, synthetic construct
  REGION 1..99: note = CD86 IgV WT v2
NETADLPCQFANSQNLSLSELVVFWQDQENLVLNEVYLGKEKFGSVHSKYMGRTSFDSDSWTLRLHNLQIKDKGLYQCIIHHKKPTGMIRIHQMNSELS

SEQ ID NO: 180 - protein, 99 aa, synthetic construct
  REGION 1..99: note = CD86 IgV WT v3
NETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELS

SEQ ID NO: 181 - protein, 99 aa, synthetic construct
  REGION 1..99: note = CD86 IgV WT v4
NETADLPCQFANSQNQSLSELVVFWQDQENLVLKEVYLGKEKFDSVHSKYMGRTSFDSDSWTLRLHNLQNKDKGLYQCIILHKKPTGMIRIHQMNSELS

SEQ ID NO: 182 - protein, 99 aa, synthetic construct
  REGION 1..99: note = CD86 IgV WT v5
NETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELS

SEQ ID NO: 183 - protein, 99 aa, synthetic construct
  REGION 1..99: note = CD86 IgV WT v6
NETADLPCQFANSQNLSLSELVVFWQDQENLVLNEVYLGKEKFDSVHSKYMGRTSFDSDSWTLRLHNLQIKDKGLYRCIILHKTPTGMIRIHQMNSELS

SEQ ID NO: 184 - protein, 99 aa, synthetic construct
  REGION 1..99: note = CD86 IgV WT v7
NETADLPCQFANSQNLSLSELVVIWQDQENLVLNEVYLGKEKFDSVHSKYMGRTSFDSDSWTLRLHNLQIKDKGLYQCIIYHKKPTGMIRIHQMNSELS

SEQ ID NO: 185 - protein, 99 aa, synthetic construct
  REGION 1..99: note = CD86 IgV WT v8
NETADLPCQFANSQNLSLSELVVFWQDQEDLVLNEVYLGKEKFDSVHSKYMGRTSFDSDSWTLRLHNLQIRDKGLYRCFILHKTPTGMIRIHQMNSELS

SEQ ID NO: 186 - protein, 99 aa, synthetic construct
  REGION 1..99: note = CD86 IgV WT v9
NETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELS

SEQ ID NO: 187 - protein, 99 aa, synthetic construct
  REGION 1..99: note = CD86 IgV WT v10
NETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELS

SEQ ID NO: 188 - protein, 99 aa, synthetic construct
  REGION 1..99: note = CD86 IgV WT v11
NETADLPCQFANSQNLSLSELVVFWQDQENMVLNEVYLGKEKFDSVHSKYMGRTSFDSDSWTLRLHNLQIKDKGLYQCIILHKKPTGMIRIHQMNSELS

SEQ ID NO: 189 - protein, 99 aa, synthetic construct
  REGION 1..99: note = CD86 IgV WT v12
NETADLPCKFANSQNLSLSELVVIWQDQENSVLNEVYLGKEKFDSVHSKYMGRTSFDSDSWTLRLHNLQIKDKGLYQCIILHKKPTGMIRIHQMNSELS

SEQ ID NO: 190 - protein, 99 aa, synthetic construct
  REGION 1..99: note = CD86 IgV WT v13
NETADLPCQFANSQNLSLSELVVFWQDQENLVLNEVYLGKEKFDSVHSKYMGRTSFDSDSWTLRLHNLQIKDKGLYKCIILHKKPTGMIRIHQMNSELS

SEQ ID NO: 191 - protein, 99 aa, synthetic construct
  REGION 1..99: note = CD86 IgV WT v14
NETADLPCQFANSQNLSLSELVVFWQDQENLVLNEVYLGKEKFDSVHSKYMGRTSFDSDSWTLRLHNPQIKDKGLYQCIIYHKKPTGMIRIHQMNSELS

SEQ ID NO: 192 - protein, 99 aa, synthetic construct
  REGION 1..99: note = CD86 IgV WT v15
NETADLPCQFANSQNLSLGELVVIWQDQENLVLNEVYLGKEKLDSVHSKYMGRTSFDSDSWTLRLHNLQIKDKGLYQCIILHKKPTGMIRIHHMNSELS

SEQ ID NO: 193 - protein, 99 aa, synthetic construct
  REGION 1..99: note = CD86 IgV WT v16
NETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELS

SEQ ID NO: 194 - protein, 99 aa, synthetic construct; identical to SEQ ID NO: 193
  REGION 1..99: note = CD86 IgV WT v17

SEQ ID NO: 195 - protein, 99 aa, synthetic construct
  REGION 1..99: note = CD86 IgV WT v18
NETADLPCQFANSQNLSLSELVVIWQDQENLVLNEVYLGKEKFDSVHSKYMGRTSFDSDSWTLRLHNLQIKDKGLYRCIIYHKKPTGMIRIHQMNSELS

SEQ ID NO: 196 - protein, 99 aa, synthetic construct
  REGION 1..99: note = CD86 IgV WT v19
NETADLPCQFANSQNLSLSELVVFWQDQENLVLNEIYLGKEKFDSVHSKYMGRTSFDSNSWTLRLHNLQIKDKGLYQCIILHKKPTGMIRIHQMNSELS

SEQ ID NO: 197 - protein, 99 aa, synthetic construct
  REGION 1..99: note = CD86 IgV WT v20
NETADLPCQFANSQNQSLSELVVFWQDQVNLVLNEVYLGKEKFDSVHSKYMGRTSFDSDSWTLRLHNLQIKDKGLYQCIIHHKKPTGMIRIHQMNSELS

SEQ ID NO: 198 - protein, 99 aa, synthetic construct
  REGION 1..99: note = CD86 IgV WT v21
NETADLPCQFANSQNQSLSELVVFWQDQENLVLNEVYLGKEKFDSVHSKYMGRTSFDSDSWTLRLHNLQIKDKGLYQCIIYHKKPTGMIRIHQMNSELS

SEQ ID NO: 199 - protein, 99 aa, synthetic construct; identical to SEQ ID NO: 198
  REGION 1..99: note = CD86 IgV WT v22

SEQ ID NO: 200 - protein, 99 aa, synthetic construct
  REGION 1..99: note = CD86 IgV WT v23
NETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELS

SEQ ID NO: 201 - protein, 99 aa, synthetic construct
  REGION 1..99: note = CD86 IgV WT v24
NETADLPCQFANSQNQSLSELVVFWQDQENLVLNEVYLGKEKFDSVHSKYMGRTSFDSDSWTLRLHNLQIEDKGLYQCIIYHKKPTGMIRIHQMNSELS

SEQ ID NO: 202 - protein, 99 aa, synthetic construct
  REGION 1..99: note = CD86 IgV WT v25
NETADLPCQFANSQNQSLSELVVFWQDQENLVLNEVYLGKEKFDSVHSKYMGRTSFDSDSWTLRLHNLQIMDKGLYQCTIHHKKPTGMIRIHQMNSELS

SEQ ID NO: 203 - protein, 99 aa, synthetic construct
  REGION 1..99: note = CD86 IgV WT v26
NETADLPCQFANSQNQSLSELVVFWQDQENLVLNEVYLGKEKFDSVHSKYMGRTSFDSDSWTLRLHNLQIKDKGLYQCIIHHIKPTGMIRIHQMNSELS

SEQ ID NO: 204 - protein, 99 aa, synthetic construct
  REGION 1..99: note = CD86 IgV WT v27
NETADLPCQFANSQNQSLSELVVFWQDQENLVLNEVYLGKEKFDSVHSKYKGRTSFDSDSWTLRLHNLQIKDKGLYQCIILHKKPTGMIRIHQMNSELS

SEQ ID NO: 205 - protein, 99 aa, synthetic construct; identical to SEQ ID NO: 193
  REGION 1..99: note = CD86 IgV WT v28

SEQ ID NO: 206 - protein, 99 aa, synthetic construct
  REGION 1..99: note = CD86 IgV WT v29
NETADLPCQFANSQNLSLSELVVIWQDQENLVLNEVYLGKEKFDSVHSKYMGRTSFDSDSWTLRLHNLQIKDKGLYRCIILHKTPTGMIRIHQMNSELS

SEQ ID NO: 207 - protein, 99 aa, synthetic construct; identical to SEQ ID NO: 180
  REGION 1..99: note = CD86 IgV WT v30

SEQ ID NO: 208 - protein, 99 aa, synthetic construct; identical to SEQ ID NO: 180
  REGION 1..99: note = CD86 IgV WT v31

SEQ ID NO: 209 - protein, 99 aa, synthetic construct; identical to SEQ ID NO: 180
  REGION 1..99: note = CD86 IgV WT v32

SEQ ID NO: 210 - protein, 99 aa, synthetic construct; identical to SEQ ID NO: 180
  REGION 1..99: note = CD86 IgV WT v33

SEQ ID NO: 211 - protein, 99 aa, synthetic construct
  REGION 1..99: note = CD86 IgV WT v34
NETADLPCQFANSQNLSLSELVVFWQDQENLVLNEVYLGKEKFDSVHSKYMGRTSFDSDSWTLRLHNLQIKDKGLYQCIIYHKKPTGMIRIHQMNSELS

SEQ ID NO: 212 - protein, 99 aa, synthetic construct
  REGION 1..99: note = CD86 IgV WT v35
NETADLPCQFANSQNLSLSELVVFWQDQENLVLNEVYLGKEKFDSVHSKYMGRTSFDSDSWTLRLHNLQIKDTGLYQCIILHKKPTGMIRIHQMNSELS

SEQ ID NO: 213 - protein, 99 aa, synthetic construct; identical to SEQ ID NO: 183
  REGION 1..99: note = CD86 IgV WT v36

SEQ ID NO: 214 - protein, 99 aa, synthetic construct
  REGION 1..99: note = CD86 IgV WT v37
NETADLPCQFANSQNQSLGELVVFWQDQENLVLNEVYLGKEKFDSVHSKYMGRTSFDSDSWTLRLHNLQIKDKGLYQCIIYHKKPTGMIRIHQMNSELS

SEQ ID NO: 215 - protein, 99 aa, synthetic construct; identical to SEQ ID NO: 182
  REGION 1..99: note = CD86 IgV WT v81

SEQ ID NO: 216 - protein, 99 aa, synthetic construct; identical to SEQ ID NO: 186
  REGION 1..99: note = CD86 IgV WT v82

SEQ ID NO: 217 - protein, 99 aa, synthetic construct
  REGION 1..99: note = CD86 IgV WT v83
NETADLPCQFANSQNLSLSELVVFWQDQENLVLNEVYLGKEKFDSVHSKYMGRTSFDSDSWTLRLHNLQIKDKGLYRCIILHKKPTGMIRIHQMNSELS

SEQ ID NO: 218 - protein, 99 aa, synthetic construct; identical to SEQ ID NO: 200
  REGION 1..99: note = CD86 IgV WT v84

SEQ ID NO: 219 - protein, 99 aa, synthetic construct; identical to SEQ ID NO: 204
  REGION 1..99: note = CD86 IgV WT v85

SEQ ID NO: 220 - protein, 99 aa, synthetic construct; identical to SEQ ID NO: 193
  REGION 1..99: note = CD86 IgV WT v86

SEQ ID NO: 221 - protein, 99 aa, synthetic construct; identical to SEQ ID NO: 180
  REGION 1..99: note = CD86 IgV WT v87

SEQ ID NO: 222 - protein, 7 aa, synthetic construct
  REGION 1..7: note = Linker
GSGGGGS

SEQ ID NO: 223 - protein, 5 aa, synthetic construct
  REGION 1..5: note = 4GS Linker
GGGGS

SEQ ID NO: 224 - protein, 15 aa, synthetic construct
  REGION 1..15: note = 3x G4S Linker
GGGGSGGGGSGGGGS

SEQ ID NO: 225 - protein, 10 aa, synthetic construct
  REGION 1..10: note = 2x G4S Linker
GGGGSGGGGS

SEQ ID NO: 226 - protein, 13 aa, synthetic construct
  REGION 1..13: note = Linker
GGGGSGGGGSAAA

SEQ ID NO: 227 - protein, 17 aa, synthetic construct
  REGION 1..17: note = GS(G4S)3 linker
GSGGGGSGGGGSGGGGS

SEQ ID NO: 228 - protein, 27 aa, synthetic construct
  REGION 1..27: note = GS(G4S)5 linker
GSGGGGSGGGGSGGGGSGGGGSGGGGS

SEQ ID NO: 229 - protein, 232 aa, synthetic construct
  REGION 1..232: note = IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 231 aa, synthetic construct
  REGION 1..231: note = Stack Fc (homodimeric)
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 231 - protein, 116 aa, synthetic construct
  REGION 1..116: note = NKp30 variant
LWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQL

SEQ ID NO: 232 - protein, 116 aa, synthetic construct
  REGION 1..116: note = NKp30 variant v2
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQL

SEQ ID NO: 233 - protein, 306 aa, synthetic construct; identical to SEQ ID NO: 56
  REGION 1..306: note = CD86 WT ECD TIP

SEQ ID NO: 234 - protein, 306 aa, synthetic construct
  REGION 1..306: note = CD86 ECD TIP v1
APLKIQAYFNETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLPVSFPDVTSNMTIFCIMETDKTRLLTSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 235 - protein, 306 aa, synthetic construct
  REGION 1..306: note = CD86 ECD TIP v2
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLANFSQPEIVPISNITENVYINLACSSIHGYPEPKKMSVLLRTKNSTIEYDGVMQKSQDNVTELYDVSISLPVSFPDVTSNMTIFCILETDKTRLLSSPFSIELEVPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 236 - protein, 306 aa, synthetic construct
  REGION 1..306: note = CD86 ECD TIP v3
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 237 - protein, 306 aa, synthetic construct
  REGION 1..306: note = CD86 ECD TIP v4
APLKIQAYFNETADLPCQFANSQNQSLSELVVFWQDQENLVLNEVYLGKEKFDSVHSKYMGRTSFDSDSWTLRLHNLQIKDKGLYQCIVLHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTVFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 238 - protein, 306 aa, synthetic construct
  REGION 1..306: note = CD86 ECD TIP v5
APLKIQAYFNETADLPCQFANSQNQSLSELVVFWQDQENLVLNEVYLGKEKFDSVHSKYKGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 239 - protein, 306 aa, synthetic construct
  REGION 1..306: note = CD86 ECD TIP v6
APLKIQAYFNETADLPCQFANSQNLSLSELVVIWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 240 - protein, 306 aa, synthetic construct
  REGION 1..306: note = CD86 ECD TIP v7
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLSVSFSDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 241 - protein, 306 aa, synthetic construct
  REGION 1..306: note = CD86 ECD TIP v8
APLKIQAYFNETADLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSIRLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 242 - protein, 400 aa, synthetic construct
  REGION 1..400: note = PAS
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPA

SEQ ID NO: 243 - protein, 22 aa, synthetic construct
  REGION 1..22: note = P2A protein
GSGATNFSLLKQAGDVEENPGP

SEQ ID NO: 244 - protein, 237 aa, synthetic construct
  REGION 1..237: note = eGFP
SKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 245 - protein, 237 aa, synthetic construct
  REGION 1..237: note = Green fluorescent protein
SKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 246 - protein, 239 aa, synthetic construct
  REGION 1..239: note = eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 247 - protein, 25 aa, synthetic construct
  REGION 1..25: note = (EAAAK)5 linker
EAAAKEAAAKEAAAKEAAAKEAAAK

SEQ ID NO: 248 - protein, 8 aa, synthetic construct
  REGION 1..8: note = FLAG-tag
DYKDDDDK

SEQ ID NO: 249 - protein, 6 aa, synthetic construct
  REGION 1..6: note = 6xHis tag
HHHHHH

SEQ ID NO: 250 - protein, 228 aa, synthetic construct
  REGION 1..228: note = rabbit Fc
APSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 251 - protein, 238 aa, synthetic construct
  REGION 1..238: note = mouse Fc2b
EPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 252 - protein, 232 aa, synthetic construct
  REGION 1..232: note = Knob Fc
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 231 aa, synthetic construct
  REGION 1..231: note = wild-type human IgG1 Fc - delK
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 232 aa, synthetic construct
  REGION 1..232: note = IgG1 Fc C220S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 232 aa, synthetic construct
  REGION 1..232: note = Fc
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 232 aa, synthetic construct
  REGION 1..232: note = Fc1.1
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 231 aa, synthetic construct
  REGION 1..231: note = Fc
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 232 aa, synthetic construct
  REGION 1..232: note = Stack Fc (homodimeric)
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 231 aa, synthetic construct
  REGION 1..231: note = SEFL
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 260 - protein, 231 aa, synthetic construct
  REGION 1..231: note = Fc1.1
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 230 aa, synthetic construct
  REGION 1..230: note = Fc
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 262 - protein, 235 aa, synthetic construct
  REGION 1..235: note = IgG2 Fc
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 263 - protein, 229 aa, synthetic construct
  REGION 1..229: note = IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 264 - protein, 229 aa, synthetic construct
  REGION 1..229: note = IgG4 Fc S228P
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 265 - protein, 5 aa, synthetic construct
  REGION 1..5: note = EAAAK linker
EAAAK

SEQ ID NO: 266 - protein, 15 aa, synthetic construct
  REGION 1..15: note = (EAAAK)3 linker
EAAAKEAAAKEAAAK

SEQ ID NO: 267 - protein, 225 aa, synthetic construct
  REGION 1..225: note = Fc1.1 delta EPKSSD
KTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 108 aa, synthetic construct
  REGION 1..108: note = NKp30 v1 IgC-like
LWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLV

SEQ ID NO: 269 - protein, 108 aa, synthetic construct
  REGION 1..108: note = NKp30 v2 IgC-like
LWVSQPPEIRTLEGSSAFLPCSFNASQGRVAIGSVTWFRDEVVPGKEVRNGTPEFRGRLAPLASSRFLHDHQAELHIRDVRGHDASIYVCRVEVLGLGVGTGNGTRLV

SEQ ID NO: 270 - protein, 108 aa, synthetic construct
  REGION 1..108: note = NKp30 v3 IgC-like
LWVSQPPEIRTLEGSSAFLPCSFNASQGRLAIGSVTWFRDEVVPGKEVRNGTPEFRGRLVPLASSRFLHDHQAELHIRDVRGHDASIYVCRVEVLGLGVGTGNGTRLV

SEQ ID NO: 271 - protein, 108 aa, synthetic construct
  REGION 1..108: note = NKp30 v4 IgC-like
LWVSQPPEIRTLEGSSAFLPCSFNASQGRLAIGSVTWFRDEVVPGKEVRNGTPEFRGRLAPLAPSRFLHDHQAELHIRDVRGHDASIYVCRVEVLGLGVGTGNGTRLV

SEQ ID NO: 272 - protein, 108 aa, synthetic construct
  REGION 1..108: note = NKp30 v5 IgC-like
LWVSQPPEIRTLEGSSAFLPCSFNASQGRLAIGSVTWFRDEVVPGKEVRNGTPEFRGRLAPLASSRFLHDHQAELHIRDVRGHDAGIYVCRVEVLGLGVGTGNGTRLV

SEQ ID NO: 273 - protein, 117 aa, synthetic construct
  REGION 1..117: note = NKp30 v1 ECD
LWVSQPPEIRTLEGSSAFLPCSFNASQGRVAIGSVTWFRDEVVPGKEVRNGTPEFRGRLVPLAPSRFLHDHQAELHIRDVRGHDAGIYVCRVEVLGLGVGTGNGTRLVVEKEHPQLG

SEQ ID NO: 274 - protein, 117 aa, synthetic construct
  REGION 1..117: note = NKp30 v2 ECD
LWVSQPPEIRTLEGSSAFLPCSFNASQGRVAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 275 - protein, 117 aa, synthetic construct
  REGION 1..117: note = NKp30 v3 ECD
LWVSQPPEIRTLEGSSAFLPCSFNASQGRLAIGSVTWFRDEVVPGKEVRNGTPEFRGRLVPLASSRFLHDHQAELHIRDVRGHDASIYVCRVEVLGLGVGTGNGTRLVVEKEHPQLG

SEQ ID NO: 276 - protein, 117 aa, synthetic construct
  REGION 1..117: note = NKp30 v4 ECD
LWVSQPPEIRTLEGSSAFLPCSFNASQGRLAIGSVTWFRDEVVPGKEVRNGTPEFRGRLAPLAPSRFLHDHQAELHIRDVRGHDASIYVCRVEVLGLGVGTGNGTRLVVEKEHPQLG

SEQ ID NO: 277 - protein, 117 aa, synthetic construct
  REGION 1..117: note = NKp30 v5 ECD
LWVSQPPEIRTLEGSSAFLPCSFNASQGRLAIGSVTWFRDEVVPGKEVRNGTPEFRGRLAPLASSRFLHDHQAELHIRDVRGHDAGIYVCRVEVLGLGVGTGNGTRLVVEKEHPQLG

SEQ ID NO: 278 - protein, 108 aa, synthetic construct
  REGION 1..108: note = NKp30 WT IgC-like
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLV

SEQ ID NO: 279 - protein, 8 aa, synthetic construct
  REGION 1..8: note = Stuffer
HMSSVSAQ

SEQ ID NO: 280 - protein, 232 aa, synthetic construct
  REGION 1..232: note = Hole Fc
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 281 - protein, 71 aa, synthetic construct
  REGION 1..71: note = CD8-derived hinge and transmembrane domain
KPTTTPAPRPPTPAPTIASQPLSLRPEASRPAAGGAVHTRGLDFASDIYIWAPLAGTCGVLLLSLVITLYC

SEQ ID NO: 282 - protein, 69 aa, synthetic construct
  REGION 1..69: note = CD8-derived hinge and transmembrane domain (2nd gen CAR)
AKPTTTPAPRPPTPAPTIASQPLSLRPEACRPAAGGAVHTRGLDFACDIYIWAPLAGTCGVLLLSLVIT

SEQ ID NO: 283 - protein, 68 aa, synthetic construct
  REGION 1..68: note = CD8 hinge and transmembrane domain
KPTTTPAPRPPTPAPTIASQPLSLRPEACRPAAGGAVHTRGLDFACDIYIWAPLAGTCGVLLLSLVIT

SEQ ID NO: 284 - protein, 112 aa, synthetic construct
  REGION 1..112: note = CD3zeta intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 285 - protein, 42 aa, synthetic construct
  REGION 1..42: note = 4-1BB-derived costimulatory domain
KRGRKKLLYIFKQPFMRPVQTTQEEDGCSCRFPEEEEGGCEL

SEQ ID NO: 286 - protein, 40 aa, synthetic construct
  REGION 1..40: note = CD28-derived costimulatory domain
SKRSRLLHSDYMNMTPRRPGPTRKHYQPYAPPRDFAAYRS

SEQ ID NO: 287 - protein, 41 aa, synthetic construct
  REGION 1..41: note = CD28-derived costimulatory domain 2
RSKRSRLLHSDYMNMTPRRPGPTRKHYQPYAPPRDFAAYRS

SEQ ID NO: 288 - protein, 44 aa, synthetic construct
  REGION 1..44: note = CD28-derived costimulatory domain 3
FWVRSKRSRLLHSDYMNMTPRRPGPTRKHYQPYAPPRDFAAYRS

SEQ ID NO: 289 - protein, 16 aa, synthetic construct
  REGION 1..16: note = CD33 Signal Peptide
MPLLLLLPLLWAGALA

SEQ ID NO: 290 - protein, 22 aa, synthetic construct
  REGION 1..22: note = IgGkappa light chain
MDMRVLAQLLGLLLLCFPGARC

SEQ ID NO: 291 - protein, 18 aa, synthetic construct
  REGION 1..18: note = HSA signal peptide
MKWVTFISLLFLFSSAYS

SEQ ID NO: 292 - protein, 22 aa, synthetic construct
  REGION 1..22: note = Ig kappa light chain
MDMRAPAGIFGFLLVLFPGYRS

SEQ ID NO: 293 - protein, 19 aa, synthetic construct
  REGION 1..19: note = human azurocidin preprotein signal sequence
MTRLTVLALLAGLLASSRA

SEQ ID NO: 294 - protein, 19 aa, synthetic construct
  REGION 1..19: note = IgG heavy chain signal peptide
MELGLSWIFLLAILKGVQC

SEQ ID NO: 295 - protein, 19 aa, synthetic construct
  REGION 1..19: note = IgG heavy chain signal peptide
MELGLRWVFLVAILEGVQC

SEQ ID NO: 296 - protein, 19 aa, synthetic construct
  REGION 1..19: note = IgG heavy chain signal peptide
MKHLWFFLLLVAAPRWVLS

SEQ ID NO: 297 - protein, 19 aa, synthetic construct
  REGION 1..19: note = IgG heavy chain signal peptide
MDWTWRILFLVAAATGAHS

SEQ ID NO: 298 - protein, 19 aa, synthetic construct
  REGION 1..19: note = IgG heavy chain signal peptide
MDWTWRFLFVVAAATGVQS

SEQ ID NO: 299 - protein, 19 aa, synthetic construct
  REGION 1..19: note = IgG heavy chain signal peptide
MEFGLSWLFLVAILKGVQC

SEQ ID NO: 300 - protein, 19 aa, synthetic construct
  REGION 1..19: note = IgG heavy chain signal peptide
MEFGLSWVFLVALFRGVQC

SEQ ID NO: 301 - protein, 26 aa, synthetic construct
  REGION 1..26: note = IgG heavy chain signal peptide
MDLLHKNMKHLWFFLLLVAAPRWVLS

SEQ ID NO: 302 - protein, 22 aa, synthetic construct
  REGION 1..22: note = IgG Kappa light chain signal sequences
MDMRVPAQLLGLLLLWLSGARC

SEQ ID NO: 303 - protein, 22 aa, synthetic construct
  REGION 1..22: note = IgG Kappa light chain signal sequences
MKYLLPTAAAGLLLLAAQPAMA

SEQ ID NO: 304 - protein, 17 aa, synthetic construct
  REGION 1..17: note = Gaussia luciferase
MGVKVLFALICIAVAEA

SEQ ID NO: 305 - protein, 18 aa, synthetic construct
  REGION 1..18: note = Human albumin
MKWVTFISLLFLFSSAYS

SEQ ID NO: 306 - protein, 18 aa, synthetic construct
  REGION 1..18: note = Human chymotrypsinogen
MAFLWLLSCWALLGTTFG

SEQ ID NO: 307 - protein, 14 aa, synthetic construct
  REGION 1..14: note = Human interleukin-2
MQLLSCIALILALV

SEQ ID NO: 308 - protein, 15 aa, synthetic construct
  REGION 1..15: note = Human trypsinogen-2
MNLLLILTFVAAAVA

SEQ ID NO: 309 - protein, 20 aa, synthetic construct
  REGION 1..20: note = T2A protein
SGEGRGSLLTCGDVEENPGP

SEQ ID NO: 310 - protein, 18 aa, synthetic construct
  REGION 1..18: note = T2A protein
EGRGSLLTCGDVEENPGP

SEQ ID NO: 311 - protein, 21 aa, synthetic construct
  REGION 1..21: note = T2A protein
GSGEGRGSLLTCGDVEENPGP

SEQ ID NO: 312 - protein, 232 aa, synthetic construct
  REGION 1..232: note = Blue fluorescent protein
SRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLWEAFTETLYPADGGLEGRNDMALKLVGGSHLIANIKTTYRSKKPAKNLKMPGVYYVDYRLERIKEANNETYVEQHEVAARYCDLPSKLGHKLN

SEQ ID NO: 313 - protein, 232 aa, synthetic construct
  REGION 1..232: note = Blue fluorescent protein
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 314 - protein, 224 aa, synthetic construct
  REGION 1..224: note = CD86 ECD v38
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 315 - protein, 122 aa, synthetic construct
  REGION 1..122: note = PD-1 v179 (6-127)
DSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 316 - protein, 361 aa, synthetic construct
  REGION 1..361: note = CD86 WT ECD/PD1v179 ECD (6-127) Stack v1
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 317 - protein, 361 aa, synthetic construct
  REGION 1..361: note = CD86 ECD v1 /PD1v179 ECD (6-127) Stack v2
APLKIQAYFNETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLANFSQPEIVPISNITENVYINLTCSSIHGYPEPKKMSVLLRTKNSTIEYDGVMQKSQDNVTELYDVSISLPVSFPDVTSNMTIFCIMETDKTRLLTSPFSIELEDPQPPPDHIPGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 318 - protein, 361 aa, synthetic construct
  REGION 1..361: note = CD86 ECD v2 /PD1v179 ECD (6-127) Stack v3
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLANFSQPEIVPISNITENVYINLACSSIHGYPEPKKMSVLLRTKNSTIEYDGVMQKSQDNVTELYDVSISLPVSFPDVTSNMTIFCILETDKTRLLSSPFSIELEVPQPPPDHIPGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 319 - protein, 361 aa, synthetic construct
  REGION 1..361: note = CD86 ECD v3 /PD1v179 ECD (6-127) Stack v4
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 320 - protein, 248 aa, synthetic construct
  REGION 1..248: note = CD86 IgV v4 /PD1v179 ECD (6-127) Stack v5
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 321 - protein, 248 aa, synthetic construct
  REGION 1..248: note = CD86 IgV v5 /PD1v179 ECD (6-127) Stack v6
APLKIQAYFNETVDLPCQFANSQNLSLSELVVFWQDQENLVLNEVYLGKEKFDSVHSKYMGRTSFDSDSWTLRLHNLQIKDKGLYQCIILHKKPTGMIRIHQMNSELSVLAGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 322 - protein, 248 aa, synthetic construct
  REGION 1..248: note = CD86 IgV v6 /PD1v179 ECD (6-127) Stack v7
APLKIQAYFNETADLPCQFANSQNLSLSELVVFWQDQENLVLNEVYLGKEKFDSVHSKYMGRTSFDSDSWTLRLHNLQIKDKGLYQCIILHKTPTGLIRIHQMNSELSVLAGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 323 - protein, 248 aa, synthetic construct
  REGION 1..248: note = CD86 IgV v7 /PD1v179 ECD (6-127) Stack v8
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGGGGSGGGGSGGGGSDSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTER

SEQ ID NO: 324 - protein, 231 aa, synthetic construct
  REGION 1..231: note = Fc knob
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 231 aa, synthetic construct
  REGION 1..231: note = Fc hole
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 326 - protein, 604 aa, synthetic construct
  REGION 1..604: note = PD1 v1 Fc CD86 ECD v10
DSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTERGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 327 - protein, 491 aa, synthetic construct
  REGION 1..491: note = PD1 v1 Fc CD86 IgV v60
DSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTERGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 328 - protein, 360 aa, synthetic construct
  REGION 1..360: note = PD1 ECD v1 FcK
DSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTERGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 329 - protein, 349 aa, synthetic construct
  REGION 1..349: note = CD86 IgV v60 FcH
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 330 - protein, 362 aa, synthetic construct
  REGION 1..362: note = FcH CD86 Igv v60
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 331 - protein, 491 aa, synthetic construct
  REGION 1..491: note = PD1 ECD v1 FcH CD86 IgV v60
DSPDRPWNPPTFSPALLVVTEGDNATFTCSFSNTSESFVLNWYRMSPSNQTDKLAAFPEDRNQPGQDRRFRVTQLPNGRDYHMSVDRARRNDSGTYLCGAITLAPKVQIDESLRVELRVTERGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 332 - protein, 231 aa, synthetic construct
  REGION 1..231: note = Fc
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 333 - protein, 231 aa, synthetic construct; identical to SEQ ID NO: 260
  REGION 1..231: note = Fc

SEQ ID NO: 334 - protein, 115 aa, synthetic construct
  REGION 1..115: note = PD-1 v94 ECD (13-127)
NPPTFSPALLVVTEGDNATFTCSFSNTSESFVLNWYRMSPSNQTDKVAAFPEDHSQPGQDCRFRVTQLPNGRDYHMSLVRARRNDSGTYLCGAISLAPKVQINESLLVELRVTER

SEQ ID NO: 335 - protein, 111 aa, synthetic construct
  REGION 1..111: note = PD-1 wt IgV
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVT

SEQ ID NO: 336 - protein, 122 aa, synthetic construct
  REGION 1..122: note = PD-1 wt ECD 6-127
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

SEQ ID NO: 337 - protein, 115 aa, synthetic construct
  REGION 1..115: note = PD-1 wt ECD (13-127)
NPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

SEQ ID NO: 338 - protein, 122 aa, synthetic construct
  REGION 1..122: note = PD-1 v201 ECD (6-127)
DSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTER

SEQ ID NO: 339 - protein, 111 aa, synthetic construct; identical to SEQ ID NO: 335
  REGION 1..111: note = PD-1 wt IgV

SEQ ID NO: 340 - protein, 449 aa, synthetic construct
  REGION 1..449: note = Pertuz HC
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 214 aa, synthetic construct
  REGION 1..214: note = Pertuz LC
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 575 aa, synthetic construct
  REGION 1..575: note = CD86 H3701 IgV HC
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 340 aa, synthetic construct
  REGION 1..340: note = CD86 H3701 IgV LC
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 579 aa, synthetic construct
  REGION 1..579: note = HC CD86 H3701 IgV
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 345 - protein, 345 aa, synthetic construct
  REGION 1..345: note = LC CD86 H3701 IgV
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 346 - protein, 445 aa, synthetic construct
  REGION 1..445: note = Panit HC
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 347 - protein, 214 aa, synthetic construct
  REGION 1..214: note = Panit LC
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 571 aa, synthetic construct
  REGION 1..571: note = CD86 H3701 IgV HC
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 349 - protein, 340 aa, synthetic construct
  REGION 1..340: note = CD86 H3701 IgV LC
APLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLAGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 575 aa, synthetic construct
  REGION 1..575: note = HC CD86 H3701 IgV
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 351 - protein, 345 aa, synthetic construct
  REGION 1..345: note = LC CD86 H3701 IgV
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAPLKIQAYFNETADLPCQFANSQNLSLSELVVFWQDQENLVLNEVYLGKEKFDSVHSKYMGRTSFDSDSWTLRLHNLQIKDKGLYRCIILHKKPTGMIRIHQMSSELSVLA

SEQ ID NO: 352 - protein, 446 aa, synthetic construct
  REGION 1..446: note = Ramuc HC
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 214 aa, synthetic construct
  REGION 1..214: note = Ramuc LC
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0193313 A1
(43) Pub. Date: Jul. 9, 2026

(54) CD86 Variant Immunomodulatory Proteins and Uses Thereof

(71) Applicant: Alpine Immune Sciences, Inc., Seattle, WA (US)

(72) Inventors: Ryan Swanson, Seattle, WA (US); Dan Ardourel, Seattle, WA (US); Joseph L. Kuijper, Seattle, WA (US); Steven Dennis Levin, Seattle, WA (US)

(73) Assignee: Alpine Immune Sciences, Inc., Seattle, WA (US)

(21) Appl. No.: 19/402,705

(22) Filed: Nov. 26, 2025

Related U.S. Application Data
(63) Continuation of application No. 17/298,506, filed on Nov. 27, 2019, now Pat. No. 12,552,853.
(60) Provisional application No. 62/774,131, filed on Nov. 30, 2018.
(60) Provisional application No. 62/862,001, filed on Jun. 14, 2019.

(51) Int. Cl. C07K 14/705; A61K 39/395; C07K 14/47; C12N 15/10

(52) U.S. Cl. CPC C07K 14/70532; A61K 39/395; C07K 14/47; C12N 15/102; C07K 2317/24; C07K 2317/52

(57) ABSTRACT

Provided herein are variant CD86 polypeptides, immunomodulatory proteins comprising variant CD86 polypeptides, and nucleic acids encoding such proteins. The immunomodulatory proteins provide therapeutic utility for a variety of immunological and oncological conditions. Compositions and methods for making and using such proteins are provided.

### CROSS-REFERENCE TO RELATED APPLICATIONS

This application is a continuation of U.S. application Ser. No. 17/298,506, filed May 28, 2021, which is a National Stage application under 35 U.S.C. § 371 of International Application No. PCT/US2019/063808, filed on Nov. 27, 2019, which claims priority from U.S. provisional application No. 62/774,131 filed Nov. 30, 2018, entitled “CD86 VARIANT IMMUNOMODULATORY PROTEINS AND USES THEREOF” and U.S. provisional application No. 62/862,001 filed Jun. 14, 2019, entitled “CD86 VARIANT IMMUNOMODULATORY PROTEINS AND USES THEREOF,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 01245-0075-00US.xml, created Jul. 11, 2025, which is 508,355 bytes in size. The information in the electronic format of the Sequence Listing is incorporated by reference in its entirety.

### FIELD

The present disclosure relates to therapeutic compositions for modulating immune response in the treatment of cancer and immunological diseases. In some aspects, the present disclosure relates to particular variants of CD86, and immunomodulatory proteins thereof, that exhibit altered binding affinity for a cognate binding partner, such as increased affinity for CD28. Also provided are methods and uses of such immunomodulatory proteins.

### BACKGROUND

Modulation of the immune response by intervening in the processes that occur in the immunological synapse (IS) formed by and between antigen-presenting cells (APCs) or target cells and lymphocytes is of increasing medical interest. Mechanistically, cell surface proteins in the IS can involve the coordinated and often simultaneous interaction of multiple protein targets with a single protein to which they bind. IS interactions occur in close association with the junction of two cells, and a single protein in this structure can interact with both a protein on the same cell (cis) as well as a protein on the associated cell (trans), likely at the same time. Although therapeutics are known that can modulate the IS, improved therapeutics are needed. Provided are immunomodulatory proteins, including soluble proteins or transmembrane immunomodulatory proteins capable of being expressed on cells, that meet such needs.

### SUMMARY

Provided herein are variant CD86 polypeptides, containing an extracellular domain or an IgV domain or specific binding fragment thereof, wherein the variant CD86 polypeptide contains one or more amino acid modifications in an unmodified CD86 polypeptide or a specific binding fragment thereof corresponding to position(s) selected from among 13, 18, 25, 28, 33, 38, 39, 40, 43, 45, 52, 53, 60, 68, 71, 77, 79, 80, 82, 86, 88, 89, 90, 92, 93, 97, 102, 104, 113, 114, 123, 128, 129, 132, 133, 137, 141, 143, 144, 148, 153, 154, 158, 170, 172, 175, 178, 180, 181, 183, 185, 192, 193, 196, 197, 198, 205, 206, 207, 212, 215, 216, 222, 223, or 224, with reference to positions set forth in SEQ ID NO:29. In some embodiments, the amino acid modifications contain amino acid substitutions, deletions or insertions. In some embodiments, the unmodified CD86 polypeptide is a mammalian CD86 polypeptide or a specific binding fragment thereof. In some embodiments, the unmodified CD86 polypeptide is a human CD86 polypeptide or a specific binding fragment thereof. In some embodiments, the variant CD86 polypeptide contains the extracellular domain of a human CD86, wherein the one or more amino acid modifications are in one or more residues of the extracellular domain of the unmodified CD86 polypeptide. In some embodiments, the unmodified CD86 polypeptide contains (i) the sequence of amino acids set forth in SEQ ID NO:29, (ii) a sequence of amino acids that has at least 95% sequence identity to SEQ ID NO:29; or (iii) a portion thereof containing an IgV domain or specific binding fragment of the IgV domain. In some embodiments, the unmodified CD86 contains the sequence of amino acids set forth in SEQ ID NO:29. In some embodiments, the portion thereof comprises amino acid residues 33-131 or 24-134 of the IgV domain or specific binding fragment of the IgV domain.

In some embodiments, the unmodified CD86 polypeptide contains (i) the sequence of amino acids set forth in SEQ ID NO: 123, (ii) a sequence of amino acids that has at least 95% sequence identity to SEQ ID NO: 123; or (iii) a portion thereof containing an IgV domain or specific binding fragment of the IgV domain. In some embodiments, the unmodified CD86 contains the sequence of amino acids set forth in SEQ ID NO:123.

In some embodiments, the unmodified CD86 polypeptide contains (i) the sequence of amino acids set forth in SEQ ID NO:122, (ii) a sequence of amino acids that has at least 95% sequence identity to SEQ ID NO:122; or (iii) or a specific binding fragment thereof. In some embodiments, the unmodified CD86 contains the sequence of amino acids set forth in SEQ ID NO:122.

In some embodiments, the specific binding fragment has a length of at least 50, 60, 70, 80, 90, 95 or more amino acids. In some embodiments, the specific binding fragment comprises a length that is at least 80% of the length of the IgV domain set forth as residues 33-131 of SEQ ID NO:2. In some embodiments, the variant CD86 comprises up to 1, 2, 3, 4, 5, 6, 7, 8, 9, 10, 11, 12, 13, 14, 15, 16, 17, 18, 19 or 20 amino acid modifications, optionally amino acid substitutions, insertions, and/or deletions. In some embodiments, the one or more amino acid modifications are substitutions. In some embodiments, the one or more amino acid modifications are insertions. In some embodiments, the one or more amino acid modifications are deletions. In some embodiments, the one or more amino acid modification are one or more amino acid substitutions selected from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative amino acid substitution thereof.

In some embodiments, the variant CD86 polypeptide contains one or more amino acid modifications selected from among Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93T/L132M/V148D/S181P/P216H, Q25L/F33I/H90Y/V128A/P141A/E158G/S181P, Q25L/N39D/K80R/Q86R/I88F/H90L/K93T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V45I/D68N/H90L/S183P/L205S, E38V/S114G/P143H, H90Y/L180S, H90Y/Y129N, I89V/H90L/I193V, K80E/H90Y/H222T/I223F/P224L, K80M/I88T, K92I/F113S, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, or S28G/H90Y. In some embodiments, the one or more amino acid modifications are at position 25 and/or position 90. In some embodiments, the one or more amino acid modifications contain Q25L, H90Y, or H90L. In some embodiments, the one or more amino acid modifications contain Q25L. In some embodiments, the one or more amino acid modification contains H90Y. In some embodiments, the one or more amino acid modifications contain H90L. In some embodiments, the one or more amino acid modifications contain modifications at position 25 and position 90. In some embodiments, the one or more amino acid modifications are selected from Q25L/H90Y or Q25L/H90L. In some embodiments, the one or more amino acid modifications contain Q25L/H90Y or Q25L/H90L and additional amino acid modifications. In some embodiments, the one or more amino acid modifications contain Q25L/H90Y or Q25L/H90L and one or more amino acid modifications selected from A13V, Q18K, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, K92I, K93T, M97L, Q102H, N104S, F113S, S114G, N123D, V128A, Y129N, L132M, T133A, 1137T, P141A, P143H, K144E, V148D, K153E, K153R, N154D, E158G, V170D, E172G, D175E, 1178T, L180S, S181P, S183P, P185S, T192N, 1193V, I196V, L197M, E198D, L205S, S206T, S207P, E212V, D215V, P216H, H222T or 1223F, or a conservative amino acid substitution thereof.

In some embodiments, the variant CD86 polypeptide contains one or more amino acid modifications selected from among Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93T/L132M/V148D/S181P/P216H, Q25L/F33I/H90Y/V128A/P141A/E158G/S181P, Q25L/N39D/K80R/Q86R/I88F/H90L/K93T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V451/D68N/H90L/S183P/L205S, H90Y/L180S, H90Y/Y129N, I89V/H90L/I193V, K80E/H90Y/H222T/I223F/P224L, M60K/H90L; Q25L/F33I/H90L; Q25L/F33I/Q86R/H90L/K93T; Q25L/H90L; Q25L/H90L/P185S; Q25L/H90L/P185S/P224L; Q25L/H90L/S179R; Q25L/H90Y/S181P/I193V; Q25L/K82T/H90L/T152S/S207P; Q25L/Q86R/H90L/K93T, S28G/H90Y, A13V/Q25L/H90L, Q25L/H90L/K93T/M97L, Q25L/Q86R/H90L or I89V/H90L.

In some embodiments, the variant CD86 polypeptide contains one or more amino acid modifications A13V/Q25L/H90L. In some embodiments, the variant CD86 polypeptide contains one or more amino acid modifications A13V/Q25L/H90L/S181P/L197M/S206T. In some embodiments, the variant CD86 polypeptide contains one or more amino acid modifications Q25L/H90L/K93T/M97L. In some embodiments, the variant CD86 polypeptide contains one or more amino acid modifications Q25L/H90L/K93T/M97L/T133A/S181P/D215V. In some embodiments, the variant CD86 polypeptide contains one or more amino acid modifications Q25L/Q86R/H90L. In some embodiments, the variant CD86 polypeptide contains one or more amino acid modifications Q25L/Q86R/H90L/N104S. In some embodiments, the variant CD86 polypeptide contains one or more amino acid modifications I89V/H90L. In some embodiments, the variant CD86 polypeptide contains one or more amino acid modifications I89V/H90L/I193V. In some embodiments, the variant CD86 polypeptide contains one or more amino acid modifications M60K/H90L. In some embodiments, the variant CD86 polypeptide contains one or more amino acid modifications Q25L/F33I/H90L. In some embodiments, the variant CD86 polypeptide contains one or more amino acid modifications Q25L/H90L/P185S.

In some embodiments, the variant CD86 polypeptide comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 29 or a specific binding fragment thereof.

In some embodiments, the variant CD86 polypeptide specifically binds to the ectodomain of CD28 with increased affinity compared to the binding of the unmodified CD86 for the same ectodomain. In some embodiments, the binding affinity is increased at least at or about 1.5-fold, at least at or about 2.0-fold, at least at or about 5.0-fold, at least at or about 10-fold, at least at or about 20-fold, at least at or about 30-fold, at least at or about 40-fold, at least at or about 50-fold, at least at or about 60-fold, at least at or about 70-fold, at least at or about 80-fold, at least at or about 90-fold, at least at or about 100-fold, or at least at or about 125-fold.

In some embodiments, the variant CD86 polypeptide specifically binds to the ectodomain of CTLA-4 with decreased affinity compared to the binding of the unmodified CD86 for the same ectodomain. In some embodiments, the decreased binding affinity is decreased at least at or about 1.2-fold, at least at or about 1.4-fold, at least at or about 1.5-fold, at least at or about 1.75-fold, at least at or about 2.0-fold, at least at or about 2.5-fold, at least at or about 3.0-fold, at least at or about 4.0-fold, or at least at or about 5.0-fold. In some embodiments, the variant CD86 polypeptide specifically binds to the ectodomain of CTLA-4 with the same or similar binding affinity as the binding of the unmodified CD86 for the same ectodomain, optionally wherein the same or similar binding affinity is from at or about 90% to 120% of the binding affinity of the unmodified CD86.

In some embodiments, the variant CD86 polypeptide contains the full extracellular domain. In some embodiments, the variant CD86 polypeptide contains the sequence of amino acids set forth in any of SEQ ID NOS: 85-121 or a specific binding fragment thereof, a sequence of amino acids that exhibits at least 95% sequence identity to any of SEQ ID NOS: 85-121 or a specific binding fragment thereof and that contains the one or more of the amino acid modifications of the respective SEQ ID NO set forth in any of SEQ ID NOS: 85-121. In some embodiments, the variant CD86 polypeptide contains the sequence of amino acids set forth in any of SEQ ID NOS: 141-177 or a specific binding fragment thereof, a sequence of amino acids that exhibits at least 95% sequence identity to any of SEQ ID NOS: 141-177 or a specific binding fragment thereof and that contains the one or more of the amino acid modifications of the respective SEQ ID NO set forth in any of SEQ ID NOS: 141-177.

In some embodiments, the CD28 is a human CD28. In some embodiments, the CTLA-4 is a human CTLA-4. In some embodiments, the variant CD86 polypeptide of is a soluble protein.

In some embodiments, the variant CD86 polypeptide lacks the CD86 transmembrane domain and intracellular signaling domain; and/or the variant CD86 polypeptide is not capable of being expressed on the surface of a cell. In some embodiments, the variant CD86 polypeptide is linked to a multimerization domain. In some embodiments, the multimerization domain is an Fc domain or a variant thereof with reduced effector function. In some embodiments, the variant CD86 polypeptide is linked to an Fc domain or a variant thereof with reduced effector function. In some embodiments, the Fc domain is a human IgG1 or is a variant thereof with reduced effector function. In some embodiments, the Fc domain contains the sequence of amino acids set forth in SEQ ID NO: 229 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 229. In some embodiments, the Fc domain is or contains the sequence of amino acids set forth in SEQ ID NO: 229.

In some embodiments, the Fc domain is a variant IgG1 Fc domain containing one or more amino acid modifications selected from among E233P, L234A, L234V, L235A, L235E, G236del, G237A, S267K, N297G, V302C and K447del, each by EU numbering. In some embodiments, the Fc domain contains the amino acid modifications L234A/L235E/G237A. In some embodiments, the Fc domain contains the amino acid modification C220S by EU numbering. In some embodiments, the Fc domain contains the amino acid modification K447del by EU numbering. In some embodiments, the Fc domain contains the sequence of amino acids set forth in SEQ ID NO: 230 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 230 and contains one or more of the respective amino acid modifications set forth in SEQ ID NO: 230 compared to human IgG1. In some embodiments, the Fc domain is or contains the sequence of amino acids set forth in SEQ ID NO: 230.

In some embodiments, the variant CD86 polypeptide is linked to the multimerization domain or Fc indirectly via a linker, optionally a G4S linker. In some embodiments, the variant CD86 polypeptide is a transmembrane immunomodulatory protein further containing a transmembrane domain, optionally wherein the transmembrane domain is linked, directly or indirectly, to the extracellular domain (ECD) or specific binding fragment thereof of the variant CD86 polypeptide. In some embodiments, the transmembrane domain contains the sequence of amino acids set forth as residues 248-268 of SEQ ID NO: 2 or a functional variant thereof that exhibits at least 85% sequence identity to residues 248-268 of SEQ ID NO: 2. In some embodiments, the variant CD86 polypeptide further contains a cytoplasmic domain, optionally wherein the cytoplasmic domain is linked, directly or indirectly, to the transmembrane domain. In some embodiments, the cytoplasmic domain is or contains a native CD86 cytoplasmic domain. In some embodiments, the cytoplasmic domain contains the sequence of amino acids set forth as residues 269-329 of SEQ ID NO: 2 or a functional variant thereof that exhibits at least 85% sequence identity to residues 269-329 of SEQ ID NO: 2. In some embodiments, the cytoplasmic domain contains an ITAM signaling motif and/or is or contains an intracellular signaling domain of CD3 zeta.

In some embodiments, the variant CD86 polypeptide does not contain a cytoplasmic signaling domain and/or is not capable of mediating or modulating an intracellular signal when expressed on a cell.

Provided herein are immunomodulatory proteins, containing a first variant CD86 polypeptide of any variant CD86 polypeptide described herein and a second variant CD86 polypeptide of any variant CD86 polypeptide described herein. In some embodiments, the first and second variant CD86 polypeptides are linked indirectly via a linker. In some embodiments, the first and second variant CD86 polypeptide are each linked to a multimerization domain, whereby the immunomodulatory protein is a multimer containing the first and second variant CD86 polypeptide. In some embodiments, the multimer is a dimer, optionally a homodimer. In some embodiments, the multimer is a homodimer. In some embodiments, the first variant CD86 polypeptide and the second variant CD86 polypeptide are the same.

Provided herein are immunomodulatory proteins, containing the any of the variant CD86 polypeptide described herein linked, directly or indirectly via a linker, to a second polypeptide containing an immunoglobulin superfamily (IgSF) domain of an IgSF family member. In some embodiments, the IgSF domain is an affinity-modified IgSF domain, said affinity-modified IgSF domain containing one or more amino acid modifications compared to the unmodified or wild-type IgSF domain of the IgSF family member. In some embodiments, the IgSF domain is an affinity modified IgSF domain that exhibits altered binding to one or more of its cognate binding partner(s) compared to the binding of the unmodified or wild-type IgSF domain of the IgSF family member to the same one or more cognate binding partner(s). In some embodiments, the IgSF domain exhibits increased binding to one or more of its cognate binding partner(s) compared to the binding of the unmodified or wild-type IgSF domain of the IgSF family member to the same one or more cognate binding partner(s).

In some embodiments, the IgSF domain of the second polypeptide is a tumor-localizing moiety that binds to a ligand expressed on a tumor or is an inflammatory-localizing moiety that binds to a cell or tissue associated with an inflammatory environment. In some embodiments, the ligand is B7H6. In some embodiments, the IgSF domain is from NKp30. In some embodiments, the immunomodulatory protein further contains a multimerization domain linked to at least one of the variant CD86 polypeptide, or the second polypeptide. In some embodiments, the immunomodulatory protein described herein further contains a third polypeptide containing an IgSF domain of an IgSF family member or an affinity-modified IgSF domain thereof, said affinity-modified IgSF domain containing one or more amino acid modifications compared to the unmodified or wild-type IgSF domain of the IgSF family member. In some embodiments, the third polypeptide is the same as the first and/or second polypeptide; or the third polypeptide is different from the first and/or second polypeptide.

In some embodiments, the immunomodulatory protein further contains a multimerization domain linked to at least one of the variant CD86 polypeptide, the second polypeptide and/or the third polypeptide. In some embodiments, the multimerization domain is an Fc domain of an immunoglobulin, optionally wherein the immunoglobulin protein is human and/or the Fc region is human. In some embodiments, the immunoglobulin protein is human and/or the Fc region is human. In some embodiments, the Fc domain is an IgG1, IgG2 or IgG4, or is a variant thereof with reduced effector function. In some embodiments, the Fc domain is an IgG1 Fc domain, optionally a human IgG1, or is a variant thereof with reduced effector function. In some embodiments, the Fc domain is a human IgG1 Fc domain. In some embodiments, the Fc domain contains the sequence of amino acids set forth in SEQ ID NO: 229 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 229. In some embodiments, the Fc domain is or contains the sequence of amino acids set forth in SEQ ID NO: 229. In some embodiments, the Fc domain is a variant IgG1 containing one or more amino acid substitutions and the one or more amino acid substitutions are selected from E233P, L234A, L234V, L235A, L235E, G236del, G237A, S267K, or N297G, each numbered according to EU index by Kabat. In some embodiments, the Fc domain contains the amino acid substitution N297G, the amino acid substitutions R292C/N297G/V302C, or the amino acid substitutions L234A/L235E/G237A, each numbered according to the EU index of Kabat. In some embodiments, the variant Fc region further contains the amino acid substitution C220S, wherein the residues are numbered according to the EU index of Kabat. In some embodiments, the Fc region contains K447del, wherein the residue is numbered according to the EU index of Kabat. The Fc region may also be referred to herein as an Fc domain.

In some embodiments, the Fc domain contains the sequence of amino acids set forth in SEQ ID NO: 230 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 230 and contains one or more of the respective amino acid modifications set forth in SEQ ID NO: 230 compared to human IgG1. In some embodiments, the Fc domain is contains the sequence of amino acids set forth in SEQ ID NO: 230.

Provided herein is an immunomodulatory protein comprising a first polypeptide and a second polypeptide, wherein: the first polypeptide comprises at least one IgSF domain linked through a linker to a first Fc domain, wherein the at least one IgSF domain comprises one or both of a variant CD86 polypeptide of any variant CD86 polypeptide provided herein or is an IgSF domain of a PD1 polypeptide or a variant thereof, and the second polypeptide comprises at least one IgSF linked through a linker to a second Fc domain, wherein the at least one IgSF domain comprises one or both of a variant CD86 polypeptide of any variant CD86 polypeptide provided herein or is an IgSF domain of a PD1 polypeptide or a variant thereof, wherein the immunomodulatory proteins comprise at least one IgSF domain of CD86 and at least one IgSF domain of PD-1 or a variant thereof.

In some of any of the provided embodiments, the at least one IgSF domain of the first polypeptide comprises a variant CD86 polypeptide that is any variant CD86 polypeptide provided herein. In some of any of the provided embodiments, the at least one IgSF domain of the second polypeptide comprises a variant PD1 polypeptide. In some of any of the provided embodiments, the at least one IgSF domain of the first polypeptide is a first IgSF domain, wherein the first IgSF domain is a variant CD86 polypeptide that is any variant CD86 polypeptide provided herein, and the first polypeptide comprises a second IgSF domain linked through a linker to the first Fc domain. In some of any of the provided embodiments, the second IgSF domain of the first polypeptide comprises a variant PD1 polypeptide. In some of any of the provided embodiments, the at least one IgSF domain of the second polypeptide is a first IgSF domain, wherein the first IgSF domain is variant CD86 polypeptide that is any variant CD86 polypeptide provided herein, and the second polypeptide comprises a second IgSF domain linked through a linker to the second Fc domain. In some of any of the provided embodiments, the second IgSF domain of the second polypeptide comprises a variant PD1 polypeptide.

In some of any of the provided embodiments, the at least one IgSF domain of the first polypeptide is linked through a linker to the N- or C-terminus of the first Fc domain; and the at least one IgSF domain of the second polypeptide is linked through a linker to the N- or C-terminus of the second Fc domain. In some of any of the provided embodiments, the second IgSF domain of the first polypeptide is linked to the first Fc domain terminus opposite to the terminus linked to the first IgSF domain. In some of any of the provided embodiments, the second IgSF domain of the second polypeptide is linked to the second Fc domain terminus opposite to the terminus linked to the first IgSF domain. In some of any of the provided embodiments, wherein the linker independently comprises the sequence of SEQ ID NO: 222 or 224, optionally wherein the linker comprises 1 to 4 repeats of the sequence of SEQ ID NO:222 or 224. In some of any of the provided embodiments, the first Fc domain and the second Fc domain are identical, optionally, wherein the first Fc domain and the second Fc domain comprise the sequence of SEQ ID NO: 230.

In some of any of the provided embodiments, wherein the first polypeptide and the second polypeptide dimerize through the first and second Fc domains to form a homodimer. In some of any of the provided embodiments, the first and second polypeptides of the homodimer comprise from left to right a variant PD1 polypeptide-linker-Fc-linker-variant CD86 polypeptide.

In some of any of the provided embodiments, the variant PD1 polypeptide comprises the sequence of SEQ ID NO: 315. In some of any of the provided embodiments, the variant CD86 polypeptide comprise the sequence of SEQ ID NO: 94 or 150. In some of any of the provided embodiments, the first Fc domain and the second Fc domain are different, optionally wherein the first and second Fc domains comprise knob-into-hole mutations, optionally wherein the first Fc domain or the second Fc domain comprises the sequence of SEQ ID NO: 346, and the other of the first Fc domain or the second Fc domain comprises the sequence of SEQ ID NO:347.

In some of any of the provided embodiments, the first polypeptide and the second polypeptide dimerize through the first and second Fc domains to form a heterodimer. In some of any of the provided embodiments, the first polypeptide of the heterodimer comprises from left to right a variant PD1 polypeptide-linker-Fc and the second polypeptide of the heterodimer comprises from left to right a variant CD86 polypeptide-linker-Fc, an Fc-linker-variant CD86 polypeptide, or a variant PD1-linker-Fc-linker-variant CD86.

In some of any of the provided embodiments, the variant PD1 polypeptide comprises the sequence of SEQ ID NO: 315. In some of any of the provided embodiments, the variant CD86 polypeptide comprise the sequence of SEQ ID NO: 94 or 150. In some of any of the provided embodiments, the first polypeptide of the heterodimer comprises the sequence of SEQ ID NO: 350; and the second polypeptide of the heterodimer comprises the sequence of SEQ ID NO: 351, 352, or 353.

Provided herein are conjugates containing any of the variant CD86 polypeptides described herein linked to a targeting moiety that specifically binds to a molecule on the surface of a cell. In some embodiments, the cell is an immune cell or is a tumor cell. In some embodiments, the moiety is a protein, a peptide, nucleic acid, small molecule or nanoparticle. In some embodiments, the moiety is an antibody or antigen-binding fragment. In some embodiments, the conjugate described herein is a fusion protein.

In some of any of the provided embodiments, the variant CD86 polypeptide is linked to the N- or C-terminus of the V_{H} or V_{L} of the antibody. In some embodiments, the variant CD86 polypeptide is linked to the N- or C-terminus of the V_{H} or V_{L} of the antibody is any variant CD86 polypeptide provided herein. In some of any of the provided embodiments, the antibody is an anti-HER2 antibody or an anti-EGFR antibody. In some of any of the provided embodiments, the anti-HER2 antibody is pertuzumab. In some of any of the provided embodiments, the variant CD86 polypeptide is linked to the N-terminus of the V_{H} of pertuzumab, the C-terminus of the V_{H} of pertuzumab, the N-terminus of the V_{L} of pertuzumab, or the C-terminus of the V_{L} of pertuzumab, optionally comprising the sequence of SEQ ID NO:342, 344, 343, or 345, respectively. In some of any of the provided embodiments, the anti-EGFR antibody is panitumumab. In some of any of the provided embodiments, the variant CD86 polypeptide is linked to the N-terminus of the VH of panitumumab, the C-terminus of the VH of panitumumab, the N-terminus of the VL of panitumumab, or the C-terminus of the VL of panitumumab, optionally comprising the sequence of SEQ ID NO:348, 350, 349, or 351, respectively, or an anti-EGFR antibody.

Provided herein are nucleic acid molecules encoding any of the variant CD86 polypeptides described herein, immunomodulatory proteins described herein, or conjugates that are fusion proteins described herein. In some embodiments, the nucleic acid molecule is a synthetic nucleic acid. In some embodiments, the nucleic acid molecule is cDNA.

Provided herein are vectors containing the nucleic acid molecule described herein. In some embodiments, the vector is an expression vector. In some embodiments, the vector is a mammalian expression vector or a viral vector.

Provided herein are cells containing the vector described herein. In some embodiments, the cell is a mammalian cell. In some embodiments, the cell is a human cell.

Provided herein are methods of producing a protein containing a variant CD86 polypeptide, including introducing the nucleic acid molecule described herein or vector described herein into a host cell under conditions to express the protein in the cell. In some embodiments, the method further includes isolating or purifying the protein from the cell.

Provided herein are methods of engineering a cell expressing a variant CD86 polypeptide, the method including introducing a nucleic acid molecule encoding the variant CD86 polypeptide described herein, immunomodulatory protein described herein or a conjugate that is a fusion protein described herein into a host cell under conditions in which the polypeptide is expressed in the cell.

Provided herein are engineered cells, containing a variant CD86 polypeptide described herein, immunomodulatory protein described herein or a conjugate that is a fusion protein as described herein, a nucleic acid molecule described herein or a vector described herein. In some embodiments, the variant CD86 polypeptide contains a transmembrane domain or is the transmembrane immunomodulatory protein described herein; and/or the protein containing the variant CD86 polypeptide is expressed on the surface of the cell. In some embodiments, the variant CD86 polypeptide does not contain a transmembrane domain and/or is not expressed on the surface of the cell; and/or the variant CD86 polypeptide is capable of being secreted from the engineered cell. In some embodiments, the protein does not contain a cytoplasmic signaling domain or transmembrane domain and/or is not expressed on the surface of the cell; and/or the protein is capable of being secreted from the engineered cell when expressed.

In some embodiments, the engineered cell is an immune cell. In some embodiments, the immune cell is a lymphocyte. In some embodiments, the lymphocyte is a T cell. In some embodiments, the T cell is a CD4+ and/or CD8+ T cell. In some embodiments, the T cell is a regulatory T cell (Treg). In some embodiments, the engineered cell is a primary cell. In some embodiments, the engineered cell is a mammalian cell. In some embodiments, the engineered cell is a human cell. In some embodiments, the engineered cell further contains a chimeric antigen receptor (CAR). In some embodiments, the engineered cell further contains an engineered T-cell receptor (TCR).

Provided herein are infectious agents containing a variant CD86 polypeptide described herein, immunomodulatory protein described herein or a conjugate that is a fusion protein described herein, a nucleic acid molecule described herein or a vector described herein. In some embodiments, the infectious agent is a bacterium or a virus. In some embodiments, the infectious agent is a virus and the virus is an oncolytic virus.

Provided herein are pharmaceutical compositions, containing a variant CD86 polypeptide described herein, immunomodulatory protein described herein or a conjugate that is a fusion protein described herein, an engineered cell described herein or an infectious agent described herein. In some embodiments, the pharmaceutical composition contains a pharmaceutically acceptable excipient. In some embodiments, the pharmaceutical composition is sterile.

Provided herein are articles of manufacture including the pharmaceutical composition described herein in a vial or a container. In some embodiments, the vial or container is sealed.

Provided herein are kits containing the pharmaceutical composition described herein or the article of manufacture described herein and instructions for use.

Provided herein are methods of modulating an immune response in a subject, the methods including administering a variant CD86 polypeptide described herein, immunomodulatory protein described herein or a conjugate that is a fusion protein described herein, an engineered cell described herein, an infectious agent described herein, or the pharmaceutical composition described herein.

Provided herein are methods of modulating an immune response in a subject, including administering the engineered cells described herein. In some embodiments, the engineered cells are autologous to the subject. In some embodiments, the engineered cells are allogenic to the subject. In some embodiments, modulating the immune response treats a disease or condition in the subject.

Provided herein are methods of treating a disease or condition in a subject in need thereof, the methods including administering a variant CD86 polypeptide described herein, immunomodulatory protein described herein or a conjugate that is a fusion protein described herein, an engineered cell described herein, an infectious agent described herein, or the pharmaceutical composition described herein.

Provided herein are methods of treating a disease or condition in a subject in need thereof, including administering the engineered cells described herein. In some embodiments, the engineered cells are autologous to the subject. In some embodiments, the engineered cells are allogenic to the subject.

In some embodiments, the immune response is increased in the subject. In some embodiments, an immunomodulatory protein or conjugate containing a variant CD86 polypeptide linked to a tumor-localizing moiety is administered to the subject. In some embodiments, the tumor-localizing moiety is or contains a binding molecule that recognizes a tumor antigen. In some embodiments, the binding molecule contains an antibody or an antigen-binding fragment thereof or contains a wild-type IgSF domain or variant thereof.

In some embodiments, a pharmaceutical composition containing the immunomodulatory protein described herein or the conjugate described herein is administered to the subject. In some embodiments, an engineered cell containing a variant CD86 polypeptide that is a transmembrane immunomodulatory protein is administered to the subject, optionally, wherein the engineered cell described herein. In some embodiments, the transmembrane immunomodulatory protein is as described herein.

In some embodiments, the disease or condition is a tumor or cancer. In some embodiments, the disease or condition is selected from melanoma, lung cancer, bladder cancer, a hematological malignancy, liver cancer, brain cancer, renal cancer, breast cancer, pancreatic cancer, colorectal cancer, spleen cancer, prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer.

In some embodiments, the immune response is decreased. In some embodiment, a variant CD86 polypeptide or immunomodulatory protein that is soluble is administered to the subject. In some embodiments, the soluble polypeptide or immunomodulatory protein is an Fc fusion protein.

In some embodiments, a pharmaceutical composition containing a variant CD86 polypeptide described herein, or the immunomodulatory protein described herein is administered to the subject. In some embodiments, an engineered cell containing a secretable variant CD86 polypeptide is administered to the subject, optionally wherein the engineered cell is any described herein.

In some embodiments, the disease or condition is an inflammatory or autoimmune disease or condition. In some embodiments, the disease or condition is an Antineutrophil cytoplasmic antibodies (ANCA)-associated vasculitis, a vasculitis, an autoimmune skin disease, transplantation, a Rheumatic disease, an inflammatory gastrointestinal disease, an inflammatory eye disease, an inflammatory neurological disease, an inflammatory pulmonary disease, an inflammatory endocrine disease, or an autoimmune hematological disease. In some embodiments, the disease or condition is selected from inflammatory bowel disease, transplant, Crohn's disease, ulcerative colitis, multiple sclerosis, asthma, rheumatoid arthritis, or psoriasis.

### BRIEF DESCRIPTION OF THE DRAWINGS

FIG. 1A shows IFN-gamma (IFNγ; top left), IL2 (top right), and TNFα (bottom) release from Mock transduced T cells and E6 TCR-transduced T cells expressing a TCR alone or co-expressing an indicated CD86 ECD TIP in supernatant following 24 hours of co-culture with varying numbers of HLA-A2+HPV+ target cells (SCC152).

FIGS. 1B and 1C show CD4+ and CD8+ T cell proliferation, respectively, 3 days after initiation of co-culture of Mock transduced T cells or E6 TCR-transduced T cells expressing a TCR alone or co-expressing an indicated CD86 ECD TIP with varying numbers of HLA-A2+HPV+ target cells (SCC152).

FIG. 1D shows killing activity of Mock transduced T cells and E6 TCR-transduced T cells expressing a TCR alone or co-expressing an indicated CD86 ECD TIP at different effector to target ratios (E:T) after 4 days of co-culturing with HLA-A2+HPV+ target cells (SCC152).

FIG. 2A depicts HER2 expression levels on CEM-T2, SCC152, and NCI-N87 cell lines.

FIG. 2B shows killing activity of Mock transduced T cells and anti-HER2 CAR-transduced T cells expressing the CAR alone or co-expressing an indicated CD86 ECD TIP at different effector to target ratios (E:T) after 24 hours of co-culturing with NCI-N87.

FIG. 2C shows killing activity of Mock transduced T cells and anti-HER2 CAR-transduced T cells expressing the CAR alone or co-expressing an indicated CD86 ECD TIP at different effector to target ratios (E:T) after 24 hours of co-culturing with SCC152.

FIG. 3 depicts an exemplary alignment of the wildtype CD86 extracellular domain (ECD) sequence set forth in SEQ ID NO: 29 containing residues 24-247 of the CD86 designated “CD86(B7-2)” (SEQ ID NO: 2) with the wildtype IgV sequence set forth in SEQ ID NO: 122 containing residues 33-131 of the CD86 designated “CD86(B7-2)” (SEQ ID NO: 2). The symbol “*” indicates that the two aligned residues are identical. The absence of a “*” between two aligned residues indicates that the aligned amino acids are not identical. The symbol “-” indicates a gap in the alignment. Exemplary, non-limiting positions in SEQ ID NO: 122 corresponding to positions with numbering set forth in SEQ ID NO: 29 are indicated by a box.

FIG. 4A and FIG. 4B depict binding of exemplary PD1-CD86 stack constructs at various concentrations (0.1 nM to 100 nM) to cognate binding partner CTLA-4, determined by Mean Fluorescence Intensity (MFI) assessed by flow cytometry.

FIG. 5A and FIG. 5B depict binding of exemplary PD1-CD86 stack constructs at various concentrations (0.1 nM to 100 nM) to cognate binding partner CD28, determined by Mean Fluorescence Intensity (MFI) assessed by flow cytometry.

FIG. 6A and FIG. 6B depict binding of exemplary PD1-CD86 stack constructs at various concentrations (0.1 nM to 100 nM) to cognate binding partner PD-L1, determined by Mean Fluorescence Intensity (MFI) assessed by flow cytometry

FIG. 7A and FIG. 7B depict the ability of exemplary variant PD1-CD86 stack constructs to deliver PD-L1 dependent costimulation of CD28 using Jurkat/IL-2 reporter cells (FIG. 7A) or Jurkat/IL-2 reporter cells expressing PD-L1 (FIG. 7B), as measured by IL-2 luminiescence relative luminescence units (RLU).

FIG. 8 and FIG. 9 depict cytokine concentrations (pg/mL) of T cell supernatants from a cytomegalovirus (CMV) antigen-specific functional assay. Supernatants were determined for IL-2 (FIG. 8) and IFNg (FIG. 9), as assessed by ELISA.

FIG. 10 depicts the binding of exemplary NKp30-CD86 stack constructs at various concentrations (100 to 100,000 pM) to CD28 and CTLA-4, determined as median hIgG PE.

FIG. 11A depicts the binding ability of exemplary NKp30-CD86 stack constructs to primary T cells, determined by Mean Flourescence Intensity (MFI) assessed by flow cytometry. FIG. 11B shows percent T cell proliferation assessed by flow cytometry using CFSE dye.

FIG. 12 depicts the concentration of IL-2 (pg/mL) harvested from T cell supernatants as assessed by ELISA.

FIG. 13 depicts exemplary NKp30-CD86 stack construct costimultion in the presence (left) and absence (right) of B7H6. Percent T cell proliferation assessed by flow cytometry.

FIGS. 14A-14D depict the structure of exemplary formatted stack constructs.

FIG. 15A and FIG. 15B depict binding of the exemplary formatted stack constructs at various concentrations (100 nM serial diluted 8 times to 1:4) to cognate binding partners PD-L1 (left) and CD28 (right) as assessed by flow cytometry and measured by Mean Fluorescence Intensity (MFI).

FIG. 16A and FIG. 16B depict the costimulatory ability of the exemplary formatted stack constructs tested in a luciferase reporter cell system and determined using Relative Luminescence Units (RLU).

FIG. 17A depicts the ability of exemplary CD86-PD-1 stack constructs to facilitate cytokine production in T cells as measured by the concentration of IFNg, IL2, and TNFα (pg/mL).

FIG. 17B depicts the ability of exemplary CD86-PD-1 stack constructs to facilitate T cell cytoxic activity against HLA-A2+HPV+ target cells at 24 hours, 48 hours, and 72 hours post incubation assessed by Relative Luminescence Units (RLU).

FIG. 18A, FIG. 18B, and FIG. 18C depict exemplary configurations of conjugates of exemplary variant CD86 IgV molecules with HER2 and EGFR targeting antibodies.

FIG. 19A and FIG. 19B depict binding of exemplary pertuzumab-CD86 conjugates to HER2 (FIG. 19A) and exemplary panitumumab-CD86 conjugates to EGFR (FIG. 19B) as determined by Mean Fluorescence Intensity.

FIG. 20A and FIG. 20B depict the ability of pertuzumab-CD86 conjugates (FIG. 20A) and exemplary panitumumab-CD86 conjugates (FIG. 20B) to provide costimulation to T cells in an IL-2 luciferase reporter assay as measured in Relative Luminescence Units (RLU).

FIG. 21A and FIG. 21B depict the ability of exemplary pertuzumab-CD86 conjugates (FIG. 21A) and exemplary panitumumab-CD86 conjugates (FIG. 21B) to facilitate T cell cytotoxic activity as tested at various effector to target ratios (E:T) of primary human T cells measured by percent killing of SCC-152 target cells.

FIG. 22A and FIG. 22B depict the ability of pertuzumab-CD86 conjugates (FIG. 22A) and exemplary panitumumab-CD86 conjugates (FIG. 22B) to facilitate cytokine production in T cells by determining the concentration of IFNg, IL2, and TNFα (nM protein) in the cellular supernatant.

FIG. 23A depicts various exemplary configurations of a stack molecule containing a first variant IgSF domain (first vIgD) and a second IgSF domain, such as a second variant IgSF domain (second vIgD). FIG. 23B depicts various exemplary configurations of a stack molecule containing a first variant IgSF domain (first vIgD), a second IgSF domain, such as a second variant IgSF domain (second vIgD), and a third IgSF domain, such as a third variant IgSF domain (third vIgD).

FIG. 24A and FIG. 24B depict various formats of the provided variant IgSF domain molecules. FIG. 24A depicts soluble molecules and FIG. 24B depicts a transmembrane immunomodulatory protein (TIP) containing a variant IgSF domain (vIgD) expressed on the surface of a cell.

FIG. 25 depicts a secreted immunomodulatory protein (SIP) in which a variant IgSF domain (vIgD) is secreted from a cell, such as a first T cell (e.g., CAR T cell).

### DETAILED DESCRIPTION

Provided herein are immunomodulatory proteins that are or contain variants or mutants of CD86 and specific binding fragments thereof that exhibit altered binding activity or affinity to at least one target ligand cognate binding partner (also called counter-structure ligand protein). In some embodiments, the variant CD86 polypeptides contain one or more amino acid modifications (e.g., amino acid substitutions, deletions, or additions) compared to an unmodified or wild-type CD86 polypeptide. In some embodiments, the variant CD86 polypeptides contain one or more amino acid modifications (e.g., substitutions) compared to an unmodified or wild-type CD86 polypeptide. In some embodiments, the one or more amino acid substitutions are in the extracellular domain, such as are in an IgSF domain (e.g., IgV of IgC), of an unmodified or wild-type CD86 polypeptide. In some embodiments, the variant CD86 polypeptides exhibit altered, such as increased or decreased, binding activity or affinity to one or more of CD28 or CTLA-4 compared to the unmodified or wild-type CD86 not containing the one or more modifications.

In some embodiments, the variant CD86 polypeptides exhibit increased binding affinity to CD28 compared to the unmodified or wild-type CD86 not containing the one or more modifications. In some embodiments, the variant CD86 polypeptides exhibit increased binding affinity to at least CD28 compared to the unmodified or wild-type CD86 not containing the one or more modifications. In some embodiments, the binding affinity is altered (e.g. increased) at least 1.2-fold, 1.4-fold, 1.5-fold, 2.0-fold, 3.0-fold, 4.0-fold, 5.0-fold, 6.0-fold, 7.0-fold, 8.0-fold, 9.0-fold, 10.0-fold, 20.0-fold, 30.0-fold, 40.0-fold, 50.0-fold, 60.0-fold, 70.0-fold, 80.0-fold, 90.0-fold, 100.0-fold, 124.0-fold or more compared to the unmodified or wild-type CD86 not containing the one or more modifications.

In some embodiments, the variant CD86 polypeptides exhibit decreased, no change, or not greater binding affinity to CTLA-4 compared to the unmodified or wild-type CD86 not containing the one or more modifications. In some embodiments, the binding affinity to CTLA-4 is decreased. In some embodiments, the binding affinity is altered (e.g. decreased) at least 1.2-fold, 1.4-fold, 1.5-fold, 2.0-fold, 3.0-fold, 4.0-fold, 5.0-fold, 6.0-fold, 7.0-fold, 8.0-fold, 9.0-fold, 10.0-fold or more compared to the unmodified or wild-type CD86 not containing the one or more modifications.

In some embodiments, the variant CD86 polypeptides and immunomodulatory proteins modulate an immunological immune response, such as increase or decrease an immune response. The particular modulation can be based on the format of the variant CD86 polypeptide, depending on whether a particular format provides an antagonist or blocking activity or an agonist activity. Also provided are various immunomodulatory protein formats of the provided variant polypeptides. As shown herein, alternative formats can facilitate manipulation of the immune response, and hence the therapeutic application. The ability to format the variant polypeptides in various configurations to, depending on the context, antagonize or agonize an immune response, offers flexibility in therapeutic applications based on the same increased binding and activity of a variant CD86 for binding partners. As an example, tethering variant CD86 proteins to a surface can deliver a localized costimulatory signal, while, in other cases, presenting CD86 in a non-localized soluble form can confer antagonistic activity. In some embodiments, the variant CD86 polypeptides and immunomodulatory proteins provided herein can be used for the treatment of diseases or conditions that are associated with a dysregulated immune response.

In some embodiments, the immunomodulatory proteins are soluble. In some embodiments, the immunomodulatory proteins are transmembrane immunomodulatory proteins capable of being expressed on the surface of cells. In some embodiments, the immunomodulatory proteins are secretable immunomodulatory proteins capable of being secreted from a cell in which it is expressed. In some embodiments, also provided herein are one or more other immunomodulatory proteins that are conjugates or fusions containing a variant CD86 polypeptide provided herein and one or more other moiety or polypeptide. In some aspects, provided are engineered cells containing the transmembrane immunomodulatory proteins or secretable immunomodulatory proteins. In some aspects, provided are infectious agents capable of delivering for expression the transmembrane immunomodulatory proteins or secretable immunomodulatory proteins into a cell in which the infectious agent infects. In some embodiments, also provided herein are one or more other immunomodulatory proteins that are conjugates or fusions containing a variant CD86 polypeptide provided herein and one or more other moiety or polypeptide.

In some embodiments, the variant CD86 polypeptide is provided in a format that exhibits agonist activity of its cognate binding partner CD28 and/or that stimulates or initiates costimulatory signaling via CD28. Included among such immunomodulatory protein formats is an engineered cell expressing a variant CD86 polypeptide as a transmembrane immunomodulatory protein. In other cases, the immunomodulatory format can include a fusion with another molecule, such as provided by certain “stack molecules” with other IgSF domains, including tumor-localizing domains (e.g. vCD86-NkP30 constructs), as well as with antibody conjugate formats (e.g. vCD86-anti-HER2 or vCD86-antiHER1 constructs). Such variant CD86 immunomodulatory proteins and formats thereof (e.g. engineered cells or fusion constructs) can be used to treat cancer, viral infections, or bacterial infections. In some embodiments, the variant CD86 immunomodulatory proteins and formats thereof (e.g. engineered cells or fusion constructs) exhibit enhanced costimulatory activity and thereby result in increased T cell activity (e.g. in vivo or in vitro), such as in a primary T cell assay, relative to a wild-type or unmodified CD86 control. In some aspects, T cell activity can be assessed by assessing production of cytokines, such as IL-2, IFN-gamma, or TNFα. In some aspects, the increase, such as the increase in IFN-gamma, IL-2 or TNFα, is by greater than or greater than about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5.0-fold, 6.0-fold, 7.0-fold, 8.0-fold, 9.0-fold, 10.0-fold or more compared to the unmodified or wild-type CD86 not containing the one or more modifications.

In some embodiments, the variant CD86 polypeptide is provided in a format that exhibits antagonist activity of its cognate binding partner CD28 and/or that blocks or inhibits costimulatory signaling via CD28. Included among such immunomodulatory protein formats is a variant CD86 polypeptide that is soluble (e.g. variant CD86-Fc fusion protein). Such variant CD86 immunomodulatory proteins can be used to treat inflammatory or autoimmune disorders. In some embodiments, the variant CD86 immunomodulatory proteins and formats thereof (e.g. soluble variant CD86-Fc fusion protein) inhibit or block costimulatory signaling and thereby result in decreased T cell activity (e.g. in vivo or in vitro), such as in a primary T cell assay, relative to a wild-type or unmodified CD86 control. In some aspects, T cell activity can be assessed by assessing production of cytokines, such as IL-2, IFN-gamma, or TNFα. In some aspects, the decrease, such as the decrease in IFN-gamma, IL-2, TNFα is by greater than or greater than about 1.1-fold, 1.2-fold, 1.3-fold, 1.4-fold, 1.5-fold, 1.6-fold, 1.7-fold, 1.8-fold, 1.9-fold, 2.0-fold, 3.0-fold, 4.0-fold, 5.0-fold, 6.0-fold, 7.0-fold, 8.0-fold, 9.0-fold, 10.0 fold or more compared to the unmodified or wild-type CD86 not containing the one or more modifications.

In some embodiments, the provided variant CD86 polypeptides modulate T cell activation, expansion, differentiation, and survival via interactions with costimulatory signaling molecules. In general, antigen specific T-cell activation generally requires two distinct signals. The first signal is provided by the interaction of the T-cell receptor (TCR) with major histocompatibility complex (MHC) associated antigens present on antigen presenting cells (APCs). The second signal is costimulatory, e.g., a CD28 costimulatory signal, to TCR engagement and necessary to avoid T-cell apoptosis or anergy.

In some embodiments, under normal physiological conditions, the T cell-mediated immune response is initiated by antigen recognition by the T cell receptor (TCR) and is regulated by a balance of co-stimulatory and inhibitory signals (e.g., immune checkpoint proteins). The immune system relies on immune checkpoints to prevent autoimmunity (i.e., self-tolerance) and to protect tissues from excessive damage during an immune response, for example during an attack against a pathogenic infection. In some cases, however, these immunomodulatory proteins can be dysregulated in diseases and conditions, including tumors, as a mechanism for evading the immune system.

In some embodiments, among known T-cell costimulatory receptors is CD28, which is the T-cell costimulatory receptor for the ligands B7-1 (CD80) and B7-2 (CD86) both of which are present on APCs. These same ligands can also bind to the inhibitory T-cell receptor CTLA4 (cytotoxic T-lymphocyte-associated protein 4) with greater affinity than for CD28; the binding to CTLA4 acts to down-modulate the immune response.

Enhancement or suppression of the activity of CD28 and CTLA-4 receptors has clinical significance for treatment of inflammatory and autoimmune disorders, cancer, and viral infections. In some cases, however, therapies to intervene and alter the costimulatory effects of both receptors are constrained by the spatial orientation requirements as well as size limitations imposed by the confines of the immunological synapse. In some aspects, existing therapeutic drugs, including antibody drugs, may not be able to interact simultaneously with the multiple target proteins involved in modulating these interactions. In addition, in some cases, existing therapeutic drugs may only have the ability to antagonize, but not agonize, an immune response. Additionally, pharmacokinetic differences between drugs that independently target one or the other of these two receptors can create difficulties in properly maintaining a desired blood concentration of such drug combinations throughout the course of treatment. The provided variant CD86 polypeptides and immunomodulatory proteins, and other formats as described, address such problems. Methods of making and using these variants of CD86 polypeptides and immunomodulatory proteins are also provided.

All publications, including patents, patent applications, scientific articles, and databases mentioned in this specification are herein incorporated by reference in their entirety for all purposes to the same extent as if each individual publication, including patent, patent application, scientific article, or database, were specifically and individually indicated to be incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms used throughout this specification are defined as follows unless otherwise limited in specific instances. As used in the specification and the appended claims, the singular forms “a,” “an,” and “the” include plural referents unless the context clearly dictates otherwise. Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names are per IUPAC-IUB nomenclature. Unless indicated otherwise, all numerical ranges are inclusive of the values defining the range as well as all integer values in-between.

The term “affinity modified” as used in the context of an immunoglobulin superfamily domain, means a mammalian immunoglobulin superfamily (IgSF) domain having an altered amino acid sequence (relative to the corresponding wild-type parental or unmodified IgSF domain) such that it has an increased or decreased binding affinity or avidity to at least one of its cognate binding partners (alternatively “counter-structures”) compared to the parental wild-type or unmodified (i.e., non-affinity modified) IgSF control domain. Included in this context is an affinity modified CD86 IgSF domain. In some embodiments, the affinity-modified IgSF domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wildtype or unmodified IgSF domain. An increase or decrease in binding affinity or avidity can be determined using well known binding assays such as flow cytometry. Larsen et al., American Journal of Transplantation, Vol 5: 443-453 (2005). See also, Linsley et al., Immunity, Vol 1(9: 793-801 (1994). An increase in a protein's binding affinity or avidity to its cognate binding partner(s) is to a value at least 10% greater than that of the wild-type IgSF domain control and in some embodiments, at least 20%, 30%, 40%, 50%, 100%, 200%, 300%, 500%, 1000%, 5000%, or 10000% greater than that of the wild-type IgSF domain control value. A decrease in a protein's binding affinity or avidity to at least one of its cognate binding partner is to a value no greater than 90% of the control but no less than 10% of the wild-type IgSF domain control value, and in some embodiments no greater than 80%, 70% 60%, 50%, 40%, 30%, or 20% but no less than 10% of the wild-type IgSF domain control value. An affinity-modified protein is altered in primary amino acid sequence by substitution, addition, or deletion of amino acid residues. The term “affinity modified IgSF domain” is not to be construed as imposing any condition for any particular starting composition or method by which the affinity-modified IgSF domain was created. Thus, the affinity modified IgSF domains of the present invention are not limited to wild type IgSF domains that are then transformed to an affinity modified IgSF domain by any particular process of affinity modification. An affinity modified IgSF domain polypeptide can, for example, be generated starting from wild type mammalian IgSF domain sequence information, then modeled in silico for binding to its cognate binding partner, and finally recombinantly or chemically synthesized to yield the affinity modified IgSF domain composition of matter. In one alternative example, an affinity modified IgSF domain can be created by site-directed mutagenesis of a wild-type IgSF domain. Thus, affinity modified IgSF domain denotes a product and not necessarily a product produced by any given process. A variety of techniques including recombinant methods, chemical synthesis, or combinations thereof, may be employed.

The term “allogeneic” as used herein means a cell or tissue that is removed from one organism and then infused or adoptively transferred into a genetically dissimilar organism of the same species. In some embodiments of the invention, the species is murine or human.

The term “autologous” as used herein means a cell or tissue that is removed from the same organism to which it is later infused or adoptively transferred. An autologous cell or tissue can be altered by, for example, recombinant DNA methodologies, such that it is no longer genetically identical to the native cell or native tissue which is removed from the organism. For example, a native autologous T-cell can be genetically engineered by recombinant DNA techniques to become an autologous engineered cell expressing a transmembrane immunomodulatory protein and/or chimeric antigen receptor (CAR), which in some cases involves engineering a T-cell or TIL (tumor infiltrating lymphocyte). The engineered cells are then infused into a patient from whom the native T-cell was isolated. In some embodiments, the organism is human or murine.

The terms “binding affinity,” and “binding avidity” as used herein means the specific binding affinity and specific binding avidity, respectively, of a protein for its counter-structure under specific binding conditions. In biochemical kinetics, avidity refers to the accumulated strength of multiple affinities of individual non-covalent binding interactions, such as between CD86 and its counter-structures CD28 and/or CTLA-4. As such, avidity is distinct from affinity, which describes the strength of a single interaction. An increase or attenuation in binding affinity of a variant CD86 containing an affinity modified CD86 IgSF domain to its counter-structure is determined relative to the binding affinity of the unmodified CD86, such as an unmodified CD86 containing the native or wild-type IgSF domain, such as IgV domain. Methods for determining binding affinity or avidity are known in art. See, for example, Larsen et al., American Journal of Transplantation, Vol. 5: 443-453 (2005). In some embodiments, a variant CD86, such as containing an affinity modified IgSF domain, specifically binds to CD28 and/or CTLA-4 measured by flow cytometry with a binding affinity that yields a Mean Fluorescence Intensity (MFI) value at least 10%, 20%, 30%, 40%, 50%, 60%, 70%, 80%, 90%, or 100% greater than an unmodified CD86 control in a binding assay. In some embodiments, a variant CD86, such as containing an affinity modified IgSF domain, specifically binds to CD28 measured by flow cytometry with a binding affinity that yields a Mean Fluorescence Intensity (MFI) value at least 10%, 20%, 30%, 40%, 50%, 60%, 70%, 80%, 90%, or 100% greater than an unmodified CD86 control in a binding assay. In some embodiments, a variant CD86, such as containing an affinity modified IgSF domain, specifically binds to CTLA-4 measured by flow cytometry with a binding affinity that yields a Mean Fluorescence Intensity (MFI) value at least 10%, 20%, 30%, 40%, 50%, 60%, 70%, 80%, 90%, or 100% less than an unmodified CD86 control in a binding assay. In some embodiments, a variant CD86, such as containing an affinity modified IgSF domain, specifically binds to CTLA-4 measured by flow cytometry with a binding affinity that yields a Mean Fluorescence Intensity (MFI) value that is not significantly different from or is not greater than the binding affinity of an unmodified CD86 control in a binding assay. In some embodiments, a variant CD86, such as containing an affinity modified IgSF domain, specifically binds to CD28 measured by flow cytometry with a binding affinity that yields a Mean Fluorescence Intensity (MFI) value at least 10%, 20%, 30%, 40%, 50%, 60%, 70%, 80%, 90%, or 100% greater than an unmodified CD86 control in a binding assay and exhibits no change in binding affinity or a binding affinity that is not greater for CTLA-4 compared to the unmodified CD86 control in a binding assay. In some embodiments, a variant CD86, such as containing an affinity modified IgSF domain, specifically binds to CD28 measured by flow cytometry with a binding affinity that yields a Mean Fluorescence Intensity (MFI) value at least 10%, 20%, 30%, 40%, 50%, 60%, 70%, 80%, 90%, or 100% greater than an unmodified CD86 control in a binding assay, and exhibits a decrease in binding affinity for CTLA-4 measured by flow cytometry with a binding affinity that yields a Mean Fluorescence Intensity (MFI) value at least 10%, 20%, 30%, 40%, 50%, 60%, 70%, 80%, 90%, or 100% less than an unmodified CD86 control in a binding assay compared to the unmodified CD86 control in a binding assay.

The term “biological half-life” refers to the amount of time it takes for a substance, such as an immunomodulatory polypeptide containing a variant CD86 polypeptide of the present invention, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). Conjugates that can be used to derivatize and increase the biological half-life of polypeptides of the invention are known in the art and include, but are not limited to, polyethylene glycol (PEG), hydroxyethyl starch (HES), XTEN (extended recombinant peptides; see, WO2013130683), human serum albumin (HSA), bovine serum albumin (BSA), lipids (acylation), poly-Pro-Ala-Ser (PAS), and polyglutamic acid (glutamylation).

The term “chimeric antigen receptor” or “CAR” as used herein refers to an artificial (i.e., man-made) transmembrane protein expressed on a mammalian cell containing at least an ectodomain, a transmembrane, and an endodomain. Optionally, the CAR protein includes a “spacer” which covalently links the ectodomain to the transmembrane domain. A spacer is often a polypeptide linking the ectodomain to the transmembrane domain via peptide bonds. The CAR is typically expressed on a mammalian lymphocyte. In some embodiments, the CAR is expressed on a mammalian cell such as a T-cell or a tumor infiltrating lymphocyte (TIL). A CAR expressed on a T-cell is referred to herein as a “CAR T-cell” or “CAR-T.” In some embodiments the CAR-T is a T helper cell, a cytotoxic T-cell, a natural killer T-cell, a memory T-cell, a regulatory T-cell, or a gamma delta T-cell. When used clinically in, e.g., adoptive cell transfer, a CAR-T with antigen binding specificity to the patient's tumor is typically engineered to express on a native T-cell obtained from the patient. The engineered T-cell expressing the CAR is then infused back into the patient. The CAR-T is thus often an autologous CAR-T although allogeneic CAR-Ts are included within the scope of the invention. The ectodomain of a CAR contains an antigen binding region, such as an antibody or antigen binding fragment thereof (e.g., scFv), that specifically binds under physiological conditions with a target antigen, such as a tumor specific antigen. Upon specific binding a biochemical chain of events (i.e., signal transduction) results in modulation of the immunological activity of the CAR-T. Thus, for example, upon specific binding by the antigen binding region of the CAR-T to its target antigen can lead to changes in the immunological activity of the T-cell activity as reflected by changes in cytotoxicity, proliferation, or cytokine production. Signal transduction upon CAR-T activation is achieved in some embodiments by the CD3-zeta chain (“CD3-z”) which is involved in signal transduction in native mammalian T-cells. CAR-Ts can further contain multiple signaling domains such as CD28, 4-1BB, or OX40, to further modulate immunomodulatory response of the T-cell. CD3-z contains a conserved motif known as an immunoreceptor tyrosine-based activation motif (ITAM) which is involved in T-cell receptor signal transduction.

The term “collectively” or “collective” when used in reference to cytokine production induced by the presence of two or more variant CD86 polypeptides in an in vitro assay, means the overall cytokine expression level irrespective of the cytokine production induced by individual variant CD86 polypeptides. In some embodiments, the cytokine being assayed is IFN-gamma or IL-2 in an in vitro primary T-cell assay.

The term “cognate binding partner” (used interchangeably with “counter-structure”) in reference to a polypeptide, such as in reference to an IgSF domain of a variant CD86, refers to at least one molecule (typically a native mammalian protein) to which the referenced polypeptide specifically binds under specific binding conditions. In some aspects, a variant CD86 containing an affinity modified IgSF domain specifically binds to the counter-structure of the corresponding native or wildtype CD86 but with increased or attenuated affinity. A species of ligand recognized and specifically binding to its cognate receptor under specific binding conditions is an example of a counter-structure or cognate binding partner of that receptor. A “cognate cell surface binding partner” is a cognate binding partner expressed on a mammalian cell surface. A “cell surface molecular species” is a cognate binding partner of ligands of the immunological synapse (IS), expressed on and by cells, such as mammalian cells, forming the immunological synapse.

As used herein, “conjugate,” “conjugation” or grammatical variations thereof refer to the joining or linking together of two or more compounds resulting in the formation of another compound, by any joining or linking methods known in the art. It can also refer to a compound which is generated by the joining or linking together two or more compounds. For example, a variant CD86 polypeptide linked directly or indirectly to one or more chemical moieties or polypeptide is an exemplary conjugate. Such conjugates include fusion proteins, those produced by chemical conjugates and those produced by any other methods.

The term “competitive binding” as used herein means that a protein is capable of specifically binding to at least two cognate binding partners but that specific binding of one cognate binding partner inhibits, such as prevents or precludes, simultaneous binding of the second cognate binding partner. Thus, in some cases, it is not possible for a protein to bind the two cognate binding partners at the same time. Generally, competitive binders contain the same or overlapping binding site for specific binding but this is not a requirement. In some embodiments, competitive binding causes a measurable inhibition (partial or complete) of specific binding of a protein to one of its cognate binding partner due to specific binding of a second cognate binding partner. A variety of methods are known to quantify competitive binding such as ELISA (enzyme linked immunosorbent assay) assays.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

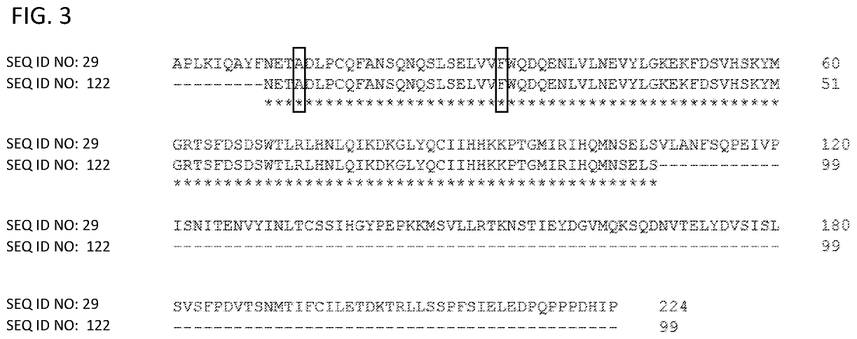

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues can be determined by alignment of a reference sequence with the sequence of wild-type CD86 set forth in SEQ ID NO: 29 (ECD domain) by structural alignment methods as described herein. By aligning the sequences, one skilled in the art can identify corresponding residues, for example, using conserved and identical amino acid residues as guides. FIG. 3 exemplifies alignment of a sequence with the reference sequence set forth in SEQ ID NO: 29 to identify corresponding residues. For example, in the exemplary alignment shown in FIG. 3, residue 13 of SEQ ID NO: 29 corresponds to residue 4 of SEQ ID NO: 122.

The terms “decrease” or “attenuate” or “suppress” as used herein means to decrease by a statistically significant amount. A decrease can be at least 10%, 20%, 30%, 40%, 50%, 60%, 70%, 80%, 90%, or 100%.

The terms “derivatives” or “derivatized” refer to modification of a protein by covalently linking it, directly or indirectly, to a composition so as to alter such characteristics as biological half-life, bioavailability, immunogenicity, solubility, toxicity, potency, or efficacy while retaining or enhancing its therapeutic benefit. Derivatives of immunomodulatory polypeptides of the invention are within the scope of the invention and can be made by, for example, glycosylation, PEGylation, lipidation, or Fc-fusion.

As used herein, detection includes methods that permit visualization (by eye or equipment) of a protein. A protein can be visualized using an antibody specific to the protein. Detection of a protein can also be facilitated by fusion of the protein with a tag including a label that is detectable or by contact with a second reagent specific to the protein, such as a secondary antibody, that includes a label that is detectable.

As used herein, domain (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains.

The term “ectodomain” as used herein refers to the region of a membrane protein, such as a transmembrane protein, that lies outside the vesicular membrane. Ectodomains often contain binding domains that specifically bind to ligands or cell surface receptors, such as via a binding domain that specifically binds to the ligand or cell surface receptor. The ectodomain of a cellular transmembrane protein is alternately referred to as an extracellular domain (ECD).

The terms “effective amount” or “therapeutically effective amount” refer to a quantity and/or concentration of a therapeutic composition of the invention, including a protein composition or cell composition, that when administered ex vivo (by contact with a cell from a patient) or in vivo (by administration into a patient) either alone (i.e., as a monotherapy) or in combination with additional therapeutic agents, yields a statistically significant decrease in disease progression as, for example, by ameliorating or eliminating symptoms and/or the cause of the disease. An effective amount may be an amount that relieves, lessens, or alleviates at least one symptom or biological response or effect associated with a disease or disorder, prevents progression of the disease or disorder, or improves physical functioning of the patient. In the case of cell therapy, the effective amount is an effective dose or number of cells administered to a patient by adoptive cell therapy. In some embodiments the patient is a mammal such as a non-human primate or human patient.

The term “endodomain” as used herein refers to the region found in some membrane proteins, such as transmembrane proteins, that extend into the interior space defined by the cell surface membrane. In mammalian cells, the endodomain is the cytoplasmic region of the membrane protein. In cells, the endodomain interacts with intracellular constituents and can be play a role in signal transduction and thus, in some cases, can be an intracellular signaling domain. The endodomain of a cellular transmembrane protein is alternately referred to as a cytoplasmic domain, which, in some cases, can be a cytoplasmic signaling domain.

The terms “enhanced” or “increased” as used herein in the context of increasing immunological activity of a mammalian lymphocyte means to increase one or more activities the lymphocyte. An increased activity can be one or more of increased cell survival, cell proliferation, cytokine production, or T-cell cytotoxicity, such as by a statistically significant amount. In some embodiments, reference to increased immunological activity means to increase interferon gamma (IFN-gamma), IL-2, or TNFα production, such as by a statistically significant amount. In some embodiments, the immunological activity can be assessed in a mixed lymphocyte reaction (MLR) assay. Methods of conducting MLR assays are known in the art. Wang et al., Cancer Immunol Res. 2014 September: 2(9):846-56. Other methods of assessing activities of lymphocytes are known in the art, including any assay as described herein. In some embodiments, an enhancement can be an increase of at least 10%, 20%, 30%, 40%, 50%, 75%, 100%, 200%, 300%, 400%, or 500% greater than a non-zero control value.

The term “engineered cell” as used herein refers to a mammalian cell that has been genetically modified by human intervention such as by recombinant DNA methods or viral transduction. In some embodiments, the cell is an immune cell, such as a lymphocyte (e.g., T cell, B cell, NK cell) or an antigen presenting cell (e.g., dendritic cell). The cell can be a primary cell from a patient or can be a cell line. In some embodiments, an engineered cell of the invention contains a variant CD86 of the invention engineered to modulate immunological activity of a T-cell expressing CD28 or CTLA-4 to which the variant CD86 polypeptide specifically binds. In some embodiments, the variant CD86 is a transmembrane immunomodulatory protein (hereinafter referred to as “TIP”) containing the extracellular domain or a portion thereof containing the IgV domain linked to a transmembrane domain (e.g., a CD86 transmembrane domain) and, optionally, an intracellular signaling domain. In some cases, the TIP is formatted as a chimeric receptor containing a heterologous cytoplasmic signaling domain or endodomain. In some embodiments, an engineered cell is capable of expressing and secreting an immunomodulatory protein as described herein. Among provided engineered cells also are cells further containing an engineered T-cell receptor (TCR) or chimeric antigen receptor (CAR).

The term “engineered T-cell” as used herein refers to a T-cell such as a T helper cell, cytotoxic T-cell (alternatively, cytotoxic T lymphocyte or CTL), natural killer T-cell, regulatory T-cell, memory T-cell, or gamma delta T-cell, that has been genetically modified by human intervention such as by recombinant DNA methods or viral transduction methods. An engineered T-cell contains a variant CD86 transmembrane immunomodulatory protein (TIP) or secreted immunomodulatory protein (SIP) of the present invention that is expressed on the T-cell and is engineered to modulate immunological activity of the engineered T-cell itself, or a mammalian cell to which the variant CD86 expressed on the T-cell specifically binds.

The term “engineered T-cell receptor” or “engineered TCR” refers to a T-cell receptor (TCR) engineered to specifically bind with a desired affinity to a major histocompatibility complex (MHC)/peptide target antigen that is selected, cloned, and/or subsequently introduced into a population of T-cells, often used for adoptive immunotherapy.

The term “expressed on” as used herein is used in reference to a protein expressed on the surface of a cell, such as a mammalian cell. Thus, the protein is expressed as a membrane protein. In some embodiments, the expressed protein is a transmembrane protein. In some embodiments, the protein is conjugated to a small molecule moiety such as a drug or detectable label. Proteins expressed on the surface of a cell can include cell-surface proteins such as cell surface receptors that are expressed on mammalian cells.

The term “half-life extending moiety” refers to a moiety of a polypeptide fusion or chemical conjugate that extends the half-life of a protein circulating in mammalian blood serum compared to the half-life of the protein that is not so conjugated to the moiety. In some embodiments, half-life is extended by greater than or greater than about 1.2-fold, 1.5-fold, 2.0-fold, 3.0-fold, 4.0-fold, 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization domain, polyethylene glycol (PEG), hydroxyethyl starch (HES), XTEN (extended recombinant peptides; see, WO2013130683), human serum albumin (HSA), bovine serum albumin (BSA), lipids (acylation), poly-Pro-Ala-Ser (PAS), and polyglutamic acid (glutamylation).

The term “immunological synapse” or “immune synapse” as used herein means the interface between a mammalian cell that expresses MHC I (major histocompatibility complex) or MHC II, such as an antigen-presenting cell or tumor cell, and a mammalian lymphocyte such as an effector T cell or a Natural Killer (NK) cell.

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). The Fc domain contains part or all of a hinge domain of an immunoglobulin molecule plus a CH2 and a CH3 domain. The Fc domain can form a dimer of two polypeptide chains joined by one or more disulfide bonds. In some embodiments, the Fc is a variant Fc that exhibits reduced (e.g., reduced greater than 30%, 40%, 50%, 60%, 70%, 80%, 90% or more) activity to facilitate an effector function. In some embodiments, reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics information System®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

An immunoglobulin Fc fusion (“Fc-fusion”), such as an immunomodulatory Fc fusion protein, is a molecule comprising one or more polypeptides (or one or more small molecules) operably linked to an Fc region of an immunoglobulin. An Fc-fusion may comprise, for example, the Fc region of an antibody (which, in some cases, facilitates pharmacokinetics) and a variant CD86 polypeptide. An immunoglobulin Fc region may be linked indirectly or directly to one or more variant CD86 polypeptides or small molecules (fusion partners). Various linkers are known in the art and can optionally be used to link an Fc to a fusion partner to generate an Fc-fusion. Fc-fusions of identical species can be dimerized to form Fc-fusion homodimers, or using non-identical species to form Fc-fusion heterodimers. In some embodiments, the Fc is a mammalian Fc such as a murine, rabbit or human Fc.

The term “host cell” refers to a cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO, DG44, Expi CHO, or CHOZN and related cell lines which grow in serum-free media or CHO strain DX-B11, which is deficient in DHFR. In some embodiments, a host cell can be a mammalian cell (e.g., a human cell, a monkey cell, a hamster cell, a rat cell, a mouse cell, or an insect cell).

The term “immunoglobulin” (abbreviated “Ig”) as used herein refers to a mammalian immunoglobulin protein including any of the five human classes of antibody: IgA (which includes subclasses IgAQ1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, such as antigen binding fragment (Fab), variable fragment (Fv) containing V_{H} and V_{L}, the single chain variable fragment (scFv) containing V_{H} and V_{L} linked together in one chain, as well as other antibody V region fragments, such as Fab′, F(ab)₂, F(ab′)₂, dsFv diabody, Fc, and Fd polypeptide fragments. Bispecific antibodies, homobispecific and heterobispecific, are included within the meaning of the term.

The term “immunoglobulin superfamily” or “IgSF” as used herein means the group of cell surface and soluble proteins that are involved in the recognition, binding, or adhesion processes of cells. Molecules are categorized as members of this superfamily based on shared structural features with immunoglobulins (i.e., antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system. Proteins in the immunological synapse are often members of the IgSF. IgSF can also be classified into “subfamilies” based on shared properties such as function. Such subfamilies typically consist of from 4 to 30 IgSF members.

The terms “IgSF domain” or “immunoglobulin domain” or “Ig domain” as used herein refers to a structural domain of IgSF proteins. Ig domains are named after the immunoglobulin molecules. They contain about 70-110 amino acids and are categorized according to their size and function. Ig-domains possess a characteristic Ig-fold, which has a sandwich-like structure formed by two sheets of antiparallel beta strands. Interactions between hydrophobic amino acids on the inner side of the sandwich and highly conserved disulfide bonds formed between cysteine residues in the B and F strands stabilize the Ig-fold. One end of the Ig domain has a section called the complementarity determining region that is important for the specificity of antibodies for their ligands. The Ig like domains can be classified (into classes) as: IgV, IgC1, IgC2, or IgI. Most Ig domains are either variable (IgV) or constant (IgC). IgV domains with 9 beta strands are generally longer than IgC domains with 7 beta strands. Ig domains of some members of the IgSF resemble IgV domains in the amino acid sequence, yet are similar in size to IgC domains. These are called IgC2 domains, while standard IgC domains are called IgC1 domains. T-cell receptor (TCR) chains contain two Ig domains in the extracellular portion; one IgV domain at the N-terminus and one IgC1 domain adjacent to the cell membrane. CD86 contains two Ig domains: IgV and IgC.

The term “IgSF species” as used herein means an ensemble of IgSF member proteins with identical or substantially identical primary amino acid sequence. Each mammalian immunoglobulin superfamily (IgSF) member defines a unique identity of all IgSF species that belong to that IgSF member. Thus, each IgSF family member is unique from other IgSF family members and, accordingly, each species of a particular IgSF family member is unique from the species of another IgSF family member. Nevertheless, variation between molecules that are of the same IgSF species may occur owing to differences in post-translational modification such as glycosylation, phosphorylation, ubiquitination, nitrosylation, methylation, acetylation, and lipidation. Additionally, minor sequence differences within a single IgSF species owing to gene polymorphisms constitute another form of variation within a single IgSF species as do wild type truncated forms of IgSF species owing to, for example, proteolytic cleavage. A “cell surface IgSF species” is an IgSF species expressed on the surface of a cell, generally a mammalian cell.

The term “immunological activity” as used herein in the context of mammalian lymphocytes such as T-cells refers to one or more cell survival, cell proliferation, cytokine production (e.g., interferon-gamma), or T-cell cytotoxicity activities. In some cases, an immunological activity can means their expression of cytokines, such as chemokines or interleukins. Assays for determining enhancement or suppression of immunological activity include the MLR (mixed lymphocyte reaction) assays measuring cytokine levels, such as interferon-gamma or IL-2, in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104). Since T cell activation is associated with secretion of cytokines, such as IFN-gamma or IL-2 cytokines, detecting such cytokine levels in culture supernatants from these in vitro human T cell assays can be assayed using commercial ELISA kits (Wu et al, Immunol Lett 2008 Apr. 15; 117(1): 57-62). Induction of an immune response results in an increase in immunological activity relative to quiescent lymphocytes. An immunomodulatory protein, such as a variant CD86 polypeptide containing an affinity modified IgSF domain, as provided herein can in some embodiments increase or, in alternative embodiments, decrease IFN-gamma (interferon-gamma) or IL-2 expression in a primary T-cell assay relative to a wild-type IgSF member or IgSF domain control. Those of skill will recognize that the format of the primary T-cell assay used to determine an increase in IFN-gamma or IL-2 expression will differ from that employed to assay for a decrease in IFN-gamma or IL-2 expression. In assaying for the ability of an immunomodulatory protein or affinity modified IgSF domain of the invention to decrease IFN-gamma or IL-2 expression in a primary T-cell assay, a Mixed Lymphocyte Reaction (MLR) assay can be used. Conveniently, a soluble form of an affinity modified IgSF domain of the invention can be employed to determine its ability to antagonize and thereby decrease the IFN-gamma or IL-2 expression in a MLR. Alternatively, in assaying for the ability of an immunomodulatory protein or affinity modified IgSF domain of the invention to increase IFN-gamma or IL-2 expression in a primary T-cell assay, a co-immobilization assay can be used. In a co-immobilization assay, a T-cell receptor signal, provided in some embodiments by anti-CD3 antibody, is used in conjunction with a co-immobilized affinity modified IgSF domain, such as a variant CD86, to determine the ability to increase IFN-gamma or IL-2 expression relative to a wild-type IgSF domain control. Methods to assay the immunological activity of engineered cells, including to evaluate the activity of a variant CD86 transmembrane immunomodulatory protein, are known in the art and include, but are not limited to, the ability to expand T cells following antigen stimulation, sustain T cell expansion in the absence of re-stimulation, and anti-cancer activities in appropriate animal models. Assays also include assays to assess cytotoxicity, including a standard ⁵¹Cr-release assay (see e.g., Milone et al., (2009) Molecular Therapy 17: 1453-1464) or flow based cytotoxicity assays, or an impedance based cytotoxicity assay (Peper et al. (2014) Journal of Immunological Methods, 405:192-198).

An “immunomodulatory polypeptide” or “immunomodulatory protein” is a polypeptide or protein molecule that modulates immunological activity. By “modulation” or “modulating” an immune response is meant that immunological activity is either increased or decreased. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric polypeptides are within the scope of the defined term. Multimeric polypeptides can be homomultimeric (of identical polypeptide chains) or heteromultimeric (of non-identical polypeptide chains). An immunomodulatory protein herein comprises a variant CD86 polypeptide.

The term “increase” as used herein means to increase by a statistically significant amount. An increase can be at least 5%, 10%, 20%, 30%, 40%, 50%, 75%, 100%, or greater than a non-zero control value.

An “isoform” of CD86 is one of a plurality of naturally occurring CD86 polypeptides that differ in amino acid sequence. Isoforms can be the product of splice variants of an RNA transcript expressed by a single gene, or the expression product of highly similar but different genes yielding a functionally similar protein such as may occur from gene duplication. As used herein, the term “isoform” of CD86 also refers to the product of different alleles of a CD86 gene.

The term “label” refers to a compound or composition which can be attached or linked, directly or indirectly to provide a detectable signal or that can interact with a second label to modify a detectable signal. The label can be conjugated directly or indirectly to a polypeptide so as to generate a labeled polypeptide. The label can be detectable by itself (e.g., radioisotope labels or fluorescent labels) or, in the case of an enzymatic label, can catalyze chemical alteration of a substrate compound composition which is detectable. Non-limiting examples of labels included fluorogenic moieties, green fluorescent protein, or luciferase.

The term “lymphocyte” as used herein means any of three subtypes of white blood cell in a mammalian immune system. They include natural killer cells (NK cells) (which function in cell-mediated, cytotoxic innate immunity), T cells (for cell-mediated, cytotoxic adaptive immunity), and B cells (for humoral, antibody-driven adaptive immunity). T cells include: T helper cells, cytotoxic T-cells, natural killer T-cells, memory T-cells, regulatory T-cells, or gamma delta T-cells. Innate lymphoid cells (ILC) are also included within the definition of lymphocyte.

The terms “mammal,” or “patient” specifically includes reference to at least one of a: human, chimpanzee, rhesus monkey, cynomolgus monkey, dog, cat, mouse, or rat.

The term “membrane protein” as used herein means a protein that, under physiological conditions, is attached directly or indirectly to a lipid bilayer. A lipid bilayer that forms a membrane can be a biological membrane such as a eukaryotic (e.g., mammalian) cell membrane or an artificial (i.e., man-made) membrane such as that found on a liposome. Attachment of a membrane protein to the lipid bilayer can be by way of covalent attachment, or by way of non-covalent interactions such as hydrophobic or electrostatic interactions. A membrane protein can be an integral membrane protein or a peripheral membrane protein. Membrane proteins that are peripheral membrane proteins are non-covalently attached to the lipid bilayer or non-covalently attached to an integral membrane protein. A peripheral membrane protein forms a temporary attachment to the lipid bilayer such that under the range of conditions that are physiological in a mammal, a peripheral membrane protein can associate and/or disassociate from the lipid bilayer. In contrast to peripheral membrane proteins, integral membrane proteins form a substantially permanent attachment to the membrane's lipid bilayer such that under the range of conditions that are physiological in a mammal, integral membrane proteins do not disassociate from their attachment to the lipid bilayer. A membrane protein can form an attachment to the membrane by way of one layer of the lipid bilayer (monotopic), or attached by way of both layers of the membrane (polytopic). An integral membrane protein that interacts with only one lipid bilayer is an “integral monotopic protein”. An integral membrane protein that interacts with both lipid bilayers is an “integral polytopic protein” alternatively referred to herein as a “transmembrane protein”.

The terms “modulating” or “modulate” as used herein in the context of an immune response, such as a mammalian immune response, refer to any alteration, such as an increase or a decrease, of existing or potential immune responses that occurs as a result of administration of an immunomodulatory polypeptide comprising a variant CD86 of the present invention or as a result of administration of engineered cells expresses an immunomodulatory protein, such as a variant CD86 transmembrane immunomodulatory protein of the present invention. Thus, it refers to an alteration, such as an increase or decrease, of an immune response as compared to the immune response that occurs or is present in the absence of the administration of the immunomodulatory protein comprising the variant CD86. Such modulation includes any induction, activation, suppression, or alteration in degree or extent of immunological activity of an immune cell. Immune cells include B cells, T cells, NK (natural killer) cells, NK T cells, professional antigen-presenting cells (APCs), non-professional antigen-presenting cells, and inflammatory cells (neutrophils, macrophages, monocytes, eosinophils, and basophils). Modulation includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response. Modulation of an immune response or modulation of immunological activity includes, for example, the following: elimination, deletion, or sequestration of immune cells; induction or generation of immune cells that can modulate the functional capacity of other cells such as autoreactive lymphocytes, antigen presenting cells, or inflammatory cells; induction of an unresponsive state in immune cells (i.e., anergy); enhancing or suppressing the activity or function of immune cells, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors or any combination of these modulatory events. Modulation can be assessed, for example, by an alteration in IFN-gamma (interferon gamma) or IL-2 expression relative to the wild-type or unmodified CD86 control in a primary T cell assay (see, Zhao and Ji, Exp Cell Res. 2016 Jan. 1; 340(1): 132-138). Modulation can be assessed, for example, by an alteration of an immunological activity of engineered cells, such as an alteration in in cytotoxic activity of engineered cells or an alteration in cytokine secretion of engineered cells relative to cells engineered with a wild-type CD86 transmembrane protein.

The term, a “multimerization domain” refers to a sequence of amino acids that promotes stable interaction of a polypeptide molecule with one or more additional polypeptide molecules, each containing a complementary multimerization domain (e.g., a first multimerization domain and a second multimerization domain), which can be the same or a different multimerization domain. The interactions between complementary multimerization domains, e.g., interaction between a first multimerization domain and a second multimerization domain, form a stable protein-protein interaction to produce a multimer of the polypeptide molecule with the additional polypeptide molecule. In some cases, the multimerization domain is the same and interacts with itself to form a stable protein-protein interaction between two polypeptide chains. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3, or IgG4 subtypes, IgA, IgE, IgD, IgM and modified forms thereof.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally-occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated (a “reference sequence”). Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The term “molecular species” as used herein means an ensemble of proteins with identical or substantially identical primary amino acid sequence. Each mammalian immunoglobulin superfamily (IgSF) member defines a collection of identical or substantially identical molecular species. Thus, for example, human CD86 is an IgSF member and each human CD86 molecule is a molecular species of CD86. Variation between molecules that are of the same molecular species may occur owing to differences in post-translational modification such as glycosylation, phosphorylation, ubiquitination, nitrosylation, methylation, acetylation, and lipidation. Additionally, minor sequence differences within a single molecular species owing to gene polymorphisms constitute another form of variation within a single molecular species as do wild type truncated forms of a single molecular species owing to, for example, proteolytic cleavage. A “cell surface molecular species” is a molecular species expressed on the surface of a mammalian cell. Two or more different species of protein, each of which is present exclusively on one or exclusively the other (but not both) of the two mammalian cells forming the IS, are said to be in “cis” or “cis configuration” with each other. Two different species of protein, the first of which is exclusively present on one of the two mammalian cells forming the IS and the second of which is present exclusively on the second of the two mammalian cells forming the IS, are said to be in “trans” or “trans configuration.” Two different species of protein each of which are present on both of the two mammalian cells forming the IS are in both cis and trans configurations on these cells.

The term “non-competitive binding” as used herein means the ability of a protein to specifically bind simultaneously to at least two cognate binding partners. Thus, the protein is able to bind to at least two different cognate binding partners at the same time, although the binding interaction need not be for the same duration such that, in some cases, the protein is specifically bound to only one of the cognate binding partners. In some embodiments, the binding occurs under specific binding conditions. In some embodiments, the simultaneous binding is such that binding of one cognate binding partner does not substantially inhibit simultaneous binding to a second cognate binding partner. In some embodiments, non-competitive binding means that binding a second cognate binding partner to its binding site on the protein does not displace the binding of a first cognate binding partner to its binding site on the protein. Methods of assessing non-competitive binding are well known in the art such as the method described in Perez de La Lastra et al., Immunology, 1999 April: 96(4): 663-670. In some cases, in non-competitive interactions, the first cognate binding partner specifically binds at an interaction site that does not overlap with the interaction site of the second cognate binding partner such that binding of the second cognate binding partner does not directly interfere with the binding of the first cognate binding partner. Thus, any effect on binding of the cognate binding partner by the binding of the second cognate binding partner is through a mechanism other than direct interference with the binding of the first cognate binding partner. For example, in the context of enzyme-substrate interactions, a non-competitive inhibitor binds to a site other than the active site of the enzyme. Non-competitive binding encompasses uncompetitive binding interactions in which a second cognate binding partner specifically binds at an interaction site that does not overlap with the binding of the first cognate binding partner but binds to the second interaction site only when the first interaction site is occupied by the first cognate binding partner.

The term “pharmaceutical composition” refers to a composition suitable for pharmaceutical use in a mammalian subject, often a human. A pharmaceutical composition typically comprises an effective amount of an active agent (e.g., an immunomodulatory polypeptide comprising a variant CD86 or engineered cells expressing a variant CD86 transmembrane immunomodulatory protein) and a carrier, excipient, or diluent. The carrier, excipient, or diluent is typically a pharmaceutically acceptable carrier, excipient or diluent, respectively.

The terms “polypeptide” and “protein” are used interchangeably herein and refer to a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylation, acetylation, phosphorylation and the like. The terms also include molecules in which one or more amino acids are amino acid analogs or non-canonical or unnatural amino acids that can be synthesized or expressed recombinantly using known protein engineering techniques. In addition, proteins can be derivatized.

The term “primary T-cell assay” as used herein refers to an in vitro assay to measure a T cell activity, such as production of cytokines, for example interferon-gamma (“IFN-gamma”) IL-2, or tumor necrosis factor alpha (TNFα) expression. A variety of such primary T-cell assays are known in the art. In some embodiments, the assay used is an anti-CD3 coimmobilization assay. In this assay, primary T cells are stimulated by anti-CD3 immobilized with or without additional recombinant proteins. Culture supernatants are harvested at timepoints, usually 24-72 hours. In another embodiment, the assay used is a mixed lymphocyte reaction (MLR). In this assay, primary T cells are simulated with allogenic APC. Culture supernatants are harvested at timepoints, usually 24-72 hours. Cytokine levels, such as levels of IFN-gamma, IL-2, or TNFα, are measured in culture supernatants by standard ELISA techniques. Commercial kits are available from vendors and the assay is performed according to manufacturer's recommendation.

The term “purified” as applied to nucleic acids, such as encoding immunomodulatory proteins of the invention, generally denotes a nucleic acid or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or protein of the invention is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as a transmembrane immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. The terms “in operable combination,” “in operable order” and “operably linked” as used herein refer to the linkage of nucleic acid sequences in such a manner or orientation that a nucleic acid molecule capable of directing the transcription of a given gene and/or the synthesis of a desired protein molecule is produced.

The term “recombinant expression vector” as used herein refers to a DNA molecule containing a desired coding sequence and appropriate nucleic acid sequences necessary for the expression of the operably linked coding sequence in a particular host cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence for the recombinant protein, such as a recombinant fusion protein, so that the expressed fusion protein can be secreted by the recombinant host cell, for easier isolation of the fusion protein from the cell, if desired. The term includes the vector as a self-replicating nucleic acid structure as well as the vector incorporated into the genome of a host cell into which it has been introduced. Among the vectors are viral vectors, such as lentiviral vectors.

The term “selectivity” refers to the preference of a subject protein, or polypeptide, for specific binding of one substrate, such as one cognate binding partner, compared to specific binding for another substrate, such as a different cognate binding partner of the subject protein. Selectivity can be reflected as a ratio of the binding activity (e.g., binding affinity) of a subject protein and a first substrate, such as a first cognate binding partner, (e.g., K_{d1}) and the binding activity (e.g., binding affinity) of the same subject protein with a second cognate binding partner (e.g., K_{d2}).

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website.

The term “soluble” as used herein in reference to proteins, means that the protein is not a membrane protein. In general, a soluble protein contains only the extracellular domain of an IgSF family member receptor, or a portion thereof containing an IgSF domain or domains or specific-binding fragments thereof, but does not contain the transmembrane domain. In some cases, solubility of a protein can be improved by linkage or attachment, directly or indirectly via a linker, to an Fc domain, which, in some cases, also can improve the stability and/or half-life of the protein. In some aspects, a soluble protein is an Fc fusion protein.

The term “species” as used herein with respect to polypeptides or nucleic acids means an ensemble of molecules with identical or substantially identical sequences. Variation between polypeptides that are of the same species may occur owing to differences in post-translational modification such as glycosylation, phosphorylation, ubiquitination, nitrosylation, methylation, acetylation, and lipidation. Slightly truncated sequences of polypeptides that differ (or encode a difference) from the full length species at the amino-terminus or carboxyl-terminus by no more than 1, 2, or 3 amino acid residues are considered to be of a single species. Such microheterogeneities are a common feature of manufactured proteins.

The term “specific binding fragment” as used herein in reference to a full-length wild-type mammalian CD86 polypeptide or an ECD, IgV or an IgC domain thereof, means a polypeptide having a subsequence of an ECD, IgV and/or IgC domain and that specifically binds in vitro and/or in vivo to a mammalian CD28 and/or mammalian CTLA-4, such as a human or murine CD28 and/or CTLA-4. In some embodiments, the specific binding fragment of the CD86 ECD, CD86 IgV, or the CD86 IgC is at least 60%, 70%, 75%, 80%, 85%, 90%, 95%, 96%, 97%, 98%, or 99% the sequence length of the full-length wild-type ECD, IgV, or IgC sequence. The specific binding fragment can be altered in sequence to form the variant CD86.

The term “specifically binds” as used herein means the ability of a protein, under specific binding conditions, to bind to a target protein such that its affinity or avidity is at least 5 times as great, but optionally at least 10, 20, 30, 40, 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a non-target molecule having a substantially similar epitope as the target molecule (e.g., paralog) is possible and does not detract from the specificity of binding which is determined relative to a statistically valid collection of unique non-targets (e.g., random polypeptides). Thus, a polypeptide of the invention may specifically bind to more than one distinct species of target molecule due to cross-reactivity. Solid-phase ELISA immunoassays or surface plasmon resonance (e.g., Biacore) measurements can be used to determine specific binding between two proteins. Generally, interactions between two binding proteins have dissociation constants (K_{d}) less than 1×10⁻⁵ M, and often as low as 1×10⁻¹² M. In certain embodiments of the present disclosure, interactions between two binding proteins have dissociation constants of 1×10⁻⁶ M, 1×10⁻⁷ M, 1×10⁻⁸ M, 1×10⁻⁹ M, 1×10⁻¹⁰ M or 1×10⁻¹¹ M.

The terms “surface expresses” or “surface expression” in reference to a mammalian cell expressing a polypeptide means that the polypeptide is expressed as a membrane protein. In some embodiments, the membrane protein is a transmembrane protein.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “targeting moiety” as used herein refers to a composition that is covalently or non-covalently attached to, or physically encapsulates, a polypeptide comprising the variant CD86. The targeting moiety has specific binding affinity for a desired counter-structure such as a cell surface receptor (e.g., CD28), or a tumor antigen such as a tumor specific antigen (TSA) or a tumor associated antigen (TAA) such as B7-H6. Typically, the desired counter-structure is localized on a specific tissue or cell-type. Targeting moieties include: antibodies, antigen binding fragment (Fab), variable fragment (Fv) containing V_{H} and V_{L}, the single chain variable fragment (scFv) containing V_{H} and V_{L} linked together in one chain, as well as other antibody V region fragments, such as Fab′, F(ab)₂, F(ab′)₂, dsFv diabody, nanobodies, soluble receptors, receptor ligands, affinity matured receptors or ligands, as well as small molecule (<500 Dalton) compositions (e.g., specific binding receptor compositions). Targeting moieties can also be attached covalently or non-covalently to the lipid membrane of liposomes that encapsulate a polypeptide of the present invention.

The term “transmembrane protein” as used herein means a membrane protein that substantially or completely spans a lipid bilayer such as those lipid bilayers found in a biological membrane such as a mammalian cell, or in an artificial construct such as a liposome. The transmembrane protein comprises a transmembrane domain (“transmembrane domain”) by which it is integrated into the lipid bilayer and by which the integration is thermodynamically stable under physiological conditions. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through the both layers of the lipid bilayer once or multiple times. A transmembrane protein includes the provided transmembrane immunomodulatory proteins described herein. In addition to the transmembrane domain, a transmembrane immunomodulatory protein of the invention further comprises an ectodomain and, in some embodiments, an endodomain.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of a therapeutic composition (e.g., containing an immunomodulatory protein or engineered cells) of the invention either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. As used herein in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS). “Preventing,” “prophylaxis,” or “prevention” of a disease or disorder as used in the context of this invention refers to the administration of an immunomodulatory polypeptide or engineered cells of the invention, either alone or in combination with another compound, to prevent the occurrence or onset of a disease or disorder or some or all of the symptoms of a disease or disorder or to lessen the likelihood of the onset of a disease or disorder.

The term “tumor specific antigen” or “TSA” as used herein refers to a counter-structure that is present primarily on tumor cells of a mammalian subject but generally not found on normal cells of the mammalian subject. A tumor specific antigen need not be exclusive to tumor cells but the percentage of cells of a particular mammal that have the tumor specific antigen is sufficiently high or the levels of the tumor specific antigen on the surface of the tumor are sufficiently high such that it can be targeted by anti-tumor therapeutics, such as immunomodulatory polypeptides of the invention, and provide prevention or treatment of the mammal from the effects of the tumor. In some embodiments, in a random statistical sample of cells from a mammal with a tumor, at least 50% of the cells displaying a TSA are cancerous. In other embodiments, at least 60%, 70%, 80%, 85%, 90%, 95%, or 99% of the cells displaying a TSA are cancerous.

The term “variant” (also “modified” or mutant”) as used in reference to a variant CD86 means a CD86, such as a mammalian (e.g., human or murine) CD86 created by human intervention. The variant CD86 is a polypeptide having an altered amino acid sequence, relative to an unmodified or wild-type CD86. The variant CD86 is a polypeptide which differs from a wild-type CD86 isoform sequence by one or more amino acid substitutions, deletions, additions, or combinations thereof. For purposes herein, the variant CD86 contains at least one affinity modified domain, whereby one or more of the amino acid differences occurs in an IgSF domain (e.g., IgV domain or IgC domain). A variant CD86 can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions. A variant CD86 polypeptide generally exhibits at least 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified CD86, such as to the sequence of SEQ ID NO: 2, a mature sequence thereof or a portion thereof containing the extracellular domain or an IgSF domain thereof. In some embodiments, a variant CD86 polypeptide exhibits at least 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified CD86 comprising the sequence set forth in SEQ ID NO: 2, SEQ ID NO: 29, SEQ ID NO: 122, or SEQ ID NO: 123.

Non-naturally occurring amino acids as well as naturally occurring amino acids are included within the scope of permissible substitutions or additions. A variant CD86 is not limited to any particular method of making and includes, for example, de novo chemical synthesis, de novo recombinant DNA techniques, or combinations thereof. A variant CD86 of the invention specifically binds to at least one or more of: CD28 and/or CTLA-4 of a mammalian species. In some embodiments, the altered amino acid sequence results in an altered (i.e., increased or decreased) binding affinity or avidity to CD28 and/or CTLA-4 compared to the unmodified or wild-type CD86 protein. An increase or decrease in binding affinity or avidity can be determined using well known binding assays such as flow cytometry. Larsen et al., American Journal of Transplantation, Vol 5: 443-453 (2005). See also, Linsley et al., Immunity, Vol 1(9): 793-801 (1994). An increase in variant CD86 binding affinity or avidity to CD28 and/or CTLA-4 can be a value at least 5% greater than that of the unmodified or wild-type CD86 and in some embodiments, at least 10%, 15%, 20%, 30%, 40%, 50%, 100% greater than that of the unmodified or wild-type CD86 control value. A decrease in CD86 binding affinity or avidity to CD28 and/or CTLA-4 is to a value no greater than 95% of the of the unmodified or wild-type CD86 control values, and in some embodiments no greater than 80%, 70% 60%, 50%, 40%, 30%, 20%, 10%, 5%, or no detectable binding affinity or avidity of the unmodified or wild-type CD86 control values. In some embodiments, no change in binding affinity or avidity is seen as a lack of significant difference between the binding affinity or avidity of the variant CD86 and the binding affinity or avidity of the unmodified or wild-type CD86. In some embodiments, binding affinity or avidity can be altered for one cognate binding partner and not the other cognate binding partner. For example, a variant CD86 may exhibit increased binding affinity or avidity for CD28, show no change binding affinity or avidity to CTLA-4 compared to the binding affinity or avidity of a wild-type or unmodified CD86 molecule. In some embodiments, binding affinity or avidity can be altered for both cognate binding partners. In some embodiments, the alteration is in the same direction (e.g., both increase or decrease). In some embodiments, the alteration is in different directions (e.g., increased for one cognate binding partner and decreased for the other cognate binding partner). For example, a variant CD86 may exhibit increased binding affinity or avidity for CD28, show decreased binding affinity or avidity to CTLA-4 compared to the binding affinity or avidity of a wild-type or unmodified CD86 molecule. In some embodiments, the CD86 variant or wild-type or unmodified polypeptide binds to the ectodomain of CD28 and/or CTLA-4. Thus, in some embodiments, affinity and avidity are determined based on binding of the CD86 variant or wild-type or unmodified polypeptide to the ectodomain of CD28 and/or CTLA-4. A variant CD86 polypeptide is altered in primary amino acid sequence by substitution, addition, or deletion of amino acid residues. The term “variant” in the context of variant CD86 polypeptide is not to be construed as imposing any condition for any particular starting composition or method by which the variant CD86 is created. A variant CD86 can, for example, be generated starting from wild type mammalian CD86 sequence information, then modeled in silico for binding to CD28 and/or CTLA-4, and finally recombinantly or chemically synthesized to yield the variant CD86. In one alternative example, the variant CD86 can be created by site-directed mutagenesis of an unmodified or wild-type CD86. Thus, variant CD86 denotes a composition and not necessarily a product produced by any given process. A variety of techniques including recombinant methods, chemical synthesis, or combinations thereof, may be employed.

The term “wild-type” or “natural” or “native” as used herein is used in connection with biological materials such as nucleic acid molecules, proteins (e.g., CD86), IgSF members, host cells, and the like, refers to those which are found in nature and not modified by human intervention.

### II. VARIANT CD86 POLYPEPTIDES

Provided herein are variant CD86 polypeptides that exhibit altered (increased or decreased) binding activity or affinity for one or more of a CD86 cognate binding partner. In some embodiments, the CD86 cognate binding partner is CD28 or CTLA-4. In some embodiments, the CD86 cognate binding partner is CD28. In some embodiments, the variant CD86 polypeptide contains one or more amino acids modifications, such as one or more substitutions (alternatively, “mutations” or “replacements”), deletions or additions, in an immunoglobulin superfamily (IgSF) domain (IgD) relative to a wild-type or unmodified CD86 polypeptide or a portion of a wild-type or unmodified CD86 containing the IgD or a specific binding fragment thereof. Thus, a provided variant CD86 polypeptide is or comprises a variant IgD (hereinafter called “vIgD”) in which the one or more amino acid modifications (e.g. substitutions) is in an IgD.

In some embodiments, the variant is modified in one more IgSF domains relative to the sequence of an unmodified CD86 sequence. In some embodiments, the unmodified CD86 sequence is a wild-type CD86. In some embodiments, the unmodified or wild-type CD86 has the sequence of a native CD86 or an ortholog thereof. In some embodiments, the unmodified CD86 is or comprises the extracellular domain (ECD) of CD86 or a portion thereof containing an IgV domain (see Table 2). In some embodiments, the variant CD86 is or contains the extracellular domain (ECD) of CD86 or a portion thereof containing an IgV domain. In some embodiments, the unmodified or wild-type CD86 polypeptide contains the IgV domain or a specific binding fragment thereof. In some embodiments, the variant CD86 polypeptide contains the IgV domain or a specific binding fragment thereof. In some embodiments, the variant CD86 is soluble and lacks a transmembrane domain. In some embodiments, the variant CD86 further comprises a transmembrane domain and, in some cases, also a cytoplasmic domain.

In some embodiments, the wild-type or unmodified CD86 sequence is a mammalian CD86 sequence. In some embodiments, the wild-type or unmodified CD86 sequence can be a mammalian CD86 that includes, but is not limited to, human, mouse, cynomolgus monkey, or rat. In some embodiments, the wild-type or unmodified CD86 sequence is human.

In some embodiments, the wild-type or unmodified CD86 sequence has (i) the sequence of amino acids set forth in SEQ ID NO: 2 or a mature form thereof lacking the signal sequence, (ii) a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or the mature form thereof, or (iii) is a portion of (i) or (ii) containing an IgV domain or specific binding fragment thereof.

In some embodiments, the wild-type or unmodified CD86 sequence is or comprises an extracellular domain or portion thereof containing the IgV of the CD86 or a specific binding fragment thereof. In some embodiments, the unmodified or wild-type CD86 polypeptide comprises the amino acid sequence set forth in SEQ ID NO: 29, 122, or 123, or an ortholog thereof. In some cases, the unmodified or wild-type CD86 polypeptide can comprise (i) the sequence of amino acids set forth in SEQ ID NO: 29, 122, or 123, (ii) a sequence of amino acids that has at least about 85%, 86%, 87%, 88%, 89%, 90%, 91%, 92%, 93%, 94%, 95%, 96%, 97%, 98%, 99% sequence identity to SEQ ID NO: 29, 122, or 123, or (iii) is a specific binding fragment of the sequence of (i) or (ii). In some embodiments, the wild-type or unmodified CD86 polypeptide comprises the amino acid sequence set forth in SEQ ID NO: 29 (corresponding to amino acid residues 24-247 of SEQ ID NO: 2), or an ortholog thereof. In some embodiments, the wild-type or unmodified CD86 polypeptide comprises the amino acid sequence set forth in SEQ ID NO: 122 (corresponding to amino acid residues 33-131 of SEQ ID NO: 2), or an ortholog thereof. In some embodiments, the wild-type or unmodified CD86 polypeptide comprises the amino acid sequence set forth in SEQ ID NO: 123 (corresponding to amino acid residues 24-134 of SEQ ID NO: 2), or an ortholog thereof. In some embodiments, the wild-type or unmodified CD86 containing the IgV domain or specific binding fragment thereof is capable of binding one or more CD86 cognate binding proteins, such as one or more of CD28 or CTLA-4.

In some embodiments, the wild-type or unmodified CD86 polypeptide contains a specific binding fragment of CD86, such as a specific binding fragment of the IgV domain. In some embodiments the specific binding fragment can bind CD28 and/or CTLA-4. In some embodiments, the specific binding fragment can bind the ectodomain of CD28 and/or CTLA-4. The specific binding fragment can have an amino acid length of at least 50 amino acids, such as at least 60, 70, 80, 90, 100, 110, 120, 130, 140, 150, 160, 170, 180, 190, 200, 210, or 220 amino acids. In some embodiments, a specific binding fragment of the IgV domain contains an amino acid sequence that is at least about 85%, 86%, 87%, 88%, 89%, 90%, 91%, 92%, 93%, 94%, 95%, 96%, 97%, 98%, 99% of the length of the IgV domain set forth as amino acids 33-131 of SEQ ID NO: 2.

In some embodiments, the variant CD86 polypeptide comprises an extracellular domain or a portion thereof comprising one or more affinity modified IgSF domains. In some embodiments, the variant CD86 polypeptides can comprise an IgV domain, or a specific binding fragment of the IgV domain in which the IgSF domain contains the one or more amino acid modifications (e.g. substitutions). In some embodiments, the variant CD86 polypeptide comprises a full-length IgV domain. In some embodiments, the variant CD86 polypeptide comprises a specific binding fragment of the IgV domain. In some embodiments, the variant CD86 polypeptide comprises a full-length extracellular domain (ECD). In some embodiments, the variant CD86 polypeptide comprises a specific binding fragment of the ECD domain. In some embodiments, the variant CD86 polypeptide comprises a specific binding fragment of the ECD domain comprising the full length IgV domain. In some embodiments, the variant CD86 polypeptide comprises a specific binding fragment of the ECD domain comprising a specific binding fragment of the IgV domain.

Generally, each of the various attributes of polypeptides are separately disclosed below (e.g., soluble and membrane bound polypeptides, affinity of CD86 for CD28 and CTLA-4, number of variations per polypeptide chain, number of linked polypeptide chains, the number and nature of amino acid alterations per variant CD86, etc.). However, as will be clear to the skilled artisan, any particular polypeptide can comprise a combination of these independent attributes. It is understood that reference to amino acids, including to a specific sequence set forth as a SEQ ID NO used to describe domain organization of an IgSF domain are for illustrative purposes and are not meant to limit the scope of the embodiments provided. It is understood that polypeptides and the description of domains thereof are theoretically derived based on homology analysis and alignments with similar molecules. Thus, the exact locus can vary, and is not necessarily the same for each protein. Hence, the specific IgSF domain, such as specific IgV domain, can be several amino acids (such as one, two, three or four) longer or shorter.

Further, various embodiments of the invention as discussed below are frequently provided within the meaning of a defined term as disclosed above. The embodiments described in a particular definition are therefore to be interpreted as being incorporated by reference when the defined term is utilized in discussing the various aspects and attributes described herein. Thus, the headings, the order of presentation of the various aspects and embodiments, and the separate disclosure of each independent attribute is not meant to be a limitation to the scope of the present disclosure.

### A. Exemplary Modifications

Provided herein are variant CD86 polypeptides containing at least one affinity-modified IgSF domain (e.g., IgV) or a specific binding fragment thereof relative to an IgSF domain contained in a wild-type or unmodified CD86 polypeptide such that the variant CD86 polypeptide exhibits altered (increased or decreased) binding activity or affinity for one or more ligands CD28 or CTLA-4 compared to a wild-type or unmodified CD86 polypeptide. In some embodiments, a variant CD86 polypeptide has a binding affinity for CD28 and/or CTLA-4 that differs from that of a wild-type or unmodified CD86 polypeptide control sequence as determined by, for example, solid-phase ELISA immunoassays, flow cytometry, ForteBio Octet or Biacore assays. In some embodiments, the variant CD86 polypeptide has an increased binding affinity for CD28, relative to a wild-type or unmodified CD86 polypeptide. In some embodiments, the variant CD86 polypeptide has a decreased binding affinity for CTLA-4, relative to a wild-type or unmodified CD86 polypeptide. In some embodiments, the variant CD86 polypeptide exhibits no change in binding affinity for CTLA-4, relative to a wild-type or unmodified CD86 polypeptide. In some embodiments, the variant CD86 polypeptide exhibits no increase in binding affinity for CTLA-4, relative to a wild-type or unmodified CD86 polypeptide. The CD28 and/or the CTLA-4 can be a mammalian protein, such as a human protein or a murine protein. In some embodiments, the variant, wild-type, and unmodified CD86 polypeptides bind to the ectodomain of CD28 and/or CTLA-4. Thus, in some embodiments, affinity or binding activity is determined with respect to the binding of variant, wild-type, and unmodified CD86 polypeptides to the ectodomain of CD28 and/or CTLA-4.

Binding affinities for each of the cognate binding partners are independent; that is, in some embodiments, a variant CD86 polypeptide has an increased binding affinity for CD28 but not CTLA-4, relative to a wild-type or unmodified CD86 polypeptide.

In some embodiments, the variant CD86 polypeptide has an increased binding affinity for CD28, relative to a wild-type or unmodified CD86 polypeptide and has a decreased binding affinity for CTLA-4, relative to a wild-type or unmodified CD86 polypeptide. In some embodiments, the variant CD86 polypeptide has an increased binding affinity for CD28, relative to a wild-type or unmodified CD86 polypeptide and has no change in binding affinity for CTLA-4, relative to a wild-type or unmodified CD86 polypeptide.

In some embodiments, a variant CD86 polypeptide with increased or greater binding affinity to CD28 will have an increase in binding affinity relative to the wild-type or unmodified CD86 polypeptide control of at least about 5%, such as at least about 10%, 15%, 20%, 25%, 35%, or 50% for CD28. In some embodiments, the increase in binding affinity relative to the wild-type or unmodified CD86 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold 40-fold, 50-fold, 60-fold, 70-fold, 80-fold, 90-fold, 100-fold, 125-fold, 150-fold, 175-fold, 200-fold, 225-fold, 250-fold, 275-fold, 300-fold, 325-fold, 350-fold 375-fold, or 400-fold. In such examples, the wild-type or unmodified CD86 polypeptide has the same sequence as the variant CD86 polypeptide except that it does not contain the one or more amino acid modifications (e.g. substitutions).

In some embodiments, a variant CD86 polypeptide with reduced or decreased binding affinity to CTLA-4 will have a decrease in binding affinity relative to the wild-type or unmodified CD86 polypeptide control of at least 5%, such as at least about 10%, 15%, 20%, 30%, 40%, 50%, 60%, 70%, 80%, 90% or more for the CTLA-4. In some embodiments, the decrease in binding affinity relative to the wild-type or unmodified CD86 polypeptide is more than 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold 40-fold or 50-fold. In some embodiments, a variant CD86 polypeptide does not show a change in binding affinity to CTLA-4 relative to the wild-type or unmodified CD86 polypeptide control. In some embodiments, a variant CD86 polypeptide does not show an increase in binding affinity to CTLA-4 relative to the wild-type or unmodified CD86 polypeptide control. In such examples, the wild-type or unmodified CD86 polypeptide has the same sequence as the variant CD86 polypeptide except that it does not contain the one or more amino acid modifications (e.g. substitutions).

In some embodiments, the equilibrium dissociation constant (K_{d}) of any of the foregoing embodiments to CD28 and/or CTLA-4 can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹M, or 1×10⁻¹² M or less.

The wild-type or unmodified CD86 sequence does not necessarily have to be used as a starting composition to generate variant CD86 polypeptides described herein. Therefore, use of the term “modification”, such as “substitution”, does not imply that the present embodiments are limited to a particular method of making variant CD86 polypeptides. Variant CD86 polypeptides can be made, for example, by de novo peptide synthesis and thus does not necessarily require a modification, such as a “substitution”, in the sense of altering a codon to encode for the modification, e.g. substitution. This principle also extends to the terms “addition” and “deletion” of an amino acid residue which likewise do not imply a particular method of making. The means by which the variant CD86 polypeptides are designed or created is not limited to any particular method. In some embodiments, however, a wild-type or unmodified CD86 encoding nucleic acid is mutagenized from wild-type or unmodified CD86 genetic material and screened for desired specific binding affinity and/or induction of IFN-gamma expression or other functional activity. In some embodiments, a variant CD86 polypeptide is synthesized de novo utilizing protein or nucleic acid sequences available at any number of publicly available databases and then subsequently screened. The National Center for Biotechnology Information provides such information and its website is publicly accessible via the internet as is the UniProtKB database.

Unless stated otherwise, as indicated throughout the present disclosure, the amino acid modification(s) are designated by amino acid position number corresponding to the numbering of positions of the unmodified ECD sequence set forth in SEQ ID NO: 29 as follows:

(SEQ ID NO: 29)
APLKIQAYFNETADLPCQFANSQNQSLSELVVFWQDQENLVLNEVYLGKE
KFDSVHSKYMGRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGVMQKSQDNVTELYDVSISLSVSFPDVTSNMTIFCILETD
KTRLLSSPFSIELEDPQPPPDHIP

Modifications provided herein can be in a wild-type or unmodified CD86 polypeptide set forth in SEQ ID NO: 29 or in a portion thereof containing an IgV domain or a specific binding fragment thereof. In some embodiments, the wild-type or unmodified CD86 polypeptide contains the IgV of CD86 as set forth in SEQ ID NO: 122. In some embodiments, the unmodified CD86 polypeptide contains an IgV that can be several amino acids longer or shorter, such as 1-20, e.g. 1, 2, 3, 4, 5, 6, 7, 8, 9, 10, 11, 12, 13, 14, 15, 16, 17, 18, 19, or 20 amino acids longer or shorter, than the IgV sequence set forth in SEQ ID NO: 122. In some embodiments, the unmodified CD86 polypeptide has 85%, 86%, 87%, 88%, 89%, 90%, 91%, 92%, 93%, 94%, 95%, 96%, 97%, 98%, 99% sequence identity to SEQ ID NO: 29, 122, or 123, or a specific binding fragment thereof. In some embodiments, the unmodified CD86 polypeptide has the sequence set forth in any of SEQ ID NOs: 29, 122, and 123.

(SEQ ID NO: 122)
NETADLPCQFANSQNQSLSELVVFWQDQENLVLNEVYLGKEKFDSVHSK
YMGRTSFDSDSWTLRLHNLQIKDKGLYQCIIHHKKPTGMIRIHQMNSEL
S
(SEQ ID NO: 123)
APLKIQAYFNETADLPCQFANSQNQSLSELVVFWQDQENLVLNEVYLGK
EKFDSVHSKYMGRTSFDSDSWTLRLHNLQIKDKGLYQCIIHHKKPTGMI
RIHQMNSELSVLA

It is within the level of a skilled artisan to identify the corresponding position of a modification, e.g. amino acid substitution, in a CD86 polypeptide, including portion thereof containing an IgV domain, such as by alignment of a reference sequence with SEQ ID NO: 29. An exemplary alignment of SEQ ID NO: 29 containing residues 24-247 of wildtype CD86 with SEQ ID NO: 122 containing residues 33-131 of wildtype CD86 is shown in FIG. 3. In the listing of modifications throughout this disclosure, the amino acid position is indicated in the middle, with the corresponding unmodified (e.g. wild-type) amino acid listed before the number and the identified variant amino acid substitution listed after the number. If the modification is a deletion of the position a “del” is indicated and if the modification is an insertion at the position an “ins” is indicated. In some cases, an insertion is listed with the amino acid position indicated in the middle, with the corresponding unmodified (e.g. wild-type) amino acid listed before and after the number and the identified variant amino acid insertion listed after the unmodified (e.g. wild-type) amino acid.

In some embodiments, the variant CD86 polypeptide has one or more amino acid modifications, e.g. substitutions, in a wild-type or unmodified CD86 sequence. The one or more amino acid modifications, e.g. substitutions, can be in the ectodomain (extracellular domain; ECD) of the wild-type or unmodified CD86 sequence. In some embodiments, the one or more amino acid modifications, e.g. substitutions, are in the IgV domain or specific binding fragment thereof. In some embodiments, the one or more amino acid modifications, e.g. substitutions, are in the IgC domain or specific binding fragment thereof. In some embodiments, the one or more amino acid modifications, e.g. substitutions, are in the ECD or specific binding fragment thereof.

In some embodiments, the variant CD86 polypeptide has up to 1, 2, 3, 4, 5, 6, 7, 8, 9, 10, 11, 12, 13, 14, 15, 16, 17, 18, 19, or 20 amino acid modifications, e.g. substitutions. The modifications (e.g. substitutions) can be in the IgV domain. In some embodiments, the modifications are in the ECD. In some embodiments, the modifications are in the ECD and IgV domain. In some embodiments, the modifications are in the IgV domain. In some embodiments, the variant CD86 polypeptide has up to 1, 2, 3, 4, 5, 6, 7, 8, 9, 10, 11, 12, 13, 14, 15, 16, 17, 18, 19, or 20 amino acid modifications, e.g. substitutions, in the IgV domain or specific binding fragment thereof. In some embodiments, the variant CD86 polypeptide has up to 1, 2, 3, 4, 5, 6, 7, 8, 9, 10, 11, 12, 13, 14, 15, 16, 17, 18, 19, or 20 amino acid modifications, e.g. substitutions, in the ECD or specific binding fragment thereof. In some embodiments, the variant CD86 polypeptide has less than 100% sequence identity and at least about 85%, 86%, 86%, 88%, 89%, 90%, 91%, 92%, 93%, 94%, 95%, 96%, 97%, 98%, or 99% sequence identity with the wild-type or unmodified CD86 polypeptide or specific binding fragment thereof, such as with the amino acid sequence of SEQ ID NO: 29, 122, or 123.

In some embodiments, the variant CD86 polypeptide has one or more amino acid modifications, e.g. substitutions, in an unmodified CD86 or specific binding fragment thereof corresponding to position(s) 13, 18, 25, 28, 33, 38, 39, 40, 43, 45, 52, 53, 60, 68, 71, 77, 79, 80, 82, 86, 88, 89, 90, 92, 93, 97, 102, 104, 113, 114, 123, 128, 129, 132, 133, 137, 141, 143, 144, 148, 153, 154, 158, 170, 172, 175, 178, 180, 181, 183, 185, 192, 193, 196, 197, 198, 205, 206, 207, 212, 215, 216, 222, 223, or 224, with reference to positions set forth in SEQ ID NO:29. In some embodiments, the modification at position 224 is a deletion. In some embodiments, such variant CD86 polypeptides exhibit altered binding affinity to one or more of CD28 and/or CTLA-4 compared to the wild-type or unmodified CD86 polypeptide. For example, in some embodiments, the variant CD86 polypeptide exhibits increased binding affinity to CD28 compared to a wild-type or unmodified CD86 polypeptide. In some embodiments, the variant CD86 polypeptide exhibits decreased binding affinity to CTLA-4 compared to a wild-type or unmodified CD86 polypeptide. In some embodiments, the variant CD86 polypeptide does not exhibit any change in binding affinity to CTLA-4 compared to a wild-type or unmodified CD86 polypeptide. In some embodiments, the variant CD86 polypeptide does not exhibit an increase in binding affinity to CTLA-4 compared to a wild-type or unmodified CD86 polypeptide.

In some embodiments, the variant CD86 polypeptide has one or more amino acid substitutions selected from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative amino acid substitution thereof. A conservative amino acid substitution is any amino acid that falls in the same class of amino acids as the substituted amino acids, other than the wild-type or unmodified amino acid. The classes of amino acids are aliphatic (glycine, alanine, valine, leucine, and isoleucine), hydroxyl or sulfur-containing (serine, cysteine, threonine, and methionine), cyclic (proline), aromatic (phenylalanine, tyrosine, tryptophan), basic (histidine, lysine, and arginine), and acidic/amide (aspartate, glutamate, asparagine, and glutamine).

In some embodiments, the variant CD86 polypeptide has two or more amino acid substitutions selected from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative amino acid substitution thereof.

In some embodiments, the variant CD86 polypeptide contains one or more modifications (e.g. amino acid substitutions) at a position corresponding to position(s) selected from 13, 18, 25, 28, 33, 38, 39, 40, 43, 45, 52, 53, 60, 68, 71, 77, 79, 80, 82, 86, 88, 89, 90, 92, 93, 97, 102, 104, 113, 114, 123, 128, 129, 132, 133, 137, 141, 143, 144, 148, 153, 154, 158, 170, 172, 175, 178, 180, 181, 183, 185, 192, 193, 196, 197, 198, 205, 206, 207, 212, 215, 216, 222, 223, or 224 with reference to positions set forth in SEQ ID NO:29. In some embodiments, the amino acid modification is one or more amino acid substitution selected from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative amino acid substitution thereof.

In some embodiments, the variant CD86 polypeptide contains one or more amino acid substitution corresponding to A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative substitution thereof.

In some embodiments, the variant CD86 polypeptide contains at least one modification (e.g. substitution) at a position selected from 25 or 90. In some embodiments, at least one amino acid substitution is Q25L, H90Y, or H90L. In some embodiments, at least one amino acid substitution is Q25L. In some embodiments, at least one amino acid substitution is H90Y or H90L.

In some embodiments, the variant CD86 polypeptide contains amino acid substitutions selected from among Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93T/L132M/V148D/S181P/P216H, Q25L/F33I/H90Y/V128A/P141A/E158G/S181P, Q25L/N39D/K80R/Q86R/I88F/H90L/K93T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V45I/D68N/H90L/S183P/L205S, E38V/S114G/P143H, H90Y/L180S, H90Y/Y129N, I89V/H90L/I193V, K80E/H90Y/H222T/I223F/P224L, K80M/I88T, K92I/F113S, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, or S28G/H90Y. In some embodiments, the variant CD86 polypeptide contains amino acid substitutions selected from among Q25L/T71A/H90Y, Q25L/D53G/E212V, Q25L/H90L, N43K/I79N/H90L/I178T/E198D, A13V/Q25L/H90L/S181P/L 197M/S206T, Q25L/Q86R/H90L/K93 T/L132M/V148D/S181P/P216H, Q25L/F33I/H90Y/V128A/P141A/E158G/S181P, Q25L/N39D/K80R/Q86R/I88F/H90L/K93 T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V451/D68N/H90L/S183P/L205S/E212X, H90Y/L180S, H90Y/Y129N, I89V/H90L/I193V, K80E/H90Y/H222T/I223F/P224L, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, S28G/H90Y, A13V/Q25L/H90L, Q25L/H90L/K93T/M97L, Q25L/Q86R/H90L, or I89V/H90L. In some embodiments, the variant CD86 polypeptide contains amino acid substitutions Q25L/H90Y or Q25L/H90L.

In some embodiments, any of the provided variant CD86 polypeptides can further contain one or more amino acid substitutions from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T, or I223F.

In some embodiments, among the provided variant CD86 polypeptides are CD86 polypeptides that have amino acid substitutions Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93 T/L132M/V148D/S181P/P216H, Q25L/F33I/H90Y/V128A/P141A/E158G/S181P, Q25L/N39D/K80R/Q86R/I88F/H90L/K93 T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V45I/D68N/H90L/S183P/L205S, E38V/S114G/P143H, H90Y/L180S, H90Y/Y129N, I89V/H90L/I193V, K80E/H90Y/H222T/I223F/P224L, K80M/I88T, K92I/F113S, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, or S28G/H90Y. In some embodiments, among the provided variant CD86 polypeptides are CD86 polypeptides that have amino acid substitutions Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93T/L132M/V148D/S181P/P216H, Q25L/F33I/H90Y/V128A/P141A/E158G/S181P, Q25L/N39D/K80R/Q86R/I88F/H90L/K93T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V451/D68N/H90L/S183P/L205S/E212X, H90Y/L180S, H90Y/Y129N, I89V/H90L/I193V, K80E/H90Y/H222T/I223F/P224L, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, S28G/H90Y, A13V/Q25L/H90L, Q25L/H90L/K93T/M97L, Q25L/Q86R/H90L, or I89V/H90L.

In some embodiments, the variant CD86 polypeptide comprises any of the substitutions (mutations) listed in Table 1. Table 1 also provides exemplary sequences by reference to SEQ ID NO for the extracellular domain (ECD) or IgV domain of wild-type CD86 or exemplary variant CD86 polypeptides. In some cases, an IgV as indicated in the Table 1 is shorter than an ECD and thus may not include all amino acid substitutions as listed in Table 1, e.g. the amino acid substitutions outside of the IgV domain. As indicated, the exact locus or residues corresponding to a given domain can vary, such as depending on the methods used to identify or classify the domain. Also, in some cases, adjacent N- and/or C-terminal amino acids of a given domain (e.g. ECD or IgV) also can be included in a sequence of a variant IgSF polypeptide, such as to ensure proper folding of the domain when expressed. Thus, it is understood that the exemplification of the SEQ ID NOs in Table 1 is not to be construed as limiting. For example, the particular domain, such as the ECD or IgV domain, of a variant CD86 polypeptide can be several amino acids longer or shorter, such as 1-20, e.g. 1, 2, 3, 4, 5, 6, 7, 8, 9, 10, 11, 12, 13, 14, 15, 16, 17, 18, 19, or 20 amino acids longer or shorter, than the sequence of amino acids set forth in the respective SEQ ID NO.

In some embodiments, the variant CD86 polypeptide is or comprises any of the sequences set forth in SEQ ID NOS: 85-121, 124-134, 141-221, and 314. In some embodiments, the variant CD86 polypeptide is or comprises a polypeptide sequence that exhibits at least 90% identity, at least 91% identity, at least 92% identity, at least 93% identity, at least 94% identity, at least 95% identity, such as at least 96% identity, 97% identity, 98% identity, or 99% identity to any of the sequences set forth in any one of SEQ ID NOS: 85-121, 124-134, 141-221, and 314, and that contains the amino acid modification(s), e.g. substitution(s), therein not present in the wild-type or unmodified CD86. In some embodiments, the variant CD86 polypeptide is or comprises a specific binding fragment of any of any one of SEQ ID NOS: 85-121, 124-134, 314, and 141-221 and contains the amino acid modification(s), e.g. substitution(s), therein not present in the wild-type or unmodified CD86. In some embodiments, the variant CD86 is or comprises the sequence set forth by SEQ ID NOS: 89, 93, 94, 107, 111, 112, 115, 117, 124-134, 145, 149, 150, 163, 167, 168, 171, 173, 182, 186, 187, 200, 204, 205, 208, 210, 215-221, or 314. In some embodiments, the variant CD86 polypeptide is or comprises a polypeptide sequence that exhibits at least 90% identity, at least 91% identity, at least 92% identity, at least 93% identity, at least 94% identity, at least 95% identity, such as at least 96% identity, 97% identity, 98% identity, or 99% identity to any of the sequences set forth in any one of SEQ ID NOS: 89, 93, 94, 107, 111, 112, 115, 117, 124-134, 145, 149, 150, 163, 167, 168, 171, 173, 182, 186, 187, 200, 204, 205, 208, 210, 215-221, or 314, and contains the amino acid modification(s), e.g. substitution(s) therein not present in the wild-type or unmodified CD86.

In some embodiments, the variant CD86 polypeptide is or comprises a specific binding fragment of any one of SEQ ID NOS: 85-121, 124-134, 141-221, or 314 and contains the amino acid modification(s), e.g. substitution(s) therein, not present in the wild-type or unmodified CD86. In some embodiments, the variant CD86 polypeptide is or comprises a specific binding fragment of any one of SEQ ID NOS: 89, 93, 94, 107, 111, 112, 115, 117, 124-134, 145, 149, 150, 163, 167, 168, 171, 173, 182, 186, 187, 200, 204, 205, 208, 210, 215-221, or 314 and contains the amino acid modification(s), e.g. substitution(s) therein, not present in the wild-type or unmodified CD86.

TABLE 1
Exemplary variant CD86 polypeptides

| | SEQ ID NO | | |
|---|---|---|---|
| | ECD | IgV | IgV |
| Mutation(s) | (24-247) | (24-134 ) | (33-131) |
| Wild-type | 29 | 123 | 122 |
| Q25L/T71A/H90Y | 85 | 141 | 178 |
| Q25L/D53G/E212V | 86 | 142 | 179 |
| Q25L/H90L | 87 | 143 | 180 |
| N43K/I79N/H90L/I178T/E198D | 88 | 144 | 181 |
| A13V/Q25L/H90L/S181P/L197M/S206T | 89 | 145 | 182 |
| Q25L/Q86R/H90L/K93T/L132M/V148D/S181P/P216H | 90 | 146 | 183 |
| Q25L/F33I/H90Y/V128A/P141A/E158G/S181P | 91 | 147 | 184 |
| Q25L/N39D/K80R/Q86R/I88F/H90L/K93T/N123D/N154D | 92 | 148 | 185 |
| Q25L/H90L/K93T/M97L/T133A/S181P/D215V | 93 | 149 | 186 |
| Q25L/Q86R/H90L/N104S | 94 | 150 | 187 |
| Q25L/L40M/H90L/L180S/S183P | 95 | 151 | 188 |
| Q18K/Q25L/F33I/L40S/H90L | 96 | 152 | 189 |
| Q25L/Q86K/H90L/I137T/S181P | 97 | 153 | 190 |
| Q25L/L77P/H90Y/K153R/V170D/S181P | 98 | 154 | 191 |
| Q25L/S28G/F33I/F52L/H90L/Q102H/I178T | 99 | 155 | 192 |
| Q25L/F33I/H90L/K144E/L180S | 100 | 156 | 193 |
| Q25L/F33I/H90L/K153E/E172G/T192N | 101 | 157 | 194 |
| Q25L/F33I/Q86R/H90Y/D175E/I196V/E198D | 102 | 158 | 195 |
| Q25L/V45I/D68N/H90L/S183P/L205S | 103 | 159 | 196 |
| E38V/S114G/P143H | 104 | 160 | 197 |
| H90Y/L180S | 105 | 161 | 198 |
| H90Y/Y129N | 106 | 162 | 199 |
| I89V/H90L/I193V | 107 | 163 | 200 |
| K80E/H90Y/H222T/I223F/P224L | 108 | 164 | 201 |
| K80M/I88T | 109 | 165 | 202 |
| K92I/F113S | 110 | 166 | 203 |
| M60K/H90L | 111 | 167 | 204 |
| Q25L/F33I/H90L | 112 | 168 | 205 |
| Q25L/F33I/Q86R/H90L/K93T | 113 | 169 | 206 |
| Q25L/H90L | 114 | 170 | 207 |
| Q25L/H90L/P185S | 115 | 171 | 208 |
| Q25L/H90L/P185S/P224L | 116 | 172 | 209 |
| Q25L/H90L/S179R | 117 | 173 | 210 |
| Q25L/H90Y/S181P/I193V | 118 | 174 | 211 |
| Q25L/K82T/H90L/T152S/S207P | 119 | 175 | 212 |
| Q25L/Q86R/H90L/K93T | 120 | 176 | 213 |
| S28G/H90Y | 121 | 177 | 214 |
| A13V/Q25L/H90L | 131 | 124 | 215 |
| Q25L/H90L/K93T/M97L | 132 | 125 | 216 |
| Q25L/Q86R/H90L | 314 | 126 | 217 |
| I89V/H90L | 133 | 127 | 218 |
| M60K/H90L | 111 | 128 | 219 |
| Q25L/F33I/H90L | 112 | 129 | 220 |
| Q25L/H90L | 134 | 130 | 221 |

In some embodiments, any of the provided variants of CD86 can be included as a polypeptide that is shorter or longer as described, such as by 1-20 amino acids, e.g. 1, 2, 3, 4, 5, 6, 7, 8, 9, 10, 11, 12, 13, 14, 15, 16, 17, 18, 19, or 20 amino acids longer or shorter, than the sequence of amino acids set forth in Table 1 as long as the CD86 polypeptide binds to CD28, including binding with increased affinity compared to the wild-type or unmodified CD86 polypeptide.

In some embodiments, the variant CD86 polypeptide exhibits increased affinity for the ectodomain of CD28 compared to the wild-type or unmodified CD86 polypeptide, such as compared to the sequence set forth in SEQ ID NO: 29, 122, or 123.

In some embodiments, the variant CD86 polypeptide exhibits increased binding affinity for binding the ectodomain of CD28 and exhibits decreased binding affinity for binding to CTLA-4 compared to the wild-type or unmodified CD86 polypeptide, such as compared to the sequence set forth in SEQ ID NO: 29, 122, or 123. In some embodiments, the variant CD86 polypeptide exhibits increased affinity for the ectodomain of CD28, and no change in affinity for the ectodomain of CTLA-4, compared to wild-type or unmodified CD86 polypeptide, such as compared to the sequence set forth in SEQ ID NO: 29, 122, or 123.

In some embodiments, a variant CD86 polypeptide exhibits increased selectivity for CD86 versus CTLA-4 compared to the unmodified CD86 polypeptide (e.g. set forth in SEQ ID NO: 29, 122, or 123) for binding CD28 versus CTLA-4, such as indicated by a ratio of CD28 binding to CTLA-4 binding (CD28:CTLA-4 binding ratio). In some embodiments, the ratio of binding is greater than 1. In some embodiments, the variant CD86 polypeptide exhibits a ratio of binding CD28 versus CTLA-4 that is greater than or greater than about or 1.1, 1.2, 1.3, 1.4, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or more.

### III. FORMAT OF VARIANT POLYPEPTIDES

The immunomodulatory polypeptide comprising a variant CD86 provided herein in which is contained a vIgD can be formatted in a variety of ways, including as a soluble protein, membrane bound protein or secreted protein. In some embodiments, the particular format can be chosen for the desired therapeutic application. In some cases, an immunomodulatory polypeptide comprising a variant CD86 polypeptide is provided in a format to antagonize or block activity of its binding partner, e.g., CTLA-4 and/or CD28. In some cases, an immunomodulatory polypeptide comprising a variant CD86 polypeptide is provided in a format to agonize or stimulate activity of its binding partner, e.g., CD28. In some embodiments, agonism of CD28 may be useful to promote immunity in oncology. A skilled artisan can readily determine the activity of a particular format, such as for antagonizing or agonizing one or more specific binding partner. Exemplary methods for assessing such activities are provided herein, including in the examples. In some embodiments, the modular format of the provided immunomodulatory proteins provides flexibility for engineering or generating immunomodulatory proteins for modulating activity of multiple counter structures (multiple cognate binding partners).

In some aspects, provided are immunomodulatory proteins comprising a vIgD of CD86 in which such proteins are soluble, e.g., fused to an Fc chain. In some aspects, one or more additional IgSF domain, such as one or more additional vIgD, may be linked to a vIgD of CD86 as provided herein (hereinafter called a “stack” or “stacked” immunomodulatory protein). In some embodiments, such “stack” molecules can be provided in a soluble format or, in some cases, may be provided as membrane bound or secreted proteins. In some embodiments, a variant CD86 immunomodulatory protein is provided as a conjugate in which is contained a vIgD of CD86 linked, directly or indirectly, to a targeting agent or moiety, e.g., to an antibody or other binding molecules that specifically binds to a ligand, e.g., an antigen, for example, for targeting or localizing the vIgD to a specific environment or cell, such as when administered to a subject. In some embodiments, the targeting agent, e.g., antibody or other binding molecule, binds to a tumor antigen, thereby localizing the variant CD86 containing the vIgD to the tumor microenvironment, for example, to modulate activity of tumor infiltrating lymphocytes (TILs) specific to the tumor microenvironment.

In some embodiments, provided immunomodulatory proteins are expressed in cells and provided as part of an engineered cellular therapy (ECT). In some embodiments, the variant CD86 polypeptide is expressed in a cell, such as an immune cell (e.g., T cell or antigen presenting cell), in membrane-bound form, thereby providing a transmembrane immunomodulatory protein (hereinafter also called a “TIP”). In some embodiments, depending on the cognate binding partner(s) recognized by the TIP, engineered cells expressing a TIP can agonize a cognate binding partner by providing a costimulatory signal, either positive to negative, to other engineered cells and/or to endogenous T cells. In some embodiments, an engineered cell expressing a TIP binds to a cognate binding partner on a different cell. In some embodiments, when an engineered cell expressing a TIP binds to a cognate binding partner on a different cell, the costimulation is referred to as costimulation in trans. In some embodiments, an engineered cell expressing a TIP binds to a cognate binding partner on itself, thereby inducing costimulating in itself. In some embodiments, when a TIP on a cell binds to a cognate binding partner on itself, the costimulation is referred to as costimulation in cis. In some aspects, the variant CD86 polypeptide is expressed in a cell, such as an immune cell (e.g., T cell or antigen presenting cell), in secretable form to thereby produce a secreted or soluble form of the variant CD86 polypeptide (hereinafter also called a “SIP”), such as when the cells are administered to a subject. In some aspects, depending on the cognate binding partner(s) recognized by the SIP, engineered cells expressing a SIP can antagonize or agonize a cognate binding partner in the environment (e.g., tumor microenvironment) in which it is secreted. In some embodiments, a variant CD86 polypeptide is expressed in an infectious agent (e.g., viral or bacterial agent) which, upon administration to a subject, is able to infect a cell in vivo, such as an immune cell (e.g., T cell or antigen presenting cell), for delivery or expression of the variant polypeptide as a TIP or a SIP in the cell.

In some embodiments, a soluble immunomodulatory polypeptide, such as a variant CD86 containing a vIgD, can be encapsulated within a liposome which itself can be conjugated to any one of or any combination of the provided conjugates (e.g., a targeting moiety). In some embodiments, the soluble or membrane bound immunomodulatory polypeptides of the invention are deglycosylated. In more specific embodiments, the variant CD86 sequence is deglycosylated. In even more specific embodiments, the IgV and/or IgC (e.g., IgC2) domain or domains of the variant CD86 is deglycosylated.

Non-limiting examples of provided formats are further described below.

### B. Soluble Protein

In some embodiments, the immunomodulatory protein containing a variant CD86 polypeptide is a soluble protein. Those of skill will appreciate that cell surface proteins typically have an intracellular, transmembrane, and extracellular domain (ECD) and that a soluble form of such proteins can be made using the extracellular domain or an immunologically active subsequence thereof. Thus, in some embodiments, the immunomodulatory protein containing a variant CD86 polypeptide lacks a transmembrane domain or a portion of the transmembrane domain. In some embodiments, the immunomodulatory protein containing a variant CD86 lacks the intracellular (cytoplasmic) domain or a portion of the intracellular domain. In some embodiments, the immunomodulatory protein containing the variant CD86 polypeptide only contains the vIgD portion containing the ECD domain or a portion thereof containing an IgV domain and/or IgC (e.g., IgC2) domain or domains or specific binding fragments thereof containing the amino acid modification(s).

In some embodiments, an immunomodulatory polypeptide comprising a variant CD86 can include one or more variant CD86 polypeptides of the invention. In some embodiments a polypeptide of the invention will comprise exactly 1, 2, 3, 4, 5 variant CD86 sequences. In some embodiments, at least two of the variant CD86 sequences are identical variant CD86 sequences.

In some embodiments, the provided immunomodulatory polypeptide comprises two or more vIgD sequences of CD86. Multiple variant CD86 polypeptides within the polypeptide chain can be identical (i.e., the same species) to each other or be non-identical (i.e., different species) variant CD86 sequences. In addition to single polypeptide chain embodiments, in some embodiments two, three, four, or more of the polypeptides of the invention can be covalently or non-covalently attached to each other. Thus, monomeric, dimeric, and higher order (e.g., 3, 4, 5, or more) multimeric proteins are provided herein. For example, in some embodiments exactly two polypeptides of the invention can be covalently or non-covalently attached to each other to form a dimer. In some embodiments, attachment is made via interchain cysteine disulfide bonds. Compositions comprising two or more polypeptides of the invention can be of an identical species or substantially identical species of polypeptide (e.g., a homodimer) or of non-identical species of polypeptides (e.g., a heterodimer). A composition having a plurality of linked polypeptides of the invention can, as noted above, have one or more identical or non-identical variant CD86 polypeptides of the invention in each polypeptide chain.

In some embodiments, the immunomodulatory protein is or contains a variant CD86 polypeptide that is in monomer form and/or that exhibits monovalent binding to its binding partner. In some aspects, a variant CD86 polypeptide as described, such as a variant CD86 that is soluble and/or that lacks a transmembrane domain and intracellular signaling domain, is linked, directly or indirectly, to a further moiety. In some embodiments, the further moiety is a protein, peptide, small molecule or nucleic acid. In some embodiments, the monovalent immunomodulatory protein is a fusion protein. In some embodiments, the moiety is a half-life extending molecule. Examples of such half-life extending molecules include, but are not limited to, albumin, an albumin-binding polypeptide, Pro/Ala/Ser (PAS), a C-terminal peptide (CTP) of the beta subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some embodiments, the immunomodulatory polypeptide comprising a variant CD86 can be linked to a moiety that includes conformationally disordered polypeptide sequences composed of the amino acids Pro, Ala, and Ser (See e.g., WO2008/155134, SEQ ID NO: 242). In some cases, the amino acid repeat is at least 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues, wherein each repeat comprises (an) Ala, Ser, and Pro residue(s). Thus, provided herein is an immunomodulatory protein that is a PASylated protein wherein the variant CD86 polypeptide is linked, directly or indirectly via a linker, to Pro/Ala/Ser (PAS). In some embodiments, one or more additional linker structures may be used.

In some embodiments, the moiety facilitates detection or purification of the variant CD86 polypeptide. In some cases, the immunomodulatory polypeptide comprises a tag or fusion domain, e.g., affinity or purification tag, linked, directly or indirectly, to the N- and/or C-terminus of the CD86 polypeptide. Various suitable polypeptide tags and/or fusion domains are known, and include but are not limited to, a poly-histidine (His) tag, a FLAG-tag (SEQ ID NO: 248), a Myc-tag, and fluorescent protein-tags (e.g., EGFP, set forth in SEQ ID NOs: 244-246). In some cases, the immunomodulatory polypeptide comprising a variant CD86 comprises at least six histidine residues (set forth in SEQ ID NO: 249). In some cases, the immunomodulatory polypeptide comprising a variant CD86 further comprises various combinations of moieties. For example, the immunomodulatory polypeptide comprising a variant CD86 further comprises one or more polyhistidine-tag and FLAG tag.

In some embodiments, the CD86 polypeptide is linked to a modified immunoglobulin heavy chain constant region (Fc) that remains in monovalent form such as set forth in SEQ ID NO: 252.

In some embodiments, the immunomodulatory protein contains a variant CD86 polypeptide that is linked, directly or indirectly, via a linker to a multimerization domain. In some aspects, the multimerization domain increases the half-life of the molecule. Interaction of two or more variant CD86 polypeptides can be facilitated by their linkage, either directly or indirectly, to any moiety or other polypeptide that are themselves able to interact to form a stable structure. For example, separate encoded variant CD86 polypeptide chains can be joined by multimerization, whereby multimerization of the polypeptides is mediated by a multimerization domain. Typically, the multimerization domain provides for the formation of a stable protein-protein interaction between a first variant CD86 polypeptide and a second variant CD86 polypeptide.

Homo- or heteromultimeric polypeptides can be generated from co-expression of separate variant CD86 polypeptides. The first and second variant CD86 polypeptides can be the same or different. In particular embodiments, the first and second variant CD86 polypeptides are the same in a homodimer, and each is linked to a multimerization domain that is the same. In other embodiments, heterodimers can be formed by linking first and second variant CD86 polypeptides that are different. In some of such embodiments, the first and second variant CD86 polypeptides are linked to different multimerization domains capable of promoting heterodimer formation.

In some embodiments, a multimerization domain includes any capable of forming a stable protein-protein interaction. The multimerization domains can interact via an immunoglobulin sequence (e.g. Fc domain; see e.g., International Patent Pub. Nos. WO 93/10151 and WO 2005/063816 US; U.S. Pub. No. 2006/0024298; U.S. Pat. No. 5,457,035); leucine zipper (e.g., from nuclear transforming proteins fos and jun or the proto-oncogene c-myc or from General Control of Nitrogen (GCN4)) (see e.g., Busch and Sassone-Corsi (1990) Trends Genetics, 6:36-40; Gentz et al., (1989) Science, 243:1695-1699); a hydrophobic region; a hydrophilic region; or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. Pat. No. 5,731,168; International Patent Pub. Nos. WO 98/50431 and WO 2005/063816; Ridgway et al. (1996) Protein Engineering, 9:617-621. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or tryptophan). Compensatory cavities of identical or similar size to the protuberances are optionally created on the interface of the second polypeptide by replacing large amino acid side chains with smaller ones (e.g., alanine or threonine). Exemplary multimerization domains are described below.

The variant CD86 polypeptide can be joined anywhere, but typically via its N- or C-terminus, to the N- or C-terminus of a multimerization domain to form a chimeric polypeptide. The linkage can be direct or indirect via a linker. The chimeric polypeptide can be a fusion protein or can be formed by chemical linkage, such as through covalent or non-covalent interactions. For example, when preparing a chimeric polypeptide containing a multimerization domain, nucleic acid encoding all or part of a variant CD86 polypeptide can be operably linked to nucleic acid encoding the multimerization domain sequence, directly or indirectly or optionally via a linker domain. In some cases, the construct encodes a chimeric protein where the C-terminus of the variant CD86 polypeptide is joined to the N-terminus of the multimerization domain. In some instances, a construct can encode a chimeric protein where the N-terminus of the variant CD86 polypeptide is joined to the C-terminus of the multimerization domain.

A polypeptide multimer contains multiple, such as two, chimeric proteins created by linking, directly or indirectly, two of the same or different variant CD86 polypeptides directly or indirectly to a multimerization domain. In some examples, where the multimerization domain is a polypeptide, a gene fusion encoding the variant CD86 polypeptide and multimerization domain is inserted into an appropriate expression vector. The resulting chimeric or fusion protein can be expressed in host cells transformed with the recombinant expression vector, and allowed to assemble into multimers, where the multimerization domains interact to form multivalent polypeptides. Chemical linkage of multimerization domains to variant CD86 polypeptides can be carried out using heterobifunctional linkers.

The resulting chimeric polypeptides, such as fusion proteins, and multimers formed therefrom, can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

In some embodiments, the multimerization domain is an Fc domain or portions thereof from an immunoglobulin. In some embodiments, the immunomodulatory protein comprises a variant CD86 polypeptide attached to an immunoglobulin Fc (yielding an “immunomodulatory Fc fusion,” such as a “variant CD86-Fc fusion,” also termed a CD86 vIgD-Fc fusion). In some embodiments, the attachment of the variant CD86 polypeptide is at the N-terminus of the Fc. In some embodiments, the attachment of the variant CD86 polypeptide is at the C-terminus of the Fc. In some embodiments, two or more CD86 variant polypeptides (the same or different) are independently attached at the N-terminus and at the C-terminus. In some embodiments, CD86-Fc variant fusion provided herein contains a variant CD86 polypeptide in accord with the description set forth in Section II above.

In some embodiments, the Fc is murine or human Fc. In some embodiments, the Fc is a mammalian or human IgG1, lgG2, lgG3, or lgG4 Fc regions. In some embodiments, the Fc is derived from IgG1, such as human IgG1. In some embodiments, the Fc comprises the amino acid sequence set forth in SEQ ID NO: 229, 230, or 253 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 229, 230, or 253.

In some embodiments, the Fc region contains one more modifications to alter (e.g., reduce) one or more of its normal functions. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided Fc fusion proteins.

In some embodiments, one or more amino acid modifications may be introduced into the Fc region of a CD86-Fc variant fusion provided herein, thereby generating an Fc region variant. In some embodiments, the Fc region variant has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided variant CD86-Fc fusions comprise an Fc region that exhibits reduced effector functions, which makes it a desirable candidate for applications in which the half-life of the CD86-Fc variant fusion in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the CD86-Fc variant fusion lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.)). Useful effector cells for such assays include peripheral blood mononuclear cells (PBMC) and Natural Killer (NK) cells. Alternatively or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the CD86-Fc variant fusion is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

CD86-Fc variant fusions with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some embodiments, the Fc region of CD86-Fc variant fusions has an Fc region in which any one or more of amino acids at positions 234, 235, 236, 237, 238, 239, 270, 297, 298, 325, and 329 (indicated by EU numbering) are substituted with different amino acids compared to the native Fc region. Such alterations of Fc region are not limited to the above-described alterations, and include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1-E233P/L234V/L235A/G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072.

Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

In some embodiments, there is provided a CD86-Fc variant fusion comprising a variant CD86 polypeptide as described herein and a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc region of a CD86-Fc variant fusion comprises one or more amino acid substitution E356D and M358L by EU numbering. In some embodiments, the Fc region of a CD86-Fc variant fusion comprises one or more amino acid substitutions C220S, C226S and/or C229S by EU numbering. In some embodiments, the Fc region of a CD86 variant fusion comprises one or more amino acid substitutions R292C and V302C. See also Duncan & Winter, Nature 322:738-40 (1988); U.S. Pat. Nos. 5,648,260; 5,624,821; and WO 94/29351 concerning other examples of Fc region variants.

In some embodiments, the wild-type IgG1 Fc can be the Fc set forth in SEQ ID NO: 229 having an allotype containing residues Glu (E) and Met (M) at positions 356 and 358 by EU numbering (e.g., f allotype). In other embodiments, the wild-type IgG1 Fc contains amino acids of the human G1 ml allotype, such as residues containing Asp (D) and Leu (L) at positions 356 and 358, e.g. as set forth in SEQ ID NO 332. Thus, in some cases, an Fc provided herein can contain amino acid substitutions E356D and M358L to reconstitute residues of allotype G1 ml (e.g., alpha allotype). In some aspects, a wild-type Fc is modified by one or more amino acid substitutions to reduce effector activity or to render the Fc inert for Fc effector function. Exemplary effectorless or inert mutations include those described herein. Among effectorless mutations that can be included in an Fc of constructs provided herein are L234A, L235E, and G237A by EU numbering. In some embodiments, a wild-type Fc is further modified by the removal of one or more cysteine residues, such as by replacement of the cysteine residues to a serine residue at position 220 (C220S) by EU numbering. Exemplary inert Fc regions having reduced effector function are set forth in SEQ ID NO: 333 or 256 and SEQ ID NO: 258 or 230, which are based on allotypes set forth in SEQ ID NO: 229 or SEQ ID NO: 332, respectively. In some embodiments, an Fc region used in a construct provided herein can further lack a C-terminal lysine residue.

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided a CD86-Fc variant fusion comprising a variant Fc region comprising one or more amino acid modifications, wherein the variant Fc region is derived from IgG1, such as human IgG1. In some embodiments, the variant Fc region is derived from the amino acid sequence set forth in SEQ ID NO: 229. In some embodiments, the Fc contains at least one amino acid substitution that is N82G by numbering of SEQ ID NO: 229 (corresponding to N297G by EU numbering). In some embodiments, the Fc further contains at least one amino acid substitution that is R77C or V87C by numbering of SEQ ID NO: 229 (corresponding to R292C or V302C by EU numbering). In some embodiments, the variant Fc region further comprises a C5S amino acid modification by numbering of SEQ ID NO: 229 (corresponding to C220S by EU numbering), such as the Fc region set forth in SEQ ID NO: 254. For example, in some embodiments, the variant Fc region comprises the following amino acid modifications: V297G and one or more of the following amino acid modifications C220S, R292C, or V302C by EU numbering (corresponding to N82G and one or more of the following amino acid modifications C5S, R77C, or V87C with reference to SEQ ID NO: 229), e.g., the Fc region comprises the sequence set forth in SEQ ID NO: 255. In some embodiments, the variant Fc region comprises one or more of the amino acid modifications C220S, L234A, L235E, or G237A, e.g., the Fc region comprises the sequence set forth in SEQ ID NO: 256. In some embodiments, the variant Fc region comprises one or more of the amino acid modifications C220S, L235P, L234V, L235A, G236del, or S267K, e.g., the Fc region comprises the sequence set forth in SEQ ID NO:257. In some embodiments, the variant Fc comprises one or more of the amino acid modifications C220S, L234A, L235E, G237A, E356D, or M358L, e.g., the Fc region comprises the sequence set forth in SEQ ID NO:258.

In some embodiments, CD86-Fc variant fusion provided herein contains a variant CD86 polypeptide in accord with the description set forth in Section II above. In some embodiments, there is provided a CD86-Fc variant fusion comprising any one of the described variant CD86 polypeptide linked to a variant Fc region, wherein the variant Fc region is not a human IgG1 Fc containing the mutations R292C, N297G, and V302C (corresponding to R77C, N82G and V87C with reference to wild-type human IgG1 Fc set forth in SEQ ID NO: 229). In some embodiments, there is provided a CD86-Fc variant fusion comprising any one of the variant CD86 polypeptide linked to an Fc region or variant Fc region, wherein the variant CD86 polypeptide is not linked to the Fc with a linker consisting of three alanines.

In some embodiments, the Fc region lacks the C-terminal lysine corresponding to position 232 of the wild-type or unmodified Fc set forth in SEQ ID NO: 229 (corresponding to K447del by EU numbering). In some aspects, such an Fc region can additionally include one or more additional modifications, e.g., amino acid substitutions, such as any as described. Examples of such an Fc region are set forth in SEQ ID NO: 255-257, 258, or 259-261.

In some embodiments, there is provided a CD86-Fc variant fusion comprising a variant Fc region in which the variant Fc comprises the sequence of amino acids set forth in any of SEQ ID NOS: 255, 258, 256, 257, 254, or 259-261 or a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 255, 258, 256, 257, 254, or 259-261.

In some embodiments, the Fc is derived from IgG2, such as human IgG2. In some embodiments, the Fc comprises the amino acid sequence set forth in SEQ ID NO: 262 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 262.

In some embodiments, the Fc comprises the amino acid sequence set forth in SEQ ID NO: 263 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 263. In some embodiments, the IgG4 Fc is a stabilized Fc in which the CH3 domain of human IgG4 is substituted with the CH3 domain of human IgG1 and which exhibits inhibited aggregate formation, an antibody in which the CH3 and CH2 domains of human IgG4 are substituted with the CH3 and CH2 domains of human IgG1, respectively, or an antibody in which arginine at position 409 indicated in the EU index proposed by Kabat et al. of human IgG4 is substituted with lysine and which exhibits inhibited aggregate formation (see e.g., U.S. Pat. No. 8,911,726). In some embodiments, the Fc is an IgG4 containing the S228P mutation, which has been shown to prevent recombination between a therapeutic antibody and an endogenous IgG4 by Fab-arm exchange (see e.g., Labrijin et al. (2009) Nat. Biotechnol., 27(8): 767-71). In some embodiments, the Fc comprises the amino acid sequence set forth in SEQ ID NO: 264 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 264.

In some embodiments, the variant CD86 polypeptide is indirectly linked to the Fc sequence, such as via a linker. In some embodiments, one or more “peptide linkers” link the variant CD86 polypeptide and the Fc domain. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the linker is a flexible linker. In some embodiments, the linker is (in one-letter amino acid code): GGGGS (“4GS” or “G₄S”; SEQ ID NO: 223) or multimers of the 4GS linker, such as repeats of 2, 3, 4, or 5 4GS linkers, such as set forth in SEQ ID NO: 225 (2×GGGGS; (G₄S)₂) or SEQ ID NO: 224 (3×GGGGS; (G₄S)₃). In some embodiments, the linker can include a series of alanine residues alone or in addition to another peptide linker (such as a 4GS linker or multimer thereof). In some embodiments, the number of alanine residues in each series is 2, 3, 4, 5, or 6 alanines. In some embodiments, the linker is three alanines (AAA). In some embodiments, the variant CD86 polypeptide is indirectly linked to the Fc sequence via a linker, wherein the linker does not consist of three alanines. In some examples, the linker is a 2×GGGGS followed by three alanines (GGGGSGGGGSAAA; SEQ ID NO: 226). In some embodiments, the linker can further include amino acids introduced by cloning and/or from a restriction site, for example the linker can include the amino acids GS (in one-letter amino acid code) as introduced by use of the restriction site BAMHI. For example, in some embodiments, the linker (in one-letter amino acid code) is GSGGGGS (SEQ ID NO:222), GS(G₄S)₃ (SEQ ID NO: 227), or GS(G₄S)₅ (SEQ ID NO: 228). In some embodiments, the linker is a rigid linker. For example, the linker is an α-helical linker. In some embodiments, the linker is (in one-letter amino acid code): EAAAK or multimers of the EAAAK linker, such as repeats of 2, 3, 4, or 5 EAAAK linkers, such as set forth in SEQ ID NO: 265 (1×EAAAK), SEQ ID NO: 266 (3×EAAAK), or SEQ ID NO: 247 (5×EAAAK). In some cases, the immunomodulatory polypeptide comprising a variant CD86 comprises various combinations of peptide linkers.

In some embodiments, the variant CD86 polypeptide is directly linked to the Fc sequence. In some embodiments, the variant CD86 polypeptide is directly linked to an Fc, such as an inert Fc, that additionally lacks all or a portion of the hinge region. An exemplary Fc, lacking a portion (6 amino acids) of the hinge region is set forth in SEQ ID NO: 267.

In some embodiments, where the CD86 polypeptide is directly linked to the Fc sequence, the CD86 polypeptide can be truncated at the C-terminus by 1, 2, 3, 4, 5, 6, 7, 8, 9, 10, 11, 12, 13, 14, 15, or more amino acids. In some embodiments, the variant CD86 polypeptide is truncated to remove 1, 2, 3, 4, 5, 6, 7, 8, 9, 10, or more amino acids that connect the IgV region to the IgC region.

In some embodiments, the variant CD86-Fc fusion protein is a dimer formed by two variant CD86 Fc polypeptides linked to an Fc domain. In some specific embodiments, identical or substantially identical species (allowing for 3 or fewer N-terminus or C-terminus amino acid sequence differences) of CD86-Fc variant fusion polypeptides will be dimerized to create a homodimer. In some embodiments, the dimer is a homodimer in which the two variant CD86 Fc polypeptides are the same. Alternatively, different species of CD86-Fc variant fusion polypeptides can be dimerized to yield a heterodimer. Thus, in some embodiments, the dimer is a heterodimer in which the two variant CD86 Fc polypeptides are different.

Also provided are nucleic acid molecules encoding the variant CD86-Fc fusion protein. In some embodiments, for production of an Fc fusion protein, a nucleic acid molecule encoding a variant CD86-Fc fusion protein is inserted into an appropriate expression vector. The resulting variant CD86-Fc fusion protein can be expressed in host cells transformed with the expression vector where assembly between Fc domains occurs by interchain disulfide bonds formed between the Fc moieties to yield dimeric, such as divalent, variant CD86-Fc fusion proteins.

The resulting Fc fusion proteins can be easily purified by affinity chromatography over Protein A or Protein G columns. For the generation of heterodimers, additional steps for purification can be necessary. For example, where two nucleic acids encoding different variant CD86 polypeptides are transformed into cells, the formation of heterodimers must be biochemically achieved since variant CD86 molecules carrying the Fc-domain will be expressed as disulfide-linked homodimers as well. Thus, homodimers can be reduced under conditions that favor the disruption of interchain disulfides, but do no effect intra-chain disulfides. In some cases, different variant CD86-Fc monomers are mixed in equimolar amounts and oxidized to form a mixture of homo- and heterodimers. The components of this mixture are separated by chromatographic techniques. Alternatively, the formation of this type of heterodimer can be biased by genetically engineering and expressing Fc fusion molecules that contain a variant CD86 polypeptide using knob-into-hole methods described below.

C. Stack Molecules with Additional IgSF Domains

In some embodiments, the immunomodulatory proteins can contain any of the variant CD86 polypeptides provided herein linked, directly or indirectly, to one or more other immunoglobulin superfamily (IgSF) domain (“stacked” immunomodulatory protein construct and also called a “Type II” immunomodulatory protein). In some aspects, this can create unique multi-domain immunomodulatory proteins that bind two or more, such as three or more, cognate binding partners, thereby providing a multi-targeting modulation of the immune synapse.

In some embodiments, an immunomodulatory protein comprises a combination (a “non-wild-type combination”) and/or arrangement (a “non-wild type arrangement” or “non-wild-type permutation”) of a variant CD86 domain with one or more other affinity modified and/or non-affinity modified IgSF domain sequences of another IgSF family member (e.g., a mammalian IgSF family member) that are not found in wild-type IgSF family members. In some embodiments, the immunomodulatory protein contains 2, 3, 4, 5 or 6 immunoglobulin superfamily (IgSF) domains, where at least one of the IgSF domains is a variant CD86 IgSF domain (vIgD of CD86) according to the provided description.

In some embodiments, the sequences of the additional IgSF domains can be a modified IgSF domain that contains one or more amino acid modifications, e.g., substitutions, compared to a wildtype or unmodified IgSF domain. In some embodiments, the IgSF domain can be non-affinity modified (e.g., wild-type) or have been affinity modified. In some embodiments, the unmodified or wild-type IgSF domain can be from mouse, rat, cynomolgus monkey, or human origin, or combinations thereof. In some embodiments, the additional IgSF domains can be an IgSF domain of an IgSF family member set forth in Table 2. In some embodiments, the additional IgSF domain can be an affinity-modified IgSF domain containing one or more amino acid modifications, e.g., substitutions, compared to an IgSF domain contained in an IgSF family member set forth in Table 2.

In some embodiments, the additional IgSF domain is an affinity or non-affinity modified IgSF domain contained in an IgSF family member of a family selected from the Signal-Regulatory Protein (SIRP) Family, Triggering Receptor Expressed On Myeloid Cells Like (TREML) Family, Carcinoembryonic Antigen-related Cell Adhesion Molecule (CEACAM) Family, Sialic Acid Binding Ig-Like Lectin (SIGLEC) Family, Butyrophilin Family, B7 family, CD28 family, V-set and Immunoglobulin Domain Containing (VSIG) family, V-set transmembrane Domain (VSTM) family, Major Histocompatibility Complex (MHC) family, Signaling lymphocytic activation molecule (SLAM) family, Leukocyte immunoglobulin-like receptor (LIR), Nectin (Nec) family, Nectin-like (NECL) family, Poliovirus receptor related (PVR) family, Natural cytotoxicity triggering receptor (NCR) family, T cell immunoglobulin and mucin (TIM) family or Killer-cell immunoglobulin-like receptors (KIR) family. In some embodiments, the additional IgSF domains are independently derived from an IgSF protein selected from the group consisting of CD80(B7-1), CD86(B7-2), CD274 (PD-L1, B7-H1), PDCD1LG2(PD-L2, CD273), ICOSLG(B7RP1, CD275, ICOSL, B7-H2), CD276(B7-H3), VTCN1(B7-H4), CD28, CTLA4, PDCD1(PD-1), ICOS, BTLA(CD272), CD4, CD8A(CD8-alpha), CD8B(CD8-beta), LAG3, HAVCR2(TIM-3), CEACAM1, TIGIT, PVR(CD155), PVRL2(CD112), CD226, CD2, CD160, CD200, CD200R1(CD200R), and NC R3 (NKp30).

The first column of Table 2 provides the name and, optionally, the name of some possible synonyms for that particular IgSF member. The second column provides the protein identifier from the UniProtKB database, a publicly available database accessible via the internet at uniprot.org or, in some cases, the GenBank Number. The Universal Protein Resource (UniProt) is a comprehensive resource for protein sequence and annotation data. The UniProt databases include the UniProt Knowledgebase (UniProtKB). UniProt is a collaboration between the European Bioinformatics Institute (EMBL-EBI), the SIB Swiss Institute of Bioinformatics and the Protein Information Resource (PIR) and supported mainly by a grant from the U.S. National Institutes of Health (NIH). GenBank is the NIH genetic sequence database, an annotated collection of all publicly available DNA sequences (Nucleic Acids Research, 2013 January; 41(D1):D36-42). The third column provides the region where the indicated IgSF domain is located. The region is specified as a range where the domain is inclusive of the residues defining the range. Column 3 also indicates the IgSF domain class for the specified IgSF region. Column 4 provides the region where the indicated additional domains are located (signal peptide, S; extracellular domain, E; transmembrane domain, T; cytoplasmic domain, C). It is understood that description of domains can vary depending on the methods used to identify or classify the domain, and may be identified differently from different sources. The description of residues corresponding to a domain in Table 2 is for exemplification only and can be several amino acids (such as one, two, three or four) longer or shorter. Column 5 indicates for some of the listed IgSF members, some of its cognate cell surface binding partners.

TABLE 2
IgSF members according to the present disclosure.

| | NCBI | | | | | | |
|---|---|---|---|---|---|---|---|
| | Protein | | | | IgSF Member Amino Acid | | |
| | Accession | | | | Sequence | | |
| | Number/ | | | Cognate Cell | (SEQ ID NO) | | |
| IgSF | UniProtKB | IgSF Region | | Surface | Precursor | | |
| Member | Protein | & Domain | Other | Binding | (mature | | |
| (Synonyms) | Identifier | Class | Domains | Partners | residues) | Mature | ECD |
| CD80 | NP_005182.1 | 35-135, 35-138, | S: 1-34, | CD28, CTLA4, | SEQ ID NO: 1 | SEQ ID | SEQ ID |
| (B7-1) | P33681 | 37-138, or | E: 35-242, | PD-L1 | (35-288) | NO: 55 | NO: 28 |
| | | 35-141 IgV, | T: 243-263, | | | | |
| | | 145-230 or | C: 264-288 | | | | |
| | | 154-232 IgC | | | | | |
| CD86 | P42081.2 | 33-131 IgV, | S: 1-23, | CD28, CTLA4 | SEQ ID NO: 2 | SEQ ID | SEQ ID |
| (B7-2) | | 150-225 IgC2 | E: 24-247, | | (24-329) | NO: 56 | NO: 29 |
| | | | T: 248-268, | | | | |
| | | | C: 269-329 | | | | |
| CD274 | Q9NZQ7.1 | 24-130 or | S: 1-18, | PD-1, B7-1 | SEQ ID NO: 3 | SEQ ID | SEQ ID |
| (PD-L1, | NP_054862.1 | 19-127 IgV, | E: 19-238, | | (19-290) | NO: 57 | NO: 30 |
| B7-H1) | | 133-225 IgC2 | T: 239-259, | | | | |
| | | | C: 260-290 | | | | |
| PDCD1LG2 | Q9BQ51.2 | 21-118 IgV, | S: 1-19, | PD-1, RGMb | SEQ ID NO: 4 | SEQ ID | SEQ ID |
| (PD-L2, | | 122-203 IgC2 | E: 20-220, | | (20-273) | NO: 58 | NO: 31 |
| CD273) | | | T: 221-241, | | | | |
| | | | C: 242-273 | | | | |
| ICOSLG | O75144.2 | 19-129 IgV, | S: 1-18, | ICOS, CD28, | SEQ ID NO: 5 | SEQ ID | SEQ ID |
| (B7RP1, | | 141-227 IgC2 | E: 19-256, | CTLA4 | (19-302) | NO: 59 | NO: 32 |
| CD275, | | | T: 257-277, | | | | |
| ICOSL, | | | C: 278-302 | | | | |
| B7-H2) | | | | | | | |
| CD276 | Q5ZPR3.1 | 29-139 IgV, | S: 1-28, | | SEQ ID NO: 6 | SEQ ID | SEQ ID |
| (B7-H3) | | 145-238 IgC2, | E: 29-466, | | (29-534) | NO: 60 | NO: 33 |
| | | 243-357 IgV2, | T: 467-487, | | | | |
| | | 363-456, | C: 488-534 | | | | |
| | | 367-453 IgC2 | | | | | |
| VTCN1 | Q7Z7D3.1 | 35-146 IgV, | S: 1-24, | | SEQ ID NO: 7 | SEQ ID | SEQ ID |
| (B7-H4) | | 153-241 IgV | E: 25-259, | | (25-282) | NO: 61 | NO: 34 |
| | | | T: 260-280, | | | | |
| | | | C: 281-282 | | | | |
| CD28 | P10747.1 | 28-137 IgV | S: 1-18, | B7-1, B7-2, | SEQ ID NO: 8 | SEQ ID | SEQ ID |
| | | | E: 19-152, | B7RP1 | (19-220) | NO: 62 | NO: 35 |
| | | | T: 153-179, | | | | |
| | | | C: 180-220 | | | | |
| CTLA-4 | P16410.3 | 39-140 IgV | S: 1-35, | B7-1, B7-2, | SEQ ID NO: 9 | SEQ ID | SEQ ID |
| | | | E: 36-161, | B7RP1 | (36-223) | NO: 63 | NO: 36 |
| | | | T: 162-182, | | | | |
| | | | C: 183-223 | | | | |
| PDCD1 | Q15116.3 | 35-145 IgV | S: 1-20, | PD-L1, PD-L2 | SEQ ID NO: 10 | SEQ ID | SEQ ID |
| (PD-1) | | | E: 21-170, | | (21-288) | NO: 64 | NO: 37 |
| | | | T: 171-191, | | | | |
| | | | C: 192-288 | | | | |
| ICOS | Q9Y6W8.1 | 30-132 IgV | S: 1-20, | B7RP1 | SEQ ID NO: 11 | SEQ ID | SEQ ID |
| | | | E: 21-140, | | (21-199) | NO: 65 | NO: 38 |
| | | | T: 141-161, | | | | |
| | | | C: 162-199 | | | | |
| BTLA | Q7Z6A9.3 | 31-132 IgV | S: 1-30, | HVEM | SEQ ID NO: 12 | SEQ ID | SEQ ID |
| (CD272) | | | E: 31-157, | | (31-289) | NO: 66 | NO: 39 |
| | | | T: 158-178, | | | | |
| | | | C: 179-289 | | | | |
| CD4 | P01730.1 | 26-125 IgV, | S: 1-25, | MHC class II | SEQ ID NO: 13 | SEQ ID | SEQ ID |
| | | 126-203 IgC2, | E: 26-396, | | (26-458) | NO: 67 | NO: 40 |
| | | 204-317 IgC2, | T: 397-418, | | | | |
| | | 317-389, | C: 419-458 | | | | |
| | | 318-374 IgC2 | | | | | |
| CD8A | P01732.1 | 22-135 IgV | S: 1-21, | MHC class I | SEQ ID NO: 14 | SEQ ID | SEQ ID |
| (CD8-alpha) | | | E: 22-182, | | (22-235) | NO: 68 | NO: 41 |
| | | | T: 183-203, | | | | |
| | | | C: 204-235 | | | | |
| CD8B | P10966.1 | 22-132 IgV | S: 1-21, | MHC class I | SEQ ID NO: 15 | SEQ ID | SEQ ID |
| (CD8-beta) | | | E: 22-170, | | (22-210) | NO: 69 | NO: 42 |
| | | | T: 171-191, | | | | |
| | | | C: 192-210 | | | | |
| LAG3 | P18627.5 | 37-167 IgV, | S: 1-28, | MHC class II | SEQ ID NO: 16 | SEQ ID | SEQ ID |
| | | 168-252 IgC2, | E: 29-450, | | (29-525) | NO: 70 | NO: 43 |
| | | 265-343 IgC2, | T: 451-471, | | | | |
| | | 349-419 IgC2 | C: 472-525 | | | | |
| HAVCR2 | Q8TDQ0.3 | 22-124 IgV | S: 1-21, | CEACAM-1, | SEQ ID NO: 17 | SEQ ID | SEQ ID |
| (TIM-3) | | | E: 22-202, | phosphatidylserine, | (22-301) | NO: 71 | NO: 44 |
| | | | T: 203-223, | Galectin-9, | | | |
| | | | C: 224-301 | HMGB1 | | | |
| CEACAM1 | P13688.2 | 35-142 IgV, | S: 1-34, | TIM-3 | SEQ ID NO: 18 | SEQ ID | SEQ ID |
| | | 145-232 IgC2, | E: 35-428, | | (35-526) | NO: 72 | NO: 45 |
| | | 237-317 IgC2, | T: 429-452, | | | | |
| | | 323-413 IgC2 | C: 453-526 | | | | |
| TIGIT | Q495A1.1 | 22-124 IgV | S: 1-21, | CD155, CD112 | SEQ ID NO: 19 | SEQ ID | SEQ ID |
| | | | E: 22-141, | | (22-244) | NO: 73 | NO: 46 |
| | | | T: 142-162, | | | | |
| | | | C: 163-244 | | | | |
| PVR | P15151.2 | 24-139 IgV, | S: 1-20, | TIGIT, CD226, | SEQ ID NO: 20 | SEQ ID | SEQ ID |
| (CD155) | | 145-237 IgC2, | E: 21-343, | CD96, | (21-417) | NO: 74 | NO: 47 |
| | | 244-328 IgC2 | T: 344-367, | poliovirus | | | |
| | | | C: 368-417 | | | | |
| PVRL2 | Q92692.1 | 32-156 IgV, | S: 1-31, | TIGIT, CD226, | SEQ ID NO: 21 | SEQ ID | SEQ ID |
| (CD112) | | 162-256 IgC2, | E: 32-360, | CD112R | (32-538) | NO: 75 | NO: 48 |
| | | 261-345 IgC2 | T: 361-381, | | | | |
| | | | C: 382-538 | | | | |
| CD226 | Q15762.2 | 19-126 IgC2, | S: 1-18, | CD155, CD112 | SEQ ID NO: 22 | SEQ ID | SEQ ID |
| | | 135-239 IgC2 | E: 19-254, | | (19-336) | NO: 76 | NO: 49 |
| | | | T: 255-275, | | | | |
| | | | C: 276-336 | | | | |
| CD2 | P06729.2 | 25-128 IgV, | S: 1-24, | CD58 | SEQ ID NO: 23 | SEQ ID | SEQ ID |
| | | 129-209 IgC2 | E: 25-209, | | (25-351) | NO: 77 | NO: 50 |
| | | | T: 210-235, | | | | |
| | | | C: 236-351 | | | | |
| CD160 | O95971.1 | 27-122 IgV | N/A | HVEM, MHC | SEQ ID NO: 24 | SEQ ID | SEQ ID |
| | | | | family of | (27-159) | NO: 78 | NO: 51 |
| | | | | proteins | | | |
| CD200 | P41217.4 | 31-141 IgV, | S: 1-30, | CD200R | SEQ ID NO: 25 | SEQ ID | SEQ ID |
| | | 142-232 IgC2 | E: 31-232, | | (31-278) | NO: 79 | NO: 52 |
| | | | T: 233-259, | | | | |
| | | | C: 260-278 | | | | |
| CD200R1 | Q8TD46.2 | 53-139 IgV, | S: 1-28, | CD200 | SEQ ID NO: 26 | SEQ ID | SEQ ID |
| (CD200R) | | 140-228 IgC2 | E: 29-243, | | (29-325) | NO: 80 | NO: 53 |
| | | | T: 244-264, | | | | |
| | | | C: 265-325 | | | | |
| NCR3 | O14931.1 | 19-126 | S: 1-18, | B7-H6 | SEQ ID NO: 27 | SEQ ID | SEQ ID |
| (NKp30) | | IgC-like | E: 19-135, | | (19-201) | NO: 81 | NO: 54 |
| | | | T: 136-156, | | | | |
| | | | C: 157-201 | | | | |
| VSIG8 | Q5VU13 | 22-141 IgV1, | S: 1-21 | VISTA | SEQ ID NO: 82 | SEQ ID | SEQ ID |
| | | 146-257 | E: 22-263 | | (22-414) | NO: 83 | NO: 84 |
| | | IgV2 | T: 264-284 | | | | |
| | | | C: 285-414 | | | | |

The number of such non-affinity modified or affinity modified IgSF domains present in a “stacked” immunomodulatory protein construct (whether non-wild type combinations or non-wild type arrangements) is at least 2, 3, 4, or 5 and in some embodiments exactly 2, 3, 4, or 5 IgSF domains (whereby determination of the number of affinity modified IgSF domains disregards any non-specific binding fractional sequences thereof and/or substantially immunologically inactive fractional sequences thereof).

In some embodiments of a stacked immunomodulatory protein provided herein, the number of IgSF domains is at least 2 wherein the number of affinity modified and the number of non-affinity modified IgSF domains is each independently at least: 0, 1, 2, 3, 4, 5, or 6. Thus, the number of affinity modified IgSF domains and the number of non-affinity modified IgSF domains, respectively, (affinity modified IgSF domain: non-affinity modified IgSF domain), can be exactly or at least: 2:0 (affinity modified: wild-type), 0:2, 2:1, 1:2, 2:2, 2:3, 3:2, 2:4, 4:2, 1:1, 1:3, 3:1, 1:4, 4:1, 1:5, or 5:1.

In some embodiments of a stacked immunomodulatory protein, at least two of the non-affinity modified and/or affinity modified IgSF domains are identical IgSF domains.

In some embodiments, a stacked immunomodulatory protein provided herein comprises at least two affinity modified and/or non-affinity modified IgSF domains from a single IgSF member but in a non-wild-type arrangement (alternatively, “permutation”). One illustrative example of a non-wild type arrangement or permutation is an immunomodulatory protein comprising a non-wild-type order of affinity modified and/or non-affinity modified IgSF domain sequences relative to those found in the wild-type CD86 whose IgSF domain sequences served as the source of the variant IgSF domains as provided herein. Thus, in one example, the immunomodulatory protein can comprise an IgV proximal and an IgC distal to the transmembrane domain albeit in a non-affinity modified and/or affinity modified form. The presence, in an immunomodulatory protein provided herein, of both non-wild-type combinations and non-wild-type arrangements of non-affinity modified and/or affinity modified IgSF domains, is also within the scope of the provided subject matter.

In some embodiments of a stacked immunomodulatory protein, the non-affinity modified and/or affinity modified IgSF domains are non-identical (i.e., different) IgSF domains. Non-identical affinity modified IgSF domains specifically bind, under specific binding conditions, different cognate binding partners and are “non-identical” irrespective of whether or not the wild-type or unmodified IgSF domains from which they are engineered was the same. Thus, for example, a non-wild-type combination of at least two non-identical IgSF domains in an immunomodulatory protein can comprise at least one IgSF domain sequence whose origin is from and unique to one CD86, and at least one of a second IgSF domain sequence whose origin is from and unique to another IgSF family member that is not CD86, wherein the IgSF domains of the immunomodulatory protein are in non-affinity modified and/or affinity modified form. However, in alternative embodiments, the two non-identical IgSF domains originate from the same IgSF domain sequence but at least one is affinity modified such that they specifically bind to different cognate binding partners.

In some embodiments, the provided immunomodulatory proteins, in addition to containing a variant CD86 polypeptide, also contains at least 1, 2, 3, 4, 5 or 6 additional immunoglobulin superfamily (IgSF) domains, such as an IgD domain of an IgSF family member set forth in Table 2. In some embodiments, the provided immunomodulatory protein contains at least one additional IgSF domain (e.g., second IgSF domain). In some embodiments, the provided immunomodulatory protein contains at least two additional IgSF domains (e.g., second and third IgSF domain). In some embodiments, the provided immunomodulatory protein contains at least three additional IgSF domains (e.g., second, third and fourth). In some embodiments, the provided immunomodulatory protein contains at least four additional IgSF domains (e.g., second, third, fourth and fifth). In some embodiments, the provided immunomodulatory protein contains at least five additional IgSF domains (e.g., second, third, fourth, fifth and sixth). In some embodiments, the provided immunomodulatory protein contains at least six additional IgSF domains (e.g., second, third, fourth, fifth, sixth, and seventh). In some embodiments, each of the IgSF domains in the immunomodulatory protein are different. In some embodiments, at least one of the additional IgSF domains is the same as at least one other IgSF domain in the immunomodulatory protein. In some embodiments, each of the IgSF domains is from or derived from a different IgSF family member. In some embodiments, at least two of the IgSF domains are from or derived from the same IgSF family member.

In some embodiments, the additional IgSF domain comprises an IgV domain or an IgC (e.g., IgC2) domain or domains, or a specific binding fragment of the IgV domain or a specific binding fragment of the IgC (e.g., IgC2) domain or domains. In some embodiments, the additional IgSF domain is or comprises a full-length IgV domain. In some embodiments, the additional IgSF domain is or comprises a full-length IgC (e.g., IgC2) domain or domains. In some embodiments, the additional IgSF domain is or comprises a specific binding fragment of the IgV domain. In some embodiments, the additional IgSF domain is or comprises a specific binding fragment of the IgC (e.g., IgC2) domain or domains. In some embodiments, the immunomodulatory protein contains at least two additional IgSF domains from a single (same) IgSF member. For example, in some aspects, the immunomodulatory protein contains an ECD or portion thereof of an IgSF member containing a full-length IgV domain and a full-length IgC (e.g., IgC2) domain or domains or specific binding fragments thereof.

In some embodiments, the provided immunomodulatory proteins contains at least one additional IgSF domain (e.g., a second or, in some cases, also a third IgSF domain and so on) in which at least one additional or second IgSF domain is an IgSF domain set forth in a wild-type or unmodified IgSF domain or a specific binding fragment thereof contained in the sequence of amino acids set forth in any of SEQ ID NOS: 2-27 and 82. In some embodiments, the wild-type or unmodified IgSF domain is an IgV domain or an IgC domain, such as an IgC1 or IgC2 domain.

In some embodiments, the provided immunomodulatory proteins, in addition to containing a variant CD86 polypeptide, also contains at least one additional affinity-modified IgSF domain (e.g., a second or, in some cases, also a third affinity-modified IgSF domain and so on) in which at least one additional IgSF domain is a vIgD that contains one or more amino acid modifications (e.g., substitution, deletion or mutation) compared to an IgSF domain in a wild-type or unmodified IgSF domain, such as an IgSF domain in an IgSF family member set forth in Table 2. In some embodiments, the additional e.g., second or third, affinity-modified IgSF domain comprises at least 85%, 86%, 87%, 88%, 89%, 90%, 91%, 92%, 93%, 94%, 95%, 96%, 97%, 98%, 99% or more sequence identity to a wild-type or unmodified IgSF domain or a specific binding fragment thereof contained in the sequence of amino acids set forth in any of SEQ ID NOS: 2-27 and 82. In some embodiments, the wild-type or unmodified IgSF domain is an IgV domain or an IgC domain, such as an IgC1 or IgC2 domain. In some embodiments, the additional, e.g., second or third, IgSF domain is an affinity-modified IgV domain and/or IgC domain. In some embodiments, the one or more additional IgSF domain is an affinity-modified IgSF domain that contains an IgV domain and/or an IgC (e.g., IgC2) domain or domains, or a specific binding fragment of the IgV domain and/or a specific binding fragment of the IgC (e.g., IgC2) domain or domains, in which the IgV and/or IgC domain contains the amino acid modification(s) (e.g., substitution(s)). In some embodiments, the one or more additional affinity-modified IgSF domain contains an IgV domain containing the amino acid modification(s) (e.g., substitution(s)). In some embodiments, the one or more additional affinity-modified IgSF domain include IgSF domains present in the ECD or a portion of the ECD of the corresponding unmodified IgSF family member, such as a full-length IgV domain and a full-length IgC (e.g., IgC2) domain or domains, or specific binding fragments thereof, in which one or both of the IgV and IgC contain the amino acid modification(s) (e.g., substitution(s)).

In some embodiments, the provided immunomodulatory protein contains at least one additional or second IgSF domain that is a vIgD that contains one or more amino acid substitutions compared to an IgSF domain (e.g., IgV) of a wild-type or unmodified IgSF domain other than CD86.

The stack molecule immunomodulatory proteins containing at least one IgSF domain of a variant CD86 and one or more second or additional IgSF domain can be provided in various construct formats as described in Section III.C.3. Non-limiting examples of constructs are set forth below.

### 1. PD-1 IgSF Domains

In some embodiments, the at least one additional (e.g., second or third) vIgD is an IgSF domain (e.g., IgV) of a variant PD-1 polypeptide that contains one or more amino acid modifications (e.g., substitutions, deletions or additions) in the IgSF domain (e.g., IgV) compared to unmodified or wild-type PD-1. In some embodiments, the IgSF domain of PD-1 comprises an IgV domain or specific binding fragment of the IgV domain. In some embodiments, the IgD can be an IgV only, including the entire extracellular domain (ECD), or any combination of Ig domains of PD-1. In some embodiments, the wild-type or unmodified PD-1 polypeptide has (i) the sequence of amino acids set forth in SEQ ID NO: 10 or a mature form thereof lacking the signal sequence, (ii) a sequence of amino acids that exhibits at least about 85%, 86%, 87%, 88%, 89%, 90%, 91%, 92%, 93%, 94%, 95%, 96%, 97%, 98%, 99% sequence identity to SEQ ID NO: 10 or a mature form thereof, or (iii) is a portion of (i) or (ii) containing an IgV domain or specific binding fragments thereof. In some embodiments, the wild-type or unmodified PD-1 polypeptide has (i) the sequence of amino acids set forth in SEQ ID NO: 37, (ii) a sequence of amino acids that exhibits at least about 85%, 86%, 87%, 88%, 89%, 90%, 91%, 92%, 93%, 94%, 95%, 96%, 97%, 98%, 99% sequence identity to SEQ ID NO: 37, or (iii) is a portion of (i) or (ii) containing an IgV domain or specific binding fragments thereof. In some embodiments, the unmodified PD-1 polypeptide has 85%, 85%, 87%, 88%, 89%, 90%, 91%, 92%, 93%, 94%, 95%, 96%, 97%, 98%, 99% sequence identity to SEQ ID NO: 37, 335, 336, or 337, or a specific binding fragment thereof. In some embodiments, the unmodified PD-1 polypeptide has the sequence set forth in any of SEQ ID NOs: 37, 335, 336, or 337.

In some embodiments, the IgSF domain of PD-1 is a variant PD-1 polypeptide containing at least one affinity-modified IgSF domain (e.g. IgV or IgC) or a specific binding fragment thereof is an IgSF domain contained in a wild-type or unmodified PD-1 polypeptide such that the variant PD-1 polypeptide exhibits altered (increased or decreased) binding activity or affinity for PD-L1 or PD-L2 compared to a wild-type or unmodified PD−1 polypeptide. In some embodiments, the variant PD-1 polypeptides containing at least one affinity-modified IgSF domain (e.g., IgV) or a specific binding fragment thereof relative to an IgSF domain contained in a wild-type or unmodified PD-1 polypeptide such that the variant PD-1 polypeptide exhibits altered (increased or decreased) binding activity or affinity for one or more ligands PD-L1 or PD-L2 compared to a wild-type or unmodified PD-1 polypeptide. In some embodiments, a variant PD-1 polypeptide has a binding affinity for PD-L1 and/or PD-L2 that differs from that of a wild-type or unmodified PD-1 polypeptide control sequence as determined by, for example, solid-phase ELISA immunoassays, flow cytometry, ForteBio Octet or Biacore assays. In some embodiments, the variant PD-1 polypeptide has an increased binding affinity for PD-L1 and/or PD-L2. In some embodiments, the variant PD-1 polypeptide has a decreased binding affinity for PD-L2, relative to a wild-type or unmodified PD-L1 polypeptide. The PD-L1 and/or the PD-L2 can be a mammalian protein, such as a human protein or a murine protein.

Binding affinities for each of the cognate binding partners are independent; that is, in some embodiments, a variant PD-1 polypeptide has an increased binding affinity for one or both of PD-L1 and/or PD-L2, and a decreased binding affinity for one or both of PD-L1 and PD-L2, relative to a wild-type or unmodified PD-1 polypeptide.

In some embodiments, the variant PD-1 polypeptide has an increased binding affinity for PD-L1, relative to a wild-type or unmodified PD-1 polypeptide. In some embodiments, the variant PD-1 polypeptide has an increased or decreased binding affinity for PD-L2, relative to a wild-type or unmodified PD-L1 polypeptide. In some embodiments, the variant PD-1 polypeptide has an increased binding affinity for PD-L1, relative to a wild-type or unmodified PD-1 polypeptide and has a decreased binding affinity for PD-L2, relative to a wild-type or unmodified PD-1polypeptide.

In some embodiments, a variant PD-1 polypeptide with increased or greater binding affinity to PD-L1 and/or PD-L2 will have an increase in binding affinity relative to the wild-type or unmodified PD-1 polypeptide control of at least about 5%, such as at least about 10%, 15%, 20%, 25%, 35%, or 50% for the PD-L1 and/or PD-L2. In some embodiments, the increase in binding affinity relative to the wild-type or unmodified PD-1 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold. In such examples, the wild-type or unmodified PD-1 polypeptide has the same sequence as the variant PD-1 polypeptide except that it does not contain the one or more amino acid modifications (e.g. substitutions).

In some embodiments, a variant PD-1 polypeptide with reduced or decreased binding affinity to PD-L2 will have decrease in binding affinity relative to the wild-type or unmodified PD-1 polypeptide control of at least 5%, such as at least about 10%, 15%, 20%, 30%, 40%, 50%, 60%, 70%, 80%, 90% or more for the PD-L2. In some embodiments, the decrease in binding affinity relative to the wild-type or unmodified PD-1 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold. In such examples, the wild-type or unmodified PD-1 polypeptide has the same sequence as the variant PD-1 polypeptide except that it does not contain the one or more amino acid modifications (e.g. substitutions).

The PD-L1 and/or PD-L2 can be a mammalian protein, such as a human protein or a murine protein. In some embodiments, the PD-L1 is a human protein. In some embodiments, the PD-L2 is a human protein.

In some embodiments, the equilibrium dissociation constant (K_{d}) of any of the foregoing embodiments to PD-L1 and/or PD-L2 can be less than 1×10⁻⁵ M, 1×10⁻⁶ M, 1×10⁻⁷ M, 1×10⁻⁸ M, 1×10⁻⁹ M, 1×10⁻¹⁰ M or 1×10⁻¹M, or 1×10⁻¹² M or less.

The wild-type or unmodified PD-1 sequence does not necessarily have to be used as a starting composition to generate variant PD-1 polypeptides described herein. Therefore, use of the term “modification”, such as “substitution” does not imply that the present embodiments are limited to a particular method of making variant PD-1 polypeptides. Variant PD-1 polypeptides can be made, for example, by de novo peptide synthesis and thus does not necessarily require a modification, such as a “substitution”, in the sense of altering a codon to encode for the modification, e.g. substitution. This principle also extends to the terms “addition” and “deletion” of an amino acid residue which likewise do not imply a particular method of making. The means by which the variant PD-1 polypeptides are designed or created is not limited to any particular method. In some embodiments, however, a wild-type or unmodified PD-1 encoding nucleic acid is mutagenized from wild-type or unmodified PD-1 genetic material and screened for desired specific binding affinity and/or induction of IFN-gamma expression or other functional activity. In some embodiments, a variant PD-1 polypeptide is synthesized de novo utilizing protein or nucleic acid sequences available at any number of publicly available databases and then subsequently screened. The National Center for Biotechnology Information provides such information and its website is publicly accessible via the internet as is the UniProtKB database as discussed previously.

Unless stated otherwise, as indicated throughout the present disclosure, the amino acid substitution(s) are designated by amino acid position number corresponding to the numbering of positions of the unmodified ECD sequence set forth in SEQ ID NO: 37 or, where applicable, the unmodified IgV sequence containing residues 35-145 of SEQ ID NO: 10.

Modifications provided herein can be in a wild-type or unmodified PD-1 polypeptide set forth in SEQ ID NO: 37 or in a portion thereof containing an IgV domain or a specific binding fragment thereof. In some embodiments, the wild-type or unmodified PD-1 polypeptide contains the IgV of PD-1 as set forth in SEQ ID NO: 335. In some embodiments, the unmodified PD-1 polypeptide contains an IgV that can be several amino acids longer or shorter, such as 1-15, e.g. 1, 2, 3, 4, 5, 6, 7, 8, or 9 amino acids longer or shorter, than the sequence of amino acids set forth by SEQ ID NO: 335. In some embodiments, the unmodified PD-1 polypeptide has 85%, 85%, 87%, 88%, 89%, 90%, 91%, 92%, 93%, 94%, 95%, 96%, 97%, 98%, 99% sequence identity to SEQ ID NO: 37, 335, 336, or 337. In some embodiments, the unmodified PD-1 polypeptide has the sequence set forth in any of SEQ ID NO: 37. In some embodiments, the unmodified PD-1 polypeptide has the sequence set forth by SEQ ID NO: 335. In some embodiments, the unmodified PD-1 polypeptide has the sequence set forth by SEQ ID NO: 336. In some embodiments, the unmodified PD-1 polypeptide has the sequence set forth by SEQ ID NO: 337. In some embodiments, the unmodified PD-1 polypeptide has the sequence set forth by SEQ ID NO: 339.

It is within the level of a skilled artisan to identify the corresponding position of a modification, e.g. amino acid substitution, in a PD-1 polypeptide, including portion thereof containing an IgSF domain (e.g. IgV) thereof, such as by alignment of a reference sequence with SEQ ID NO: 37. For example, following alignment, residue 112 of SEQ ID NO: 37 corresponds to residue 107 of SEQ ID NO: 336.

In some embodiments, the variant PD-1 polypeptide has one or more amino acid modifications, e.g. substitutions, in a wild-type or unmodified PD-1 sequence. The one or more amino acid modifications, e.g. substitutions, can be in the ectodomain (extracellular domain) of the wild-type or unmodified PD-1 sequence. In some embodiments, the one or more amino acid modifications, e.g. substitutions, are in the IgV domain or specific binding fragment thereof.

In some embodiments, the variant PD-1 polypeptide has up to 1, 2, 3, 4, 5, 6, 7, 8, 9, 10, 11, 12, 13, 14, 15, 16, 17, 18, 19, or 20 amino acid modifications, e.g. substitutions. The modifications (e.g. substitutions) can be in the IgV domain. In some embodiments, the variant PD-1 polypeptide has up to 1, 2, 3, 4, 5, 6, 7, 8, 9, 10, 11, 12, 13, 14, 15, 16, 17, 18, 19, or 20 amino acid modifications, e.g. substitutions, in the IgV domain or specific binding fragment thereof. In some embodiments, the variant PD-1 polypeptide has less than 100% sequence identity and at least about 85%, 86%, 86%, 88%, 89%, 90%, 91%, 92%, 93%, 94%, 95%, 96%, 97%, 98%, or 99% sequence identity with the wild-type or unmodified PD-1 polypeptide or specific binding fragment thereof, such as with the amino acid sequence of SEQ ID NO: 37, 335, 336, 337, or 339.

In some embodiments, the variant PD-1 polypeptide has one or more amino acid modifications, e.g. substitutions, in an unmodified PD-1 or specific binding fragment thereof corresponding to position(s) 8, 9, 11, 12, 13, 14, 16, 17, 18, 20, 21, 22, 23, 24, 25, 28, 29, 30, 31, 33, 34, 35, 36, 37, 38, 39, 40, 41, 42, 43, 44, 45, 46, 48, 50, 51, 52, 53, 54, 55, 56, 57, 58, 59, 60, 64, 66, 67, 68, 69, 70, 71, 72, 73, 75, 76, 77, 78, 79, 80, 81, 84, 85, 86, 87, 89, 90, 91, 92, 93, 94, 95, 96, 100, 102, 104, 105, 107, 109, 111, 112, 113, 114, 115, 116, 119, 120, 125, 127, 128, 129, 130, 131, 132, 133, 134, 135, 136, 137, 138, 139, 140, 141, 142, 143, or 144, with reference to positions set forth in SEQ ID NO: 37. In some embodiments, such variant PD-1 polypeptides exhibit altered binding affinity to one or more of PD-L1 and/or PD-L2 compared to the wild-type or unmodified PD-1 polypeptide. For example, in some embodiments, the variant PD-1 polypeptide exhibits increased binding affinity to PD-L1 and/or PD-L2 compared to a wild-type or unmodified PD-1 polypeptide. In some embodiments, the variant PD-1 polypeptide exhibits decreased binding affinity to PD-L1 or PD-L2 compared to a wild-type or unmodified PD-1 polypeptide.

In some embodiments, the variant PD-1 polypeptide has one or more amino acid substitutions selected from P8T, D9E, D9G, D9N, D9V, P11 Å, W12G, W12L, W12R, N13D, N13S, N13Y, P14H, P14L, P14S, T16A, T16I, T16S, F17I, F17L, F17V, F17Y, S18T, A20S, A20T, A20V, L21V, L22I, V23E, V23G, V24L, T25A, D28E, N29D, N29S, A30V, T311, T3IN, T31S, T33I, C34Y, S35N, F36I, F36L, F36Y, S37P, S37T, N38D, N38S, N38T, T39A, T39R, T39S, S40P, S40T, E41D, E41V, S42G, S42R, F43L, F43Y, V44H, V44M, V44R, L45I, L45V, N46I, N46V, Y48F, Y48H, Y48N, R49Y, R49L, M50D, M50E, M50I, M50L, M50Q, M50V, M50T, S51G, P52A, P52L, S53D, S53G, S53L, S53N, S53T, S53V, N54C, N54H, N54D, N54G, N54S, N54Y, Q55E, Q55H, Q55K, Q55R, T56A, T56L, T56M, T56P, T56S, T56V, D57F, D57R, D57V, D57Y, K58L, K58R, K58T, L59M, L59R, L59V, A61L, A61S, E64D, E64K, R66H, R66S, S67C, S67G, S67I, S67N, S67R, Q68E, Q68I, Q68L, Q68P, Q68R, Q68T, P69H, P69L, P69S, G70C, G70E, G70F, G70I, G70L, G70N, G70R, G70V, G70S, Q71H, Q71K, Q71L, Q71P, Q71R, D72A, D72G, D72N, C73A, C73G, C73H, C73P, C73S, C73R, C73Y, F75Y, R76G, R76H, R76S, V77D, V77I, T78I, T78S, Q79A, Q79P, Q79R, L80Q, P81S, N82S, R84H, R84Q, D85G, D85N, F86Y, H87L, H87Q, H87R, M88L, M88F, S89G, S89N, V90L, V90M, V91A, V91D, V91I, R92G, R92N, R92S, A93V, R94Q, R95L, R95K, R95G, N96D, N96S, N96T, T100A, T100I, T100S, Y101F, L102F, L102I, L102Y, L102V, G104A, G104T, G104S, G104V, A105C, A105G, A105I, A105L, A105V, I106L, S107A, S107F, S107L, S107T, S107V, L108F, L108I, L108T, L108Y, A109D, A109G, A109H, A109S, P110A, K111E, K111G, K111I, K111M, K111N, K111R, K111T, K111V, A112I, A112P, A112V, Q113R, Q113W, I114T, K115D, K115E, K115IN, K115N, K115Q, K115R, E116D, R119G, R119H, R119L, R119P, R119Q, R119W, A120V, T125A, T125K, T125I, T125S, T125V, R127F, R127L, R127K, R127S, R127V, R128G, R128M, A129S, E130K, V131A, V131E, V131I, V131R, P132H, P132R, P132S, P132T, T133A, T133R, T133S, A134D, A134V, H135N, H135R, H135Y, P136L, P136T, S137C, P138S, P138T, S139T, P140A, P140L, P140R, R141G, R141M, R141S, R141W, P142A, P142L, P142R, P142T, A143D, A143S, A143V, G144D, or G144S, or a conservative amino acid substitution thereof.

In some embodiments, the variant PD-1 is a variant PD-1 that contains one or more amino acid substitutions from N13D, N13S, F17L, T25A, N29S, A30V, N38D, T39A, V44H, V44R, L45I, L45V, N46I, N46V, Y48F, Y48H, R49Y, R49L, M50D, M50E, M50I, M50L, M50Q, M50V, S53D, S53G, S53L, S53N, S53V, N54C, N54D, N54G, N54S, N54Y, Q55E, Q55H, Q55K, T56A, T56L, T56V, D57F, D57R, D57V, D57Y, K58L, K58T, A61L, A61S, S67G, Q68E, Q68I, Q68L, Q68P, Q68R, Q68T, P69L, P69S, G70F, G70I, G70L, G70N, G70R, G70V, Q71P, Q71R, D72A, D72G, C73S, C73R, R76G, V77I, T78I, Q79A, Q79R, N82S, H87Q, H87R, M88L, M88F, R92G, R95K, R95G, N96D, N96S, Y101F, L102I, L102Y, L102V, G104S, A105I, A105V, S107A, S107F, S107L, S107T, S107V, L108F, L108I, L108Y, A109D, A109H, A109S, P110A, K111E, K111G, K111I, K111R, K111T, K111V, A112I, A112P, A112V, K115R, R119G, A120V, T125A, T125I, T125V, R127F, R127L, R127K, R127V, R128G, V1311, V131R, or a conservative amino acid substitution thereof. In some embodiments, the variant PD-1 polypeptide contains the amino acid substitutions S67N/C73R/F86Y/V91D/S107T/A112V/K115D/A120V. In some embodiments, the variant PD-1 polypeptide has the sequence of amino acids set forth in SEQ ID NO: 315, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 315. In some embodiments, the variant PD-1 polypeptide contains the amino acid substitutions V44H/L45V/N46I/Y48H/M50E/N54G/K58T/L102V/A105V/A112I. In some embodiments, the variant PD-1 polypeptide has the sequence of amino acids set forth in SEQ ID NO:334, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:334. Such variant PD-1 polypeptides can be linked, directly or indirectly, to one or more other immunoglobulin superfamily (IgSF) domains as described.

Provided herein are immunomodulatory proteins containing a variant CD86 polypeptide, such as any described in Section II, and an IgSF domain of a PD-1 polypeptide or variant thereof that binds to PD-L1 and/or PD-L2 (CD86/PD-1 immunomodulatory protein). In some embodiments, the variant CD86 polypeptide is or contains the extracellular domain of CD86 or an IgSF (e.g. IgV) domain thereof or a specific binding fragment thereof containing one or more modifications (e.g. substitutions), such as any as described herein. In some embodiments, the variant PD-1 polypeptide is or contains the extracellular domain of PD-1 or an IgSF (e.g. IgV) domain thereof or a specific binding fragment thereof containing one or more modifications (e.g. substitutions), such as any as described herein. The CD86/PD-1 immunomodulatory proteins can be provided in various construct formats as described in Section III.C.3.

### 2. Tumor Antigen Binding IgSF Domains

In some embodiments, the one or more additional IgSF domain (e.g., second or third IgSF) domain is an IgSF domain (e.g., IgV) of another IgSF family member that binds or recognizes a tumor antigen. In such embodiments, the IgSF family member serves as a tumor-localizing moiety, thereby bringing the vIgD of CD86 in close proximity to immune cells in the tumor microenvironment. In some embodiments, the additional IgSF domain (e.g., second IgSF) is an IgSF domain of NKp30, which binds or recognizes B7-H6 expressed on a tumor cell.

In some embodiments, the at least one additional (e.g., second) IgSF domain, e.g., NKp30, is an affinity-modified IgSF domain or vIgD that contains one or more amino acid modifications (e.g., substitutions, deletions or additions). In some embodiments, the one or more amino acid modifications increase binding affinity and/or selectivity to B7-H6 compared to unmodified IgSF domain, e.g., NKp30, such as by at least or at least about 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold. Exemplary amino acid modifications, such as substitutions, deletions or additions, in an IgSF domain (e.g., IgC-like or full ECD) of a variant NKp30 polypeptide are set forth in Table 2. Among the exemplary polypeptides is an NKp30 variant that contains the mutations L30V/A60V/S64P/S86G with reference to positions in the NKp30 extracellular domain corresponding to positions set forth in SEQ ID NO: 54. In some embodiments, there is provided an immunomodulatory protein containing any of the provided variant CD86 polypeptides and a variant NKp30 polypeptide containing an IgC-like domain including any of the amino acid modifications set forth in Table 3, such as the IgC-like domain set forth in any of SEQ ID NOS: 268-272 or an IgV domain that has at least 85%, 86%, 87%, 88%, 89%, 90%, 91%, 92%, 93%, 94%, 95%, 96%, 97%, 98%, 99% to any of SEQ ID NOS: 268-272 and contains the one or more amino acid modifications. In some embodiments, there is provided an immunomodulatory protein containing any of the provided variant CD86 polypeptides and a variant NKp30 polypeptide containing an ECD or a portion thereof containing an IgSF domain or domains, in which is contained any of the amino acid modifications set forth in Table 3, such as the ECD set forth in any of SEQ ID NOS: 273-277 or an ECD that contains at least 85%, 86%, 87%, 88%, 89%, 90%, 91%, 92%, 93%, 94%, 95%, 96%, 97%, 98%, 99% to any of SEQ ID NOS: 273-277 and contains the one or more amino acid modifications.

Table 3 provides exemplary polypeptides containing one or more affinity-modified IgSF domains that can be used in stack constructs provided herein.

TABLE 3
Exemplary variant NKp30 polypeptides

| | ECD | IgC |
|---|---|---|
| Mutation(s) | SEQ ID NO | SEQ ID NO |
| Wild-type | 54 | 278 |
| L30V/A60V/S64P/S86G | 273 | 268 |
| L30V | 274 | 269 |
| A60V | 275 | 270 |
| S64P | 276 | 271 |
| S86G | 277 | 272 |
| L30V/A60V/S64P/S86G/G117del | 231 | 268 |

Provided herein are immunomodulatory proteins containing a variant CD86 polypeptide, such as any described in Section II, and an NKp30 polypeptide or variant thereof that binds to B7-H6 (CD86/NkP30 immunomodulatory protein). In some embodiments, the variant CD86 polypeptide is or contains the extracellular domain of CD86 or an IgSF (e.g. IgV) domain thereof or a specific binding fragment thereof containing one or more modifications (e.g. substitutions), such as any as described herein. In some embodiments, the variant NkP30 polypeptide is or contains the extracellular domain of Nkp30 or an IgSF (e.g. IgV) domain thereof or a specific binding fragment thereof containing one or more modifications (e.g. substitutions), such as any as described herein. The CD86/Nkp30 immunomodulatory proteins can be provided in various construct formats as described in Section III.C.3. In some embodiments, the CD86/Nkp30 immunomodulatory proteins exhibit at least 85%, 86%, 87%, 88%, 89%, 90%, 91%, 92%, 93%, 94%, 95%, 96%, 97%, 98% or 99% sequence identity to a sequence set forth in any of SEQ ID NOS:135, 136, 137, 138, 139 or 140. In some embodiments, the variant CD86/Nkp30 immunomodulatory protein has the sequence set forth in SEQ ID NOS: 135, 136, 137, 138, 139 or 140.

### 3. Constructs

In some embodiments, the two or more IgSF domain, including a vIgD of CD86 and one or more additional IgSF domain (e.g., second or third variant IgSF domain) from another IgSF family member, are covalently or non-covalently linked. A plurality of non-affinity modified and/or affinity modified IgSF domains in a stacked immunomodulatory protein polypeptide chain need not be covalently linked directly to one another. In some embodiments, the two or more IgSF domains are linked directly or indirectly, such as via a linker. In some embodiments, an intervening span of one or more amino acid residues indirectly covalently bonds IgSF domains to each other. The linkage can be via the N-terminal to C-terminal residues. In some embodiments, the linkage can be made via side chains of amino acid residues that are not located at the N-terminus or C-terminus of the IgSF domain(s). Thus, linkages can be made via terminal or internal amino acid residues or combinations thereof.

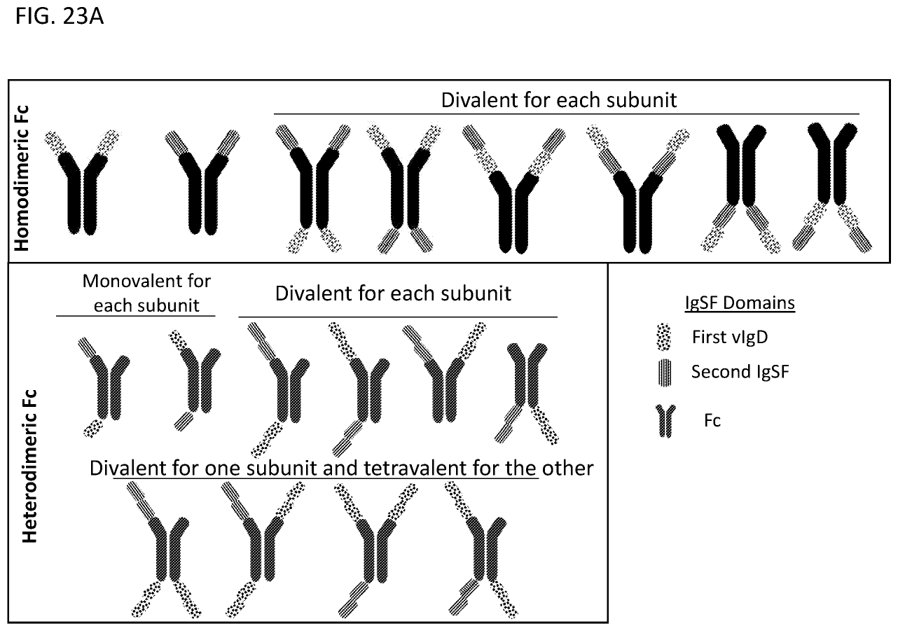

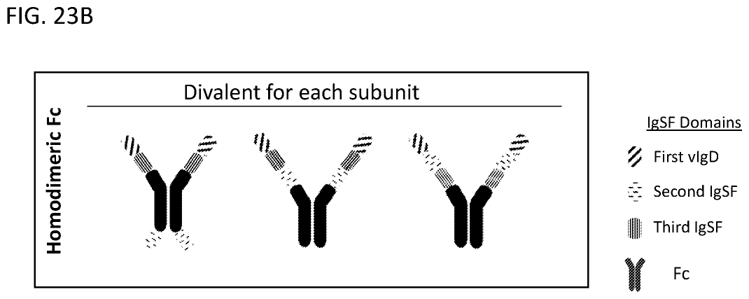

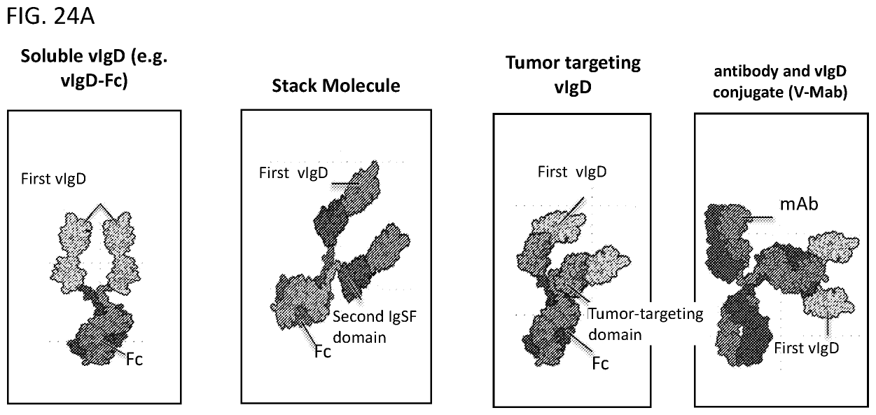

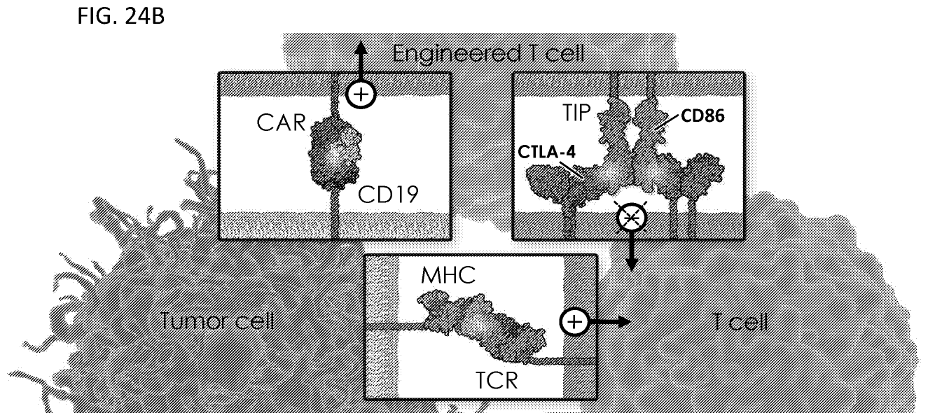

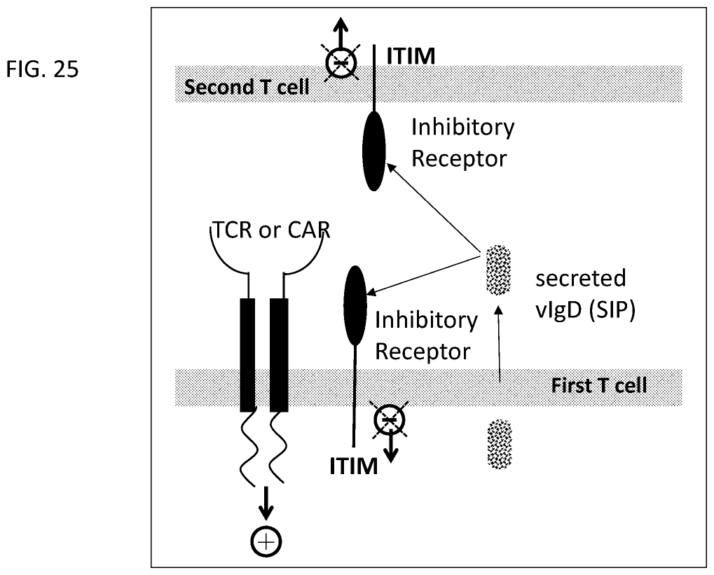

In some embodiments, the immunomodulatory protein contains at least two IgSF domains, each linked directly or indirectly via a linker. In some embodiments, the immunomodulatory protein contains at least three immunomodulatory proteins, each linked directly or indirectly via a linker. Various configurations are shown in FIGS. 23A and 23B.

In some embodiments, one or more “peptide linkers” link the vIgD of CD86 and one or more additional IgSF domain (e.g., second or third variant IgSF domain). In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the linker is a flexible linker. In some embodiments, the linker is (in one-letter amino acid code): GGGGS (“4GS”) or multimers of the 4GS linker, such as repeats of 2, 3, 4, or 5 4GS linkers. In some embodiments, the peptide linker is (GGGGS)₂ (SEQ ID NO: 225) or (GGGGS)₃ (SEQ ID NO: 224). In some embodiments, the linker also can include a series of alanine residues alone or in addition to another peptide linker (such as a 4GS linker or multimer thereof). In some embodiments, the number of alanine residues in each series is: 2, 3, 4, 5, or 6 alanines. In some embodiments, the linker also can include a series of alanine residues alone or in addition to another peptide linker (such as a 4GS linker or multimer thereof). In some embodiments, the number of alanine residues in each series is: 2, 3, 4, 5, or 6 alanines. In some embodiments, the linker is a rigid linker. For example, the linker is an α-helical linker. In some embodiments, the linker is (in one-letter amino acid code): EAAAK or multimers of the EAAAK linker, such as repeats of 2, 3, 4, or 5 EAAAK linkers, such as set forth in SEQ ID NO: 265 (1×EAAAK), SEQ ID NO: 266 (3×EAAAK) or SEQ ID NO: 247 (5×EAAAK). In some embodiments, the linker can further include amino acids introduced by cloning and/or from a restriction site, for example the linker can include the amino acids GS (in one-letter amino acid code) as introduced by use of the restriction site BAMHI. For example, in some embodiments, the linker (in one-letter amino acid code) is GSGGGGS (SEQ ID NO:222), GS(G₄S)₃ (SEQ ID NO: 227), or GS(G₄S)₅ (SEQ ID NO: 228). In some examples, the linker is a 2×GGGGS followed by three alanines (GGGGSGGGGSAAA; SEQ ID NO: 226). In some cases, the immunomodulatory polypeptide comprising a variant CD86 comprises various combinations of peptide linkers.

In some embodiments, the immunomodulatory protein includes a variant CD86 molecule and a variant NKp30 molecule. In some embodiments, the immunomodulatory protein includes or has a sequence having at least 70, 75, 80, 85, 90, 91, 92, 93, 94, 95, 96, 97, 98, or 99% sequence identity to the sequence set forth by SEQ ID NO: 135, 136, 137, 138, 139, or 140. In some embodiments, the immunomodulatory protein includes or has a sequence set forth by SEQ ID NO: 135, 136, 137, 138, 139, or 140. In some embodiments, any of the foregoing sequences form a homodimer. In some embodiments, the homodimer is formed via a multimerization domain that is an Fc domain contained in the immunomodulatory protein. In some embodiments, the homodimer includes the sequence of SEQ ID NO: SEQ ID NO: 135. In some embodiments, the homodimer includes the sequence of SEQ ID NO: SEQ ID NO: 136. In some embodiments, the homodimer includes the sequence of SEQ ID NO: SEQ ID NO: 137. In some embodiments, the homodimer includes the sequence of SEQ ID NO: SEQ ID NO: 138. In some embodiments, the homodimer includes the sequence of SEQ ID NO: SEQ ID NO: 139. In some embodiments, the homodimer includes the sequence of SEQ ID NO: SEQ ID NO: 140.

In some embodiments, the immunomodulatory protein includes a variant CD86 molecule and a variant PD-1 molecule. In some embodiments, the immunomodulatory protein includes or has a sequence having at least 70, 75, 80, 85, 90, 91, 92, 93, 94, 95, 96, 97, 98, or 99% sequence identity to the sequence set forth by SEQ ID NO: 316, 317, 318, 319, 320, 321, 322, or 323. In some embodiments, the immunomodulatory protein includes or has a sequence set forth by SEQ ID NO: 316, 317, 318, 319, 320, 321, 322, or 323. In some embodiments, the immunomodulatory protein includes or has a sequence having at least 70, 75, 80, 85, 90, 91, 92, 93, 94, 95, 96, 97, 98, or 99% sequence identity to the sequence set forth by SEQ ID NO: 326 or 327. In some embodiments, the immunomodulatory protein includes or has the sequence set forth by SEQ ID NO: 326 or 327. In some embodiments, any of the foregoing sequences form a homodimer. In some embodiments, the homodimer is formed via a multimerization domain that is an Fc domain contained in the immunomodulatory protein. In some embodiments, the homodimer includes or has the sequence of SEQ ID NO: 326. In some embodiments, the homodimer includes or has the sequence of SEQ ID NO: 327.

In some embodiments, the immunomodulatory protein includes or has a sequence having at least 70, 75, 80, 85, 90, 91, 92, 93, 94, 95, 96, 97, 98, or 99% sequence identity to the sequence set forth by SEQ ID NO: 328, 329, 330, or 331. In some embodiments, the immunomodulatory protein includes or has the sequence set forth by SEQ ID NO: 328, 329, 330, or 331. In some embodiments any of the foregoing sequences form a heterodimer. In some embodiments, the heterodimer is formed via a multimerization domain that is an Fc domain contained in the immunomodulatory protein. In some embodiments, the first polypeptide of the heterodimer comprises the sequence of SEQ ID NO: 350 and the second polypeptide of the heterodimer comprises the sequence of SEQ ID NO: 351. In some embodiments, the first polypeptide of the heterodimer comprises the sequence of SEQ ID NO: 350 and the second polypeptide of the heterodimer comprises the sequence of SEQ ID NO: 352. In some embodiments, the first polypeptide of the heterodimer comprises the sequence of SEQ ID NO: 350 and the second polypeptide of the heterodimer comprises the sequence of SEQ ID NO: 353.

In some embodiments, the non-affinity modified and/or affinity modified IgSF domains are linked by “wild-type peptide linkers” inserted at the N-terminus and/or C-terminus of a non-affinity modified and/or affinity modified IgSF domains. These linkers are also called leading sequences (N-terminal to non-affinity modified or affinity modified IgSF domain) or trailing sequences (C-terminal to non-affinity modified or affinity modified IgSF domain), and sequences that exist in the wild-type protein that span immediately outside the structural prediction of the Ig fold of the IgSF. In some embodiments, the “wild-type linker” is an amino acid sequence that exists after the signal sequence, but before in the IgSF domain, such as the defined IgV domain, in the amino acid sequence of the wild-type protein. In some embodiments, the “wild-type” linker is an amino acid sequence that exists immediately after the IgSF domain, such as immediately after the defined IgV domain but before the IgC domain, in the amino acid sequence of the wild-type protein. These linker sequences can contribute to the proper folding and function of the neighboring IgSF domain(s). In some embodiments, there is present a leading peptide linker inserted at the N-terminus of the first IgSF domain and/or a trailing sequence inserted at the C-terminus of the first non-affinity modified and/or affinity modified IgSF domain. In some embodiments, there is present a second leading peptide linker inserted at the N-terminus of the second IgSF domain and/or a second trailing sequence inserted at the C-terminus of the second non-affinity modified and/or affinity modified IgSF domain. When the first and second non-affinity modified and/or affinity modified IgSF domains are derived from the same parental protein and are connected in the same orientation, wild-type peptide linkers between the first and second non-affinity modified and/or affinity modified IgSF domains are not duplicated. For example, when the first trailing wild-type peptide linker and the second leading wild-type peptide linker are the same, the Type II immunomodulatory protein does not comprise either the first trailing wild-type peptide linker or the second leading wild-type peptide linker.

In some embodiments, the Type II immunomodulatory protein comprises a first leading wild-type peptide linker inserted at the N-terminus of the first non-affinity modified and/or affinity modified IgSF domain, wherein the first leading wild-type peptide linker comprises at least 5 (such as at least about any of 6, 7, 8, 9, 10, 11, 12, 13, 14, 15, or more) consecutive amino acids from the intervening sequence in the wild-type protein from which the first non-affinity modified and/or affinity modified IgSF domain is derived between the parental IgSF domain and the immediately preceding domain (such as a signal peptide or an IgSF domain). In some embodiments, the first leading wild-type peptide linker comprises the entire intervening sequence in the wild-type protein from which the first non-affinity modified and/or affinity modified IgSF domain is derived between the parental IgSF domain and the immediately preceding domain (such as a signal peptide or an IgSF domain).

In some embodiments, the Type II immunomodulatory protein further comprises a first trailing wild-type peptide linker inserted at the C-terminus of the first non-affinity modified and/or affinity modified IgSF domain, wherein the first trailing wild-type peptide linker comprises at least 5 (such as at least about any of 6, 7, 8, 9, 10, 11, 12, 13, 14, 15, or more) consecutive amino acids from the intervening sequence in the wild-type protein from which the first non-affinity modified and/or affinity modified IgSF domain is derived between the parental IgSF domain and the immediately following domain (such as an IgSF domain or a transmembrane domain). In some embodiments, the first trailing wild-type peptide linker comprises the entire intervening sequence in the wild-type protein from which the first non-affinity modified and/or affinity modified IgSF domain is derived between the parental IgSF domain and the immediately following domain (such as an IgSF domain or a transmembrane domain).

In some embodiments, the Type II immunomodulatory protein further comprises a second leading wild-type peptide linker inserted at the N-terminus of the second non-affinity modified and/or affinity modified IgSF domain, wherein the second leading wild-type peptide linker comprises at least 5 (such as at least about any of 6, 7, 8, 9, 10, 11, 12, 13, 14, 15, or more) consecutive amino acids from the intervening sequence in the wild-type protein from which the second non-affinity modified and/or affinity modified IgSF domain is derived between the parental IgSF domain and the immediately preceding domain (such as a signal peptide or an IgSF domain). In some embodiments, the second leading wild-type peptide linker comprises the entire intervening sequence in the wild-type protein from which the second non-affinity modified and/or affinity modified IgSF domain is derived between the parental IgSF domain and the immediately preceding domain (such as a signal peptide or an IgSF domain).

In some embodiments, the Type II immunomodulatory protein further comprises a second trailing wild-type peptide linker inserted at the C-terminus of the second non-affinity modified and/or affinity modified IgSF domain, wherein the second trailing wild-type peptide linker comprises at least 5 (such as at least about any of 6, 7, 8, 9, 10, 11, 12, 13, 14, 15, or more) consecutive amino acids from the intervening sequence in the wild-type protein from which the second non-affinity modified and/or affinity modified IgSF domain is derived between the parental IgSF domain and the immediately following domain (such as an IgSF domain or a transmembrane domain). In some embodiments, the second trailing wild-type peptide linker comprises the entire intervening sequence in the wild-type protein from which the second non-affinity modified and/or affinity modified IgSF domain is derived between the parental IgSF domain and the immediately following domain (such as an IgSF domain or a transmembrane domain).

In some embodiments, the two or more IgSF domain, including a vIgD of CD86 and one or more additional IgSF domain (e.g., second and/or third variant IgSF domain) from another IgSF family member, are linked or attached to an Fc to form an Fc fusion, which, upon expression in a cell can, in some aspects, produce a dimeric multi-domain stack immunomodulatory protein. Thus, also provided are dimeric multi-domain immunomodulatory proteins.

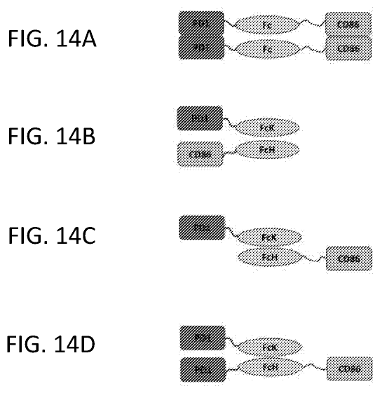

In some embodiments, the variant CD86 polypeptide and one or more IgSF domain are independently linked, directly or indirectly, to the N- or C-terminus of an Fc region. In some embodiments, the variant CD86 polypeptide and at least one of the one or more additional IgSF domain are linked, directly or indirectly, and one of the variant CD86 and one of the one or more additional IgSF domain is also linked, directly or indirectly, to the N- or C-terminus of an Fc region. In some embodiments, the N- or C-terminus of the Fc region is linked to the variant CD86 polypeptide or the one or more additional IgSF domain and the other of the N- or C-terminus of the Fc region is linked to the other of the CD86 variant or another of the one or more additional IgSF domain. In some embodiments, linkage to the Fc is via a peptide linker, e.g., a peptide linker, such as described above. In some embodiments, linkage between the variant CD86 and the one or more additional IgSF domain is via a peptide linker, e.g., a peptide linker, such as described above. In some embodiments, the vIgD of CD86, the one or more additional IgSF domains, and the Fc domain can be linked together in any of numerous configurations. Exemplary configurations are described in the Examples. See for example, FIGS. 14A-14D.

In some embodiments, the stacked immunomodulatory protein is a dimer formed by two immunomodulatory Fc fusion polypeptides. Also provided are nucleic acid molecules encoding any of the stacked immunomodulatory proteins. In some embodiments, the dimeric multi-domain stack immunomodulatory protein can be produced in cells by expression, or in some cases co-expression, of stack immunomodulatory Fc fusion polypeptides, such as described above in accord with generating dimeric Fc fusion proteins.

In some embodiments, the dimeric multi-domain stack immunomodulatory protein is divalent for each Fc region, monovalent for each subunit, or divalent for one subunit and tetravalent for the other.

In some embodiments, the dimeric multi-domain stack immunomodulatory protein is a homodimeric multi-domain stack Fc protein. In some embodiments, the dimeric multi-domain stack immunomodulatory protein comprises a first stack immunomodulatory Fc fusion polypeptide and a second stack immunomodulatory Fc fusion polypeptide in which the first and second polypeptide are the same. In some embodiments, the multi-domain stack molecule contains a first Fc fusion polypeptide containing a variant CD86 and a second IgSF domain and a second Fc fusion polypeptide containing the variant CD86 and the second IgSF domain. In some embodiments, the multi-domain stack molecule contains a first Fc fusion polypeptide containing a variant CD86, a second IgSF domain, and a third IgSF domain and a second Fc fusion polypeptide containing the variant CD86, the second IgSF domain, and the third IgSF domain. In some embodiments, the Fc portion of the first and/or second fusion polypeptide can be any Fc as described above. In some embodiments, the Fc portion or region of the first and second fusion polypeptide is the same.

In some embodiments, the multi-domain stack molecule is heterodimeric, comprising two different Fc fusion polypeptides, e.g., a first and a second Fc fusion polypeptide, wherein at least one is an Fc fusion polypeptide containing at least one variant CD86 polypeptide and/or at least one is an Fc fusion polypeptide containing a second IgSF domain (e.g., second variant IgSF domain). In some embodiments, the first or second Fc fusion polypeptide further contains a third IgSF domain (e.g., third variant IgSF domain). In some embodiments, the multi-domain stack molecule contains a first Fc fusion polypeptide containing a variant CD86 and a second Fc fusion polypeptide containing a second IgSF domain, in which, in some cases, the first or second Fc fusion polypeptide additionally contains a third IgSF domain. In some embodiments, the multi-domain stack molecule contains a first Fc fusion polypeptide containing a variant CD86, a second IgSF domain, and in some cases, a third IgSF domain and a second Fc fusion polypeptide that is not linked to either a variant CD86 polypeptide or an additional IgSF domain. In some embodiments, the Fc portion or region of the first and second fusion polypeptide is the same. In some embodiments, the Fc portion or region of the first and second fusion polypeptide is different.

In some embodiments, the multi-domain stack molecule contains a first Fc fusion polypeptide containing 1, 2, 3, 4 or more variant CD86 polypeptides and 1, 2, 3, 4 or more additional IgSF domains, wherein the total number of IgSF domains in the first stack Fc fusion polypeptide is greater than 2, 3, 4, 5, 6 or more. In one example of such an embodiment, the second stack Fc fusion polypeptide contains 1, 2, 3, 4 or more variant CD86 polypeptides and 1, 2, 3, 4 or more additional IgSF domains, wherein the total number of IgSF domains in the first stack Fc fusion polypeptide is greater than 2, 3, 4, 5, 6 or more. In another example of such an embodiment, the second Fc fusion polypeptide is not linked to either a variant CD86 polypeptide or additional IgSF domain.

In some embodiments, the heterodimeric stack molecule contains a first stack immunomodulatory Fc fusion polypeptide and a second stack immunomodulatory Fc fusion polypeptide in which the first and second polypeptide are different. In some embodiments, a heterodimeric stack molecule contains a first Fc polypeptide fusion containing an Fc region and a first variant CD86 polypeptide and/or second IgSF domain (e.g., second variant IgSF domain) and a second Fc polypeptide fusion containing an Fc region and the other of the first variant CD86 polypeptide or the second IgSF domain. In some embodiments, a heterodimeric stack molecule contains a first Fc polypeptide fusion containing an Fc region and a first variant CD86 polypeptide and/or second IgSF domain (e.g., second variant IgSF domain) and a second Fc polypeptide fusion containing an Fc region and both the first variant CD86 polypeptide and second IgSF domain (e.g., second variant IgSF domain) but in a different orientation or configuration from the first Fc region. In some embodiments, the first and/or second Fc fusion polypeptide also contains a third IgSF domain (e.g., third variant IgSF domain).

In some embodiments, the Fc domain of one or both of the first and second stacked immunomodulatory Fc fusion polypeptide comprises a modification (e.g., substitution) such that the interface of the Fc molecule is modified to facilitate and/or promote heterodimerization. In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide.

In some embodiments, a sequence of amino acids is added preceding the Fc sequence for constructs in which the Fc sequence is the N-terminal portion of the sequence. In some cases, the sequence of amino acids HMSSVSAQ (SEQ ID NO: 279) is added immediately preceding the Fc sequence for constructs in which the Fc sequence is the N-terminal portion of the sequence. In some embodiments, a heterodimeric stack molecule contains a first Fc polypeptide fusion containing an Fc region (knob; e.g., the Fc sequence set forth in SEQ ID NOS: 252 or 324) and a first variant polypeptide and/or second IgSF domain (e.g., second variant IgSF domain) and a second Fc polypeptide fusion containing an Fc region (hole; e.g., the Fc sequence set forth in SEQ ID NO: 280 or 325) and a stuffer sequence HMSSVSAQ (SEQ ID NO:279) is added immediately preceding both Fc regions of the first and second Fc polypeptide fusion.

In some embodiments, a first polypeptide that is modified to contain protuberance (knob) amino acids includes replacement of a native or original amino acid with an amino acid that has at least one side chain which projects from the interface of the first polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T), and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, the heterodimeric molecule contains a T366W mutation in the CH3 domain of the “knob chain” and T366S, L368A, Y407V mutations in the CH3 domain of the “hole chain”. In some cases, an additional interchain disulfide bridge between the CH3 domains can also be used (Merchant, A. M., et al., Nature Biotech. 16 (1998) 677-681) e.g., by introducing a Y349C mutation into the CH3 domain of the “knob” or “hole” chain and a E356C mutation or a S354C mutation into the CH3 domain of the other chain. In some embodiments, the heterodimeric molecule contains S354CT366W mutations in one of the two CH3 domains and Y349C, T366S, L368A, Y407V mutations in the other of the two CH3 domains. In some embodiments, the heterodimeric molecule comprises E356C, T366W mutations in one of the two CH3 domains and Y349C, T366S, L368A, Y407V mutations in the other of the two CH3 domains. In some embodiments, the heterodimeric molecule comprises Y349C, T366W mutations in one of the two CH3 domains and E356C, T366S, L368A, Y407V mutations in the other of the two CH3 domains. In some embodiments, the heterodimeric molecule comprises Y349C, T366W mutations in one of the two CH3 domains and S354C, T366S, L368A, Y407V mutations in the other of the two CH3 domains. Examples of other knobs-in-holes technologies are known in the art, e.g., as described by EP 1 870 459 A1.

In some embodiments, the Fc regions of the heterodimeric molecule additionally can contain one or more other Fc mutation, such as any described above. In some embodiments, the heterodimer molecule contains an Fc region with a mutation that reduces effector function.

In some embodiments, an Fc variant containing CH3 protuberance (knob) or cavity (hole) modifications can be joined to a stacked immunomodulatory polypeptide anywhere, but typically via its N- or C-terminus, to the N- or C-terminus of a first and/or second stacked immunomodulatory polypeptide, such as to form a fusion polypeptide. The linkage can be direct or indirect via a linker. Typically, a knob and hole molecule is generated by co-expression of a first stacked immunomodulatory polypeptide linked to an Fc variant containing CH3 protuberance modification(s) with a second stacked immunomodulatory polypeptide linked to an Fc variant containing CH3 cavity modification(s).

### D. Conjugates and Fusions of Variant Polypeptides and Immunomodulatory Proteins

In some embodiments, the variant polypeptides provided herein, which are immunomodulatory proteins comprising variants of an Ig domain of the IgSF family (vIgD), can be conjugated with or fused with a moiety, such as an effector moiety, such as another protein, directly or indirectly, to form a conjugate (“IgSF conjugate”). In some embodiments, the attachment can be covalent or non-covalent, e.g., via a biotin-streptavidin non-covalent interaction. In some embodiments of a CD86-Fc variant fusion, any one or combination of any two or more of the foregoing conjugates can be attached to the Fc or to the variant CD86 polypeptide or to both.

In some embodiments, the moiety can be a targeting moiety, a small molecule drug (non-polypeptide drug of less than 500 Daltons molar mass), a toxin, a cytostatic agent, a cytotoxic agent, an immunosuppressive agent, a radioactive agent suitable for diagnostic purposes, a radioactive metal ion for therapeutic purposes, a prodrug-activating enzyme, an agent that increases biological half-life, or a diagnostic or detectable agent.

In some embodiments, the effector moiety is a therapeutic agent, such as a cancer therapeutic agent, which is either cytotoxic, cytostatic, or otherwise provides some therapeutic benefit. In some embodiments, the effector moiety is a targeting moiety or agent, such as an agent that targets a cell surface antigen, e.g., an antigen on the surface of a tumor cell. In some embodiments, the effector moiety is a label, which can generate a detectable signal, either directly or indirectly. In some embodiments, the effector moiety is a toxin. In some embodiments, the effector moiety is a protein, peptide, nucleic acid, small molecule, or nanoparticle.

In some embodiments, 1, 2, 3, 4, 5 or more effector moieties, which can be the same or different, are conjugated, linked or fused to the variant polypeptide or protein to form an IgSF conjugate. In some embodiments, such effector moieties can be attached to the variant polypeptide or immunomodulatory protein using various molecular biological or chemical conjugation and linkage methods known in the art and described below. In some embodiments, linkers such as peptide linkers, cleavable linkers, non-cleavable linkers or linkers that aid in the conjugation reaction, can be used to link or conjugate the effector moieties to the variant polypeptide or immunomodulatory protein.

In some embodiments, the IgSF conjugate comprises the following components: (protein or polypeptide), (L)_{q} and (effector moiety)ₘ, wherein the protein or polypeptide is any of the described variant polypeptides or immunomodulatory proteins capable of binding one or more cognate counter structure ligands as described; L is a linker for linking the protein or polypeptide to the moiety; m is at least 1; q is 0 or more; and the resulting IgSF conjugate binds to the one or more counter structure ligands. In particular embodiments, m is 1 to 4 and q is 0 to 8.

In some embodiments, there is provided an IgSF conjugate comprising a variant polypeptide or immunomodulatory protein provided herein conjugated with a targeting agent that binds to a cell surface molecule, for example, for targeted delivery of the variant polypeptide or immunomodulatory protein to a specific cell. In some embodiments, the targeting agent is a molecule(s) that has the ability to localize and bind to a molecule present on a normal cell/tissue and/or tumor cell/tumor in a subject. In other words, IgSF conjugates comprising a targeting agent can bind to a ligand (directly or indirectly), which is present on a cell, such as a tumor cell. The targeting agents of the invention contemplated for use include antibodies, polypeptides, peptides, aptamers, other ligands, or any combination thereof, that can bind a component of a target cell or molecule.

In some embodiments, the targeting agent binds a tumor cell(s) or can bind in the vicinity of a tumor cell(s) (e.g., tumor vasculature or tumor microenvironment) following administration to the subject. The targeting agent may bind to a receptor or ligand on the surface of the cancer cell. In another aspect of the invention, a targeting agent is selected which is specific for a noncancerous cells or tissue. For example, a targeting agent can be specific for a molecule present normally on a particular cell or tissue. Furthermore, in some embodiments, the same molecule can be present on normal and cancer cells. Various cellular components and molecules are known. For example, if a targeting agent is specific for EGFR, the resulting IgSF conjugate can target cancer cells expressing EGFR as well as normal skin epidermal cells expressing EGFR. Therefore, in some embodiments, an IgSF conjugate of the invention can operate by two separate mechanisms (targeting cancer and non-cancer cells).

In various aspects of the invention disclosed herein an IgSF conjugate of the invention comprises a targeting agent which can bind/target a cellular component, such as a tumor antigen, a bacterial antigen, a viral antigen, a mycoplasma antigen, a fungal antigen, a prion antigen, an antigen from a parasite. In some aspects, a cellular component, antigen, or molecule can each be used to mean a desired target for a targeting agent. For example, in various embodiments, a targeting agent is specific for or binds to a component, which includes but is not limited to, epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family, e.g., ROR1; CD171 (L1CAM); B7-H6 (NCR3LG1); CD80, tumor glycosylation antigen, e.g., sTn or Tn, such as sTn Ag of MUC1; LHR (LHCGR); phosphatidylserine, discoidin domain receptor (DDR) family; RET receptor family; KLG receptor family; RYK receptor family; MuSK receptor family; Transforming growth factor-α (TGF-α) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTCl, breakpoint cluster region-Abelson (Bcr-abl) fusion products, B-RAF, caspase-5 (CASP-5), caspase-8 (CASP-8), β-catenin (CTNNBl), cell division cycle 27 (CDC27), cyclin-dependent kinase 4 (CDK4), CDKN2A, COA-I, dek-can fusion protein, EFTUD-2, Elongation factor 2 (ELF2), Ets variant gene 6/acute myeloid leukemia 1 gene ETS (ETC6-AML1) fusion protein, fibronectin (FN), e.g., the extradomain A (EDA) of fibronectin, GPNMB, low density lipid receptor/GDP-L fucose: 3-D-galactose 2-α-L-fucosyltransferase (LDLR/FUT) fusion protein, HLA-A2, arginine to isoleucine exchange at residue 170 of the α-helix of the α2-domain in the HLA-A2gene (HLA-A*201-R170I), HLA-Al 1, heat shock protein 70-2 mutated (HSP70-2M), K1AA0205, MART2, melanoma ubiquitous mutated 1, 2, 3 (MUM-I, 2, 3), prostatic acid phosphatase (PAP), neo-PAP, Myosin class I, NFYC, OGT, OS-9, pml-RARα fusion protein, PRDX5, PTPRK, K-ras (KRAS2), N-ras (NRAS), HRAS, RBAF600, SIRT2, SNRPD1, SYT-SSXl or -SSX2 fusion protein, Triosephosphate Isomerase, BAGE, BAGK-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-I, LAGE, LAGE-I, CTL-recognized antigen on melanoma (CAMEL), MAGE-A1 (MAGE-I), MAGE-A2, MAGE-A3, MAGE-A4, MAGE-A5, MAGE-A6, MAGE-A8, MAGE-A9, MAGE-AlO, MAGE-AI 1, MAGE-A12, MAGE-3, MAGE-Bi, MAGE-B2, MAGE-B5, MAGE-B6, MAGE-Cl, MAGE-C2, mucin 1 (MUC1), MART-1/Melan-A (MLANA), gplOO, gplOO/Pmell7 (SILV), tyrosinase (TYR), TRP-I, HAGE, NA-88, NY-ESO-I, NY-ESO-1/LAGE-2, SAGE, Spl7, SSX-1,2,3,4, TRP2-INT2, carcino-embryonic antigen (CEA), Kallikrein 4, mammaglobin-A, OAl, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBBl), HER-2/neu (ERBB2), interleukin 13 receptor α2 chain (IL13Rα2), IL-6 receptor, intestinal carboxyl esterase (iCE), alpha-feto protein (AFP), M-CSF, mdm-2, MUC1, p53 (TP53), PBF, PRAME, PSMA, RAGE-I, RNF43, RU2AS, SOX1O, STEAP1, survivin (BIRC5), human telomerase reverse transcriptase (hTERT), telomerase, Wilms' tumor gene (WTl), SYCP1, BRDT, SPANX, XAGE, ADAM2, PAGE-5, LIPI, CTAGE-I, CSAGE, MMAl, CAGE, BORIS, HOM-TES-85, AF15ql4, HCA661, LDHC, MORC, SGY-I, SPOl 1, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD 19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), j-human chorionic gonadotropin, 3-2 microglobulin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryonic antigen peptide-1 (CAP-I), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins.

In some embodiments, an IgSF conjugate, through its targeting agent, will bind a cellular component of a tumor cell, tumor vasculature or tumor microenvironment, thereby promoting killing of targeted cells via modulation of the immune response, (e.g., by activation of co-stimulatory molecules or inhibition of negative regulatory molecules of immune cell activation), inhibition of survival signals (e.g., growth factor or cytokine or hormone receptor antagonists), activation of death signals, and/or immune-mediated cytotoxicity, such as through antibody dependent cellular cytotoxicity. Such IgSF conjugates can function through several mechanisms to prevent, reduce or eliminate tumor cells, such as to facilitate delivery of conjugated effector moieties to the tumor target, such as through receptor-mediated endocytosis of the IgSF conjugate; or such conjugates can recruit, bind, and/or activate immune cells (e.g., NK cells, monocytes/macrophages, dendritic cells, T cells, B cells). Moreover, in some instances one or more of the foregoing pathways may operate upon administration of one or more IgSF conjugates of the invention.

In some embodiments, an IgSF conjugate, through its targeting agent, will be localized to, such as bind to, a cellular component of a tumor cell, tumor vasculature or tumor microenvironment, thereby modulating cells of the immune response in the vicinity of the tumor. In some embodiments, the targeting agent facilitates delivery of the conjugated IgSF (e.g., vIgD) to the tumor target, such as to interact with its cognate binding partner to alter signaling of immune cells (e.g., NK cells, monocytes/macrophages, dendritic cells, T cells, B cells) bearing the cognate binding partner.

In some embodiments, the targeting agent is an immunoglobulin. As used herein, the term “immunoglobulin” includes natural or artificial mono- or polyvalent antibodies including, but not limited to, polyclonal, monoclonal, multispecific, human, humanized or chimeric antibodies, single chain antibodies, Fab fragments, F(ab′) fragments, fragments produced by a Fab expression library, single chain Fv (scFv); anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, e.g., molecules that contain an antigen binding site that immunospecifically binds an antigen. The immunoglobulin molecules of the invention can be of any type (e.g., IgG, IgE, IgM, IgD, IgA, and IgY), class (e.g., IgG1, IgG2, IgG3, IgG4, IgAQ1, and IgA2) or subclass of immunoglobulin molecule.

In some embodiments, an IgSF conjugate, through its antibody targeting moiety, will bind a cellular component of a tumor cell, tumor vasculature, or tumor microenvironment, thereby promoting apoptosis of targeted cells via modulation of the immune response, (e.g., by activation of co-stimulatory molecules or inhibition of negative regulatory molecules of immune cell activation), inhibition of survival signals (e.g., growth factor or cytokine or hormone receptor antagonists), activation of death signals, and/or immune-mediated cytotoxicity, such as through antibody dependent cellular cytotoxicity. Such IgSF conjugates can function through several mechanisms to prevent, reduce, or eliminate tumor cells, such as to facilitate delivery of conjugated effector moieties to the tumor target, such as through receptor-mediated endocytosis of the IgSF conjugate; or such conjugates can recruit, bind, and/or activate immune cells (e.g., NK cells, monocytes/macrophages, dendritic cells, T cells, B cells).

In some embodiments, an IgSF conjugate, through its antibody targeting moiety, will bind a cellular component of a tumor cell, tumor vasculature or tumor microenvironment, thereby modulating the immune response (e.g., by activation of co-stimulatory molecules or inhibition of negative regulatory molecules of immune cell activation). In some embodiments, such conjugates can recognize, bind, and/or modulate (e.g., inhibit or activate) immune cells (e.g., NK cells, monocytes/macrophages, dendritic cells, T cells, B cells).

Antibody targeting moieties of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Also included in the invention are Fc fragments, antigen-Fc fusion proteins, and Fc-targeting moiety conjugates or fusion products (Fc-peptide, Fc-aptamer). The antibody targeting moieties of the invention may be from any animal origin including birds and mammals. In one aspect, the antibody targeting moieties are human, murine (e.g., mouse and rat), donkey, sheep, rabbit, goat, guinea pig, camel, horse, or chicken. Further, such antibodies may be humanized or chimeric versions of animal antibodies. The antibody targeting moieties of the invention may be monospecific, bispecific, trispecific, or of greater multispecificity.

In various embodiments, an antibody/targeting moiety recruits, binds, and/or activates immune cells (e.g., NK cells, monocytes/macrophages, dendritic cells) via interactions between Fc (in antibodies) and Fc receptors (on immune cells) and via the conjugated variant polypeptides or immunomodulatory proteins provided herein. In some embodiments, an antibody/targeting moiety recognizes or binds a tumor agent and localizes to the tumor cell via the conjugated variant polypeptides or immunomodulatory proteins provided herein to facilitate modulation of immune cells in the vicinity of the tumor.

Examples of antibodies which can be incorporated into IgSF conjugates include but are not limited to antibodies such as Pertuzumab (Perjeta®), Cetuximab (IMC-C225; Erbitux®), Trastuzumab (Herceptin®), Rituximab (Rituxan®; MabThera®), Bevacizumab (Avastin®), Alemtuzumab (Campath®; Campath-1H®; Mabcampath®), Panitumumab (ABX-EGF; Vectibix®), Ranibizumab (Lucentis®), Ibritumomab, Ibritumomab tiuxetan, (Zevalin®), Tositumomab, Iodine 1131 Tositumomab (BEXXAR®), Catumaxomab (Removab®), Gemtuzumab, Gemtuzumab ozogamicine (Mylotarg®), Abatacept (CTLA4-Ig; Orencia®), Belatacept (L104EA29YIg; LEA29Y; LEA), Ipilimumab (MDX-010; MDX-101), Tremelimumab (ticilimumab; CP-675,206), PRS-010, PRS-050, Aflibercept (VEGF Trap, AVE005), Volociximab (M200), F200, MORAb-009, SS1P (CAT-5001), Cixutumumab (IMC-A12), Matuzumab (EMD72000), Nimotuzumab (h-R3), Zalutumumab (HuMax-EGFR), Necitumumab IMC-11F8, mAb806/ch806, Sym004, mAb-425, Panorex @(17-1A) (murine monoclonal antibody); Panorex @(17-1A) (chimeric murine monoclonal antibody); IDEC-Y2B8 (murine, anti-CD20 MAb); BEC2 (anti-idiotypic MAb, mimics the GD epitope) (with BCG); Oncolym (Lym-1 monoclonal antibody); SMART MI95 Ab, humanized 13′ I LYM-I (Oncolym), Ovarex (B43.13, anti-idiotypic mouse MAb); MDX-210 (humanized anti-HER-2 bispecific antibody); 3622W94 MAb that binds to EGP40 (17-1A) pancarcinoma antigen on adenocarcinomas; Anti-VEGF, Zenapax (SMART Anti-Tac (IL-2 receptor); SMART MI95 Ab, humanized Ab, humanized); MDX-210 (humanized anti-HER-2 bispecific antibody); MDX-447 (humanized anti-EGF receptor bispecific antibody); NovoMAb-G2 (pancarcinoma specific Ab); TNT (chimeric MAb to histone antigens); TNT (chimeric MAb to histone antigens); Gliomab-H (Monoclon s-Humanized Abs); GNI-250 Mab; EMD-72000 (chimeric-EGF antagonist); LymphoCide (humanized LL2 antibody); and MDX-260 bispecific, targets GD-2, ANA Ab, SMART IDlO Ab, SMART ABL 364 Ab or ImmuRAIT-CEA. As illustrated by the forgoing list, it is conventional to make antibodies to a particular target epitope.

In some embodiments, the antibody or antigen-binding fragment of the provided conjugates, including fusion molecules, is cetuximab, panitumumab, zalutumumab, nimotuzumab, trastuzumab, Ado-trastuzumab emtansine, Tositumomab (Bexxar @), Rituximab (Rituxan, Mabthera), Ibritumomab tiuxetan (Zevalin), Daclizumab (Zenapax), Gemtuzumab (Mylotarg), Alemtuzumab, CEA-scan Fab fragment, OC125 monoclonal antibody, ab75705, B72.3, Bevacizumab (Avastin®), Afatinib, Axitinib, Bosutinib, Cabozantinib, Ceritinib, Crizotinib, Dabrafenib, Dasatinib, Dinutuximab (Unituxin™), Erlotinib, Everolimus, Ibrutinib, Imatinib, Lapatinib, Lenvatinib, Nilotinib, Olaparib, Olaratumab (Lartruvo™), Palbociclib, Pazopanib, Pertuzumab (Perjeta®), Ramucirumab (Cyramza®), Regorafenib, Ruxolitinib, Sorafenib, Sunitinib, Temsirolimus, Trametinib, Vandetanib, Vemurafenib, Vismodegib, Basiliximab, Ipilimumab, Nivolumab, pembrolizumab, MPDL3280A, Pidilizumab (CT-011), AMP-224, MSB001078C, or MEDI4736, BMS-935559, LY3300054, atezolizumab, avelumab or durvalumab or is an antigen-binding fragment thereof. In some the antibody or antigen-binding fragment of the provided conjugates, including fusion molecules, is Pertuzumab (Perjeta®), panitumumab or an antigen-binding fragment thereof. In some embodiments, the antibody targeting moiety is a full length antibody, or antigen-binding fragment thereof, containing an Fc domain. In some embodiments, the variant polypeptide or immunomodulatory protein is conjugated to the Fc portion of the antibody targeting moiety, such as by conjugation to the N-terminus of the Fc portion of the antibody.

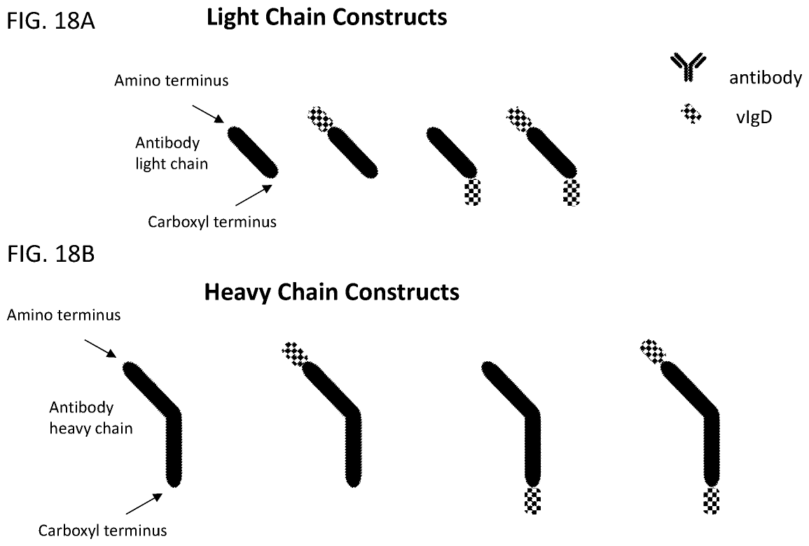

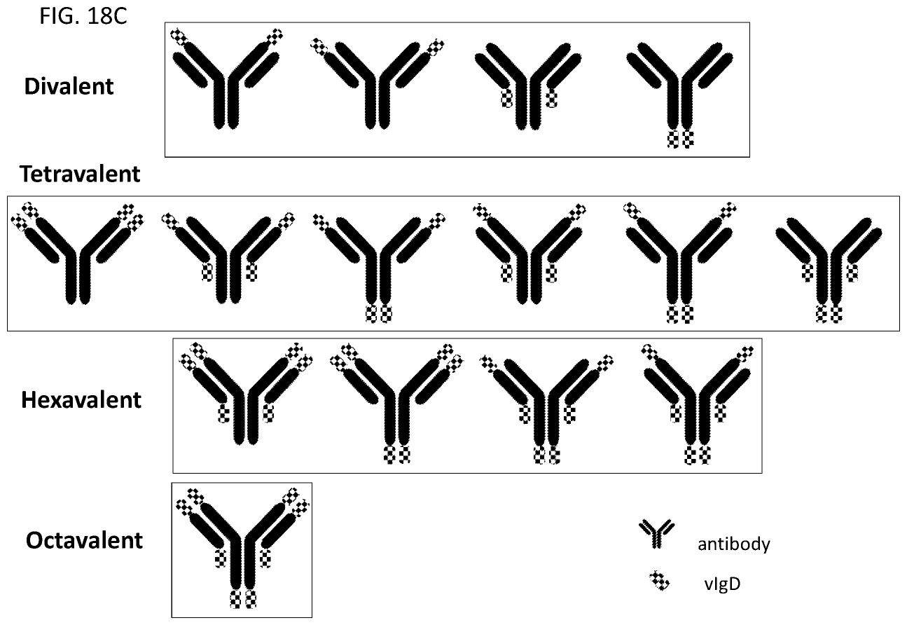

In some embodiments, the vIgD is linked, directly or indirectly, to the N- or C-terminus of the light and/or heavy chain of the antibody. In some embodiments, linkage can be via a peptide linker, such as any described above. In some embodiments, the linker can further include amino acids introduced by cloning and/or from a restriction site. In some embodiments, the linker may include additional amino acids on either end introduced by a restriction site. For example, the linker can include additional amino acids such as SA (in one-letter amino acid code) as introduced by use of the restriction site AFEI. Various configurations can be constructed. FIGS. 18A-18C depict exemplary configurations. In some embodiments, the antibody conjugate can be produced by co-expression of the heavy and light chain of the antibody in a cell.

In some embodiments, HER2 antibodies or antigen binding fragments thereof can be incorporated into the IgSF conjugates. Examples of HER2 antibodies which can be incorporated into IgSF conjugates include but are not limited to antibodies such as Pertuzumab (Perjeta®) and traztuzumab. In some embodiments, the vIgD is linked, directly or indirectly, to the N- or C-terminus of the light and/or heavy chain of an anti-HER2 antibody. In some embodiments, the anti-HER2 antibody is Pertuzumab (Perjeta®). An exemplary light chain and heavy chain of an anti-HER2 antibody Pertuzumab are set forth in SEQ ID NO: 341 and 340, respectively. In some embodiments, the variant CD86 polypeptide described herein is linked to the to the N- or C-terminus of the light and/or heavy chain of Pertuzumab. In some embodiments, a conjugate including Pertuzumab includes or has the V_{H} sequence of SEQ ID NO:342. In some embodiments, a conjugate including Pertuzumab includes or has the V_{L} sequence of SEQ ID NO:343. In some embodiments, a conjugate including Pertuzumab includes or has the V_{H}sequence of SEQ ID NO:344. In some embodiments, a conjugate including Pertuzumab includes or has the V_{L} sequence of SEQ ID NO:345. In some embodiments, a conjugate including Pertuzumab includes a heavy chain sequence having at least 70, 75, 80, 85, 90, 91, 92, 93, 94, 95, 96, 97, 98, or 99% sequence identity to SEQ ID NO: 342 and a light chain sequence having at least 70, 75, 80, 85, 90, 91, 92, 93, 94, 95, 96, 97, 98, or 99% sequence identity to SEQ ID NO: 341. In some embodiments, a conjugate including Pertuzumab include a heavy chain sequence having at least 70, 75, 80, 85, 90, 91, 92, 93, 94, 95, 96, 97, 98, or 99% sequence identity to SEQ ID NO: 340 and a light chain sequence having at least 70, 75, 80, 85, 90, 91, 92, 93, 94, 95, 96, 97, 98, or 99% sequence identity to SEQ ID NO: 343. In some embodiments, a conjugate including Pertuzumab includes a heavy chain sequence having at least 70, 75, 80, 85, 90, 91, 92, 93, 94, 95, 96, 97, 98, or 99% sequence identity to SEQ ID NO: 344 and a light chain sequence having at least 70, 75, 80, 85, 90, 91, 92, 93, 94, 95, 96, 97, 98, or 99% sequence identity to SEQ ID NO: 341. In some embodiments, a conjugate including Pertuzumab includes the heavy chain sequence having at least 70, 75, 80, 85, 90, 91, 92, 93, 94, 95, 96, 97, 98, or 99% sequence identity to SEQ ID NO: 340 and a light chain sequence having at least 70, 75, 80, 85, 90, 91, 92, 93, 94, 95, 96, 97, 98, or 99% sequence identity to SEQ ID NO: 345. In some embodiments, a conjugate including Pertuzumab includes a heavy chain sequence of SEQ ID NO: 342 and a light chain sequence of SEQ ID NO: 341. In some embodiments, a conjugate including Pertuzumab includes a heavy chain sequence of SEQ ID NO: 340 and a light chain sequence of SEQ ID NO: 343. In some embodiments, a conjugate including Pertuzumab includes a heavy chain sequence of SEQ ID NO: 344 and a light chain sequence of SEQ ID NO: 341. In some embodiments, a conjugate including Pertuzumab includes a heavy chain sequence of SEQ ID NO: 340 and a light chain sequence of SEQ ID NO: 345.

In some embodiments, EGFR (HER1) antibodies or antigen binding fragments thereof can be incorporated into the IgSF conjugates. Examples of EGFR antibodies which can be incorporated into IgSF conjugates include but are not limited to antibodies such as panitumumab and cetuximab. In some embodiments, the vIgD is linked, directly or indirectly, to the N- or C-terminus of the light and/or heavy chain of an anti-EGFR antibody. In some embodiments, the anti-EGFR antibody is Panitumumab. In some embodiments, the variant CD86 polypeptide described herein is linked to the to the N- or C-terminus of the light and/or heavy chain of Panitumumab. An exemplary light chain and heavy chain of an anti-EGFR antibody Panitumumab are set forth in SEQ ID NO: 347 and 346, respectively. In some embodiments, a conjugate including Panitumumab includes or has the V_{H} sequence of SEQ ID NO:348. In some embodiments, a conjugate including Panitumumab includes or has the V_{L} sequence of SEQ ID NO:349. In some embodiments, a conjugate including Panitumumab includes or has the V_{H}sequence of SEQ ID NO:350. In some embodiments, a conjugate including Panitumumab includes or has the V_{L} sequence of SEQ ID NO:351. In some embodiments, a conjugate including Panitumumab includes a heavy chain sequence having at least 70, 75, 80, 85, 90, 91, 92, 93, 94, 95, 96, 97, 98, or 99% sequence identity to SEQ ID NO: 348 and a light chain sequence having at least 70, 75, 80, 85, 90, 91, 92, 93, 94, 95, 96, 97, 98, or 99% sequence identity to SEQ ID NO: 347. In some embodiments, a conjugate including Panitumumab include a heavy chain sequence having at least 70, 75, 80, 85, 90, 91, 92, 93, 94, 95, 96, 97, 98, or 99% sequence identity to SEQ ID NO: 346 and a light chain sequence having at least 70, 75, 80, 85, 90, 91, 92, 93, 94, 95, 96, 97, 98, or 99% sequence identity to SEQ ID NO: 349. In some embodiments, a conjugate including Panitumumab includes a heavy chain sequence having at least 70, 75, 80, 85, 90, 91, 92, 93, 94, 95, 96, 97, 98, or 99% sequence identity to SEQ ID NO: 350 and a light chain sequence having at least 70, 75, 80, 85, 90, 91, 92, 93, 94, 95, 96, 97, 98, or 99% sequence identity to SEQ ID NO: 347. In some embodiments, a conjugate including Panitumumab includes the heavy chain sequence having at least 70, 75, 80, 85, 90, 91, 92, 93, 94, 95, 96, 97, 98, or 99% sequence identity to SEQ ID NO: 346 and a light chain sequence having at least 70, 75, 80, 85, 90, 91, 92, 93, 94, 95, 96, 97, 98, or 99% sequence identity to SEQ ID NO: 351. In some embodiments, a conjugate including Panitumumab includes a heavy chain sequence of SEQ ID NO: 348 and a light chain sequence of SEQ ID NO: 347. In some embodiments, a conjugate including Panitumumab includes a heavy chain sequence of SEQ ID NO: 346 and a light chain sequence of SEQ ID NO: 349. In some embodiments, a conjugate including Panitumumab includes a heavy chain sequence of SEQ ID NO: 350 and a light chain sequence of SEQ ID NO: 347. In some embodiments, a conjugate including Panitumumab includes a heavy chain sequence of SEQ ID NO: 346 and a light chain sequence of SEQ ID NO: 351.

In one aspect of the invention, the targeting agent is an aptamer molecule. For example, in some embodiments, the aptamer is comprised of nucleic acids that function as a targeting agent. In various embodiments, an IgSF conjugate of the invention comprises an aptamer that is specific for a molecule on a tumor cell, tumor vasculature, and/or a tumor microenvironment. In some embodiments, the aptamer itself can comprise a biologically active sequence, in addition to the targeting module (sequence), wherein the biologically active sequence can induce an immune response to the target cell. In other words, such an aptamer molecule is a dual use agent. In some embodiments, an IgSF conjugate of the invention comprises conjugation of an aptamer to an antibody, wherein the aptamer and the antibody are specific for binding to separate molecules on a tumor cell, tumor vasculature, tumor microenvironment, and/or immune cells.

The term “aptamer” includes DNA, RNA, or peptides that are selected based on specific binding properties to a particular molecule. For example, an aptamer(s) can be selected for binding a particular gene or gene product in a tumor cell, tumor vasculature, tumor microenvironment, and/or an immune cell, as disclosed herein, where selection is made by methods known in the art and familiar to one of skill in the art.

In some aspects of the invention the targeting agent is a peptide. For example, the variant polypeptides or immunomodulatory proteins provided herein can be conjugated to a peptide which can bind with a component of a cancer or tumor cells. Therefore, such IgSF conjugates of the invention comprise peptide targeting agents which binds to a cellular component of a tumor cell, tumor vasculature, and/or a component of a tumor microenvironment. In some embodiments, targeting agent peptides can be an antagonist or agonist of an integrin. Integrins, which comprise an alpha and a beta subunit, include numerous types well known to a skilled artisan.

In one embodiment, the targeting agent is Vvβ3. Integrin Vvβ3 is expressed on a variety of cells and has been shown to mediate several biologically relevant processes, including adhesion of osteoclasts to bone matrix, migration of vascular smooth muscle cells, and angiogenesis. Suitable targeting molecules for integrins include RGD peptides or peptidomimetics as well as non-RGD peptides or peptidomimetics (see, e.g., U.S. Pat. Nos. 5,767,071 and 5,780,426) for other integrins such as V4. βi (VLA-4), V4-P7 (see, e.g., U.S. Pat. No. 6,365,619; Chang et al, Bioorganic & Medicinal Chem Lett, 12:159-163 (2002); Lin et al., Bioorganic & Medicinal Chem Lett, 12:133-136 (2002)), and the like.

In some embodiments, there is provided an IgSF conjugate comprising a variant polypeptide or immunomodulatory protein provided herein conjugated with a therapeutic agent. In some embodiments, the therapeutic agent includes, for example, daunomycin, doxorubicin, methotrexate, and vindesine (Rowland et al., Cancer Immunol. Immunother. 21:183-187, 1986). In some embodiments, the therapeutic agent has an intracellular activity. In some embodiments, the IgSF conjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the IgSF conjugate must be internalized upon binding to the target cell in order for the protein to be cytotoxic to the cells.

In some embodiments, there is provided an IgSF conjugate comprising a variant polypeptide or immunomodulatory protein provided herein conjugated with a toxin. In some embodiments, the toxin includes, for example, bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition.

In some embodiments, there is provided an IgSF conjugate comprising a variant polypeptide or immunomodulatory protein provided herein conjugated with a label, which can generate a detectable signal, indirectly or directly. These IgSF conjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example 99Tc or 1231, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983). In some embodiments, the IgSF conjugate is detectable indirectly. For example, a secondary antibody that is specific for the IgSF conjugate and contains a detectable label can be used to detect the IgSF conjugate.

The IgSF conjugates may be prepared using any methods known in the art. See, e.g., WO 2009/067800, WO 2011/133886, and U.S. Patent Application Publication No. 2014322129, incorporated by reference herein in their entirety.

The variant polypeptides or immunomodulatory proteins of an IgSF conjugate may be “attached to” the effector moiety by any means by which the variant polypeptides or immunomodulatory proteins can be associated with, or linked to, the effector moiety. For example, the variant polypeptides or immunomodulatory proteins of an IgSF conjugate may be attached to the effector moiety by chemical or recombinant means. Chemical means for preparing fusions or conjugates are known in the art and can be used to prepare the IgSF conjugate. The method used to conjugate the variant polypeptides or immunomodulatory proteins and effector moiety must be capable of joining the variant polypeptides or immunomodulatory proteins with the effector moiety without interfering with the ability of the variant polypeptides or immunomodulatory proteins to bind to their one or more counter structure ligands.

The variant polypeptides or immunomodulatory proteins of an IgSF conjugate may be linked indirectly to the effector moiety. For example, the variant polypeptides or immunomodulatory proteins of an IgSF conjugate may be directly linked to a liposome containing the effector moiety of one of several types. The effector moiety(s) and/or the variant polypeptides or immunomodulatory proteins may also be bound to a solid surface.

In some embodiments, the variant polypeptides or immunomodulatory proteins of an IgSF conjugate and the effector moiety are both proteins and can be conjugated using techniques well known in the art. There are several hundred crosslinkers available that can conjugate two proteins. (See for example “Chemistry of Protein Conjugation and Crosslinking,” 1991, Shans Wong, CRC Press, Ann Arbor). The crosslinker is generally chosen based on the reactive functional groups available or inserted on the variant polypeptides or immunomodulatory proteins and/or effector moiety. In addition, if there are no reactive groups, a photoactivatable crosslinker can be used. In certain instances, it may be desirable to include a spacer between the variant polypeptides or immunomodulatory proteins and the effector moiety. Crosslinking agents known to the art include the homobifunctional agents: glutaraldehyde, dimethyladipimidate and Bis(diazobenzidine) and the heterobifunctional agents: m Maleimidobenzoyl-N-Hydroxysuccinimide and Sulfo-m Maleimidobenzoyl-N-Hydroxysuccinimide.

In some embodiments, the variant polypeptides or immunomodulatory proteins of an IgSF conjugate may be engineered with specific residues for chemical attachment of the effector moiety. Specific residues used for chemical attachment of molecule known to the art include lysine and cysteine. The crosslinker is chosen based on the reactive functional groups inserted on the variant polypeptides or immunomodulatory proteins, and available on the effector moiety.

An IgSF conjugate may also be prepared using recombinant DNA techniques. In such a case a DNA sequence encoding the variant polypeptides or immunomodulatory proteins is fused to a DNA sequence encoding the effector moiety, resulting in a chimeric DNA molecule. The chimeric DNA sequence is transfected into a host cell that expresses the fusion protein. The fusion protein can be recovered from the cell culture and purified using techniques known in the art.

Examples of attaching an effector moiety, which is a label, to the variant polypeptides or immunomodulatory proteins include the methods described in Hunter, et al., Nature 144:945 (1962); David, et al., Biochemistry 13:1014 (1974); Pain, et al., J. Immunol. Meth. 40:219 (1981); Nygren, J. Histochem. and Cytochem. 30:407 (1982); Wensel and Meares, Radioimmunoimaging And Radioimmunotherapy, Elsevier, N.Y. (1983); and Colcher et al., “Use Of Monoclonal Antibodies As Radiopharmaceuticals For The Localization Of Human Carcinoma Xenografts In Athymic Mice”, Meth. Enzymol., 121:802-16 (1986).

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as 99Tc or 1231, 186Re, 188Re and 111In can be attached via a cysteine residue in the peptide. Yttrium-90 can be attached via a lysine residue. The IODOGEN method (Fraker et al., Biochem. Biophys. Res. Commun. 80:49-57 (1978)) can be used to incorporate iodine-123. “Monoclonal Antibodies in Immunoscintigraphy” (Chatal, CRC Press 1989) describes other methods in detail.

Conjugates of the variant polypeptides or immunomodulatory proteins and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-p-isothiocyanatobenzyl-3-methyldiethylenetriaminepentaacetic acid (MX-DTPA) is an exemplary chelating agent for conjugation of radionucleotide to the antibody. See, e.g., WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The IgSF conjugates of the invention expressly contemplate, but are not limited to, drug conjugates prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-IBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL, U.S.A). See pages 467-498, 2003-2004 Applications Handbook and Catalog.

### E. Transmembrane and Secretable Immunomodulatory Proteins and Engineered Cells

Provided herein are engineered cells which express the immunomodulatory variant CD86 polypeptides (alternatively, “engineered cells”). In some embodiments, the expressed immunomodulatory variant CD86 polypeptide is a transmembrane protein and is surface expressed. In some embodiments, the expressed immunomodulatory variant CD86 polypeptide is expressed and secreted from the cell.

### 1. Transmembrane Immunomodulatory Proteins

In some embodiments, an immunomodulatory polypeptide comprising a variant CD86 can be a membrane bound protein. As described in more detail below, the immunomodulatory polypeptide can be a transmembrane immunomodulatory polypeptide comprising a variant CD86 in which is contained: an ectodomain containing at least one affinity modified IgSF domain (IgV or IgC), a transmembrane domain and, optionally, a cytoplasmic domain. In some embodiments, the transmembrane immunomodulatory protein can be expressed on the surface of an immune cell, such as a mammalian cell, including on the surface of a lymphocyte (e.g., T cell or NK cell) or antigen presenting cell. In some embodiments, the transmembrane immunomodulatory protein is expressed on the surface of a mammalian T-cell, including such T-cells as: a T helper cell, a cytotoxic T-cell (alternatively, cytotoxic T lymphocyte or CTL), a natural killer T-cell, a regulatory T-cell, a memory T-cell, or a gamma delta T-cell. In some embodiments, the mammalian cell is an antigen presenting cell (APC). Typically, but not exclusively, the ectodomain (alternatively, “extracellular domain”) comprises the one or more amino acid variations (e.g., amino acid substitutions) of the variant CD86 of the invention. Thus, for example, in some embodiments a transmembrane protein will comprise an ectodomain that comprises one or more amino acid substitutions of a variant CD86 of the invention.

In some embodiments, the engineered cells express variant CD86 polypeptides that are transmembrane immunomodulatory polypeptides (TIPs) that can be a membrane protein such as a transmembrane protein. In typical embodiments, the ectodomain of a membrane protein comprises an extracellular domain or IgSF domain thereof of a variant CD86 provided herein in which is contained one or more amino acid substitutions in at least one IgSF domain as described. The transmembrane immunomodulatory proteins provided herein further contain a transmembrane domain linked to the ectodomain. In some embodiments, the transmembrane domain results in an encoded protein for cell surface expression on a cell. In some embodiments, the transmembrane domain is linked directly to the ectodomain. In some embodiments, the transmembrane domain is linked indirectly to the ectodomain via one or more linkers or spacers. In some embodiments, the transmembrane domain contains predominantly hydrophobic amino acid residues, such as leucine and valine.

In some embodiments, a full length transmembrane anchor domain can be used to ensure that the TIPs will be expressed on the surface of the engineered cell, such as engineered T cell. Conveniently, this could be from a particular native protein that is being affinity modified (e.g., CD86 or other native IgSF protein), and simply fused to the sequence of the first membrane proximal domain in a similar fashion as the native IgSF protein (e.g., CD86). In some embodiments, the transmembrane immunomodulatory protein comprises a transmembrane domain of the corresponding wild-type or unmodified IgSF member, such as a transmembrane domain contained in the sequence of amino acids set forth in SEQ ID NO:2 (Table 2). In some embodiments, the membrane bound form comprises a transmembrane domain of the corresponding wild-type or unmodified polypeptide, such as corresponding to residues 248-268 of SEQ ID NO:2.

In some embodiments, the transmembrane domain is a non-native transmembrane domain that is not the transmembrane domain of native CD86. In some embodiments, the transmembrane domain is derived from a transmembrane domain from another non-CD86 family member polypeptide that is a membrane-bound or is a transmembrane protein. In some embodiments, a transmembrane anchor domain from another protein on T cells can be used. In some embodiments, the transmembrane domain is derived from CD8. In some embodiments, the transmembrane domain can further contain an extracellular portion of CD8 that serves as a spacer domain. An exemplary CD8 derived transmembrane domain is set forth in SEQ ID NO: 281, 282, or 283 or a portion thereof containing the CD8 transmembrane domain. In some embodiments, the transmembrane domain is a synthetic transmembrane domain.

In some embodiments, the transmembrane immunomodulatory protein further contains an endodomain, such as a cytoplasmic signaling domain, linked to the transmembrane domain. In some embodiments, the cytoplasmic signaling domain induces cell signaling. In some embodiments, the endodomain of the transmembrane immunomodulatory protein comprises the cytoplasmic domain of the corresponding wild-type or unmodified polypeptide, such as a cytoplasmic domain contained in the sequence of amino acids set forth in SEQ ID NO:2, such as amino acids 269-329 of SEQ ID NO:2 (see Table 2).

In some embodiments, a provided transmembrane immunomodulatory protein that is or comprises a variant CD86 comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 56 and contains an ectodomain comprising at least one affinity-modified CD86 IgSF domain as described herein and a transmembrane domain. In some embodiments, the transmembrane immunomodulatory protein contains any one or more amino acid substitutions in an IgSF domain (e.g., IgV domain) as described. In some embodiments, the transmembrane immunomodulatory protein can further comprise a cytoplasmic domain as described.

Provided herein are CD86 transmembrane immunomodulatory proteins. In some embodiments the CD86 TIP exhibits at least 85%, 86%, 87%, 88%, 89%, 90%, 91%, 92%, 93%, 94%, 95%, 96%, 97%, 98% or 99% sequence identity to SEQ ID NO: 233. In some embodiments, the CD86 TIP is a variant CD86 TIP that contains one or more amino acid modifications (e.g. substitutions) in the ecotodomain (extracellular portion) or an IgSF (e.g. IgV) domain thereof or a specific binding portion thereof. Exemplary amino acid modifications (e.g. substitutions) include any as described in Section II. In some embodiments, the variant CD86 TIP exhibits at least 85%, 86%, 87%, 88%, 89%, 90%, 91%, 92%, 93%, 94%, 95%, 96%, 97%, 98% or 99% sequence identity to a sequence set forth in any of SEQ ID NOS: 234, 235, 236, 237, 238, 239, 240, or 241. In some embodiments, the variant CD86 TIP has the sequence set forth in SEQ ID NOS: 234, 235, 236, 237, 238, 239, 240, or 241.

In some embodiments, the transmembrane immunomodulatory protein can further contain a signal peptide. In some embodiments, the signal peptide is the native signal peptide of wild-type IgSF member, such as contained in the sequence of amino acids set forth in SEQ ID NO: 2 (see e.g., Table 2).

Also provided is a nucleic acid molecule encoding such transmembrane immunomodulatory proteins. In some embodiments, a nucleic acid molecule encoding a transmembrane immunomodulatory protein comprises a nucleotide sequence that encodes a sequence of amino acids that exhibits at least 85%, 86%, 87%, 88%, 89%, 90%, 91%, 92%, 93%, 94%, 95%, 96%, 97%, 98% or 99% sequence identity to SEQ ID NOS: 56 and contains an ectodomain comprising at least one affinity-modified IgSF domain as described herein, a transmembrane domain and, optionally, a cytoplasmic domain. In some embodiments, the nucleic acid molecule can further comprise a sequence of nucleotides encoding a signal peptide. In some embodiments, the signal peptide is the native signal peptide of the corresponding wild-type IgSF member (see e.g., Table 2). In some embodiments, the signal peptide is a heterologous signal peptide, such as any set forth in Table 4.

In some embodiments, provided are CAR-related transmembrane immunomodulatory proteins in which the endodomain of a transmembrane immunomodulatory protein comprises a cytoplasmic signaling domain that comprises at least one ITAM (immunoreceptor tyrosine-based activation motif)-containing signaling domain. ITAM is a conserved motif found in a number of protein signaling domains involved in signal transduction of immune cells, including in the CD3-zeta chain (“CD3-z”) involved in T-cell receptor signal transduction. In some embodiments, the endodomain comprises at CD3-zeta signaling domain. In some embodiments, the CD3-zeta signaling domain comprises the sequence of amino acids set forth in SEQ ID NO: 284 or a sequence of amino acids that exhibits at least 85%, 86%, 87%, 88%, 89%, 90%, 91%, 92%, 93%, 94%, 95%, 96%, 97%, 98% or 99% to SEQ ID NO:284 and retains the activity of T cell signaling. In some embodiments, the endodomain of a CAR-related transmembrane immunomodulatory protein can further comprise a costimulatory signaling domain to further modulate immunomodulatory responses of the T-cell. In some embodiments, the costimulatory signaling domain is CD28, ICOS, 4-1BB, or OX40. In some embodiments, the costimulatory signaling domain is a derived from CD28 or 4-1BB and comprises the sequence of amino acids set forth in any of SEQ ID NOS: 285-288 or a sequence of amino acids that exhibits at least 85%, 86%, 87%, 88%, 89%, 90%, 91%, 92%, 93%, 94%, 95%, 96%, 97%, 98% or 99% to SEQ ID NO: 285-288 and retains the activity of T cell costimulatory signaling. In some embodiments, the provided CAR-related transmembrane immunomodulatory proteins have features of CARs to stimulate T cell signaling upon binding of an affinity modified IgSF domain to a cognate binding partner or counter structure. In some embodiments, upon specific binding by the affinity-modified IgSF domain to its counter structure can lead to changes in the immunological activity of the T-cell activity as reflected by changes in cytotoxicity, proliferation or cytokine production.

In some embodiments, the transmembrane immunomodulatory protein does not contain an endodomain capable of mediating cytoplasmic signaling. In some embodiments, the transmembrane immunomodulatory protein lacks the signal transduction mechanism of the wild-type or unmodified polypeptide and therefore does not itself induce cell signaling. In some embodiments, the transmembrane immunomodulatory protein lacks an intracellular (cytoplasmic) domain or a portion of the intracellular domain of the corresponding wild-type or unmodified polypeptide, such as a cytoplasmic signaling domain contained in the sequence of amino acids set forth in SEQ ID NO:2 (see Table 2). In some embodiments, the transmembrane immunomodulatory protein does not contain an ITIM (immunoreceptor tyrosine-based inhibition motif), such as contained in certain inhibitory receptors, including inhibitory receptors of the IgSF family (e.g., PD-1 or TIGIT). Thus, in some embodiments, the transmembrane immunomodulatory protein only contains the ectodomain and the transmembrane domain, such as any as described.

### 2. Secreted Immunomodulatory Proteins and Engineered Cells

In some embodiments, the CD86 variant immunomodulatory polypeptide containing any one or more of the amino acid mutations as described herein, is secretable, such as when expressed from a cell. Such a variant CD86 immunomodulatory protein does not comprise a transmembrane domain. In some embodiments, the variant CD86 immunomodulatory protein is not conjugated to a half-life extending moiety (such as an Fc domain or a multimerization domain). In some embodiments, the variant CD86 immunomodulatory protein comprises a signal peptide, e.g., an antibody signal peptide or other efficient signal sequence to get domains outside of the cell. When the immunomodulatory protein comprises a signal peptide and is expressed by an engineered cell, the signal peptide causes the immunomodulatory protein to be secreted by the engineered cell. Generally, the signal peptide, or a portion of the signal peptide, is cleaved from the immunomodulatory protein with secretion. The immunomodulatory protein can be encoded by a nucleic acid (which can be part of an expression vector). In some embodiments, the immunomodulatory protein is expressed and secreted by a cell (such as an immune cell, for example a primary immune cell).

Thus, in some embodiments, there are provided variant CD86 immunomodulatory proteins that further comprises a signal peptide. In some embodiments, provided herein is a nucleic acid molecule encoding the variant CD86 immunomodulatory protein operably connected to a secretion sequence encoding the signal peptide.

A signal peptide is a sequence on the N-terminus of an immunomodulatory protein that signals secretion of the immunomodulatory protein from a cell. In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from the corresponding wild-type CD86 (see Table 2). In some embodiments, the signal peptide is a non-native signal peptide. For example, in some embodiments, the non-native signal peptide is a mutant native signal peptide from the corresponding wild-type CD86, and can include one or more (such as 2, 3, 4, 5, 6, 7, 8, 9, or 10 or more) substitutions insertions or deletions. In some embodiments, the non-native signal peptide is a signal peptide or mutant thereof of a family member from the same IgSF family as the wild-type IgSF family member. In some embodiments, the non-native signal peptide is a signal peptide or mutant thereof from an IgSF family member from a different IgSF family than the wild-type IgSF family member. In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2) or CD33), a serum albumin protein (e.g., HSA or albumin), a human azurocidin preprotein signal sequence, a luciferase, a trypsinogen (e.g., chymotrypsinogen or trypsinogen), or other signal peptide able to efficiently secrete a protein from a cell. Exemplary signal peptides include any described in the Table 4.

TABLE 4
Exempary Signal Peptides

| SEQ ID NO | Signal Peptide | Peptide Sequence |
|---|---|---|
| 289 | CD33 Signal Peptide | MPLLLLLPLLWAGALA |
| 290 | IgGkappa light chain: | MDMRVLAQLL GLLLLCFPGA RC |
| 291 | HSA signal peptide | MKWVTFISLLFLFSSAYS |
| 292 | Ig kappa light chain | MDMRAPAGIFGFLLVLFPGYRS |
| 293 | human azurocidin preprotein signal | MTRLTVLALLAGLLASSRA |
| | sequence | |
| 294 | IgG heavy chain signal peptide | MELGLSWIFLLAILKGVQC |
| 295 | IgG heavy chain signal peptide | MELGLRWVFLVAILEGVQC |
| 296 | IgG heavy chain signal peptide | MKHLWFFLLLVAAPRWVLS |
| 297 | IgG heavy chain signal peptide | MDWTWRILFLVAAATGAHS |
| 298 | IgG heavy chain signal peptide | MDWTWRFLFVVAAATGVQS |
| 299 | IgG heavy chain signal peptide | MEFGLSWLFLVAILKGVQC |
| 300 | IgG heavy chain signal peptide | MEFGLSWVFLVALFRGVQC |
| 301 | IgG heavy chain signal peptide | MDLLHKNMKHLWFFLLLVAAPRWVLS |
| 302 | IgG Kappa light chain signal sequences: | MDMRVPAQLLGLLLLWLSGARC |
| 303 | IgG Kappa light chain signal sequences: | MKYLLPTAAAGLLLLAAQPAMA |
| 304 | Gaussia luciferase | MGVKVLFALICIAVAEA |
| 305 | Human albumin | MKWVTFISLLFLFSSAYS |
| 306 | Human chymotrypsinogen | MAFLWLLSCWALLGTTFG |
| 307 | Human interleukin-2 | MQLLSCIALILALV |
| 308 | Human trypsinogen-2 | MNLLLILTFVAAAVA |

In some embodiments of a secretable variant CD86 immunomodulatory protein, the immunomodulatory protein comprises a signal peptide when expressed, and the signal peptide (or a portion thereof) is cleaved from the immunomodulatory protein upon secretion.

In some embodiments, the engineered cells express variant CD86 polypeptides that are secreted from the cell. In some embodiments, such a variant CD86 polypeptide is encoded by a nucleic acid molecule encoding an immunomodulatory protein under the operable control of a signal sequence for secretion. In some embodiments, the encoded immunomodulatory protein is secreted when expressed from a cell. In some embodiments, the immunomodulatory protein encoded by the nucleic acid molecule does not comprise a transmembrane domain. In some embodiments, the immunomodulatory protein encoded by the nucleic acid molecule does not comprise a half-life extending moiety (such as an Fc domain or a multimerization domain). In some embodiments, the immunomodulatory protein encoded by the nucleic acid molecule comprises a signal peptide. In some embodiments, a nucleic acid of the invention further comprises nucleotide sequence that encodes a secretory or signal peptide operably linked to the nucleic acid encoding the immunomodulatory protein, thereby allowing for secretion of the immunomodulatory protein.

### 3. Cells and Engineering Cells

Provided herein are engineered cells expressing any of the provided immunomodulatory polypeptides. In some embodiments, the engineered cells express on their surface any of the provided transmembrane immunomodulatory polypeptides. In some embodiments, the engineered cells express and are capable of or are able to secrete the immunomodulatory protein from the cells under conditions suitable for secretion of the protein. In some embodiments, the immunomodulatory protein is expressed on a lymphocyte such as a tumor infiltrating lymphocyte (TIL), T-cell or NK cell, or on a myeloid cell. In some embodiments, the engineered cells are antigen presenting cells (APCs). In some embodiments, the engineered cells are engineered mammalian T-cells or engineered mammalian antigen presenting cells (APCs). In some embodiments, the engineered T-cells or APCs are human or murine cells.

In some embodiments, engineered T-cells include, but are not limited to, T helper cell, cytotoxic T-cell (alternatively, cytotoxic T lymphocyte or CTL), natural killer T-cell, regulatory T-cell, memory T-cell, or gamma delta T-cell. In some embodiments, the engineered T cells are CD4+ or CD8+. In addition to the signal of the MHC, engineered T-cells also require a co-stimulatory signal. In some embodiments, engineered T cells also can be modulated by an inhibitory signal, which, in some cases, is provided by a variant CD86 transmembrane immunomodulatory polypeptide expressed in membrane bound form as discussed previously.

In some embodiments, the engineered APCs include, for example, MHC II expressing APCs such as macrophages, B cells, and dendritic cells, as well as artificial APCs (aAPCs) including both cellular and acellular (e.g., biodegradable polymeric microparticles) aAPCs. Artificial APCs (aAPCs) are synthetic versions of APCs that can act in a similar manner to APCs in that they present antigens to T-cells as well as activate them. Antigen presentation is performed by the MHC (Class I or Class II). In some embodiments, in engineered APCs such as aAPCs, the antigen that is loaded onto the MHC is, in some embodiments, a tumor specific antigen or a tumor associated antigen. The antigen loaded onto the MHC is recognized by a T-cell receptor (TCR) of a T cell, which, in some cases, can express CTLA-4, CD28, or other molecules recognized by the variant CD86 polypeptides provided herein. Materials which can be used to engineer an aAPC include: poly (glycolic acid), poly(lactic-co-glycolic acid), iron-oxide, liposomes, lipid bilayers, sepharose, and polystyrene.

In some embodiments a cellular aAPC can be engineered to contain a TIP and TCR agonist which is used in adoptive cellular therapy. In some embodiments, a cellular aAPC can be engineered to contain a TIP and TCR agonist which is used in ex vivo expansion of human T cells, such as prior to administration, e.g., for reintroduction into the patient. In some aspects, the aAPC may include expression of at least one anti-CD3 antibody clone, e.g., such as, for example, OKT3 and/or UCHT1. In some aspects, the aAPCs may be inactivated (e.g., irradiated). In some embodiments, the TIP can include any variant IgSF domain that exhibits binding affinity for a cognate binding partner on a T cell.

In some embodiments, an immunomodulatory protein provided herein, such as a transmembrane immunomodulatory protein or a secretable immunomodulatory protein, is co-expressed or engineered into a cell that expresses an antigen-binding receptor, such as a recombinant receptor, such as a chimeric antigen receptor (CAR) or T cell receptor (TCR). In some embodiments, the engineered cell, such as an engineered T cell, recognizes a desired antigen associated with cancer, inflammatory and autoimmune disorders, or a viral infection. In specific embodiments, the antigen-binding receptor contains an antigen-binding moiety that specifically binds a tumor specific antigen or a tumor associated antigen. In some embodiments, the engineered T-cell is a CAR (chimeric antigen receptor) T-cell that contains an antigen-binding domain (e.g., scFv) that specifically binds to an antigen, such as a tumor specific antigen or tumor associated antigen. In some embodiments, the TIP protein is expressed in an engineered T-cell receptor cell or an engineered chimeric antigen receptor cell. In such embodiments, the engineered cell co-expresses the TIP and the CAR or TCR. In some embodiments, the SIP protein is expressed in an engineered T-cell receptor cell or an engineered chimeric antigen receptor cell. In such embodiments, the engineered cell co-expresses the SIP and the CAR or TCR.

Chimeric antigen receptors (CARs) are recombinant receptors that include an antigen-binding domain (ectodomain), a transmembrane domain, and an intracellular signaling region (endodomain) that is capable of inducing or mediating an activation signal to the T cell after the antigen is bound. In some examples, CAR-expressing cells are engineered to express an extracellular single chain variable fragment (scFv) with specificity for a particular tumor antigen linked to an intracellular signaling part comprising an activating domain and, in some cases, a costimulatory domain. The costimulatory domain can be derived from, e.g., CD28, OX-40, 4-1BB/CD137, inducible T cell costimulator (ICOS). The activating domain can be derived from, e.g., CD3, such as CD3 zeta, epsilon, delta, gamma, or the like. In certain embodiments, the CAR is designed to have two, three, four, or more costimulatory domains. The CAR scFv can be designed to target an antigen expressed on a cell associated with a disease or condition, e.g., a tumor antigen, such as, for example, HER2, which is an oncogene shown to play a role in the development and progression of certain types of aggressive breast cancer.

In some aspects, the antigen-binding domain is an antibody or antigen-binding fragment thereof, such as a single chain fragment (scFv). In some embodiments, the antigen is expressed on a tumor or cancer cell. Exemplary of an antigen is HER2. Exemplary of a CAR is an anti-HER2 CAR, such as a CAR containing an anti-HER2 scFv. Other exemplary CARs include anti-CD19 CARs, anti-BCMA CARs, anti-CD22 CARs and other CARs specific to tumor-associated antigens. In some embodiments, the CAR further contains a spacer, a transmembrane domain, and an intracellular signaling domain or region comprising an ITAM signaling domain, such as a CD3-zeta signaling domain. In some embodiments, the CAR further includes a costimulatory signaling domain. In some embodiments, the spacer and transmembrane domain are the hinge and transmembrane domain derived from CD8, such as having an exemplary sequence set forth in SEQ ID NO: 281, 282, or 283 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 281, 282, or 283. In some embodiments, the endodomain comprises a CD3-zeta signaling domain. In some embodiments, the CD3-zeta signaling domain comprises the sequence of amino acids set forth in SEQ ID NO: 284 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO:284 and retains the activity of T-cell signaling. In some embodiments, the endodomain of a CAR can further comprise a costimulatory signaling domain or region to further modulate immunomodulatory responses of the T-cell. In some embodiments, the costimulatory signaling domain is or comprises a costimulatory region, or is derived from a costimulatory region, of CD28, ICOS, 4-1BB or OX40. In some embodiments, the costimulatory signaling domain is a derived from CD28 or 4-1BB and comprises the sequence of amino acids set forth in any of SEQ ID NOS: 285-288 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 285-288 and retains the activity of T cell costimulatory signaling.

In some embodiments, the construct encoding the CAR further encodes a second protein, such as a marker, e.g., detectable protein, separated from the CAR by a self-cleaving peptide sequence. In some embodiments, the self-cleaving peptide sequence is an F2A, T2A, E2A, or P2A self-cleaving peptide. Exemplary sequences of a T2A self-cleaving peptide are set for the in any one of SEQ ID NOS: 309, 310, 311 or a sequence of amino acids that exhibits at least 85%, 86%, 87%, 88%, 89%, 90%, 91%, 92%, 93%, 94%, 95%, 96%, 97%, 98% or 99% or more sequence identity to any of SEQ ID NOS: 309, 310, 311. In some embodiments, the T2A is encoded by the sequence of nucleotides set forth in SEQ ID NO: 311 or a sequence that exhibits at least 85%, 86%, 87%, 88%, 89%, 90%, 91%, 92%, 93%, 94%, 95%, 96%, 97%, 98% or 99% or more sequence identity to any of SEQ ID NO: 311. An exemplary sequence of a P2A self-cleaving peptide is set in SEQ ID NO: 243 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 243. In some cases, a nucleic acid construct that encodes more than one P2A self-cleaving peptide (such as a P2A1 and P2A2), in which the nucleotide sequence P2A1 and P2A2 each encode the P2A set forth in SEQ ID NO: 243, the nucleotide sequence may be different to avoid recombination between sequences.

In some embodiments, the marker is a detectable protein, such as a fluorescent protein, e.g., a green fluorescent protein (GFP) or blue fluorescent protein (BFP). Exemplary sequences of a fluorescent protein marker are set forth in SEQ ID NO: 313, 312, 244-246, or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 313, 312, 244-246.

In some embodiments, the CAR comprises an anti-HER scFv, a CD8 hinge region, and transmembrane signaling domains derived from 4-1BB and CD3-zeta signalling domains. In some embodiments, the CAR comprises an scFv containing a variable heavy and light chains of trastuzumab.

In another embodiment, the engineered T-cell possesses a TCR, including a recombinant or engineered TCR. In some embodiments, the TCR can be a native TCR. Those of skill in the art will recognize that generally native mammalian T-cell receptors comprise an alpha and a beta chain (or a gamma and a delta chain) involved in antigen specific recognition and binding. In some embodiments, the TCR is an engineered TCR that is modified. In some embodiments, the TCR of an engineered T-cell specifically binds to a tumor associated or tumor specific antigen presented by an APC. In some embodiments, the TCR is an HPV16 E6 peptide (E6 TCR). In some embodiments, the TCR is an HPV16 E7 peptide (E7 TCR). Exemplary IHPV TCRs include those described in International published PCT Appl. No. WO2015009606 or WO2015184228.

In some embodiments, the immunomodulatory polypeptides, such as transmembrane immunomodulatory polypeptides or secretable immunomodulatory polypeptides, can be incorporated into engineered cells, such as engineered T cells or engineered APCs, by a variety of strategies such as those employed for recombinant host cells. A variety of methods to introduce a DNA construct into primary T-cells are known in the art. In some embodiments, viral transduction or plasmid electroporation are employed. In typical embodiments, the nucleic acid molecule encoding the immunomodulatory protein, or the expression vector, comprises a signal peptide that localizes the expressed transmembrane immunomodulatory proteins to the cellular membrane or for secretion. In some embodiments, a nucleic acid encoding a transmembrane immunomodulatory protein of the invention is sub-cloned into a viral vector, such as a retroviral vector, which allows expression in the host mammalian cell. The expression vector can be introduced into a mammalian host cell and, under host cell culture conditions, the immunomodulatory protein is expressed on the surface or is secreted.

In an exemplary example, primary T-cells can be purified ex vivo (CD4+ cells or CD8+ cells or both) and stimulated with an activation protocol consisting of various TCR/CD28 agonists, such as anti-CD3/anti-CD28 coated beads. After a 2 or 3 day activation process, a recombinant expression vector containing an immunomodulatory polypeptide can be stably introduced into the primary T-cells through art standard lentiviral or retroviral transduction protocols or plasmid electroporation strategies. Cells can be monitored for immunomodulatory polypeptide expression by, for example, flow cytometry using an anti-epitope tag or antibodies that cross-react with native parental molecule and polypeptides comprising variant CD86. T-cells that express the immunomodulatory polypeptide can be enriched through sorting with anti-epitope tag antibodies or enriched for high or low expression depending on the application.

Upon immunomodulatory polypeptide expression the engineered T-cell can be assayed for appropriate function by a variety of means. The engineered CAR or TCR co-expression can be validated to show that this part of the engineered T-cell was not significantly impacted by the expression of the immunomodulatory protein. Once validated, standard in vitro cytotoxicity, proliferation, or cytokine assays (e.g., IFN-gamma, IL2, TNFα expression) can be used to assess the function of engineered T-cells. Exemplary standard endpoints are percent lysis of the tumor line, proliferation of the engineered T-cell, or IFN-gamma protein expression in culture supernatants. An engineered construct which results in statistically significant increased lysis of tumor line, increased proliferation of the engineered T-cell, or increased IFN-gamma expression over the control construct can be selected for. Additionally, non-engineered, such as native primary or endogenous T-cells could also be incorporated into the same in vitro assay to measure the ability of the immunomodulatory polypeptide construct expressed on the engineered cells, such as engineered T-cells, to modulate activity, including, in some cases, to activate and generate effector function in bystander, native T-cells. Increased expression of activation or differentiation markers such as CD25, CD69, or CD44 could be monitored on endogenous T cells, and increased proliferation and/or cytokine production could indicate desired activity of the immunomodulatory protein expressed on the engineered T cells.

In some embodiments, similar assays can be used to compare the function of engineered T cells containing the CAR or TCR alone to those containing the CAR or TCR and a TIP construct. Typically, these in vitro assays are performed by plating various ratios of the engineered T cell and a “tumor” cell line containing the cognate CAR or TCR antigen together in culture. Standard endpoints are percent lysis of the tumor line, proliferation of the engineered T cell, or IFN-gamma production in culture supernatants. An engineered immunomodulatory protein which resulted in statistically significant increased lysis of tumor line, increased proliferation of the engineered T cell, or increased IFN-gamma production over the same TCR or CAR construct alone, can be selected for. Engineered human T cells can be analyzed in immunocompromised mice, like the NSG strain, which lacks mouse T, NK, and B cells. Engineered human T cells in which the CAR or TCR binds a target counter-structure on the xenograft and is co-expressed with the TIP affinity modified IgSF domain can be adoptively transferred in vivo at different cell numbers and ratios compared to the xenograft. In a common embodiment, the xenograft is introduced into the murine model, followed by the engineered T cells several days later. Engineered T cells containing the immunomodulatory protein can be assayed for increased survival, tumor clearance, or expanded engineered T cells numbers relative to engineered T cells containing the CAR or TCR alone. As in the in vitro assay, endogenous, native (i.e., non-engineered) human T cells could be co-adoptively transferred to look for successful epitope spreading in that population, resulting in better survival or tumor clearance.

In some embodiments, provided engineered T cells expressing a provided immunomodulatory protein exhibits one or more improved properties or activities compared to reference cells that have not been so engineered with an immunomodulatory protein (e.g. TIP) as described herein. In some embodiments, the reference cell, such as a reference T cells, reference CAR-engineered T cells, or reference TCR-engineered T cells, are cells that have been produced or engineered by similar ex vivo procedures but that do not express or have not been engineered to express the immunomodulatory protein. In some embodiments, the property or activity is associated with or related to T-cell function. In some embodiments, the one or more properties or activities include, but are not limited to, cellular proliferation, cytototoxic activity, cytokine production (e.g. IFN-gamma, IL-2 or TNF-alpha), and/or expression of one or more activation markers (e.g. CD69 or CD25). In some embodiments, the activity or property is increased by at least or at least about 1.2-fold, 1.4-fold, 1.5-fold, 1.6-fold, 1.7-fold, 1.8-fold, 1.9-fold, 2.0-fold, 2.5-fold, 3.0-fold, 4.0-fold, 5.0-fold, or more compared to the reference cell or reference cell composition

### F. Infectious Agents Expressing Variant Polypeptides and Immunomodulatory Proteins

Also provided are infectious agents that contain nucleic acids encoding any of the variant polypeptides, such as CD86 vIgD polypeptides, including secretable or transmembrane immunomodulatory proteins described herein. In some embodiments, such infectious agents can deliver the nucleic acids encoding the variant immunomodulatory polypeptides described herein, such as CD86 vIgD polypeptides, to a target cell in a subject, e.g., immune cell and/or antigen-presenting cell (APC) or tumor cell in a subject. Also provided are nucleic acids contained in such infectious agents, and/or nucleic acids for generation or modification of such infectious agents, such as vectors and/or plasmids, and compositions containing such infectious agents.

In some embodiments, the infectious agent is a microorganism or a microbe. In some embodiments, the infectious agent is a virus or a bacterium. In some embodiments, the infectious agent is a virus. In some embodiments, the infectious agent is a bacterium. In some embodiments, such infectious agents can deliver nucleic acid sequences encoding any of the variant polypeptides, such as CD86 vIgD polypeptides, including secretable or transmembrane immunomodulatory proteins, described herein. Thus, in some embodiments, the cell in a subject that is infected or contacted by the infectious agents can be rendered to express on the cell surface or secrete the variant immunomodulatory polypeptides. In some embodiments, the infectious agent can also deliver one or more other therapeutics or nucleic acids encoding other therapeutics to the cell and/or to an environment within the subject. In some embodiments, other therapeutics that can be delivered by the infectious agents include cytokines or other immunomodulatory molecules.

In some embodiments, the infectious agent, e.g., virus or bacteria, contains nucleic acid sequences that encode any of the variant polypeptides, such as CD86 vIgD polypeptides, including secretable or transmembrane immunomodulatory proteins, described herein, and by virtue of contact and/or infection of a cell in the subject, the cell expresses the variant polypeptides, such as CD86 vIgD polypeptides, including secretable or transmembrane immunomodulatory proteins, encoded by the nucleic acid sequences contained in the infectious agent. In some embodiments, the infectious agent can be administered to the subject. In some embodiments, the infectious agent can be contacted with cells from the subject ex vivo.

In some embodiments, the variant polypeptides, such as CD86 vIgD polypeptides, including transmembrane immunomodulatory proteins, expressed by the cell infected by the infectious agent is a transmembrane protein and is surface expressed. In some embodiments, the variant polypeptides, such as CD86 vIgD polypeptides, including secretable immunomodulatory proteins, expressed by the cell infected by the infectious agent is expressed and secreted from the cell. The transmembrane immunomodulatory protein or secreted immunomodulatory protein can be any described herein.

In some embodiments, the cell in the subject that is targeted by the infectious agent include a tumor cell, an immune cell, and/or an antigen-presenting cell (APC). In some embodiments, the infectious agent targets a cell in the tumor microenvironment (TME). In some embodiments, the infectious agent delivers the nucleic acids encoding the variant polypeptides, such as CD86 vIgD polypeptides, including secretable or transmembrane immunomodulatory proteins, to an appropriate cell (for example, an APC, such as a cell that displays a peptide/MHC complex on its cell surface, such as a dendritic cell) or tissue (e.g., lymphoid tissue) that will induce and/or augment the desired effect, e.g., immunomodulation and/or a specific cell-medicated immune response, e.g., CD4 and/or CD8 T-cell response, which CD8 T-cell response may include a cytotoxic T-cell (CTL) response. In some embodiments, the infectious agent targets an APC, such as a dendritic cell (DC). In some embodiments, the nucleic acid molecule delivered by the infectious agents described herein include appropriate nucleic acid sequences necessary for the expression of the operably linked coding sequences encoding the variant immunomodulatory polypeptides, in a particular target cell, e.g., regulatory elements such as promoters.

In some embodiments, the infectious agent that contains nucleic acid sequences encoding the immunomodulatory polypeptides can also contain nucleic acid sequences that encode one or more additional gene products, e.g., cytokines, prodrug converting enzymes, cytotoxins, and/or detectable gene products. For example, in some embodiments, the infectious agent is an oncolytic virus and the virus can include nucleic acid sequences encoding additional therapeutic gene products (see, e.g., Kim et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398 and 7,754,221 and U.S. Pat. Publ. Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650. In some embodiments, the additional gene product can be a therapeutic gene product that can result in death of the target cell (e.g., tumor cell) or gene products that can augment or boost or regulate an immune response (e.g., cytokine). Exemplary gene products also include among an anticancer agent, an anti-metastatic agent, an antiangiogenic agent, an immunomodulatory molecule, an immune checkpoint inhibitor, an antibody, a cytokine, a growth factor, an antigen, a cytotoxic gene product, a pro-apoptotic gene product, an anti-apoptotic gene product, a cell matrix degradative gene, genes for tissue regeneration or reprogramming human somatic cells to pluripotency, and other genes described herein or known to one of skill in the art. In some embodiments, the additional gene product is Granulocyte-macrophage colony-stimulating factor (GM-CSF).

### 1. Viruses

In some embodiments, the infectious agent is a virus. In some embodiments, the infectious agent is an oncolytic virus, or a virus that targets particular cells, e.g., immune cells. In some embodiments, the infectious agent targets a tumor cell and/or cancer cell in the subject. In some embodiments, the infectious agent targets an immune cell or an antigen-presenting cell (APC).

In some embodiments, the infectious agent is an oncolytic virus. Oncolytic viruses are viruses that accumulate in tumor cells and replicate in tumor cells. By virtue of replication in the cells, and optional delivery of nucleic acids encoding variant immunomodulatory variant CD86 polypeptides or immunomodulatory proteins described herein, tumor cells are lysed, and the tumor shrinks and can be eliminated. Oncolytic viruses can also have a broad host and cell type range. For example, oncolytic viruses can accumulate in immunoprivileged cells or immunoprivileged tissues, including tumors and/or metastases, and also including wounded tissues and cells, thus allowing the delivery and expression of nucleic acids encoding the variant immunomodulatory polypeptides described herein in a broad range of cell types. Oncolytic viruses can also replicate in a tumor cell specific manner, resulting in tumor cell lysis and efficient tumor regression.

Exemplary oncolytic viruses include adenoviruses, adeno-associated viruses, herpes viruses, Herpes Simplex Virus, Reovirus, Newcastle Disease virus, parvovirus, measles virus, vesicular stomatitis virus (VSV), Coxsackie virus and Vaccinia virus. In some embodiments, oncolytic viruses can specifically colonize solid tumors, while not infecting other organs, and can be used as an infectious agent to deliver the nucleic acids encoding the variant immunomodulatory polypeptides described herein to such solid tumors.

Oncolytic viruses for use in delivering the nucleic acids encoding variant CD86 polypeptides or immunomodulatory proteins described herein, can be any of those known to one of skill in the art and include, for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175; herpes simplex virus, see, e.g., U.S. Pat. Nos. 7,897,146, 7,731,952, 7,550,296, 7,537,924, 6,723,316, 6,428,968 and U.S. Pat. Pub. Nos., 2014/0154216, 2011/0177032, 2011/0158948, 2010/0092515, 2009/0274728, 2009/0285860, 2009/0215147, 2009/0010889, 2007/0110720, 2006/0039894, 2004/0009604, 2004/0063094, International Patent Pub. Nos., WO 2007/052029, WO 1999/038955; retroviruses, see, e.g., U.S. Pat. Nos. 6,689,871, 6,635,472, 5,851,529, 5,716,826, 5,716,613 and U.S. Pat. Pub. No. 20110212530; vaccinia viruses, see, e.g., 2016/0339066, and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

Oncolytic viruses also include viruses that have been genetically altered to attenuate their virulence, to improve their safety profile, enhance their tumor specificity, and they have also been equipped with additional genes, for example cytotoxins, cytokines, prodrug converting enzymes to improve the overall efficacy of the viruses (see, e.g., Kim et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398 and 7,754,221 and U.S. Pat. Publ. Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917 and 2011/0064650). In some embodiments, the oncolytic viruses can be those that have been modified so that they selectively replicate in cancerous cells, and, thus, are oncolytic. For example, the oncolytic virus is an adenovirus that has been engineered to have modified tropism for tumor therapy and also as gene therapy vectors. Exemplary of such is ONYX-015, H101 and Ad5ΔCR (Hallden and Portella (2012) Expert Opin Ther Targets, 16:945-58) and TNFerade (McLoughlin et al. (2005) Ann. Surg. Oncol., 12:825-30), or a conditionally replicative adenovirus Oncorine®.

In some embodiments, the infectious agent is a modified herpes simplex virus. In some embodiments, the infectious agent is a modified version of Talimogene laherparepvec (also known as T-Vec, Imlygic or OncoVex GM-CSF), that is modified to contain nucleic acids encoding any of the variant immunomodulatory polypeptides described herein, such as any of the variant CD86 polypeptides or immunomodulatory proteins described herein. In some embodiments, the infectious agent is a modified herpes simplex virus that is described, e.g., in WO 2007/052029, WO 1999/038955, US 2004/0063094, US 2014/0154216, or, variants thereof.

In some embodiments, the infectious agent is a virus that targets a particular type of cells in a subject that is administered the virus, e.g., a virus that targets immune cells or antigen-presenting cells (APCs). Dendritic cells (DCs) are essential APCs for the initiation and control of immune responses. DCs can capture and process antigens, migrate from the periphery to a lymphoid organ, and present the antigens to resting T cells in a major histocompatibility complex (MHC)-restricted fashion. In some embodiments, the infectious agent is a virus that specifically can target DCs to deliver nucleic acids encoding the variant CD86 polypeptides or immunomodulatory proteins for expression in DCs. In some embodiments, the virus is a lentivirus or a variant or derivative thereof, such as an integration-deficient lentiviral vector. In some embodiments, the virus is a lentivirus that is pseudotyped to efficiently bind to and productively infect cells expressing the cell surface marker dendritic cell-specific intercellular adhesion molecule-3-grabbing non-integrin (DC-SIGN), such as DCs. In some embodiments, the virus is a lentivirus pseudotyped with a Sindbis virus E2 glycoprotein or modified form thereof, such as those described in WO 2013/149167. In some embodiments, the virus allows for delivery and expression of a sequence of interest (e.g., a nucleic acid encoding any of the variant CD86 polypeptides or immunomodulatory proteins described herein) to a DC. In some embodiments, the virus includes those described in WO 2008/011636 or US 2011/0064763, Tareen et al. (2014) Mol. Ther., 22:575-587, or variants thereof. Exemplary of a dendritic cell-tropic vector platform is ZVex™.

### 2. Bacteria

In some embodiments, the infectious agent is a bacterium. For example, in some embodiments, the bacteria can deliver nucleic acids encoding any of the variant immunomodulatory polypeptides described herein, e.g., variant CD86 polypeptide or immunomodulatory protein, to a target cell in the subject, such as a tumor cell, an immune cell, an antigen-presenting cell and/or a phagocytic cell. In some embodiments, the bacterium can be preferentially targeted to a specific environment within a subject, such as a tumor microenvironment (TME), for expression and/or secretion of the variant immunomodulatory polypeptides and/or to target specific cells in the environment for expression of the variant immunomodulatory polypeptides.

In some embodiments, the bacterium delivers the nucleic acids to the cells via bacterial-mediated transfer of plasmid DNA to mammalian cells (also referred to as “bactofection”). For example, in some embodiments, delivery of genetic material is achieved through entry of the entire bacterium into target cells. In some embodiments, spontaneous or induced bacterial lysis can lead to the release of plasmid for subsequent eukaryotic cell expression. In some embodiments, the bacterium can deliver nucleic acids to non-phagocytic mammalian cells (e.g., tumor cells) and/or to phagocytic cells, e.g., certain immune cells and/or APCs. In some embodiments, the nucleic acids delivered by the bacterium can be transferred to the nucleus of the cell in the subject for expression. In some embodiments, the nucleic acids also include appropriate nucleic acid sequences necessary for the expression of the operably linked sequences encoding the variant immunomodulatory polypeptides in a particular host cell, e.g., regulatory elements such as promoters or enhancers. In some embodiments, the infectious agent that is a bacterium can deliver nucleic acids encoding the immunomodulatory proteins in the form of an RNA, such as a pre-made translation-competent RNA delivered to the cytoplasm of the target cell for translation by the target cell's machinery.

In some embodiments, the bacterium can replicate and lyse the target cells, e.g., tumor cells. In some embodiments, the bacterium can contain and/or release nucleic acid sequences and/or gene products in the cytoplasm of the target cells, thereby killing the target cell, e.g., tumor cell. In some embodiments, the infectious agent is a bacterium that can replicate specifically in a particular environment in the subject, e.g., tumor microenvironment (TME). For example, in some embodiments, the bacterium can replicate specifically in anaerobic or hypoxic microenvironments. In some embodiments, conditions or factors present in particular environments, e.g., aspartate, serine, citrate, ribose or galactose produced by cells in the TME, can act as chemoattractants to attract the bacterium to the environment. In some embodiments, the bacterium can express and/or secrete the immunomodulatory proteins described herein in the environment, e.g., TME.

In some embodiments, the infectious agent is a bacterium that is a Listeria sp., a Bifidobacterium sp., an Escherichia sp., a Clostridium sp., a Salmonella sp., a Shigella sp., a Vibrio sp. or a Yersinia sp. In some embodiments, the bacterium is selected from among one or more of Listeria monocytogenes, Salmonella typhimurium, Salmonella choleraesuis, Escherichia coli, Vibrio cholera, Clostridium perfringens, Clostridium butyricum, Clostridium novyi, Clostridium acetobutylicum, Bifidobacterium infantis, Bifidobacterium longum and Bifidobacterium adolescentis. In some embodiments, the bacterium is an engineered bacterium. In some embodiments, the bacterium is an engineered bacterium such as those described in, e.g., Seow and Wood (2009) Molecular Therapy 17(5):767-777; Baban et al. (2010) Bioengineered Bugs 1:6, 385-394; Patyar et al. (2010) J Biomed Sci 17:21; Tangney et al. (2010) Bioengineered Bugs 1:4, 284-287; van Pijkeren et al. (2010) Hum Gene Ther. 21(4):405-416; WO 2012/149364; WO 2014/198002; U.S. Pat. Nos. 9,103,831; 9,453,227; US 2014/0186401; US 2004/0146488; US 2011/0293705; US 2015/0359909 and EP 3020816. The bacterium can be modified to deliver nucleic acid sequences encoding any of the variant immunomodulatory polypeptides, conjugates and/or fusions provided herein, and/or to express such variant immunomodulatory polypeptides in the subject.

### G. Nucleic Acids, Vectors and Methods for Producing the Polypeptides or Cells

Provided herein are isolated or recombinant nucleic acids collectively referred to as “nucleic acids” which encode any of the various provided embodiments of the variant CD86 polypeptides or immunomodulatory polypeptides provided herein. In some embodiments, nucleic acids provided herein, including all described below, are useful in recombinant production (e.g., expression) of variant CD86 polypeptides or immunomodulatory polypeptides provided herein. In some embodiments, nucleic acids provided herein, including all described below, are useful in expression of variant CD86 polypeptides or immunomodulatory polypeptides provided herein in cells, such as in engineered cells, e.g., immune cells, or infectious agent cells. The nucleic acids provided herein can be in the form of RNA or in the form of DNA, and include mRNA, cRNA, recombinant or synthetic RNA and DNA, and cDNA. The nucleic acids provided herein are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided.

Also provided herein are recombinant expression vectors and recombinant host cells useful in producing the variant CD86 polypeptides or immunomodulatory polypeptides provided herein.

Also provided herein are engineered cells, such as engineered immune cells, containing any of the provided immunomodulatory polypeptides, such as any of the transmembrane immunomodulatory polypeptides or secretable immunomodulatory polypeptides.

Also provided herein are infectious agents, such as bacterial or viral cells, containing any of the provided immunomodulatory polypeptides, such as any of the transmembrane immunomodulatory polypeptides or secretable immunomodulatory polypeptides.

In any of the above provided embodiments, the nucleic acids encoding the immunomodulatory polypeptides provided herein can be introduced into cells using recombinant DNA and cloning techniques. To do so, a recombinant DNA molecule encoding an immunomodulatory polypeptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidite method. Also, a combination of these techniques could be used. In some instances, a recombinant or synthetic nucleic acid may be generated through polymerase chain reaction (PCR). In some embodiments, a DNA insert can be generated encoding one or more variant CD86 polypeptides containing at least one affinity-modified IgSF domain and, in some embodiments, a signal peptide, a transmembrane domain and/or an endodomain in accord with the provided description. This DNA insert can be cloned into an appropriate transduction/transfection vector as is known to those of skill in the art. Also provided are expression vectors containing the nucleic acid molecules.

In some embodiments, the expression vectors are capable of expressing the immunomodulatory proteins in an appropriate cell under conditions suited to expression of the protein. In some aspects, a nucleic acid molecule or an expression vector comprises the DNA molecule that encodes the immunomodulatory protein operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

In some embodiments, expression of the immunomodulatory protein is controlled by a promoter or enhancer to control or regulate expression. The promoter is operably linked to the portion of the nucleic acid molecule encoding the variant polypeptide or immunomodulatory protein. In some embodiments, the promotor is a constitutively active promotor (such as a tissue-specific constitutively active promotor or other constitutive promotor). In some embodiments, the promotor is an inducible promotor, which may be responsive to an inducing agent (such as a T-cell activation signal).

In some embodiments, a constitutive promoter is operatively linked to the nucleic acid molecule encoding the variant polypeptide or immunomodulatory protein. Exemplary constitutive promoters include the Simian vacuolating virus 40 (SV40) promoter, the cytomegalovirus (CMV) promoter, the ubiquitin C (UbC) promoter, and the EF-1 alpha (EF1a) promoter. In some embodiments, the constitutive promoter is tissue specific. For example, in some embodiments, the promoter allows for constitutive expression of the immunomodulatory protein in specific tissues, such as immune cells, lymphocytes, or T-cells. Exemplary tissue-specific promoters are described in U.S. Pat. No. 5,998,205, including, for example, a fetoprotein, DF3, tyrosinase, CEA, surfactant protein, and ErbB2 promoters.

In some embodiments, an inducible promoter is operatively linked to the nucleic acid molecule encoding the variant polypeptide or immunomodulatory protein such that expression of the nucleic acid is controllable by controlling the presence or absence of the appropriate inducer of transcription. For example, the promoter can be a regulated promoter and transcription factor expression system, such as the published tetracycline-regulated systems or other regulatable systems (see, e.g., published International PCT Appl. No. WO 01/30843), to allow regulated expression of the encoded polypeptide. An exemplary regulatable promoter system is the Tet-On (and Tet-Off) system available, for example, from Clontech (Palo Alto, CA). This promoter system allows the regulated expression of the transgene controlled by tetracycline or tetracycline derivatives, such as doxycycline. Other regulatable promoter systems are known (see e.g., published U.S. Application No. 2002-0168714, entitled “Regulation of Gene Expression Using Single-Chain, Monomeric, Ligand Dependent Polypeptide Switches,” which describes gene switches that contain ligand binding domains and transcriptional regulating domains, such as those from hormone receptors).

In some embodiments, the promotor is responsive to an element responsive to T-cell activation signaling. Solely by way of example, in some embodiments, an engineered T cell comprises an expression vector encoding the immunomodulatory protein and a promotor operatively linked to control expression of the immunomodulatory protein. The engineered T cell can be activated, for example by signaling through an engineered T cell receptor (TCR) or a chimeric antigen rector (CAR), and thereby triggering expression and secretion of the immunomodulatory protein through the responsive promotor.

In some embodiments, an inducible promoter is operatively linked to the nucleic acid molecule encoding the immunomodulatory protein such that the immunomodulatory protein is expressed in response to a nuclear factor of activated T-cells (NFAT) or nuclear factor kappa-light-chain enhancer of activated B cells (NF-κB). For example, in some embodiments, the inducible promoter comprises a binding site for NFAT or NF-κB. For example, in some embodiments, the promoter is an NFAT or NF-κB promoter or a functional variant thereof. Thus, in some embodiments, the nucleic acids make it possible to control the expression of immunomodulatory protein while also reducing or eliminating the toxicity of the immunomodulatory protein. In particular, engineered immune cells comprising the nucleic acids of the invention express and secrete the immunomodulatory protein only when the cell (e.g., a T-cell receptor (TCR) or a chimeric antigen receptor (CAR) expressed by the cell) is specifically stimulated by an antigen and/or the cell (e.g., the calcium signaling pathway of the cell) is non-specifically stimulated by, e.g., phorbol myristate acetate (PMA)/Ionomycin. Accordingly, the expression and, in some cases, secretion, of immunomodulatory protein can be controlled to occur only when and where it is needed (e.g., in the presence of an infectious disease-causing agent, cancer, or at a tumor site), which can decrease or avoid undesired immunomodulatory protein interactions.

In some embodiments, the nucleic acid encoding an immunomodulatory protein described herein comprises a suitable nucleotide sequence that encodes a NFAT promoter, NF-κB promoter, or a functional variant thereof “NFAT promoter” as used herein means one or more NFAT responsive elements linked to a minimal promoter. “NF-κB promoter” refers to one or more NF-κB responsive elements linked to a minimal promoter. In some embodiments, the minimal promoter of a gene is a minimal human IL-2 promoter or a CMV promoter. The NFAT responsive elements may comprise, e.g., NFAT1, NFAT2, NFAT3, and/or NFAT4 responsive elements. The NFAT promoter, NF-κB promoter, or a functional variant thereof may comprise any number of binding motifs, e.g., at least two, at least three, at least four, at least five, or at least six, at least seven, at least eight, at least nine, at least ten, at least eleven, or up to twelve binding motifs.

The resulting recombinant expression vector having the DNA molecule thereon is used to transform an appropriate host. This transformation can be performed using methods well known in the art. In some embodiments, a nucleic acid provided herein further comprises nucleotide sequence that encodes a secretory or signal peptide operably linked to the nucleic acid encoding an immunomodulatory polypeptide such that a resultant soluble immunomodulatory polypeptide is recovered from the culture medium, host cell, or host cell periplasm. In other embodiments, the appropriate expression control signals are chosen to allow for membrane expression of an immunomodulatory polypeptide. Furthermore, commercially available kits as well as contract manufacturing companies can also be utilized to make engineered cells or recombinant host cells provided herein.

In some embodiments, the resulting expression vector having the DNA molecule thereon is used to transform, such as transduce, an appropriate cell. The introduction can be performed using methods well known in the art. Exemplary methods include those for transfer of nucleic acids encoding the receptors, including via viral, e.g., retroviral or lentiviral, transduction, transposons, and electroporation. In some embodiments, the expression vector is a viral vector. In some embodiments, the nucleic acid is transferred into cells by lentiviral or retroviral transduction methods.

Any of a large number of publicly available and well-known mammalian host cells, including mammalian T-cells or APCs, can be used in the preparing the polypeptides or engineered cells. The selection of a cell is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all cells can be equally effective for the expression of a particular DNA sequence.

In some embodiments, the host cells can be a variety of eukaryotic cells, such as in yeast cells, or with mammalian cells such as Chinese hamster ovary (CHO) or HEK293 cells. In some embodiments, the host cell is a suspension cell and the polypeptide is engineered or produced in cultured suspension, such as in cultured suspension CHO cells, e.g., CHO-S cells. In some examples, the cell line is a CHO cell line that is deficient in DHFR (DHFR-), such as DG44 and DUXB11. In some embodiments, the cell is deficient in glutamine synthase (GS), e.g., CHO-S cells, CHOK1 SV cells, and CHOZN((R)) GS−/− cells. In some embodiments, the CHO cells, such as suspension CHO cells, may be CHO-S-2H2 cells, CHO-S-clone 14 cells, or ExpiCHO-S cells.

In some embodiments, host cells can also be prokaryotic cells, such as with E. coli. The transformed recombinant host is cultured under polypeptide expressing conditions, and then purified to obtain a soluble protein. Recombinant host cells can be cultured under conventional fermentation conditions so that the desired polypeptides are expressed. Such fermentation conditions are well known in the art. Finally, the polypeptides provided herein can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, and affinity chromatography. Protein refolding steps can be used, as desired, in completing configuration of the mature protein. Finally, high performance liquid chromatography (HPLC) can be employed in the final purification steps.

In some embodiments, the cell is an immune cell, such as any described above in connection with preparing engineered cells. In some embodiments, such engineered cells are primary cells. In some embodiments, the engineered cells are autologous to the subject. In some embodiment, the engineered cells are allogeneic to the subject. In some embodiments, the engineered cells are obtained from a subject, such as by leukapheresis, and transformed ex vivo for expression of the immunomodulatory polypeptide, e.g., transmembrane immunomodulatory polypeptide or secretable immunomodulatory polypeptide.

Also provided are nucleic acids encoding any of the variant immunomodulatory polypeptides contained in infectious agents described herein. In some embodiments, the infectious agents deliver the nucleic acids to a cell in the subject, and/or permit expression of the encoded variant polypeptides in the cell. Also provided are nucleic acids that are used to generate, produce or modify such infectious agents. For example, in some embodiments, provided are vectors and/or plasmids that contain nucleic acids encoding the variant immunomodulatory polypeptides, for generation of the infectious agents, delivery to the cells in a subject and/or expression of the variant immunomodulatory polypeptides in the cells in the subject.

In some embodiments, the provided nucleic acids are recombinant viral or bacterial vectors containing nucleic acid sequences encoding the variant immunomodulatory polypeptides. In some embodiments, the recombinant vectors can be used to produce an infectious agent that contains nucleic acid sequences encoding the variant immunomodulatory polypeptides and/or to be delivered to a target cell in the subject for expression by the target cell. In some embodiments, the recombinant vector is an expression vector. In some embodiments, the recombinant vector includes appropriate sequences necessary for generation and/or production of the infectious agent and expression in the target cell.

In some embodiments, the recombinant vector is a plasmid or cosmid. Plasmid or cosmid containing nucleic acid sequences encoding the variant immunomodulatory polypeptides, as described herein, is readily constructed using standard techniques well known in the art. For generation of the infectious agent, the vector or genome can be constructed in a plasmid form that can then be transfected into a packaging or producer cell line or a host bacterium. The recombinant vectors can be generated using any of the recombinant techniques known in the art. In some embodiments, the vectors can include a prokaryotic origin of replication and/or a gene whose expression confers a detectable or selectable marker such as a drug resistance for propagation and/or selection in prokaryotic systems.

In some embodiments, the recombinant vector is a viral vector. Exemplary recombinant viral vectors include a lentiviral vector genome, poxvirus vector genome, vaccinia virus vector genome, adenovirus vector genome, adenovirus-associated virus vector genome, herpes virus vector genome, and alpha virus vector genome. Viral vectors can be live, attenuated, replication conditional or replication deficient, non-pathogenic (defective), replication competent viral vector, and/or is modified to express a heterologous gene product, e.g., the variant immunomodulatory polypeptides provided herein. Vectors for generation of viruses also can be modified to alter attenuation of the virus, which includes any method of increasing or decreasing the transcriptional or translational load.

Exemplary viral vectors that can be used include modified vaccinia virus vectors (see, e.g., Guerra et al., J. Virol. 80:985-98 (2006); Tartaglia et al., AIDS Research and Human Retroviruses 8: 1445-47 (1992); Gheradi et al., J. Gen. Virol. 86:2925-36 (2005); Mayr et al., Infection 3:6-14 (1975); Hu et al., J. Virol. 75: 10300-308 (2001); U.S. Pat. Nos. 5,698,530, 6,998,252, 5,443,964, 7,247,615 and 7,368,116); adenovirus vector or adenovirus-associated virus vectors (see., e.g., Molin et al., J. Virol. 72:8358-61 (1998); Narumi et al., Am J. Respir. Cell Mol. Biol. 19:936-41 (1998); Mercier et al., Proc. Natl. Acad. Sci. USA 101:6188-93 (2004); U.S. Pat. Nos. 6,143,290; 6,596,535; 6,855,317; 6,936,257; 7,125,717; 7,378,087; 7,550,296); retroviral vectors including those based upon murine leukemia virus (MuLV), gibbon ape leukemia virus (GaLV), ecotropic retroviruses, simian immunodeficiency virus (SIV), human immunodeficiency virus (HIV), and combinations (see, e.g., Buchscher et al., J. Virol. 66:2731-39 (1992); Johann et al., J. Virol. 66: 1635-40 (1992); Sommerfelt et al., Virology 176:58-59 (1990); Wilson et al., J. Virol. 63:2374-78 (1989); Miller et al., J. Virol. 65:2220-24 (1991); Miller et al., Mol. Cell Biol. 10:4239 (1990); Kolberg, NIH Res. 4:43 1992; Cornetta et al., Hum. Gene Ther. 2:215 (1991)); lentiviral vectors including those based upon Human Immunodeficiency Virus (HIV-1), HIV-2, feline immunodeficiency virus (FIV), equine infectious anemia virus, Simian Immunodeficiency Virus (SIV), and maedi/visna virus (see, e.g., Pfeifer et al., Annu. Rev. Genomics Hum. Genet. 2: 177-211 (2001); Zufferey et al., J. Virol. 72: 9873, 1998; Miyoshi et al., J. Virol. 72:8150, 1998; Philpott and Thrasher, Human Gene Therapy 18:483, 2007; Engelman et al., J. Virol. 69: 2729, 1995; Nightingale et al., Mol. Therapy, 13: 1121, 2006; Brown et al., J. Virol. 73:9011 (1999); WO 2009/076524; WO 2012/141984; WO 2016/011083; McWilliams et al., J. Virol. 77: 11150, 2003; Powell et al., J. Virol. 70:5288, 1996) or any, variants thereof, and/or vectors that can be used to generate any of the viruses described above. In some embodiments, the recombinant vector can include regulatory sequences, such as promoter or enhancer sequences, that can regulate the expression of the viral genome, such as in the case for RNA viruses, in the packaging cell line (see, e.g., U.S. Pat. Nos. 5,385,839 and 5,168,062).

In some embodiments, the recombinant vector is an expression vector, e.g., an expression vector that permits expression of the encoded gene product when delivered into the target cell, e.g., a cell in the subject, e.g., a tumor cell, an immune cell and/or an APC. In some embodiments, the recombinant expression vectors contained in the infectious agent are capable of expressing the immunomodulatory proteins in the target cell in the subject, under conditions suited to expression of the protein.

In some aspects, nucleic acids or an expression vector comprises a nucleic acid sequence that encodes the immunomodulatory protein operatively linked to appropriate expression control sequences. Methods of affecting this operative linking, either before or after the nucleic acid sequence encoding the immunomodulatory protein is inserted into the vector, are well known. Expression control sequences include promoters, activators, enhancers, operators, ribosomal binding sites, start signals, stop signals, cap signals, polyadenylation signals, and other signals involved with the control of transcription or translation. The promoter can be operably linked to the portion of the nucleic acid sequence encoding the immunomodulatory protein. In some embodiments, the promotor is a constitutively active promotor in the target cell (such as a tissue-specific constitutively active promotor or other constitutive promotor). For example, the recombinant expression vector may also include, lymphoid tissue-specific transcriptional regulatory elements (TRE) such as a B lymphocyte, T lymphocyte, or dendritic cell specific TRE. Lymphoid tissue specific TRE are known in the art (see, e.g., Thompson et al., Mol. Cell. Biol. 12:1043-53 (1992); Todd et al., J. Exp. Med. 177:1663-74 (1993); Penix et al., J. Exp. Med. 178:1483-96 (1993)). In some embodiments, the promotor is an inducible promotor, which may be responsive to an inducing agent (such as a T cell activation signal). In some embodiments, nucleic acids delivered to the target cell in the subject, e.g., tumor cell, immune cell and/or APC, can be operably linked to any of the regulatory elements described above.

In some embodiments, the vector is a bacterial vector, e.g., a bacterial plasmid or cosmid. In some embodiments, the bacterial vector is delivered to the target cell, e.g., tumor cells, immune cells and/or APCs, via bacterial-mediated transfer of plasmid DNA to mammalian cells (also referred to as “bactofection”). In some embodiments, the delivered bacterial vector also contains appropriate expression control sequences for expression in the target cells, such as a promoter sequence and/or enhancer sequences, or any regulatory or control sequences described above. In some embodiments, the bacterial vector contains appropriate expression control sequences for expression and/or secretion of the encoded variant polypeptides in the infectious agent, e.g., the bacterium.

In some embodiments, polypeptides provided herein can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Peptides can then be assembled into the polypeptides as provided herein.

### IV. METHODS OF ASSESSING ACTIVITY IMMUNE MODULATION OF VARIANT CD86 POLYPEPTIDES AND IMMUNOMODULATORY PROTEINS

In some embodiments, the variant CD86 polypeptides provided herein (full-length and/or specific binding fragments or conjugates, stack constructs or fusion thereof or engineered cells) exhibit immunomodulatory activity to modulate T cell activation. In some embodiments, CD86 polypeptides modulate cytokine production, such as IFN-gamma, TNFα, or IL-2 expression, in a T cell assay relative to a wild-type or unmodified CD86 control. In some cases, modulation of expression cell activity can increase or decrease cytokine production, such as IFN-gamma, TNFα, or IL-2 expression, by or from T cells relative to the control CD86. Assays to determine specific binding and cytokine product, such as IFN-gamma, TNFα, or IL-2 expression, are well-known in the art and include the MLR (mixed lymphocyte reaction) assays measuring cytokine levels in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104).

In some embodiments, a variant CD86 polypeptide can in some embodiments increase or, in alternative embodiments, decrease cytokine production, such as IFN-gamma (interferon-gamma), TNFα, or IL-2 expression, in a primary T-cell assay relative to a wild-type CD86 control. In some embodiments, such activity may depend on whether the variant CD86 polypeptide is provided in a form for antagonist activity or in a form for agonist activity. Those of skill will recognize that different formats of the primary T-cell assay used to determine an increase or decrease in IFN-gamma, TNFα, or IL-2 expression exist.

In assaying for the ability of a variant CD86 to increase or decrease IFN-gamma, TNFα, or IL-2 expression in a primary T-cell assay, a Mixed Lymphocyte Reaction (MLR) assay can be used. In some embodiments, a variant CD86 polypeptide or immunomodulatory protein provided in antagonist form, such as soluble form, e.g., variant CD86-Fc or secretable immunomodulatory protein, block activity of the CD28 and thereby decreases MLR activity in the assay, such as observed by decreased production of IFN-gamma, TNFα, or IL-2 in the assay. In some embodiments, a variant CD86 polypeptide or immunomodulatory protein provided in agonist form, such as a localizing vIgD stack or conjugate containing a tumor-localizing moiety or an engineered cell expressing a transmembrane immunomodulatory protein as provided, may stimulate activity of CD28 and thereby increase MLR activity, such as evidenced by increased IFN-gamma, TNFα, or IL-2 production.

Alternatively, in assaying for the ability of a variant CD86 to modulate an increase or decrease in IFN-gamma or IL-2 expression in a primary T-cell assay, a co-immobilization assay can be used. In a co-immobilization assay, a TCR signal, provided in some embodiments by anti-CD3 antibody, is used in conjunction with a co-immobilized variant CD86 to determine the ability to increase or decrease IFN-gamma, TNFα, or IL-2 expression relative to a CD86 unmodified or wild-type control. In some embodiments, a variant CD86 polypeptide or immunomodulatory protein, e.g., a co-immobilized variant CD86 (e.g., CD86-Fc), increases IFN-gamma production in a co-immobilization assay.

In some embodiments, in assaying for the ability of a variant CD86 to modulate an increase or decrease in IFN-gamma, TNFα, or IL-2 expression a T cell reporter assay can be used. In some embodiments, the T cell is a Jurkat T cell line or is derived from Jurkat T cell lines. In reporter assays, the reporter cell line (e.g., Jurkat reporter cell) also can be generated to overexpress an inhibitory receptor, e.g. CTLA-4, that is a cognate binding partner of the variant IgSF domain polypeptide. In some embodiments, the reporter T cells also contain a reporter construct containing an inducible promoter responsive to T cell activation operably linked to a reporter. In some embodiments, the reporter is a fluorescent or luminescent reporter. In some embodiments, the reporter is luciferase. In some embodiments, the promoter is responsive to CD3 signaling. In some embodiments, the promoter is an NFAT promoter. In some embodiments, the promoter is responsive to costimulatory signaling, e.g., CD28 costimulatory signaling. In some embodiments, the promoter is an IL-2 promoter.

In aspects of a reporter assay, a reporter cell line is stimulated, such as by co-incubation with antigen presenting cells (APCs) expressing the wild-type ligand, e.g., CD86. In some embodiments, the APCs are artificial APCs. Artificial APCs are well known to a skilled artisan. In some embodiments, artificial APCs are derived from one or more mammalian cell line, such as K562, CHO or 293 cells. In some embodiments, the artificial APCs are engineered to express an anti-CD3 antibody and, in some cases, a costimulatory ligand. In some embodiments, the artificial APC is generated to overexpress the cognate binding partner of the variant IgSF domain polypeptide. For example, in the case of a variant CD86, the reporter cell line (e.g., Jurkat reporter cell) is generated to overexpress the ligandCD28. In some embodiments, the Jurkat reporter cells are co-incubated with artificial APCs overexpressing in the presence of the variant IgSF domain molecule or immunomodulatory protein, e.g., variant CD86 polypeptide or immunomodulatory protein. In some embodiments, reporter expression is monitored, such as by determining the luminescence or fluorescence of the cells. Agonist or antagonist (blocking) activity of a cognate binding partner can be monitored.

Use of proper controls is known to those of skill in the art, however, in the aforementioned embodiments, a control typically involves use of the unmodified CD86, such as a wild-type of native CD86 isoform from the same mammalian species from which the variant CD86 was derived or developed. In some embodiments, the wild-type or native CD86 is of the same form or corresponding form as the variant. For example, if the variant CD86 is a soluble form containing a variant ECD fused to an Fc protein, then the control is a soluble form containing the wild-type or native ECD of CD86 fused to the Fc protein.

In some embodiments, a variant CD86 polypeptide or immunomodulatory protein, increases IFN-gamma, TNFα, or IL-2 expression (i.e., protein expression) relative to a wild-type or unmodified CD86 control by at least: 5%, 10%, 20%, 30%, 40%, 50%, 60%, 70%, 80%, 90%, or higher. In other embodiments, a variant CD86 or immunomodulatory protein decreases IFN-gamma, TNFα, or IL-2 expression (i.e. protein expression) relative to a wild-type or unmodified CD86 control by at least: 5%, 10%, 20%, 30%, 40%, 50%, 60%, 70%, 80%, 90%, or higher. In some embodiments, the wild-type CD86 control is murine CD86, such as would typically be used for a variant CD86 altered in sequence from that of a wild-type murine CD86 sequence. In some embodiments, the wild-type CD86 control is human CD86, such as would typically be used for a variant CD86 altered in sequence from that of a corresponding wild-type human CD86 sequence such as an CD86 sequence comprising the sequence of amino acids of SEQ ID NO: 2, SEQ ID NO: 122 or SEQ ID NO: 123.

### V. PHARMACEUTICAL FORMULATIONS

Provided herein are compositions containing any of the variant CD86 polypeptides, immunomodulatory proteins, conjugates, engineered cells or infectious agents described herein. The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example variant CD86 polypeptides dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

In some embodiments, provided are pharmaceutical compositions containing the transmembrane immunomodulatory proteins, including engineered cells expressing such transmembrane immunomodulatory proteins. In some embodiments, the pharmaceutical compositions and formulations include one or more optional pharmaceutically acceptable carrier or excipient. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Such a formulation may, for example, be in a form suitable for intravenous infusion. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is administered to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data. A number of biomarkers or physiological markers for therapeutic effect can be monitored including T cell activation or proliferation, cytokine synthesis or production (e.g., production of TNF-α, IFN-γ, IL-2), induction of various activation markers (e.g., CD25, IL-2 receptor), inflammation, joint swelling or tenderness, serum level of C-reactive protein, anti-collagen antibody production, and/or T cell-dependent antibody response(s).

In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the pharmaceutical composition is about 1 μg of protein per kg subject body mass or more (such as about 2 μg of protein per kg subject body mass or more, about 5 μg of protein per kg subject body mass or more, about 10 μg of protein per kg subject body mass or more, about 25 μg of protein per kg subject body mass or more, about 50 μg of protein per kg subject body mass or more, about 100 μg of protein per kg subject body mass or more, about 250 μg of protein per kg subject body mass or more, about 500 μg of protein per kg subject body mass or more, about 1 mg of protein per kg subject body mass or more, about 2 mg of protein per kg subject body mass or more, or about 5 mg of protein per kg subject body mass or more).

In some embodiments, a therapeutic amount of a cell composition is administered. Typically, precise amount of the compositions of the present invention to be administered can be determined by a physician with consideration of individual differences in age, weight, tumor size, extent of infection or metastasis, and condition of the patient (subject). It can generally be stated that a pharmaceutical composition comprising engineered cells, e.g., T cells, as described herein may be administered at a dosage of 10⁴ to 10⁹ cells/kg body weight, such as 10⁵ to 10⁶ cells/kg body weight, including all integer values within those ranges. Engineered cell compositions, such as T cell compositions, may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

In some embodiments, the pharmaceutical composition contains infectious agents containing nucleic acid sequences encoding the immunomodulatory variant polypeptides. In some embodiments, the pharmaceutical composition contains a dose of infectious agents suitable for administration to a subject that is suitable for treatment. In some embodiments, the pharmaceutical composition contains an infectious agent that is a virus, at a single or multiple dosage amount, of between about between or between about 1×10⁵ and about 1×10¹² plaque-forming units (pfu), 1×10⁶ and 1×10¹⁰ pfu, or 1×10⁷ and 1×10¹⁰ pfu, each inclusive, such as at least or at least about or at about 1×10⁶, 1×10⁷, 1×10⁸, 1×10⁹, 2×10⁹, 3×10⁹, 4×10⁹, 5×10⁹pfu or about 1×10¹⁰ pfu. In some embodiments, the pharmaceutical composition can contain a virus concentration of from or from about 10⁵ to about 10¹⁰ pfu/mL, for example, 5×10⁶ to 5×10⁹ or 1×10⁷ to 1×10⁹ pfu/mL, such as at least or at least about or at about 10⁶ pfu/mL, 10⁷ pfu/mL, 108 pfu/mL or 10⁹ pfu/mL. In some embodiments, the pharmaceutical composition contains an infectious agent that is a bacterium, at a single or multiple dosage amount, of between about between or between about 1×10³ and about 1×10⁹ colony-forming units (cfu), 1×10⁴ and 1×10⁹ cfu, or 1×10⁵ and 1×10⁷ cfu, each inclusive, such as at least or at least about or at about 1×10⁴, 1×10⁵, 1×10⁶, 1×10⁷, 1×10⁸ or 1×10⁹ cfu. In some embodiments, the pharmaceutical composition can contain a bacterial concentration of from or from about 103 to about 10⁸ cfu/mL, for example, 5×10⁵ to 5×10⁷ or 1×10⁶ to 1×10⁷ cfu/mL, such as at least or at least about or at about 10⁵ cfu/mL, 10⁶ cfu/mL, 10⁷ cfu/mL or 10⁸ cfu/mL.

A variety of means are known for determining if administration of a therapeutic composition of the invention sufficiently modulates immunological activity by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of immunological activity include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T-cell proliferation assays and pepscan analysis based on 3H-thymidine incorporation following stimulation with anti-CD3 antibody, anti-T-cell receptor antibody, anti-CD28 antibody, calcium ionophores, PMA (phorbol 12-myristate 13-acetate) antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes such as improvement of autoimmune, neurodegenerative, and other diseases involving self-proteins or self-polypeptides (clinical scores, requirements for use of additional therapies, functional status, imaging studies) for example, by measuring relapse rate or disease severity (using clinical scores known to the ordinarily skilled artisan) in the case of multiple sclerosis, measuring blood glucose in the case of type I diabetes, or joint inflammation in the case of rheumatoid arthritis.

### VI. ARTICLES OF MANUFACTURE AND KITS

Also provided herein are articles of manufacture that comprise the pharmaceutical compositions described herein in suitable packaging. Suitable packaging for compositions (such as ophthalmic compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Further provided are kits comprising the pharmaceutical compositions (or articles of manufacture) described herein, which may further comprise instruction(s) on methods of using the composition, such as uses described herein. The kits described herein may also include other materials desirable from a commercial and user standpoint, including other buffers, diluents, filters, needles, syringes, and package inserts with instructions for performing any methods described herein.

### VII. THERAPEUTIC APPLICATIONS

Provided herein are methods using the provided pharmaceutical compositions containing a variant CD86 polypeptides immunomodulatory protein, engineered cell or infectious agent described herein, for modulating an immune response, including in connection with treating a disease or condition in a subject, such as in a human patient. The pharmaceutical compositions described herein (including pharmaceutical composition comprising the variant CD86 polypeptides, the immunomodulatory proteins, the conjugates, and the engineered cells described herein) can be used in a variety of therapeutic applications, such as the treatment of a disease. For example, in some embodiments the pharmaceutical composition is used to treat inflammatory or autoimmune disorders, cancer, organ transplantation, viral infections, and/or bacterial infections in a mammal. The pharmaceutical composition can modulate (e.g., increase or decrease) an immune response to treat the disease. In some embodiments, the methods are carried out with variant CD86 polypeptides in a format to increase an immune response in a subject. In some such aspects, increasing an immune response treats a disease or condition in the subject, such as a tumor or cancer. In some embodiments, the methods are carried out with variant CD86 polypeptides in a format to decrease an immune response in a subject. In some such aspects, decreasing an immune response treats a disease or condition in a subject, such as an inflammatory disease or condition, e.g. an autoimmune disease.

In some embodiments, the provided methods are applicable to therapeutic administration of variant CD86 polypeptides, the immunomodulatory proteins, the conjugates, the engineered cells and infectious agents described herein. It is within the level of a skilled artisan, in view of the provided disclosure, to choose a format for the indication depending on the type of modulation of the immune response, e.g., increase or decrease that is desired.

In some embodiments, a pharmaceutical composition provided herein that stimulates or increases the immune response is administered, which can be useful, for example, in the treatment of cancer, viral infections, or bacterial infections. In some embodiments, the pharmaceutical composition contains a variant CD86 polypeptide in a format that exhibits agonist activity of its cognate binding partner CD28 and/or that stimulates or initiates costimulatory signaling via CD28. Exemplary formats of a CD86 polypeptide for use in connection with such therapeutic applications include, for example, an immunomodulatory protein or “stack” of a variant CD86 polypeptide and an IgSF domain or variant thereof that binds to a tumor antigen (e.g. Nkp30 or affinity-modified variant) (also called a “tumor-localizing IgSF domain), a conjugate containing a variant CD86 polypeptide linked to a tumor-targeting moiety (also called a tumor-localizing moiety), an engineered cell expressing a transmembrane immunomodulatory protein, or an infectious agent comprising a nucleic acid molecule encoding a transmembrane immunomodulatory protein, such as for expression of the transmembrane immunomodulatory protein in an infected cell (e.g. tumor cell or APC, e.g. dendritic cell).

The provided methods to modulate an immune response can be used to treat a disease or condition, such as a tumor or cancer. In some embodiments, the pharmaceutical composition can be used to inhibit growth of mammalian cancer cells (such as human cancer cells). A method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with cancer that does not get worse), or overall survival (also called “survival rate;” i.e., the percentage of people in a study or treatment group who are alive for a certain period of time after they were diagnosed with or treated for cancer) relative to treatment with a control.

The cancers that can be treated by the pharmaceutical compositions and the treatment methods described herein include, but are not limited to, melanoma, bladder cancer, hematological malignancies (leukemia, lymphoma, myeloma), liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer. In some embodiments, the cancer is Ewing's sarcoma. In some embodiments, the cancer is selected from melanoma, lung cancer, bladder cancer, and a hematological malignancy. In some embodiments, the cancer is a lymphoma, lymphoid leukemia, myeloid leukemia, cervical cancer, neuroblastoma, or multiple myeloma. In some embodiments, the cancer is breast cancer.

In some embodiments, the pharmaceutical composition is administered as a monotherapy (i.e., as a single agent) or as a combination therapy (i.e., in combination with one or more additional anticancer agents, such as a chemotherapeutic drug, a cancer vaccine, or an immune checkpoint inhibitor. In some embodiments, the pharmaceutical composition can also be administered with radiation therapy. In some aspects of the present disclosure, the immune checkpoint inhibitor is nivolumab, Tremelimumab, pembrolizumab, ipilimumab, or the like.

In some embodiments, the pharmaceutical composition suppresses an immune response, which can be useful in the treatment of inflammatory or autoimmune disorders, or organ transplantation. In some embodiments, the pharmaceutical composition contains a variant CD86 polypeptide in a format that exhibits antagonist activity of its cognate binding partner CD28 and/or that blocks or inhibits costimulatory signaling via CD28. Exemplary formats of a CD86 polypeptide for use in connection with such therapeutic applications include, for example, a variant CD86 polypeptide that is soluble (e.g. variant CD86-Fc fusion protein), an immunomodulatory protein or “stack” of a variant CD86 polypeptide and another IgSF domain, including soluble forms thereof that are Fc fusions, an engineered cell expressing a secretable immunomodulatory protein, or an infectious agent comprising a nucleic acid molecule encoding a secretable immunomodulatory protein, such as for expression and secretion of the secretable immunomodulatory protein in an infected cell (e.g. tumor cell or APC, e.g. dendritic cell).

In some embodiments, the pharmaceutical composition contains a variant CD86 polypeptide in a format that exhibits agonizes activity of its cognate binding partner CD28 and/or that facilitates costimulatory signaling via CD28. Exemplary formats of a CD86 polypeptide for use in connection with such therapeutic applications include, for example, a variant CD86 polypeptide that is a transmembrane immunomodulatory polypeptide, an engineered cell expressing a transmembrane immunomodulatory polypeptide, or an infectious agent comprising a nucleic acid molecule encoding a transmembrane immunomodulatory polypeptide, such as for expressing the transmembrane immunomodulatory protein on an infected cell (e.g. T cell, APC, e.g. dendritic cell).

In some embodiments, the inflammatory or autoimmune disorder is antineutrophil cytoplasmic antibodies (ANCA)-associated vasculitis, a vasculitis, an autoimmune skin disease, transplantation, a Rheumatic disease, an inflammatory gastrointestinal disease, an inflammatory eye disease, an inflammatory neurological disease, an inflammatory pulmonary disease, an inflammatory endocrine disease, or an autoimmune hematological disease.

In some embodiments, the inflammatory and autoimmune disorders that can be treated by the pharmaceutical composition described herein is Addison's Disease, allergies, alopecia areata, Alzheimer's, anti-neutrophil cytoplasmic antibodies (ANCA)-associated vasculitis, ankylosing spondylitis, antiphospholipid syndrome (Hughes Syndrome), asthma, atherosclerosis, rheumatoid arthritis, autoimmune hemolytic anemia, autoimmune hepatitis, autoimmune inner ear disease, autoimmune lymphoproliferative syndrome, autoimmune myocarditis, autoimmune oophoritis, autoimmune orchitis, azoospermia, Behcet's Disease, Berger's Disease, bullous pemphigoid, cardiomyopathy, cardiovascular disease, celiac Sprue/coeliac disease, chronic fatigue immune dysfunction syndrome (CFIDS), chronic idiopathic polyneuritis, chronic inflammatory demyelinating, polyradicalneuropathy (CIDP), chronic relapsing polyneuropathy (Guillain-Barre syndrome), Churg-Strauss Syndrome (CSS), cicatricial pemphigoid, cold agglutinin disease (CAD), COPD (chronic obstructive pulmonary disease), CREST syndrome, Crohn's disease, dermatitis, herpetiformus, dermatomyositis, diabetes, discoid lupus, eczema, epidermolysis bullosa acquisita, essential mixed cryoglobulinemia, Evan's Syndrome, exopthalmos, fibromyalgia, Goodpasture's Syndrome, Graves' Disease, Hashimoto's thyroiditis, idiopathic pulmonary fibrosis, idiopathic thrombocytopenia purpura (ITP), IgA nephropathy, immunoproliferative disease or disorder, inflammatory bowel disease (IBD), interstitial lung disease, juvenile arthritis, juvenile idiopathic arthritis (JIA), Kawasaki's Disease, Lambert-Eaton Myasthenic Syndrome, lichen planus, lupus nephritis, lymphocytic hypophysitis, Meniere's Disease, Miller Fish Syndrome/acute disseminated encephalomyeloradiculopathy (EMR), mixed connective tissue disease, multiple sclerosis (MS), muscular rheumatism, myalgic encephalomyelitis (ME), myasthenia gravis, ocular inflammation, pemphigus foliaceus, pemphigus vulgaris, pernicious anemia, polyarteritis nodosa, polychondritis, polyglandular syndromes (Whitaker's syndrome), polymyalgia rheumatica, polymyositis, primary agammaglobulinemia, primary biliary cirrhosis/autoimmune cholangiopathy, psoriasis, psoriatic arthritis, Raynaud's Phenomenon, Reiter's Syndrome/reactive arthritis, restenosis, rheumatic fever, rheumatic disease, sarcoidosis, Schmidt's syndrome, scleroderma, Sjörgen's Syndrome, stiff-man syndrome, systemic lupus erythematosus (SLE), systemic scleroderma, Takayasu arteritis, temporal arteritis/giant cell arteritis, thyroiditis, Type 1 diabetes, ulcerative colitis, uveitis, vasculitis, vitiligo, interstitial bowel disease or Wegener's Granulomatosis. In some embodiments, the inflammatory or autoimmune disorder is selected from interstitial bowel disease, transplant, Crohn's disease, ulcerative colitis, multiple sclerosis, asthma, rheumatoid arthritis, and psoriasis.

In some embodiments, the pharmaceutical composition is administered to modulate an autoimmune condition. For example, suppressing an immune response can be beneficial in methods for inhibiting rejection of a tissue, cell, or organ transplant from a donor by a recipient. Accordingly, in some embodiments, the pharmaceutical compositions described herein are used to limit or prevent graft-related or transplant related diseases or disorders, such as graft versus host disease (GVHD). In some embodiments, the pharmaceutical compositions are used to suppress autoimmune rejection of transplanted or grafted bone marrow, organs, skin, muscle, neurons, islets, or parenchymal cells.

Pharmaceutical compositions comprising engineered cells and the methods described herein can be used in adoptive cell transfer applications. In some embodiments, cell compositions comprising engineered cells can be used in associated methods to, for example, modulate immunological activity in an immunotherapy approach to the treatment of, for example, a mammalian cancer or, in other embodiments the treatment of autoimmune disorders. The methods employed generally comprise a method of contacting a TIP of the present invention with a mammalian cell under conditions that are permissive to specific binding of the affinity modified IgSF domain and modulation of the immunological activity of the mammalian cell. In some embodiments, immune cells (such as tumor infiltrating lymphocytes (TILs), T-cells (including CD8+ or CD4+ T-cells), or APCs) are engineered to express as a membrane protein and/or as a soluble variant CD86 polypeptide, immunomodulatory protein, or conjugate as described herein. The engineered cells can then be contact a mammalian cell, such as an APC, a second lymphocyte or tumor cell in which modulation of immunological activity is desired and under conditions that are permissive of specific binding of the affinity modified IgSF domain to a counter-structure on the mammalian cell such that immunological activity can be modulated in the mammalian cell. Cells can be contacted in vivo or ex vivo.

In some embodiments, the engineered cells are autologous cells. In other embodiments, the cells are allogeneic. In some embodiments, the cells are autologous engineered cells reinfused into the mammal from which it was isolated. In some embodiments, the cells are allogenic engineered cells infused into the mammal. In some embodiments, the cells are harvested from a patient's blood or tumor, engineered to express a polypeptide (such as the variant CD86 polypeptide, immunomodulatory protein, or conjugate as described herein), expanded in an in vitro culture system (for example, by stimulating the cells), and reinfused into the patient to mediate tumor destruction. In some embodiments, the method is conducted by adoptive cell transfer wherein cells expressing the TIP (e.g., a T-cell) are infused back into the patient. In some embodiments, the therapeutic compositions and methods of the invention are used in the treatment of a mammalian patient of cancers such as lymphoma, lymphoid leukemia, myeloid leukemia, cervical cancer, neuroblastoma, or multiple myeloma.

In some embodiments, the provided methods are for treating a subject that is or is suspected of having the disease or condition for which the therapeutic application is directed. In some cases, the subject for treatment can be selected prior to treatment based on one or more features or parameters, such as to determine suitability for the therapy or to identify or select subjects for treatment in accord with any of the provided embodiments, including treatment with any of the provided variant CD86 polypeptides, immunomodulatory proteins, conjugates, engineered cells or infectious agents.

### VIII. EXEMPLARY EMBODIMENTS

Among the provided embodiments are:

1. A variant CD86 polypeptide, comprising an extracellular domain or an IgV domain or specific binding fragment thereof, wherein the variant CD86 polypeptide comprises one or more amino acid modifications in an unmodified CD86 polypeptide or a specific binding fragment thereof corresponding to position(s) selected from among 13, 18, 25, 28, 33, 38, 39, 40, 43, 45, 52, 53, 60, 68, 71, 77, 79, 80, 82, 86, 88, 89, 90, 92, 93, 97, 102, 104, 113, 114, 123, 128, 129, 132, 133, 137, 141, 143, 144, 148, 153, 154, 158, 170, 172, 175, 178, 180, 181, 183, 185, 192, 193, 196, 197, 198, 205, 206, 207, 212, 215, 216, 222, 223, or 224, with reference to positions set forth in SEQ ID NO:29.

2. The variant CD86 polypeptide of embodiment 1, wherein the amino acid modifications comprise amino acid substitutions, deletions or insertions.

3. The variant CD86 polypeptide of embodiment 1 or embodiment 2, wherein the unmodified CD86 polypeptide is a mammalian CD86 polypeptide or a specific binding fragment thereof.

4. The variant CD86 polypeptide of embodiment 3, wherein the unmodified CD86 polypeptide is a human CD86 polypeptide or a specific binding fragment thereof.

5. The variant CD86 polypeptide of any of embodiments 1-4, wherein the variant CD86 polypeptide comprises the extracellular domain of a human CD86, wherein the one or more amino acid modifications are in one or more residues of the extracellular domain of the unmodified CD86 polypeptide.

6. The variant CD86 polypeptide of any of embodiments 1-5, wherein the unmodified CD86 polypeptide comprises (i) the sequence of amino acids set forth in SEQ ID NO:29, (ii) a sequence of amino acids that has at least 95% sequence identity to SEQ ID NO:29; or (iii) a portion thereof comprising an IgV domain or specific binding fragment of the IgV domain.

7. The variant CD86 polypeptide of any of embodiments 1-6, wherein the unmodified CD86 comprises the sequence of amino acids set forth in SEQ ID NO:29.

8. The variant CD86 polypeptide of embodiment 6, wherein the portion thereof comprises amino acid residues 33-131 or 24-134 of the IgV domain or specific binding fragment of the IgV domain.

9. The variant CD86 polypeptide of any of embodiments 1-6 and embodiment 8, wherein the unmodified CD86 polypeptide comprises (i) the sequence of amino acids set forth in SEQ ID NO: 123, (ii) a sequence of amino acids that has at least 95% sequence identity to SEQ ID NO: 123; or (iii) a portion thereof comprising an IgV domain or specific binding fragment of the IgV domain.

10. The variant CD86 polypeptide of any of embodiments 1-6, wherein the unmodified CD86 comprises the sequence of amino acids set forth in SEQ ID NO:123.

11. The variant CD86 polypeptide of any of embodiments 1-6, 8 and 9, wherein the unmodified CD86 polypeptide comprises (i) the sequence of amino acids set forth in SEQ ID NO:122, (ii) a sequence of amino acids that has at least 95% sequence identity to SEQ ID NO:122; or (iii) or a specific binding fragment thereof.

12. The variant CD86 polypeptide of any of embodiments 1-6, 8, 9 and 11, wherein the unmodified CD86 comprises the sequence of amino acids set forth in SEQ ID NO: 122.

13. The variant CD86 polypeptide of any of embodiments 1-12, wherein:
the specific binding fragment has a length of at least 50, 60, 70, 80, 90, 95 or more amino acids; or
the specific binding fragment comprises a length that is at least 80% of the length of the IgV domain set forth as residues 33-131 of SEQ ID NO:2.

14. The variant CD86 polypeptide of any of embodiments 1-13, wherein the variant CD86 comprises up to 1, 2, 3, 4, 5, 6, 7, 8, 9, 10, 11, 12, 13, 14, 15, 16, 17, 18, 19 or 20 amino acid modifications, optionally amino acid substitutions, insertions and/or deletions.

15. The variant CD86 polypeptide of any of embodiments 1-14, wherein the one or more amino acid modification are one or more amino acid substitutions selected from A13V, Q18K, Q25L, S28G, F33I, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F, or a conservative amino acid substitution thereof.

16. The variant CD86 polypeptide of any of embodiments 1-15, comprising one or more amino acid modifications selected from among Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93 T/L132M/V148D/S181P/P216H, Q25L/F33I/H90Y/V128A/P141A/E158G/S181P, Q25L/N39D/K80R/Q86R/I88F/H90L/K93 T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V45I/D68N/H90L/S183P/L205S, E38V/S114G/P143H, H90Y/L180S, H90Y/Y129N, I89V/H90L/I193V, K80E/H90Y/H222T/I223F/P224L, K80M/I88T, K92I/F113S, M60K/H90L, Q25L/F33I/H90L, Q25L/F33I/Q86R/H90L/K93T, Q25L/H90L, Q25L/H90L/P185S, Q25L/H90L/P185S/P224L, Q25L/H90L/S179R, Q25L/H90Y/S181P/I193V, Q25L/K82T/H90L/T152S/S207P, Q25L/Q86R/H90L/K93T, or S28G/H90Y.

17. The variant CD86 polypeptide of any of embodiments 1-14, wherein the one or more amino acid modifications are at position 25 and/or position 90.

18. The variant CD86 polypeptide of any of embodiments 1-14 and 17, wherein the one or more amino acid modifications comprise Q25L, H90Y, or H90L.

19. The variant CD86 polypeptide of any of embodiments 1-14 and 17, wherein the one or more amino acid modifications comprise modification at position 25 and position 90.

20. The variant CD86 polypeptide of embodiment 19, wherein the one or more amino acid modifications are selected from Q25L/H90Y or Q25L/H90L.

21. The variant CD86 polypeptide of any of embodiments 1-20, comprising one or more amino acid modifications selected from among Q25L/T71A/H90Y, Q25L/D53G/E212V, Q25L/H90L, N43K/I79N/H90L/I178T/E198D, A13V/Q25L/H90L/S181P/L197M/S206T, Q25L/Q86R/H90L/K93 T/L132M/V148D/S181P/P216H, Q25L/F33I/H90Y/V128A/P141A/E158G/S181P, Q25L/N39D/K80R/Q86R/I88F/H90L/K93 T/N123D/N154D, Q25L/H90L/K93T/M97L/T133A/S181P/D215V, Q25L/Q86R/H90L/N104S, Q25L/L40M/H90L/L180S/S183P, Q18K/Q25L/F33I/L40S/H90L, Q25L/Q86K/H90L/I137T/S181P, Q25L/L77P/H90Y/K153R/V170D/S181P, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/I196V/E198D, Q25L/V451/D68N/H90L/S183P/L205S/E212X, H90Y/L180S, H90Y/Y129N, I89V/H90L/I193V, K80E/H90Y/H222T/I223F/P224L, M60K/H90L; Q25L/F33I/H90L; Q25L/F33I/Q86R/H90L/K93T; Q25L/H90L; Q25L/H90L/P185S; Q25L/H90L/P185S/P224L; Q25L/H90L/S179R; Q25L/H90Y/S181P/I193V; Q25L/K82T/H90L/T152S/S207P; Q25L/Q86R/H90L/K93T, S28G/H90Y, A13V/Q25L/H90L, Q25L/H90L/K93T/M97L, Q25L/Q86R/H90L or I89V/H90L.

22. The variant CD86 polypeptide of any of embodiments 1-21, comprising one or more amino acid modifications A13V/Q25L/H90L.

23. The variant CD86 polypeptide of any of embodiments 1-22, comprising one or more amino acid modifications A13V/Q25L/H90L/S181P/L197M/S206T.

24. The variant CD86 polypeptide of any of embodiments 1-21, comprising one or more amino acid modifications Q25L/H90L/K93T/M97L.

25. The variant CD86 polypeptide of any of embodiments 1-21 and 24, comprising one or more amino acid modifications Q25L/H90L/K93T/M97L/T133A/S181P/D215V.

26. The variant CD86 polypeptide of any of embodiments 1-21 and 24, comprising one or more amino acid modifications Q25L/Q86R/H90L.

27. The variant CD86 polypeptide of any of embodiments 1-21 and 26, comprising one or more amino acid modifications Q25L/Q86R/H90L/N104S.

28. The variant CD86 polypeptide of any of embodiments 1-21, comprising one or more amino acid modifications I89V/H90L.

29. The variant CD86 polypeptide of any of embodiments 1-21 and 28, comprising one or more amino acid modifications I89V/H90L/I193V.

30. The variant CD86 polypeptide of any of embodiments 1-21, comprising one or more amino acid modifications M60K/H90L.

31. The variant CD86 polypeptide of any of embodiments 1-21, comprising one or more amino acid modifications Q25L/F33I/H90L.

32. The variant CD86 polypeptide of any of embodiments 1-21, comprising one or more amino acid modifications Q25L/H90L/P185S.

33. The variant CD86 polypeptide of any of embodiments 1-32, wherein the variant CD86 polypeptide comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO:29 or a specific binding fragment thereof.

34. The variant CD86 polypeptide of any of embodiments 1-33, wherein the variant CD86 polypeptide specifically binds to the ectodomain of CD28 with increased affinity compared to the binding of the unmodified CD86 for the same ectodomain.

35. The variant CD86 polypeptide of embodiment 34, wherein the binding affinity is increased at least at or about 1.5-fold, at least at or about 2.0-fold, at least at or about 5.0-fold, at least at or about 10-fold, at least at or about 20-fold, at least at or about 30-fold, at least at or about 40-fold, at least at or about 50-fold, at least at or about 60-fold, at least at or about 70-fold, at least at or about 80-fold, at least at or about 90-fold, at least at or about 100-fold, or at least at or about 125-fold.

36. The variant CD86 polypeptide of any of embodiments 1-35, wherein the variant CD86 polypeptide specifically binds to the ectodomain of CTLA-4 with decreased affinity compared to the binding of the unmodified CD86 for the same ectodomain.

37. The variant CD86 polypeptide of embodiment 36, wherein the decreased binding affinity is decreased at least at or about 1.2-fold, at least at or about 1.4-fold, at least at or about 1.5-fold, at least at or about 1.75-fold, at least at or about 2.0-fold, at least at or about 2.5-fold, at least at or about 3.0-fold, at least at or about 4.0-fold, or at least at or about 5.0-fold.

38. The variant CD86 polypeptide of any of embodiments 1-37, wherein the variant CD86 polypeptide specifically binds to the ectodomain of CTLA-4 with the same or similar binding affinity as the binding of the unmodified CD86 for the same ectodomain, optionally wherein the same or similar binding affinity is from at or about 90% to 120% of the binding affinity of the unmodified CD86.

39. The variant CD86 polypeptide of any of embodiments 1-38, wherein the variant CD86 polypeptide comprises the full extracellular domain.

40. The variant CD86 polypeptide of any of embodiments 1-39, wherein the variant CD86 polypeptide comprises the sequence of amino acids set forth in any of SEQ ID NOS: 85-121 or a specific binding fragment thereof, a sequence of amino acids that exhibits at least 95% sequence identity to any of SEQ ID NOS: 85-121 or a specific binding fragment thereof and that contains the one or more of the amino acid modifications of the respective SEQ ID NO set forth in any of SEQ ID NOS: 85-121.

41. The variant CD86 polypeptide of any of embodiments 1-40, wherein the variant CD86 polypeptide comprises the sequence of amino acids set forth in any of SEQ ID NOS: 141-177 or a specific binding fragment thereof, a sequence of amino acids that exhibits at least 95% sequence identity to any of SEQ ID NOS: 141-177 or a specific binding fragment thereof and that contains the one or more of the amino acid modifications of the respective SEQ ID NO set forth in any of SEQ ID NOS: 141-177.

42. The variant CD86 polypeptide of any of embodiments 34-41, wherein the CD28 is a human CD28.

43. The variant CD86 polypeptide of any of embodiments 34-42, wherein the CTLA-4 is a human CTLA-4.

44. The variant CD86 polypeptide of any of embodiments 1-43 that is a soluble protein.

45. The variant CD86 polypeptide of any of embodiments 1-44, wherein:
the variant CD86 polypeptide lacks the CD86 transmembrane domain and intracellular signaling domain; and/or
the variant CD86 polypeptide is not capable of being expressed on the surface of a cell.

46. The variant CD86 polypeptide of any of embodiments 1-45 that is linked to a multimerization domain.

47. The variant CD86 polypeptide of embodiment 46, wherein the multimerization domain is an Fc domain or a variant thereof with reduced effector function.

48. The variant CD86 polypeptide of any of embodiments 1-47 that is linked to an Fc domain or a variant thereof with reduced effector function.

49. The variant CD86 polypeptide of embodiment 47 or embodiment 48, wherein the Fc domain is a human IgG1 or is a variant thereof with reduced effector function.

50. The variant CD86 polypeptide of any of embodiments 47-49, wherein the Fc domain comprises the sequence of amino acids set forth in SEQ ID NO: 229 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 229.

51. The variant CD86 polypeptide of any of embodiments 47-50, wherein the Fc domain is or comprises the sequence of amino acids set forth in SEQ ID NO: 229.

52. The variant CD86 polypeptide of any of embodiments 47-50, wherein the Fc domain is a variant IgG1 Fc domain comprising one or more amino acid modifications selected from among E233P, L234A, L234V, L235A, L235E, G236del, G237A, S267K, N297G, V302C and K447del, each by EU numbering.

53. The variant CD86 polypeptide of any of embodiments 47-50 and 52, wherein the Fc domain comprises the amino acid modifications L234A/L235E/G237A.

54. The variant CD86 polypeptide of any of embodiments 47-50, 52 and 53, wherein the Fc domain comprises the amino acid modification C220S by EU numbering.

55. The variant CD86 polypeptide of any of embodiments 47-50 and 52-54, wherein the Fc domain comprises the amino acid modification K447del by EU numbering.

56. The variant CD86 polypeptide of any of embodiments 47-50 and 52-55, wherein the Fc domain comprises the sequence of amino acids set forth in SEQ ID NO: 230 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 230 and comprises one or more of the respective amino acid modifications set forth in SEQ ID NO: 230 compared to human IgG1.

57. The variant CD86 polypeptide of any of embodiments 47-50 and 52-56, wherein the Fc domain is or comprises the sequence of amino acids set forth in SEQ ID NO: 230.

58. The variant CD86 polypeptide of any of embodiments 47-57, wherein the variant CD86 polypeptide is linked to the multimerization domain or Fc indirectly via a linker, optionally a G4S linker.

59. The variant CD86 polypeptide of any of embodiments 1-43, wherein the variant CD86 polypeptide is a transmembrane immunomodulatory protein further comprising a transmembrane domain, optionally wherein the transmembrane domain is linked, directly or indirectly, to the extracellular domain (ECD) or specific binding fragment thereof of the variant CD86 polypeptide.

60. The variant CD86 polypeptide of embodiment 59, wherein the transmembrane domain comprises the sequence of amino acids set forth as residues 248-268 of SEQ ID NO:2 or a functional variant thereof that exhibits at least 85% sequence identity to residues 248-268 of SEQ ID NO:2.

61. The variant CD86 polypeptide of embodiment 59 or embodiment 60, further comprising a cytoplasmic domain, optionally wherein the cytoplasmic domain is linked, directly or indirectly, to the transmembrane domain.

62. The variant CD86 polypeptide of embodiment 61, wherein the cytoplasmic domain is or comprises a native CD86 cytoplasmic domain.

63. The variant CD86 polypeptide of embodiment 61 or embodiment 62, wherein the cytoplasmic domain comprises the sequence of amino acids set forth as residues 269-329 of SEQ ID NO:2 or a functional variant thereof that exhibits at least 85% sequence identity to residues 269-329 of SEQ ID NO:2.

64. The variant CD86 polypeptide of embodiment 61, wherein the cytoplasmic domain comprises an ITAM signaling motif and/or is or comprises an intracellular signaling domain of CD3 zeta.

65. The variant CD86 polypeptide of embodiment 59 or embodiment 60, wherein the polypeptide does not comprise a cytoplasmic signaling domain and/or is not capable of mediating or modulating an intracellular signal when expressed on a cell.

66. An immunomodulatory protein, comprising a first variant CD86 polypeptide of any of embodiments 1-65 and second variant CD86 polypeptide of any of embodiments 1-58.

67. The immunomodulatory protein of embodiment 66, wherein the first and second variant CD86 polypeptides are linked indirectly via a linker.

68. The immunomodulatory protein of embodiment 66 or embodiment 67, wherein the first and second variant CD86 polypeptide are each linked to a multimerization domain, whereby the immunomodulatory protein is a multimer comprising the first and second variant CD86 polypeptide.

69. The immunomodulatory protein of embodiment 68, wherein the multimer is a dimer, optionally a homodimer.

70. The immunomodulatory protein of any of embodiments 66-69, wherein the first variant CD86 polypeptide and the second variant CD86 polypeptide are the same.

71. An immunomodulatory protein, comprising the variant CD86 polypeptide of any of embodiments 1-65 linked, directly or indirectly via a linker, to a second polypeptide comprising an immunoglobulin superfamily (IgSF) domain of an IgSF family member.

72. The immunomodulatory protein of embodiment 71, wherein the IgSF domain is an affinity-modified IgSF domain, said affinity-modified IgSF domain comprising one or more amino acid modifications compared to the unmodified or wild-type IgSF domain of the IgSF family member.

73. The immunomodulatory protein of embodiment 72, wherein the IgSF domain is an affinity modified IgSF domain that exhibits altered binding to one or more of its cognate binding partner(s) compared to the binding of the unmodified or wild-type IgSF domain of the IgSF family member to the same one or more cognate binding partner(s).

74. The immunomodulatory protein of embodiment 73, wherein the IgSF domain exhibits increased binding to one or more of its cognate binding partner(s) compared to the binding of the unmodified or wild-type IgSF domain of the IgSF family member to the same one or more cognate binding partner(s).

75. The immunomodulatory protein of any of embodiments 71-74, wherein the IgSF domain of the second polypeptide is a tumor-localizing moiety that binds to a ligand expressed on a tumor or that binds to a ligand expressed on a tumor or is an inflammatory-localizing moiety that binds to a cell or tissue associated with an inflammatory environment.

76. The immunomodulatory polypeptide of embodiment 75, wherein the ligand is B7H6.

77. The immunomodulatory polypeptide of embodiment 75 or embodiment 76, wherein the IgSF domain is from NKp30.

78. The immunomodulatory protein of any of embodiments 71-77 wherein the immunomodulatory protein further comprises a multimerization domain linked to at least one of the variant CD86 polypeptide, or the second polypeptide.

79. The immunomodulatory protein of any of embodiments 71-78, further comprising a third polypeptide comprising an IgSF domain of an IgSF family member or an affinity-modified IgSF domain thereof, said affinity-modified IgSF domain comprising one or more amino acid modifications compared to the unmodified or wild-type IgSF domain of the IgSF family member.

80. The immunomodulatory protein of embodiment 79, wherein:
the third polypeptide is the same as the first and/or second polypeptide; or
the third polypeptide is different from the first and/or second polypeptide.

81. The immunomodulatory protein of embodiment 79 or embodiment 80, wherein the immunomodulatory protein further comprises a multimerization domain linked to at least one of the variant CD86 polypeptide, the second polypeptide and/or the third polypeptide.

82. The immunomodulatory protein of any of embodiments 68-70, 78 and 81, wherein the multimerization domain is an Fc domain of an immunoglobulin, optionally wherein the immunoglobulin protein is human and/or the Fc region is human.

83. The immunomodulatory protein of embodiment 82, wherein the Fc domain is an IgG1, IgG2 or IgG4, or is a variant thereof with reduced effector function.

84. The immunomodulatory protein of embodiment 83, wherein the Fc domain is an IgG1 Fc domain, optionally a human IgG1, or is a variant thereof with reduced effector function.

85. The immunomodulatory protein of any of embodiments 82-84, wherein the Fc domain comprises the sequence of amino acids set forth in SEQ ID NO: 229 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 229.

86. The immunomodulatory protein of any of embodiments 82-85, wherein the Fc domain is or comprises the sequence of amino acids set forth in SEQ ID NO: 229.

87. The immunomodulatory protein of embodiment 84 or embodiment 85, wherein the Fc domain is a variant IgG1 comprising one or more amino acid substitutions and the one or more amino acid substitutions are selected from E233P, L234A, L234V, L235A, L235E, G236del, G237A, S267K, or N297G, each numbered according to EU index by Kabat.

88. The immunomodulatory protein of embodiment 87, wherein the Fc domain comprises the amino acid substitution N297G, the amino acid substitutions R292C/N297G/V302C, or the amino acid substitutions L234A/L235E/G237A, each numbered according to the EU index of Kabat.

89. The immunomodulatory protein of embodiment 87 or embodiment 88, wherein the variant Fc region further comprises the amino acid substitution C220S, wherein the residues are numbered according to the EU index of Kabat.

90. The immunomodulatory protein of any of embodiments 87-89, wherein the Fc region comprises K447del, wherein the residue is numbered according to the EU index of Kabat.

91. The immunomodulatory protein of any of embodiments 84, 85 and 87-90, wherein the Fc domain comprises the sequence of amino acids set forth in SEQ ID NO: 230 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 230 and comprises one or more of the respective amino acid modifications set forth in SEQ ID NO: 230 compared to human IgG1.

92. The immunomodulatory protein of any of embodiments 84, 85 and 87-91, wherein the Fc domain is comprises the sequence of amino acids set forth in SEQ ID NO: 230.

93. A conjugate, comprising a variant CD86 polypeptide of any of embodiments 1-65 linked to a targeting moiety that specifically binds to a molecule on the surface of a cell.

94. The conjugate of embodiment 93, wherein the cell is an immune cell or is a tumor cell.

95. The conjugate of embodiment 93 or embodiment 94, wherein the moiety is a protein, a peptide, nucleic acid, small molecule or nanoparticle.

96. The conjugate of any of embodiments 93-95, wherein the moiety is an antibody or antigen-binding fragment.

97. The conjugate of any of embodiments 93-96 that is a fusion protein.

98. A nucleic acid molecule(s) encoding a variant CD86 polypeptide of any of embodiments 1-65, an immunomodulatory protein of any of embodiments 66-92 or a conjugate that is a fusion protein of any of embodiments 93-97.

99. The nucleic acid molecule of embodiment 98 that is a synthetic nucleic acid.

100. The nucleic acid molecule of embodiment 98 or embodiment 99 that is a cDNA.

101. A vector, comprising the nucleic acid molecule of any of embodiments 98-100.

102. The vector of embodiment 101 that is an expression vector.

103. The vector of embodiment 101 or embodiment 102, wherein the vector is a mammalian expression vector or a viral vector.

104. A cell, comprising the vector of any of embodiments 101-103.

105. The cell of embodiment 104 that is a mammalian cell.

106. The cell of embodiment 104 or embodiment 105 that is a human cell.

107. A method of producing a protein comprising a variant CD86 polypeptide, comprising introducing the nucleic acid molecule of any of embodiments 98-100 or vector of any of embodiments 101-103 into a host cell under conditions to express the protein in the cell.

108. The method of embodiment 107, further comprising isolating or purifying the protein from the cell.

109. A method of engineering a cell expressing a variant CD86 polypeptide, the method comprising introducing a nucleic acid molecule encoding the variant CD86 polypeptide of any of embodiments 1-65, immunomodulatory protein of any of embodiments 66-92 or a conjugate that is a fusion protein of any of embodiments 93-97 into a host cell under conditions in which the polypeptide is expressed in the cell.

110. An engineered cell, comprising a variant CD86 polypeptide of any of embodiments 1-65, immunomodulatory protein of any of embodiments 66-92 or a conjugate that is a fusion protein of any of embodiments 93-97, a nucleic acid molecule of any of embodiments 98-100 or a vector of any of embodiments 101-103.

111. The engineered cell of embodiment 110, wherein:
the variant CD86 polypeptide comprises a transmembrane domain or is the transmembrane immunomodulatory protein of any of embodiments 59-65; and/or
the protein comprising the variant CD86 polypeptide is expressed on the surface of the cell.

112. The engineered cell of embodiment 110, wherein:
the variant CD86 polypeptide does not comprise a transmembrane domain and/or is not expressed on the surface of the cell; and/or
the variant CD86 polypeptide is capable of being secreted from the engineered cell.

113. The engineered cell of embodiment 110 or embodiment 112, wherein:
the protein does not comprise a cytoplasmic signaling domain or transmembrane domain and/or is not expressed on the surface of the cell; and/or
the protein is capable of being secreted from the engineered cell when expressed.

114. The engineered cell of any of embodiments 110-113, wherein the cell is an immune cell.

115. The engineered cell of embodiment 114, wherein the immune cell is a lymphocyte.

116. The engineered cell of embodiment 115, wherein the lymphocyte is a T cell.

117. The engineered cell of embodiment 116, wherein the T cell is a CD4+ and/or CD8+ T cell.

118. The engineered cell of embodiment 116 or embodiment 117, wherein the T cell is a regulatory T cell (Treg).

119. The engineered cell of any of embodiments 110-118 that is a primary cell.

120. The engineered cell of any of embodiments 110-119, wherein the cell is a mammalian cell.

121. The engineered cell of any of embodiments 110-120, wherein the cell is a human cell.

122. The engineered cell of any of embodiments 110-121, further comprising a chimeric antigen receptor (CAR).

123. The engineered cell of any of embodiments 110-121, further comprising an engineered T-cell receptor (TCR).

124. An infectious agent, comprising a variant CD86 polypeptide of any of embodiments 1-65, immunomodulatory protein of any of embodiments 66-92 or a conjugate that is a fusion protein of any of embodiments 93-97, a nucleic acid molecule of any of embodiments 98-100 or a vector of any of embodiments 101-103.

125. The infectious agent of embodiment 124, wherein the infectious agent is a bacterium or a virus.

126. The infectious agent of embodiment 125, wherein the infectious agent is a virus and the virus is an oncolytic virus.

127. A pharmaceutical composition, comprising a variant CD86 polypeptide of any of embodiments 1-65, immunomodulatory protein of any of embodiments 66-92 or a conjugate that is a fusion protein of any of embodiments 93-97, an engineered cell of any of embodiment 110-123 or an infectious agent of any of embodiments 124-126.

128. The pharmaceutical composition of embodiment 127, comprising a pharmaceutically acceptable excipient.

129. The pharmaceutical composition of embodiment 127 or embodiment 128, wherein the pharmaceutical composition is sterile.

130. An article of manufacture comprising the pharmaceutical composition of any of embodiments 127-129 in a vial or a container.

131. The article of manufacture of embodiment 130, wherein the vial or container is sealed.

132. A kit comprising the pharmaceutical composition of any of embodiments 127-129 or the article of manufacture of embodiment 131 or embodiment 132 and instructions for use.

133. A method of modulating an immune response in a subject, the method comprising administering a variant CD86 polypeptide of any of embodiments 1-65, immunomodulatory protein of any of embodiments 66-92 or a conjugate that is a fusion protein of any of embodiments 93-97, an engineered cell of any of embodiment 110-123, an infectious agent of any of embodiments 124-126, or the pharmaceutical composition of any of embodiments 127-129.

134. A method of modulating an immune response in a subject, comprising administering the engineered cells of any of embodiments 110-123.

135. The method of embodiment 134, wherein the engineered cells are autologous to the subject.

136. The method of embodiment 134, wherein the engineered cells are allogenic to the subject.

137. The method of any of embodiments 133-136, wherein modulating the immune response treats a disease or condition in the subject.

138. A method of treating a disease or condition in a subject in need thereof, the method comprising administering a variant CD86 polypeptide of any of embodiments 1-65, immunomodulatory protein of any of embodiments 66-92 or a conjugate that is a fusion protein of any of embodiments 93-97, an engineered cell of any of embodiment 110-123, an infectious agent of any of embodiments 124-126, or the pharmaceutical composition of any of embodiments 127-129.

139. A method of treating a disease or condition in a subject in need thereof, comprising administering the engineered cells of any of embodiments 110-123.

140. The method of embodiment 139, wherein the engineered cells are autologous to the subject.

141. The method of embodiment 139, wherein the engineered cells are allogenic to the subject.

142. The method of any of embodiments 133-141, wherein the immune response is increased in the subject.

143. The method of any of embodiments 133, 137, 138 and 142, wherein an immunomodulatory protein or conjugate comprising a variant CD86 polypeptide linked to a tumor-localizing moiety is administered to the subject.

144. The method of embodiment 143, wherein the tumor-localizing moiety is or comprises a binding molecule that recognizes a tumor antigen.

145. The method of embodiment 144, wherein the binding molecule comprises an antibody or an antigen-binding fragment thereof or comprises a wild-type IgSF domain or variant thereof.

146. The method of any of embodiments 133 and 137-145, wherein a pharmaceutical composition comprising the immunomodulatory protein of any of embodiments 71-90 or the conjugate of any of embodiments 93-97 is administered to the subject.

147. The method of any of embodiments 133-142, wherein an engineered cell comprising a variant CD86 polypeptide that is a transmembrane immunomodulatory protein is administered to the subject, optionally, wherein the engineered cell is of embodiment 110, 111 and 114-123.

148. The method of any of embodiment 147, wherein the transmembrane immunomodulatory protein is of any of embodiments 59-65.

149. The method of any of embodiments 137-148, wherein the disease or condition is a tumor or cancer.

150. The method of any one of embodiments 137-149, wherein the disease or condition is selected from melanoma, lung cancer, bladder cancer, a hematological malignancy, liver cancer, brain cancer, renal cancer, breast cancer, pancreatic cancer, colorectal cancer, spleen cancer, prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer.

151. The method of any of embodiments 133-141, wherein the immune response is decreased.

152. The method of any of embodiments 133, 137, 138 and 151, wherein a variant CD86 polypeptide or immunomodulatory protein that is soluble is administered to the subject.

153. The method of embodiment 152, wherein the soluble polypeptide or immunomodulatory protein is an Fc fusion protein.

154. The method of any of embodiments 133, 137, 138 and 151-153, wherein a pharmaceutical composition comprising a variant CD86 polypeptide of any of embodiments 1-58, or the immunomodulatory protein of any of embodiments 66-74 and 78-91 is administered to the subject.

155. The method of any of embodiments 133, 137, 138 and 151, wherein an engineered cell comprising a secretable variant CD86 polypeptide is administered to the subject, optionally wherein the engineered cell is of any of embodiments 110 and 112-123.

156. The method of any of embodiments any of embodiments 133, 137, 138 and 151-155, wherein the disease or condition is an inflammatory or autoimmune disease or condition.

157. The method of any of embodiments 133, 137, 138 and 151-155, wherein the disease or condition is an Antineutrophil cytoplasmic antibodies (ANCA)-associated vasculitis, a vasculitis, an autoimmune skin disease, transplantation, a Rheumatic disease, an inflammatory gastrointestinal disease, an inflammatory eye disease, an inflammatory neurological disease, an inflammatory pulmonary disease, an inflammatory endocrine disease, or an autoimmune hematological disease.

158. The method of embodiment 156 or embodiment 157, wherein the disease or condition is selected from inflammatory bowel disease, transplant, Crohn's disease, ulcerative colitis, multiple sclerosis, asthma, rheumatoid arthritis, or psoriasis.

### IX. EXAMPLES

The following examples are included for illustrative purposes only and are not intended to limit the scope of the invention.

### Example 1

### Generation of Mutant DNA Constructs of CD86 IgSF Domains

Example 1 describes the generation of mutant DNA constructs of human CD86 IgSF domains for translation and expression on the surface of yeast as yeast display libraries, introduction of DNA libraries into yeast, and selection of yeast cells expressing affinity-modified variants of CD86 ECD.

Constructs were generated based on a wildtype human CD86 sequence set forth in SEQ ID NO: 29 containing the extracellular domain (ECD; corresponding to residues 24-247 as set forth in UniProt Accession No. P42081), designated “CD86 ECD (24-247)” as follows:

CD86 ECD (24-247) (SEQ ID NO: 29):
APLKIQAYFNETADLPCQFANSQNQSLSELVVFWQDQENLVLNEVYLGKE
KFDSVHSKYMGRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGVMQKSQDNVTELYDVSISLSVSFPDVTSNMTIFCILETD
KTRLLSSPFSIELEDPQPPPDHIP

Random DNA libraries were constructed to identify variants of the ECD of CD86 set forth in SEQ ID NO: 29. DNA encoding the wild-type ECD domain was cloned between the BamHI and KpnI sites of the modified yeast expression vector PBYDS03 (Life Technologies USA) which places the CD86 ECD N-terminal to the yeast surface anchoring domain Sag1 (the C-terminal domain of yeast α-agglutinin) with an in-frame HA fusion tag N-terminal to the CD86 ECD sequence and a c-Myc fusion tag C-terminal to the CD86 ECD sequence. Expression in this vector is driven off of the inducible gal-1 promoter. After verification of the correct DNA sequence, and proper display of the wild-type CD86 ECD protein on the yeast surface, the wild type DNA construct was used as template for error-prone PCR to introduce random mutations across the CD86 ECD sequence. After error-prone PCR, the mutagenized CD86 ECD DNA was gel purified and then PCR amplified using primers containing 40 bp overlap regions homologous to the upstream sequence of BamHI and the downstream sequence of KpnI in pBYDS03 for preparation of large scale yeast electroporation. The gel-purified, mutated CD86 ECD DNA insert was resuspended in sterile, deionized water.

The mutated CD86 library DNA was inserted into electroporation-competent BJ5464 yeast cells (ATCC) along with BamHI and KpnI digested pBYDS03 vector DNA by electroporation using a BTX ECM399 electroporation system at 2500V. Library size was determined by plating serial dilutions of freshly recovered cells on SCD-Leu agar plates. The remainder of the electroporated culture was grown to saturation under selection in SCD-Leu selection medium. Cells from this culture were subcultured 1/100 into the same medium once more and grown to saturation to minimize the fraction of untransformed cells and to allow for segregation of plasmid from cells that may contain two or more library variants. To maintain library diversity, this subculturing step was carried out using an inoculum that contained at least 10× more cells than the calculated library size. Cells from the second saturated culture were resuspended in fresh medium and frozen and stored at −80° C. (frozen library stock).

Cells from the library were thawed from individual library stocks and grown overnight. The next day cells were resuspending in galactose containing induction media (SCDG-Leu media) and grown overnight at 30° C. to induce expression of library proteins on the yeast cell surface. One liter of SCDG-Leu induction media contained 5.4 grams Na₂HPO₄, 8.56 grams NaH₂PO₄·H₂0, 20 grams galactose, 2.0 grams dextrose, 6.7 grams yeast nitrogen base, and 1.6 grams yeast synthetic drop out media supplement without leucine dissolved in water and sterilized through a 0.22 μm membrane filter device.

10× induced library cells were sorted once using Protein A magnetic beads (New England Biolabs, USA) loaded with CD28-Fc to reduce non-binders and enrich for all CD86 ECD variants with the ability to bind their exogenous recombinant counter-structure proteins. This was then followed by three rounds of positive CD28 selection by protein staining with decreasing concentrations of CD28-Fc (20 nM, 1 nM or 250 pM) and fluorescence activated cell sorting (FACS) to enrich the fraction of yeast cells that displayed improved binders. Magnetic bead enrichment and selections by flow cytometry were carried out essentially as described in Miller K. D. et al., Current Protocols in Cytometry 4.7.1-4.7.30, July 2008. Hits were chosen from the third round of positive selection yeast cell outputs described above.

A second cycle of random mutagenesis was carried out from yeast cell outputs from the third round of CD28 positive selected cells. Further hits were chosen following three rounds of FACs positive selection using decreasing concentrations of CD28-Fc as described above. From yeast cell outputs from the third round of CD28-Fc positive selection, a further negative FACS selection of CTLA-4 was preformed after protein staining with 100 nM CTLA-4 Fc.

Inserts from selected FACS outputs were subcloned into an Fc fusion vector for sequence analysis of individual clones. Hits were chosen for protein production and binding and functional activity screening based on the frequency of variant amino acids that were enriched over the selection process as described in Example 2.

### Example 2

### Reformatting Selection Outputs as Fc-Fusions and in Various Immunomodulatory Protein Formats

Exemplary Hits chosen in Example 1 were reformatted as immunomodulatory proteins containing an affinity modified (variant) CD86 fused to an Fc molecule.

Output cell pools from selected CD86 FACS sorts were grown to terminal density in SCD-Leu selection medium and plasmid DNA was isolated using a yeast plasmid DNA isolation kit (Zymoresearch, USA). For generation of Fc fusions, the affinity matured CD86 ECD variants were PCR amplified with primers containing 40 bp homologous regions on either end with an AfeI and BamHI digested Fc fusion vector encoding and in-frame with the Fc region to carry out in vitro recombination using Gibson Assembly Master Mix (New England Biolabs). The Gibson Assembly reaction was added to the E. coli strain NEB5alpha (New England Biolabs, USA) for heat shock transformation following the manufacturer's instructions.

Dilutions of transformation reactions were plated onto LB-agar containing 100 g/mL carbenicillin (Teknova, USA) to isolate single colonies for selection. Generally, up to 96 colonies from each transformation were then grown in 96 well plates to saturation overnight at 37° C. in LB-broth containing 100 g/mL carbenicillin (Teknova cat #L8112) and a small aliquot from each well was submitted for DNA sequencing to identify mutation(s) in all clones.

After sequence analysis and identification of clones of interest, plasmid DNA was prepared using the MidiPlus kit (Qiagen).

The DNA encoded generated affinity-modified (variant) CD86 Fc fusion proteins as follows: variant CD86 domain followed by a linker of 7 amino acids (GSGGGGS) followed by a human IgG1 effectorless Fc sequence set forth in SEQ ID NO: 230 containing the mutations L234A, L235E and G237A, by the Eu Index numbering system for immunoglobulin proteins. Since the construct does not include any antibody light chains that can form a covalent bond with a cysteine, the human IgG1 Fc also contained replacement of the cysteine residues to a serine residue at position 220 (C220S) by Eu Index numbering system for immunoglobulin proteins (corresponding to position 5 (C5S) with reference to the wild-type or unmodified Fc set forth in SEQ ID NO: 299). The Fc region also lacked the C-terminal lysine at position 447 (designated K447del) normally encoded in the wild type human IgG1 constant region gene (corresponding to position 232 of the wild-type or unmodified Fc) set forth in SEQ ID NO: 229.

SEQ ID NO: 230
EPKSSDKTHTCPPCPAPEAEGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

Recombinant variant Fc fusion proteins were produced from suspension-adapted human embryonic kidney (HEK) 293 cells using the Expi293 expression system (Invitrogen, USA).Supernatant was harvested and the Fc Protein was captured on Mab SelectSure. (GE Healthcare cat. no. 17543801) Protein was eluted from the column using 50 mM Acetate pH3.6. The MabSelect Sure eluate is pooled and the pH is adjusted to above pH5.0. This material was then polished on a Preparative SEC column, to generate highly purified monomeric material. This material is buffer exchanged into 10 mM Acetate, 9% Sucrose pH 5.0. (A5Su) The protein purity is assessed by analytic SEC. Material is vialed and stored at −80.

### Example 3

### Assessment of Binding of Affinity-Matured IgSF Domain-Containing Molecules to Cell-Expressed Counter Structure

This Example describes Fc-fusion binding studies of purified proteins from the above Examples to assess specificity and affinity of CD86 domain variant immunomodulatory proteins for a cognate binding partner.

Binding of the CD86 domain variants described above were assayed for binding to CD28 using Jurkat cells or to CTLA-4 using Chinese Hamster Ovary (CHO) cells that were transduced to stably express CTLA-4 (CHO/CTLA-4). For staining by flow cytometry, approximately 100,000 ligand-expressing cells were incubated with various concentrations of each candidate CD86 variant Fc fusion protein. Controls included an extracellular domain (ECD) of wild-type CD86 (“Wt CD86-Fc”) and an Fc only control. To assess binding, cells were stained with an anti-human Fc secondary antibody (Jackson ImmunoResearch, USA), and samples were analyzed on an LSRII (BD Biosciences, Inc., USA) flow cytometer.

Mean Fluorescence Intensity (MFI) was calculated and compared to binding of wildtype CD86 ECD-Fc control with FlowJo Version 10 (FlowJo Version 10, USA). Results for the binding studies for binding of 11 nM of exemplary tested variant CD86 ECD-Fc fusion molecules for CD28- or CTLA-4-expressing cells are shown in Table E1. The Table also indicates amino acid substitutions in the ECD of the variant CD86 selected in the screening described above. In the Table, the exemplary amino acid substitutions and insertions in the ECD domain are designated by amino acid position number corresponding to amino acid positions in the respective reference unmodified mature CD86 extracellular domain (ECD) sequence set forth in SEQ ID NO:29. The amino acid position is indicated in the middle, with the corresponding unmodified (e.g. wild-type) amino acid listed before the number and the identified variant amino acid substitution listed after the number. Column 2 sets forth the SEQ ID NO identifier for each variant ECD domain contained in the variant ECD-Fc fusion molecule.

As shown in Table E1, the selections resulted in the identification of a number of CD86 IgSF (e.g. ECD) domain variants that were affinity-modified to exhibit increased binding for CD28. The selected variants, in some cases, exhibited altered (e.g. decreased) binding to CTLA4.

TABLE E1
Binding of CD86 ECD Variants to Cognate Binding Partners

| | | | | Binding to | |
|---|---|---|---|---|---|
| | | | | CHO/CTLA4 | |
| | | | | Transfectants (11 nM) | |
| | | Binding to CD28 on | | | Fold |
| | | Jurkat Cells(11 nM) | | | Increase |
| | | | Fold Increase | | over WT |
| | SEQ ID NO | | over WT | | CD86 |
| Mutations | (ECD) | MFI | CD86 ECD | MFI | ECD |
| Q25L, T71A, H90Y | 85 | 4062 | 286.1 | 20435 | 1.0 |
| Q25L, D53G, E212V | 86 | 166 | 11.7 | 23346 | 1.2 |
| Q25L, H90L | 87 | 3464 | 243.9 | 16961 | 0.8 |
| N43K, I79N, H90L, I178T, | 88 | 11.6 | 0.8 | 13204 | 0.7 |
| E198D | | | | | |
| A13V, Q25L, H90L, S181P, | 89 | 2235 | 157.4 | 18271 | 0.9 |
| L197M, S206T | | | | | |
| Q25L, Q86R, H90L, K93T, | 90 | 1639 | 115.4 | 21502 | 1.1 |
| L132M, V148D, S181P, | | | | | |
| P216H | | | | | |
| Q25L, F33I, H90Y, V128A, | 91 | 1445 | 101.8 | 17459 | 0.9 |
| P141A, E158G, S181P | | | | | |
| Q25L, N39D, K80R, Q86R, | 92 | 1928 | 135.8 | 19889 | 1.0 |
| I88F, H90L, K93T, N123D, | | | | | |
| N154D | | | | | |
| Q25L, H90L, K93T, M97L, | 93 | 373 | 26.3 | 17275 | 0.9 |
| T133A, S181P, D215V | | | | | |
| Q25L, Q86R, H90L, N104S | 94 | 3834 | 270.0 | 19636 | 1.0 |
| Q25L, L40M, H90L, L180S, | 95 | 2482 | 174.8 | 19802 | 1.0 |
| S183P | | | | | |
| Q18K, Q25L, F33I, L40S, | 96 | 3781 | 266.3 | 18971 | 0.9 |
| H90L | | | | | |
| Q25L, Q86K, H90L, I137T, | 97 | 387 | 27.3 | 19575 | 1.0 |
| S181P | | | | | |
| Q25L, L77P, H90Y, K153R, | 98 | 15.7 | 1.1 | 18797 | 0.9 |
| V170D, S181P | | | | | |
| Q25L, S28G, F33I, F52L, | 99 | 749 | 52.7 | 21177 | 1.0 |
| H90L, Q102H, I178T | | | | | |
| Q25L, F33I, H90L, K144E, | 100 | 1636 | 115.2 | 23546 | 1.2 |
| L180S | | | | | |
| Q25L, F33I, H90L, K153E, | 101 | 13.2 | 0.9 | 8657 | 0.4 |
| E172G, T192N | | | | | |
| Q25L, F33I, Q86R, H90Y, | 102 | 528 | 37.2 | 22641 | 1.1 |
| D175E, 1196V, E198D | | | | | |
| Q25L, V45I, D68N, H90L, | 103 | 466 | 32.8 | 17446 | 0.9 |
| S183P, L205S | | | | | |
| E38V, S114G, P143H | 104 | No Protein | | | |
| H90Y, L180S | 105 | 11.4 | 0.8 | 6078 | 0.3 |
| H90Y, Y129N | 106 | 154 | 10.8 | 17640 | 0.9 |
| I89V, H90L, I193V | 107 | 1248 | 87.9 | 22070 | 1.1 |
| K80E, H90Y, H222T, I223F, | 108 | No Protein | | | |
| P224L | | | | | |
| K80M, I88T | 109 | 1966 | 138.5 | 18532 | 0.9 |
| K92I, F113S | 110 | 369 | 26.0 | 20421 | 1.0 |
| M60K, H90L | 111 | 1049 | 73.9 | 17319 | 0.9 |
| Q25L, F33I, H90L | 112 | 115 | 8.1 | 20888 | 1.0 |
| Q25L, F33I, Q86R, H90L, | 113 | 1316 | 92.7 | 14705 | 0.7 |
| K93T | | | | | |
| Q25L, H90L | 114 | 1810 | 127.5 | 21873 | 1.1 |
| Q25L, H90L, P185S | 115 | 135 | 9.5 | 18546 | 0.9 |
| Q25L, H90L, P185S, P224L | 116 | 11.9 | 0.8 | 9355 | 0.5 |
| Q25L, H90L, S179R | 117 | 2397 | 168.8 | 23282 | 1.1 |
| Q25L, H90Y, S181P, I193V | 118 | 256 | 18.0 | 16648 | 0.8 |
| Q25L, K82T, H90L, T152S, | 119 | 1027 | 72.3 | 18161 | 0.9 |
| S207P | | | | | |
| Q25L, Q86R, H90L, K93T | 120 | 1500 | 105.6 | 19777 | 1.0 |
| S28G, H90Y | 121 | No Protein | | | |
| CD86 WT ECD-Fc | 29 | 14.2 | 1.0 | 20294 | 1.0 |
| Fe only control | 230 | 11.4 | 0.8 | 32.9 | 0.0 |

### Example 4

### Generation and Binding Activity of CD86 IgV-Fc Immunomodulatory Proteins

Exemplary CD86 ECD variants identified and generated in Examples 1-3 were converted to an immunomodulatory protein containing the IgV domain as the only IgSF domain of the molecule. The generated variant IgV constructs were based on a wildtype human CD86 sequence set forth in SEQ ID NO: 123 containing the IgV domain (corresponding to residues 24-134 as set forth in UniProt Accession No. P42081), designated “CD86 IgV (24-134)” as follows:

CD86 IgV (24-134) (SEQ ID NO: 123):
APLKIQAYFNETADLPCQFANSQNQSLSELVVFWQDQENLVLNEVYLGK
EKFDSVHSKYMGRTSFDSDSWTLRLHNLQIKDKGLYQCIIHHKKPTGMI
RIHQMNSELSVLA

The variant CD86 IgV molecules were formatted as Fc fusion proteins as described in Example 2, and tested for binding to CD28 or CTLA-4 as described in Example 3. Results for the binding studies for binding of 25 nM of exemplary tested variant CD86 IgV-Fc fusion molecules for CD28- or CTLA-4-expressing cells are shown in Table E2. In the Table, the exemplary amino acid substitutions and insertions in the IgV domain are designated by amino acid position number corresponding to amino acid positions in the respective reference unmodified mature CD86 extracellular domain (ECD) sequence set forth in SEQ ID NO:29. The amino acid position is indicated in the middle, with the corresponding unmodified (e.g. wild-type) amino acid listed before the number and the identified variant amino acid substitution listed after the number. Column 2 sets forth the SEQ ID NO identifier for each variant IgV domain contained in the variant IgV-Fc fusion molecule.

TABLE E2
Binding of CD86 IgV Variants to Cognate Binding Partners

| | | Binding to CD28 on | | | |
|---|---|---|---|---|---|
| | | Jurkat Cells (25 nM) | | | |
| | | | Fold | Binding to CHO/CTLA4 | |
| | | | Increase | Transfectants (25 nM) | |
| | | | over WT | | Fold Increase |
| | SEQ ID NO | | CD86 | | over WT CD86 |
| Mutations | (IgV) | MFI | ECD | MFI | ECD |
| A13V, Q25L, H90L | 124 | 1370 | 111.4 | 3980 | 1.0 |
| Q25L, H90L, K93T, M97L | 125 | 1425 | 115.9 | 4239 | 1.1 |
| Q25L, Q86R, H90L | 126 | 1569 | 127.6 | 4940 | 1.3 |
| I89V, H90L | 127 | 213 | 17.3 | 1660 | 0.4 |
| M60K, H90L | 128 | 20 | 1.6 | 134 | 0.0 |
| Q25L, F33I, H90L | 129 | 1383 | 112.4 | 5901 | 1.5 |
| Q25L, H90L | 130 | 1194 | 97.1 | 3587 | 0.9 |
| WT CD86 IgV-Fc | 123 | 12.3 | 1.0 | 87 | 0.0 |
| WT CD86 ECD-Fc | 29 | 12.3 | 1.0 | 3951 | 1.0 |
| Fc control | 230 | 10.5 | 0.9 | 35.9 | 0.0 |

### Example 5

### Assessment of Bioactivity of Affinity-Matured CD86 IgSF Domain-Containing Molecules Using a Jurkat/IL2 Reporter Assay

This Example describes a Jurkat/IL2 reporter assay to assess bioactivity of CD86 domain variant immunomodulatory proteins for CD28 costimulation.

The day before the assay, the assay plate was prepared. To prepare the assay plate, 10 nM anti-CD3 antibody (clone OKT3; BioLegend, catalog no. 317315) was combined with a titration of CD86-Fc variants (concentrations ranging from 200 nM to 12 pM) or control proteins (WT CD86-Fc and Fc control) in PBS. 100 L/well of OKT3+CD86-Fc was aliquoted into a white, flat-bottom 96-well plate (Costar). The plate was incubated overnight at 4° C. to allow the antibody and CD86-Fc variant protein to adhere to the surface of the plate. The next day, the wells of the assay plate were washed twice with 150 μL PBS prior to the assay.

The day of the assay, Jurkat effector cells expressing IL-2-luciferase reporter were counted and resuspended in assay buffer to a concentration of 1×10⁶ cells/mL. 100 μL/well of the Jurkat cell suspension were then added assay plate.

The assay plate was briefly spun down (10 seconds at 1200 RPM) and incubated at 37° C. for 5 hours. After the 5 hour incubation, the plate was removed and equilibrated to room temperature for 15 minutes. 100 L of Bio-Glo (Promega) were added/well of the assay plate, which was then placed on an orbital shaker for 10 minutes. Luminescence was measured with a 1 second per well integration time using a BioTek Cytation 3 luminometer.

An average relative luminescence value was determined for each variant CD86 ECD-Fc and variant CD86 IgV-Fc and a fold increase in IL-2 reporter signal was calculated for each variant compared to wildtype CD86 ECD-Fc protein. The results for the 50 nM concentrations are provided in Table E3 (CD86 ECD-Fc) or Table E4 (CD86 IgV-Fc) below. As shown, co-culturing exemplary variant CD86 ECD-Fc or variant CD86 IgV-Fc molecules with Jurkat effector cells expressing IL-2-luciferase reporter, resulted in increased CD28 costimulation (i.e., agonism) compared to WT CD86 ECD-Fc or the Fc-only negative control.

TABLE E3
Luciferase Activity (CD86 ECD Variants)

| | | Jurkat/IL2 Luciferase Assay | |
|---|---|---|---|
| | | (10 nM OKT3 + | |
| | | 50 nM CD86 ECD-Fc) | |
| | SEQ | Relative | Fold Increase |
| | ID NO | luminescence | over |
| Mutations | (ECD) | units (RLU) | WT CD86 ECD |
| A13V, Q25L, H90L, S181P, | 89 | 1125 | 8.5 |
| L197M, S206T | | | |
| Q25L, H90L, K93T, M97L, | 93 | 997 | 7.6 |
| T133A, S181P, D215V | | | |
| Q25L, Q86R, H90L, N104S | 94 | 717 | 5.4 |
| I89V, H90L, I193V | 107 | 218 | 1.7 |
| M60K, H90L | 111 | 202 | 1.5 |
| Q25L, F33I, H90L | 112 | 428 | 3.2 |
| Q25L, H90L, P185S | 113 | 416 | 3.2 |
| WT CD86 ECD-Fc | 29 | 132 | 1.0 |
| Fc control | 230 | 112 | 0.8 |

TABLE E4
Luciferase Activity (CD86 IgV Variants)

| | | Jurkat/IL2 Luciferase Assay | |
|---|---|---|---|
| | | (10 nM OKT3 + 50 nM CD86 IgV-Fc) | |
| | | Relative luminescence | Fold Increase over |
| Mutations | SEQ ID NO (IgV) | units (RLU) | WT CD86 ECD |
| A13V, Q25L, H90L | 124 | 719 | 5.4 |
| Q25L, H90L, K93T, M97L | 125 | 406 | 3.1 |
| Q25L, Q86R, H90L | 126 | 800 | 6.1 |
| I89V, H90L | 127 | 562 | 4.3 |
| M60K, H90L | 128 | 818 | 6.2 |
| Q25L, F33I, H90L | 129 | 1137 | 8.6 |
| Q25L, H90L | 130 | 691 | 5.2 |
| WT CD86 ECD-Fc | 29 | 132 | 1.0 |
| Fc control | 230 | 112 | 0.8 |

### Example 6

### Generation and Assessment of Engineered Cells Expressing a Transmembrane Immunomodulatory Protein and a T Cell Receptor or Chimeric Antigen Receptor (CAR)

This Example describes the expression of variant CD86 IgSF domain-containing transmembrane immunomodulatory proteins (TIPs) with an exemplary recombinant HPV16 E6-specific T cell receptor (TCR) or anti-HER2 CAR in human T cells. The CD86-TIPs had an affinity-modified ECD domain and also included a wild-type CD86 transmembrane and cytoplasmic domain corresponding to residues 248-268 and 269-329 of SEQ ID NO: 2, respectively. Exemplary TIPs included a CD86 TIP containing amino acid mutations corresponding to either Q25L/Q86R/H90L/N104S (SEQ ID NO:94), I89V/H90L/I193V (SEQ ID NO:107) or Q25L/H90L/K93T/M97L/T133A/S181P/D215V (SEQ ID NO:93), wherein numbering of mutations is with reference to positions in the CD86 extracellular domain set forth in SEQ ID NO: 29.

The nucleic acid molecules encoding the TIPs were individually cloned into a lentiviral vector, which was used to transduced T cells. Briefly, lentivirus particles containing the nucleic acid sequences were produced after co-transfection of HEK293 cells with the vectors and lentivirus packaging constructs. The lentivirus particles were collected with the culture medium. Human pan T cells were isolated from peripheral blood mononuclear cells (PBMC) of normal blood donors using EasySep™ Human T Cell Isolation Kit. The pan T cells were cultured with anti-CD3 and anti-CD28 magnetic beads and IL-2 for 6 hours, and then were co-transduced with the TIP lentivirus and with lentivirus for expression of either an exemplary TCR recognizing the MHC class I presented HPV16 E6 peptide (E6 TCR) or an anti-HER2 CAR. For co-transduction, the lentiviruses were transduced at a 1:1 ratio in the presence of polybrene. Transduction was performed by using spinfection at 1000×G for 30 minutes at 30° C.

Lentivirus was removed and replaced with fresh IL-2 containing media the next day. Culture media were changed with fresh IL-2 every 2 days and transduced T cells were expanded for 10 days. After 10 days of culture, engineered cells were accessed for activity as described below. As a control, activity was compared to control T cells transduced with a WT CD86 TIP (SEQ ID NO:2), a CAR or TCR only, or to mock transduced T cells.

### A. CD86 TIPs TCR Engineered T Cells

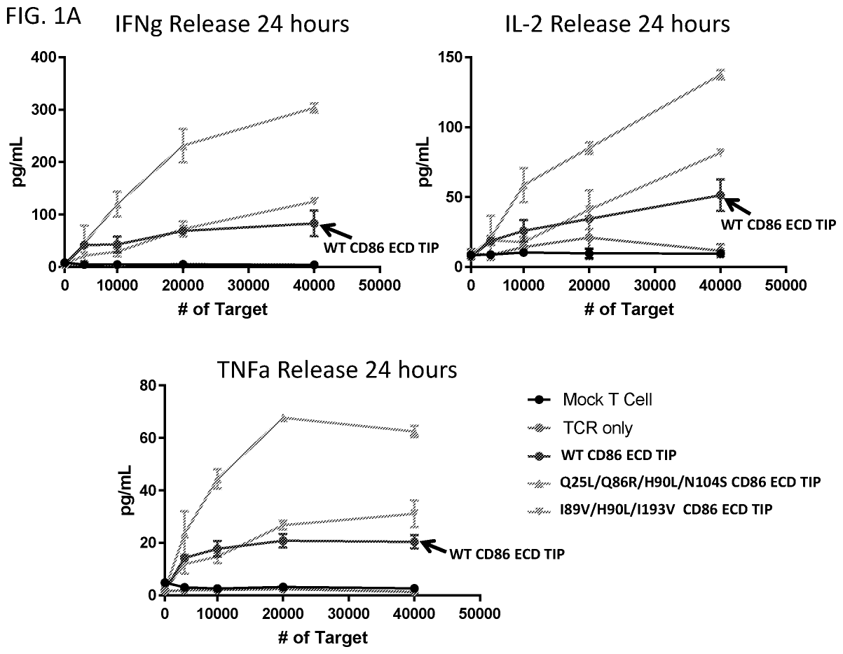

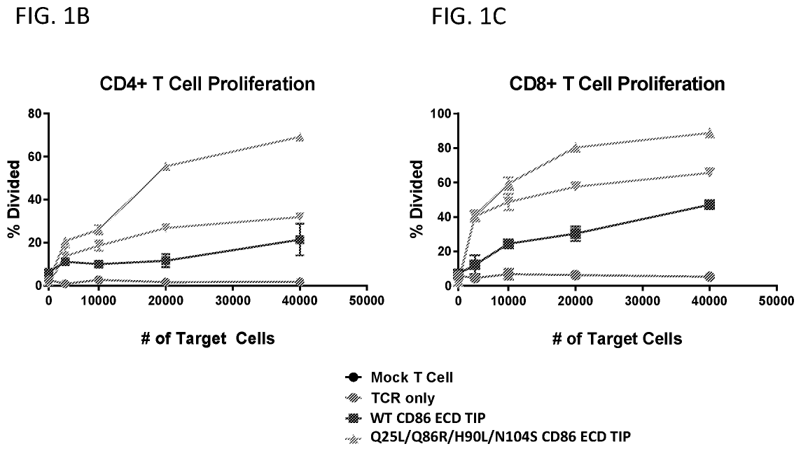

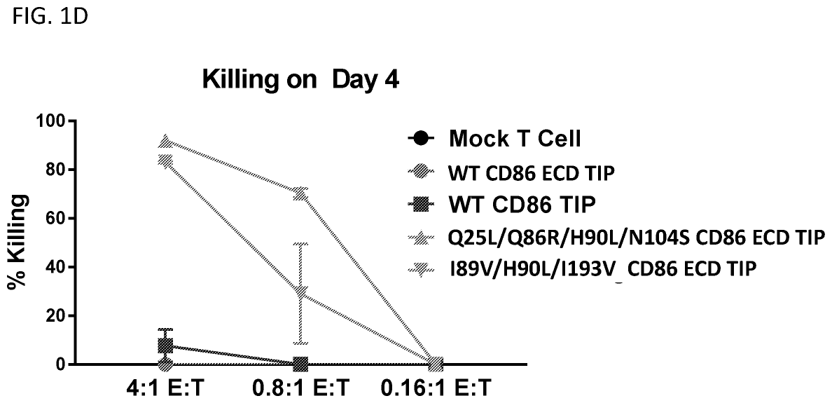

After 10 days of culture, T cells co-transduced with a CD86 TIP and E6 TCR, or control TCR only, TIP only or mock transduced T cells, were analyzed for cytokine production following co-culture with target antigen-expressing cells using MAGPIX® Luminex, which measures the amount of cytokine in the culture media. HLA-A2+HPV+ target cells line (SCC152) were seeded into a 96-well plate starting at 40,000 cells per well, titrated at 1:2 dilution for 4-point titrations. Target cells were cultured for 6 hours to form a mono cell layer. The transduced or control T cells were added to the wells at 40,000 cells per well and cultured for another 24 hours. Culture supernatant were collected and analyzed by MAGPIX®. The results were displayed and recorded as the raw value of cytokine production in pg/mL. FIG. 1A shows release of Interferon-gamma, IL-2, and TNFα from engineered cells that co-expressed variant CD86 TIPS, plotted as pg/mL versus target cell number. Error bars represent standard deviation from triplicate wells. Increased cytokine production was observed from E6 TCR-expressing cells that co-expressed a CD86 TIP. Greater cytokine production was observed in cells co-transduced with the exemplary variant CD86 TIP compared to the WT CD86 TIP. Table E5 shows exemplary raw values of IFNg detected in supernatant following 24 hour incubation of engineered cells expressing TCR alone or in combination with wild-type or variant CD86 TIPs as indicated.

TABLE E5
IFNg Cytokine Production (pg/mL)

| | SEQ ID NO | Target Cell # | | | | |
|---|---|---|---|---|---|---|
| Mutations | (CD86 ECD) | 0 | 5000 | 10000 | 20000 | 40000 |
| TCR only | | 1.2 | 1.8 | 2.0 | 3.5 | 24.5 |
| WT CD86 and TCR | 29 | 1.2 | 1.4 | 2.9 | 7.5 | 44.7 |
| A13V/Q25L/H90L/S181P/L197M, | 89 | 1.2 | 2.3 | 3.1 | 11.1 | 56.2 |
| S206T (CD86 ECD) and TCR | | | | | | |
| Q25L/90L/93T/97L/133A/S181P/ | 93 | 1.2 | 1.6 | 2.9 | 17.3 | 93.0 |
| 215V (CD86 ECD) and TCR | | | | | | |
| Q25L/Q86R/H90L/N104S | 94 | 1.2 | 2.6 | 5.7 | 49.5 | 162.2 |
| (CD86 ECD) and TCR | | | | | | |
| I89V/H90L/I193V (CD86 ECD) | 107 | 1.3 | 2.0 | 2.7 | 10.6 | 82.2 |
| and TCR | | | | | | |
| M60K/H90L (CD86 ECD) and | 111 | 1.4 | 2.1 | 3.8 | 7.9 | 31.3 |
| TCR | | | | | | |
| Q25L/F33I/H90L (CD86 ECD) | 112 | 1.2 | 1.5 | 2.4 | 10.4 | 36.3 |
| and TCR | | | | | | |
| Q25L/H90L/P185S (CD86 ECD) | 115 | 1.1 | 1.7 | 3.4 | 11.5 | 74.6 |
| and TCR | | | | | | |
| Q25L/H90L/S179R (CD86 ECD) | 117 | 1.3 | 1.4 | 2.0 | 10.2 | 102.4 |
| and TCR | | | | | | |

Transduced cells were also Cell Trace Violet (CTV) labeled and incubated with titrated amount of SCC152 target cells and proliferation was evaluated after 3 days on TCR+CD4+ (FIG. 1B) or TCR+CD8+ (FIG. 1C) T cells. % Divided are plotted+/−standard deviation of triplicate wells. As shown in FIGS. 1B-1C, engineered cells that co-expressed variant CD86 TIPS had increased proliferation in response to the target SCC-152 cell line compared to control or cells expressing the wildtype CD86 TIP.

To assess cytotoxic activity of the engineered T cells, the HLA-A2+HPV+ tumor target cell line SCC-152 was stably transduced with a virus encoding a constitutively active expression vector, which drives expression of firefly luciferase. Transduced T cells or control T cells were seeded at varying numbers to give a range of effector to target ratios (E:T ratios). T cells were incubated with target cells for 4 days at 37° C. and luciferase activity was measured. The percent killing was calculated by the formula: % Killing=(1−(luciferase signal in T cells+targets/luciferase signal from target cells only))*100%, and was plotted versus the E:T ratio. As shown in FIG. 1D, killing was observed with engineered cells that co-expressed a CD86 TIP, with little killing detected in TCR only expressing T cells. Compared to co-expression of WT CD86 TIP, co-expression of variant CD86 TIPs resulted in substantially enhanced killing activity by E6 TCR-expressing T cells. Data is shown with +/−standard deviation of triplicate wells.

### B. CD86 TIP CAR Engineered T Cells

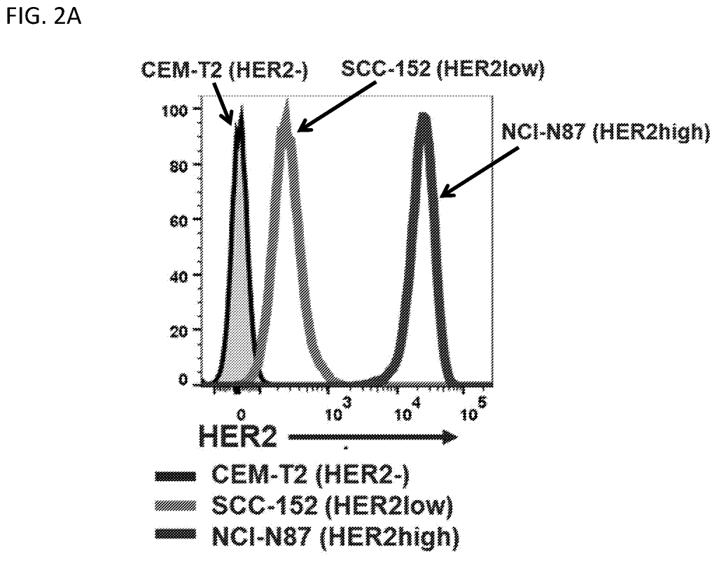

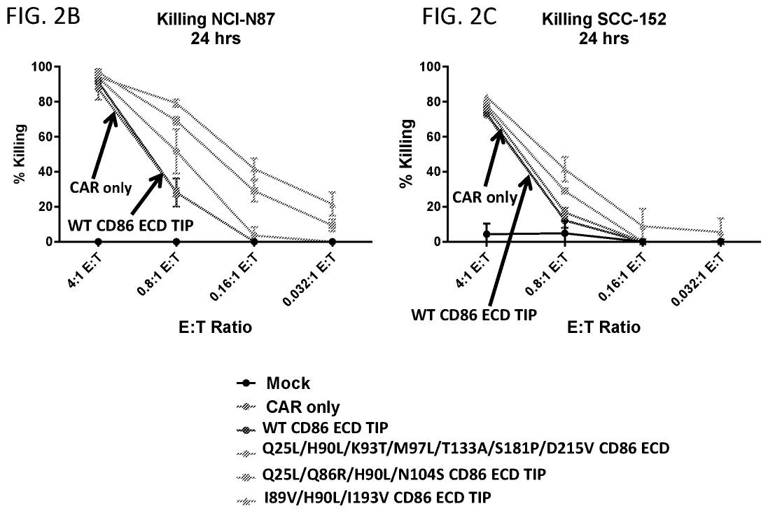

To assess cytotoxic activity, the engineered cells were incubated with target cells expressing high (NCI-N87) or low (SCC-152) levels of HER2 antigen (FIG. 2A). The target cells were stably transduced with a virus encoding a constitutively active expression vector, which drives expression of firefly luciferase, and cytotoxic activity was assessed similar to above. Anti-HER2 CAR transduced T cells were seeded at varying numbers to give a range of effector to target ratios (E:T ratios). T cells were incubated with target cells NCI-N87 or SCC-152 at 37° C. and killing activity was assessed after 24 hours by measuring luciferase activity. The percent killing was calculated by the formula: % Killing=(1-(luciferase signal in T cells+targets/luciferase signal from target cells only))*100%, and was plotted versus the E:T ratios.

As shown in FIGS. 2B and 2C, expression of a variant CD86 TIPs enhanced anti-HER2 CAR-T cell killing of target cells expressing either high antigen (FIG. 2B) or low antigen (FIG. 2C). Data is shown with +/−standard deviation of triplicate wells.

### Example 7

### Generation and Assessment of CD86 Stacked Molecules Containing Different Affinity-Modified Domains

Selected variant CD86 molecules described in Examples 1-4 that were affinity-modified for increased binding to CD28 were used to generate “stack” Fc fusion molecules with a variant NKp30 IgV domain as a tumor localizing domain (designated “L”). The exemplary variant NKp30 was identified from screening a yeast display library containing mutagenized DNA encoding NKp30 variants for increased binding affinity to B7-H6. Following two rounds of FACS screening by staining with recombinant B7H6.Fc (rB7H6.Fc), outputs gave MFI values of 533 when stained with 16.6 nM rB7H6.Fc, whereas the parental NKp30 strain MFI was measured at 90 when stained with the same concentration of rB7H6.Fc (6-fold improvement). Among the NKp30 variants that were identified, was a variant that contained mutations L30V/A60V/S64P/S86G (SEQ ID NO:231), with reference to positions in the NKp30 extracellular domain corresponding to positions set forth in SEQ ID NO:54. The generated CD86-Nkp30 stack constructs were assessed for binding and activity.

### A. Generation of CD86-NkP30 Stack Constructs

Stack constructs were obtained by combining PCR products or geneblocks (Integrated DNA Technologies, Coralville, IA) that encoded the stack in a format that enabled its fusion to Fc by standard Gibson assembly using a Gibson assembly kit (New England Biolabs, USA). The stacks were generated in a homodimer format containing two identical polypeptides by fusion with a human IgG1 effectorless Fc sequence (e.g. set forth in SEQ ID NO: 230). The encoding nucleic acid molecule of all stacks was generated to encode a protein designed as follows: a first variant CD86 ECD or IgV, followed by a 15 amino acid linker composed of three GGGGS(G4S) motifs (SEQ ID NO: 224), followed by a variant NKp30 IgV domain, followed by a GSGGGGS linker (SEQ ID NO: 222), followed by the human IgG1 effectorless Fc sequence set forth in SEQ ID NO: 230 as described above.

Expression constructs encoding Fc fusion proteins were transfected into Expi293 HEK293 cells (e.g. Invitrogen). Supernatants were harvested and protein was captured and eluted from a Protein A column using an AKTA protein purification system.

TABLE E5
CD86-NKp30 Stacks

| SEQ ID NO of | IgSF components of Stack Construct | |
|---|---|---|
| stack construct | CD86 | NKp30 |
| 135 | CD86 ECD: | SEQ ID |
| | Q25L/H90L/K93T/M97L/T133A/S181P/D215V | NO: 231 |
| | (ECD set forth in SEQ ID NO: 93) | |
| 136 | CD86 ECD Q25L/Q86R/H90L/N104S | SEQ ID |
| | (ECD set forth in SEQ ID NO: 94) | NO: 231 |
| 137 | CD86ECD M60K/H90L | SEQ ID |
| | (ECD set forth in SEQ ID NO: 111) | NO: 231 |
| 138 | CD86 ECD I89V/H90L/I193V | SEQ ID |
| | (ECD set forth in SEQ ID NO: 107) | NO: 231 |
| 139 | CD86ECD Q25L/H90L/S179R | SEQ ID |
| | (ECD set forth in SEQ ID NO: 117) | NO: 231 |
| 140 | CD86 IgV Q25L, Q86R, H90L, N104S | SEQ ID |
| | (IgV set forth in SEQ ID NO: 150) | NO: 231 |

### B. Binding

Jurkat/IL-2 cells expressing CD28 and CHO cells transduced to express CTLA-4 were used to test binding of the variant CD86 domain of the stack constructs to its cognate binding partners CD28 and CTLA-4. Cells were incubated with various concentrations (100,000 pM to 98 pM, 6 points, 1:4 dilution series) of the exemplary constructs set forth in Table E5. For comparison, binding of multiple exemplary variant CD86-Fc constructs, as described in Example 2 and Table E1, and effectorless Fc alone (SEQ ID NO: 230) were also tested. Binding was assessed by flow cytometry using an anti-Fc antibody-PE and Mean Fluorescence Intensity (MFI) was determined.

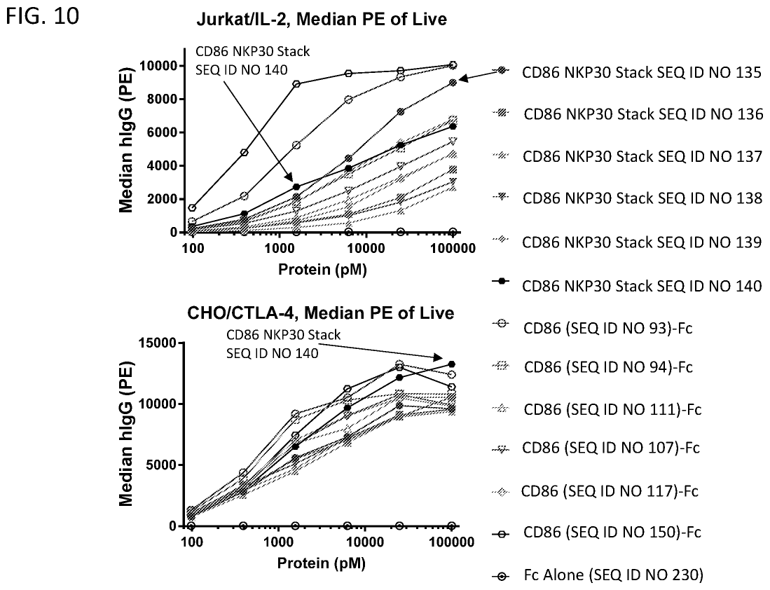

As shown in FIG. 10, exemplary NKp30-CD86 stack constructs retain binding to CD28 and CTLA-4 compared to exemplary variant CD86-Fc constructs and Fc alone. Although the stack constructs generally bound less well than the corresponding variant CD86 vIgD-Fc construct, binding to CD28 and CTLA-4 was observed for all stack constructs with particularly high binding shown for the stack construct set forth in SEQ ID NO: 135.

### C. T Cell Costimulation

Exemplary variant NKp30-CD86 stack constructs were assessed for their ability to costimulate primary T cells in a coimmobilization assay. Flat-bottom 96-well plates were coated with 5 nM anti-human CD3 (OKT3) and 40 nM B7H6 diluted in PBS and incubated overnight at 4° C. The following day, plates were rinsed 3 times with sterile PBS, and 1×10⁵ CFSE-labelled human Pan T cells were added to each well. T cells were incubated for 72 hrs with 100 nM, 8.333 nM, or 0.694 nM of the exemplary NKp30-CD86 stack constructs (see Table E5). For comparison, an exemplary variant CD86 set forth by SEQ ID NO: 93 (generated as a Fc fusion as described in Example 4), an exemplary variant NKp30 set forth by SEQ ID NO: 231 (generated as a Fc fusion as described in Example 4), and effectorless Fc alone (SEQ ID NO: 230) were also tested. Experiments were performed in triplicate.

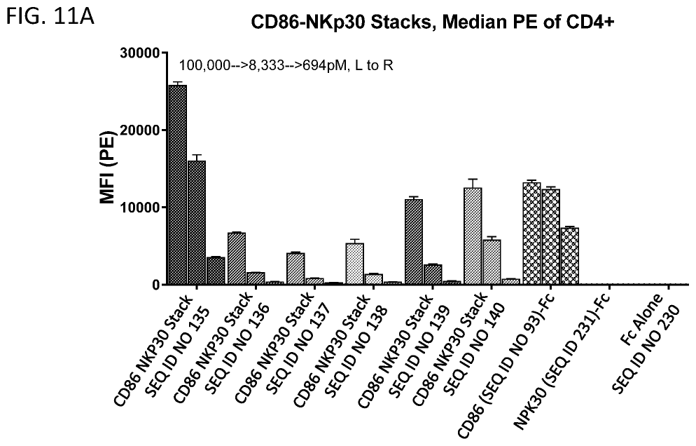

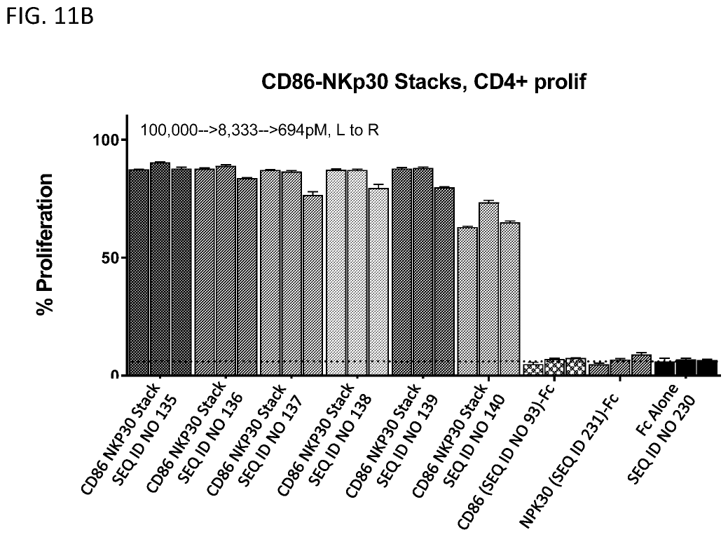

Binding of stack constructs to primary T cells was determined by flow cytometry after staining with an anti-Fc antibody-PE and mean fluorescence intensity (MFI) was determined. T cell proliferation was assessed by flow cytometric measurement of CFSE dye dilution and percent proliferation was determined. FIG. 11A shows binding of exemplary stack constructs to primary human CD4+ T cells. As shown in FIG. 11B, incubation with the exemplary stack constructs at each concentration induced T cell proliferation compared to the exemplary variant CD86-Fc, the exemplary variant NKp30-Fc, and Fc alone. Similar binding and proliferation results were observed for CD8+ T cells.

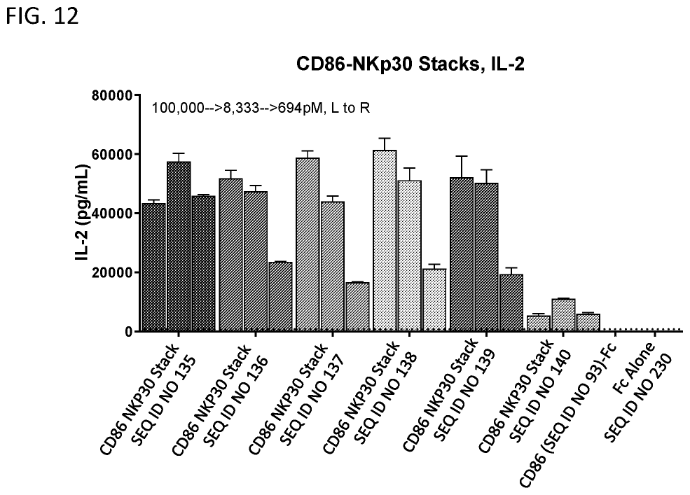

To assess T cell cytokine production, supernatant was harvested after 24 hrs of incubation and IL-2 concentration was determined by ELISA. As shown in FIG. 12, incubation with exemplary stack constructs increased IL-2 production compared to the exemplary variant CD86-Fc and Fc alone.

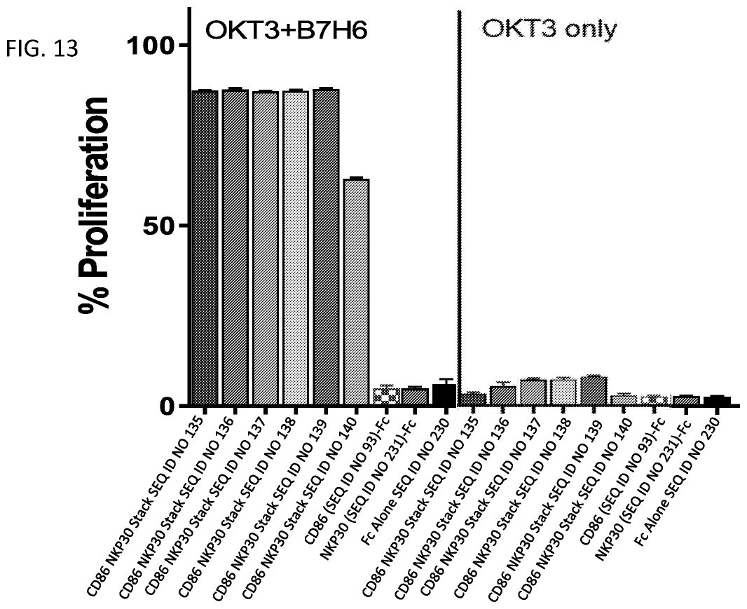

Together the above results demonstrate that the stack constructs are able to bind to primary T cells and provide costimulation. To assess whether stack construct activity depended on the presence of B7H6, the cognate binding partner of NKp30, a separate coimmobilization experiment was performed as described above, but plates were coated with anti-CD3 (OKT3) only. FIG. 13 shows that primary T cells incubated with exemplary stack constructs (100 nM) in the absence of B7H6 resulted in reduced CD4+ T cell proliferative activity compared to incubation in the presence of B7H6. Similar results were observed for CD8+ T cells. These data indicate that costimulation activity of CD86-NKp30 stack constructs is B7H6-dependent.

### Example 8

### Generation of Stacked Molecules Containing Variant PD-1 Molecules and Variant CD86 Molecules

Exemplary variant CD86 molecules described in Examples 1-5 that were affinity-modified were used to generate “stack” Fc fusion molecules with a an exemplary variant PD-1 IgSF domain that was affinity-modified with increased binding to PD-L1 (e.g. set forth in SEQ ID NO:315). The stack constructs contained either a wild-type CD86 extracellular domain (ECD; SEQ ID NO: 29), a wild-type CD86 IgV domain (SEQ ID NO: 123), or a CD86 ECD or IgV domain affinity-modified for increased binding to CD28 as described in Table E18. Stack constructs were obtained via overlap PCR of CD86 and PD-1 sequences that encoded the stack in a format that enabled its fusion to Fc by standard Gibson assembly using a Gibson assembly kit (New England Biolabs, USA). The encoding nucleic acid molecule of all stacks was generated to encode a protein designed as follows: a first wild-type or variant CD86 ECD or IgV domain, followed by a 15 amino acid linker composed of three GGGGS(G4S) motifs (SEQ ID NO: 224), followed by a variant PD-1 domain, followed by a GSGGGGS linker (SEQ ID NO: 222), followed by the human IgG1 effectorless Fc sequence set forth in SEQ ID NO: 230 as described above.

Table E6 sets forth exemplary generated CD86-PD-1 Stacks.

TABLE E6
CD86-PD-1 Stacks

| SEQ ID NO of | IgSF components of Stack Construct | |
|---|---|---|
| stack construct | CD86 | PD-1 |
| 316 | WT CD86 ECD | SEQ ID |
| | (ECD set forth in SEO ID NO: 29) | NO: 315 |
| 317 | CD86 ECD: | SEQ ID |
| | A13V/Q25L/H90L/S181P/L197M/S206T (ECD | NO: 315 |
| | set forth in SEQ ID NO: 89 ) | |
| 318 | CD86 ECD: | SEQ ID |
| | Q25L/H90L/K93T/M97L/T133A/S181P/D215V | NO: 315 |
| | (ECD set forth in SEQ ID NO: 93) | |
| 319 | CD86 ECD: | SEQ ID |
| | Q25L/Q86R/H90L/N104S | NO: 315 |
| | (ECD set forth in SEQ ID NO: 94) | |
| 320 | CD86 IgV domain | SEQ ID |
| | (IgV set forth in SEQ ID NO: 123) | NO: 315 |
| 321 | CD86 IgV: | SEQ ID |
| | A13V/Q25L/H90L/S181P/L197M/S206T | NO: 315 |
| | (IgV set forth in SEQ ID NO: 145) | |
| 322 | CD86 IgV: | SEQ ID |
| | Q25L/H90L/K93T/M97L/T133A/S181P/D215V | NO: 315 |
| | (IgV set forth in SEQ ID NO: 149) | |
| 323 | CD86 IgV: | SEQ ID |
| | Q25L/Q86R/H90L/N104S | NO: 315 |
| | (IgV set forth in SEQ ID NO: 150) | |

### Example 9

### Assessment of Binding and Activity of Stacked Molecules Containing Variant PD-1 Molecules and Variant CD86 Molecules

Exemplary stack constructs were generated substantially as described in Example 8, containing a variant PD-1 IgSF domain (e.g. SEQ ID NO:315) with either a wild-type CD86 extracellular domain (ECD; e.g. SEQ ID NO: 29), a wild-type CD86 IgV domain (e.g. SEQ ID NO: 123), or a variant CD86 IgSF domain (ECD, e.g. SEQ ID NO: 89 or 93 or 94; or IgV, e.g SEQ ID NO: 145, 149 or 150), and were assessed for binding and activity.

### A. Binding

To assess binding to cognate binding partners, cells were transduced to express huCTLA, huCD28 and huPD-L1 full-length mammalian proteins. Cells were incubated with various concentrations (0.1 nM to 100 nM) of the exemplary constructs set forth in Table E6. For comparison, binding of wild-type CD80-Fc was also tested. For binding to PD-L1, binding also was compared to anti-PD-1 antibodies (Imfinzi and Atezolizumab). An Fc only molecule was also tested as a control. Binding was assessed by flow cytometry and mean Fluorescence Intensity (MFI) was determined.

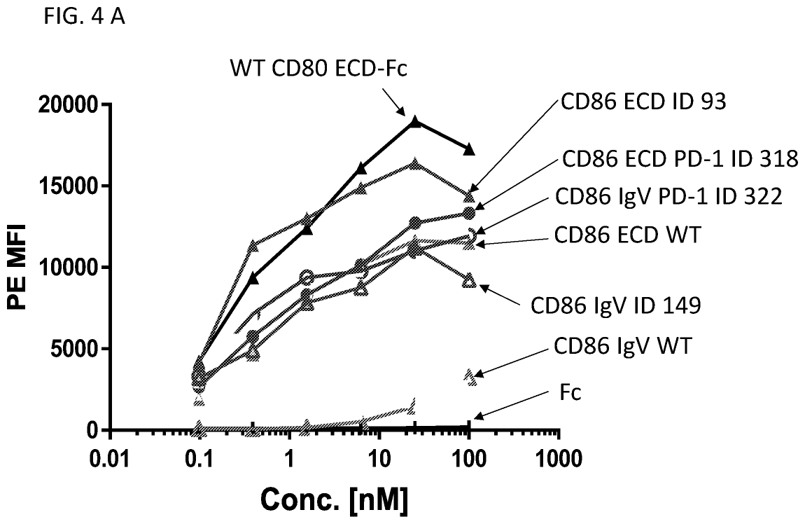

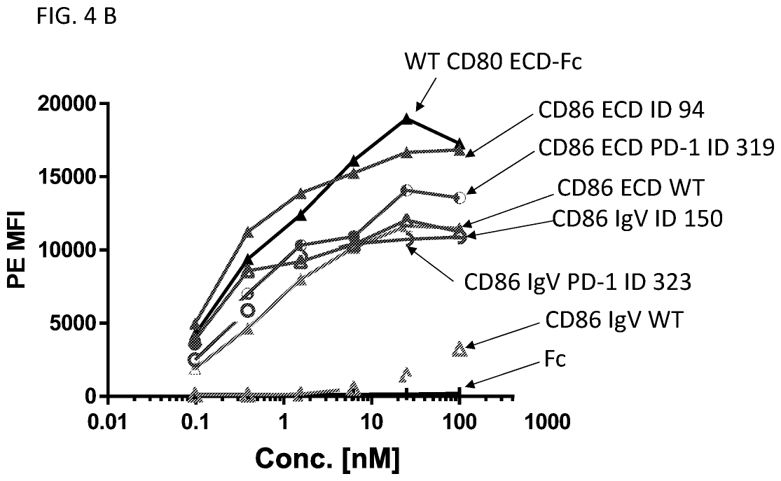

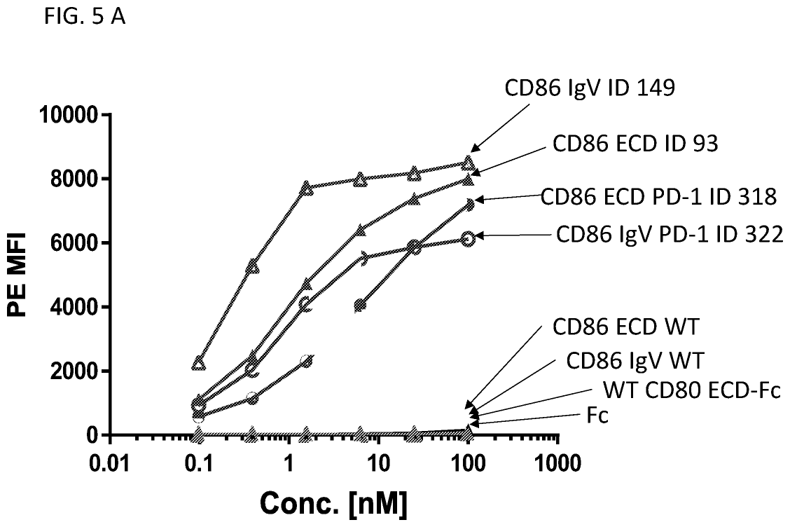

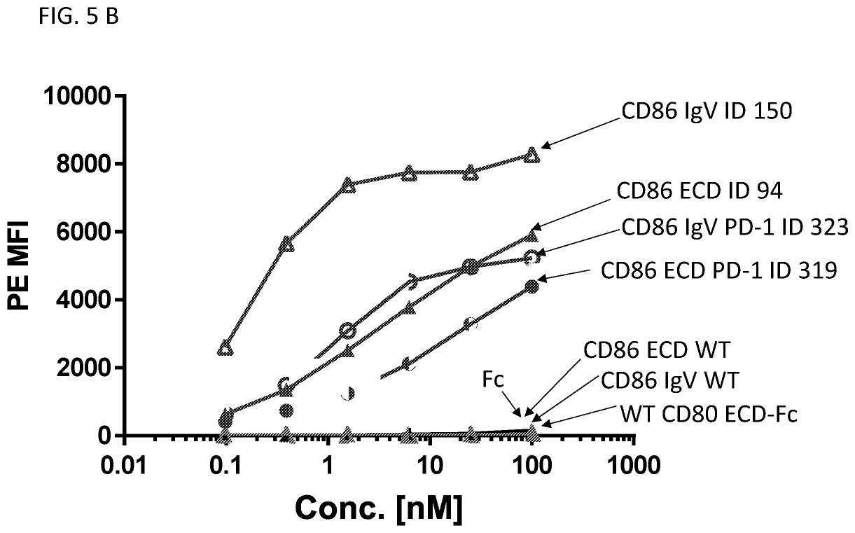

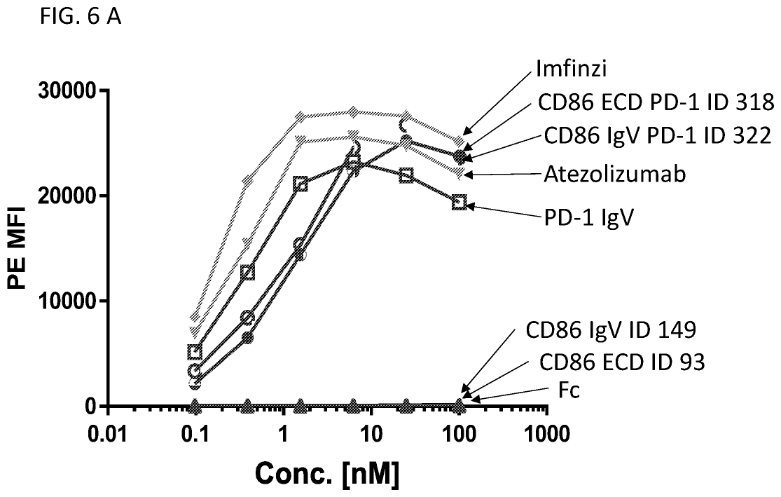

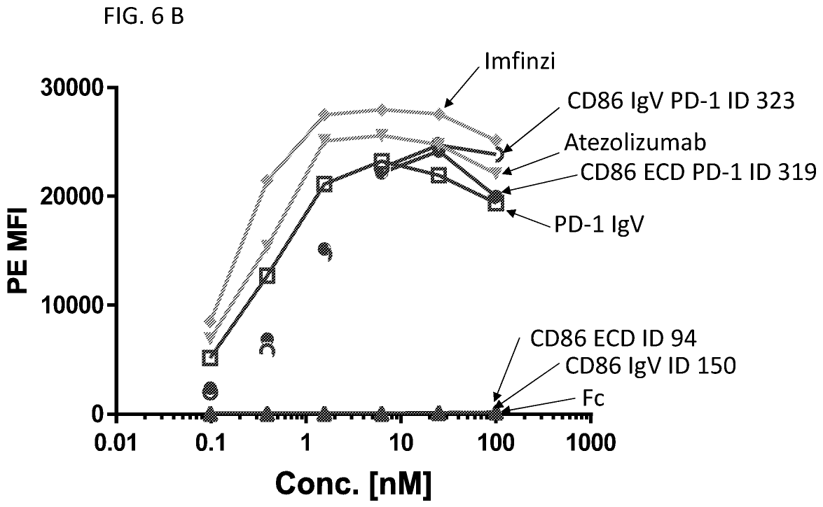

As shown in FIGS. 4A-6B, exemplary PD1-CD86 stack constructs retain binding to CTLA-4, CD28 and PD-L1 compared to molecules containing only individual wild-type or variant IgSF domains. Stack constructs containing mutations Q25L/H90L/K93T/M97L/T133A/S181P/D215V in IgV (SEQ ID NO:149, e.g. stack set forth in SEQ ID NO:322) or ECD (SEQ ID NO:93, e.g. stack set forth in SEQ ID NO:318) retain binding to CTLA-4 (FIG. 4A), CD28 (FIG. 5A) and PD-L1 (FIG. 6A), compared to molecules containing only individual wild-type or variant IgSF domains. Stack constructs containing mutations Q25L/Q86R/H90L/N104S in IgV (SEQ ID NO:150, e.g. stack set forth in SEQ ID NO:323) or ECD (SEQ ID NO:94, e.g. stack set forth in SEQ ID NO:319) retain binding to CTLA-4 (FIG. 4B), CD28 (FIG. 5B) and PD-L1 (FIG. 6B),

### B. PD-L1-Dependent CD28 Costimulation

Exemplary variant stack constructs were assessed for their ability to deliver PD-L1 dependent costimulation of CD28 using Jurkat/IL-2 reporter cells expressing PD-1. K562/OKT3/PD-L1 or K562/OKT3 artificial antigen presenting cells (aAPCs) were plated at 20,000 cells/well and pre-incubated with various amounts of exemplary variant stack constructs from 0.01 nM to 100 nM. An Fc only molecule was also tested as control. Jurkat effector cells expressing an IL-2-luciferase reporter were added at a total of 100,000 cells per well, such that each well had a final ratio of 1:5 K562: Jurkat cells. Jurkat cells, K562 cells, and exemplary stack constructs were incubated for 6 hours at 37 degrees Celsius. 100 μL of a cell lysis and luciferase substrate solution (BioGlo luciferase reagent, Promega) were added to each well and luminescence was measured.

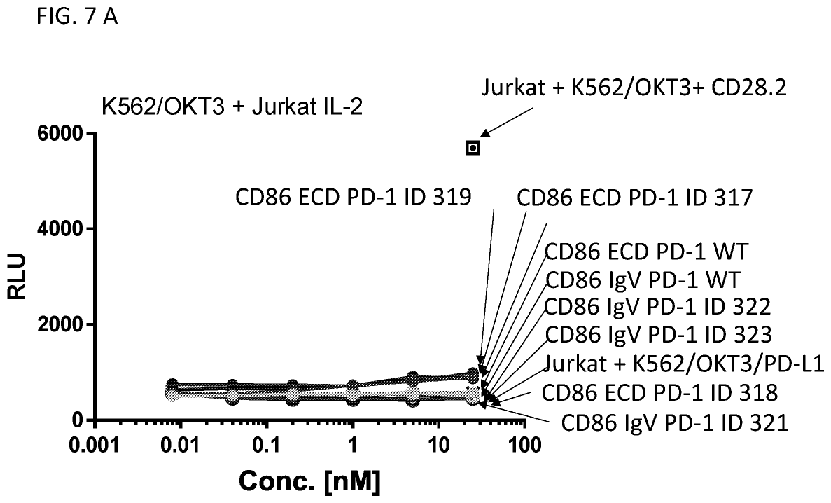

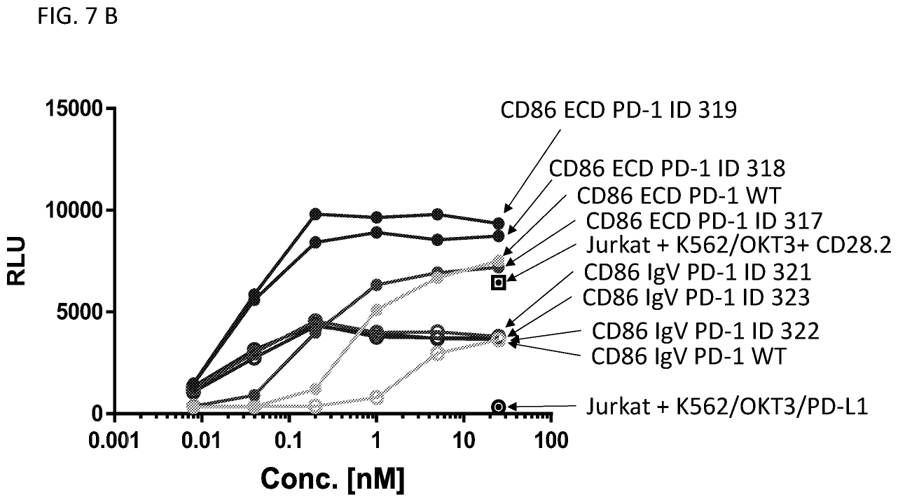

As shown in FIG. 7A, the addition of the exemplary variant stack constructs in the absence of PD-L1 exhibited little to no co-stimulatory signal consistent with the observation that PD-1/CD86 containing stack proteins require PD-L1 binding to induce a costimulatory signal via CD28. As shown in FIG. 7B, the addition of both ECD and IgV exemplary variant stack constructs in the presence of PD-L1 agonized CD28 dependent luminescent activity, as measured by IL-2 luminiescence relative luminescence units (RLU). The level of costimulation correlated with the CD28 and/or PD-L1 binding affinity of the variant molecules. This result is consistent with the activity of the variant PD-1-containing stack immunomodulatory proteins to exhibit PD-L1-dependent CD28-mediated costimulation.

### C. T Cell Response

A cytomegalovirus (CMV) antigen-specific functional assay was used to assess the effect of exemplary variant stack molecules on T cell responses.

Peripheral blood mononuclear cells (PBMC) obtained from CMV seropositive donor were thawed and CMV lysate added at i pg/mL to 250,000/well PBMC in the presence of tested exemplary variant stack constructs (diluted at 1:3 dilutions from 100,000 pM to 46 μm). An Fc only molecule was also tested as control. Supernatant was collected 48 hours after incubation to assay IL-2 by ELISA, and 96 hours after incubation to assay IFNg by ELISA.

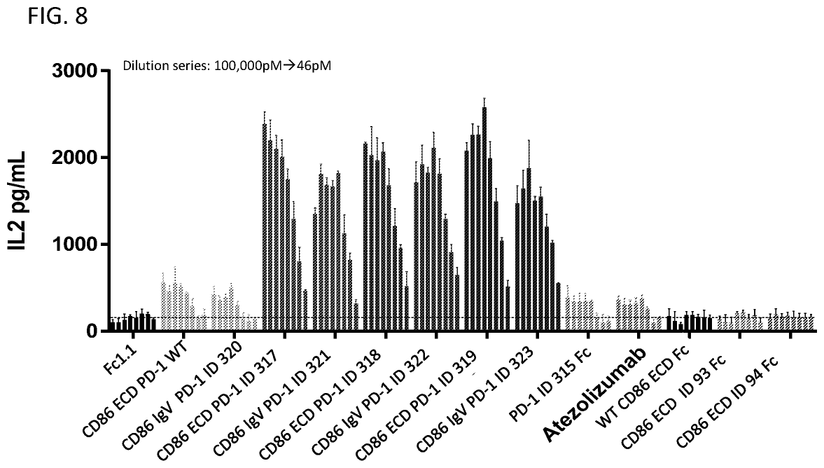

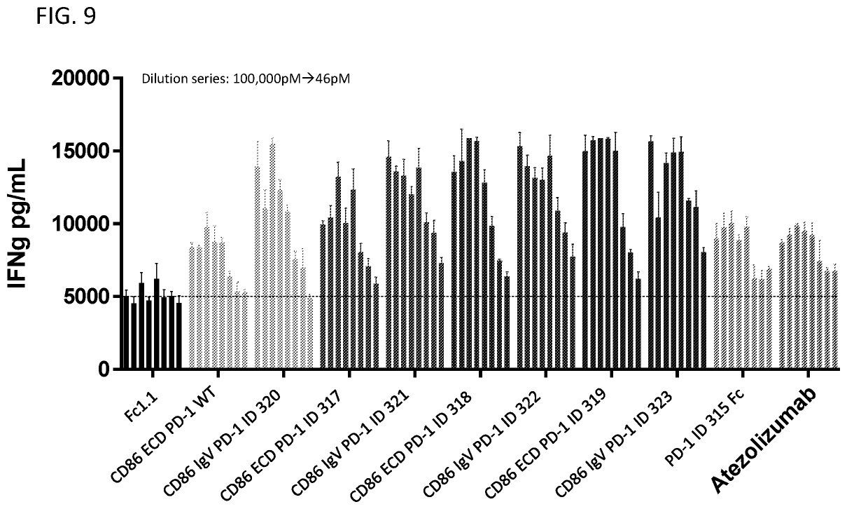

The exemplary PD1-CD86 stack constructs showed a concentration dependent increase in IL-2 production (FIG. 8) and IFNg production (FIG. 9). The PD1-CD86 stack constructs stimulated the production of cytokines to a greater degree than PD-L1 control antibody (atezolizumab) or the individual variant PD1 IgV-Fc molecule. The PD1-CD86 stack constructs also stimulated the production of cytokines to a greater degree than wild-type CD86 ECD-Fc or the individual variant CD86 ECD-Fc molecules.

These results are consistent with PD1-CD86 stack molecules displaying costimulatory effects to stimulate CD28 in a PD-L1-dependent manner. These results were observed for constructs with varying degrees of binding to CD28 on Jurkat cells as shown in Example 18.B above, including stack constructs containing a wild-type CD86 ECD that was observed to have low detectable binding to CD28.

### Example 10

### Generation and Assessment of Binding and Activity of Formatted Stacked Molecules Containing Variant PD-1 Molecules and Variant CD86 Molecules

Exemplary variant CD86 molecules and exemplary variant PD-1 molecules that were affinity-modified as described in Examples 1-5 and Example 8, respectively, were used to generate “stack” Fc fusion molecules. The generated CD86-PD1 stack constructs were assessed for binding and activity.

### A. Generation of CD86-PD-1 Stack Constructs

The stacks were generated by combining PCR products or geneblocks (Integrated DNA Technologies, Coralville, IA) that encoded the stack in a format that enabled its fusion to Fc by standard Gibson assembly using a Gibson assembly kit (New England Biolabs, USA). Stacks constructs had a homodimer format containing two identical polypeptides by fusion with a human IgG1 effectorless Fc sequence (e.g., set forth in SEQ ID NO: 230). Alternatively, stack constructs had a heterodimer format by “knobs-into-hole” Fc engineering to promote association of two different individual chains of the heterodimer, in which one polypeptide was fused to a “knob” Fc subunit (SEQ ID NO:346) and the second polypeptide was fused to a “hole” Fc subunit (SEQ ID NO:347).

Expression constructs encoding Fc fusion proteins were transfected into Expi293 HEK293 cells (e.g. Invitrogen). Supernatants were harvested and protein was captured and eluted from a Protein A column using an AKTA protein purification system.

Table E7 sets forth exemplary generated PD-1-CD86 stacks and formats. FIGS. 14A-14D depicts the structure of exemplary formatted stack constructs.

TABLE E7
Exemplary CD86-PD1 Stacks Formatted

| Exemplary | SEQ ID NOs | | | | | |
|---|---|---|---|---|---|---|
| Format | of stack | | | | | |
| (FIG.) | construct | Domain 1 | Linker | Domain 2 | Linker | Domain 3 |
| FIG. 14A | 326 | Variant PD1 | GSG4S | Fc | four | Variant |
| | | ECD | (SEQ ID | (SEQ ID | GGGGS | CD86 |
| | | (SEQ ID | NO: 222) | NO: 230) | (G4S) | ECD |
| | | NO: 315) | | | motifs | (SEQ ID |
| | | | | | (SEQ ID | NO: 94) |
| | | | | | NO: 224) | |
| | 326 | Variant PD1 | GSG4S | Fc | four | Variant |
| | | ECD | (SEQ ID | (SEQ ID | GGGGS | CD86 |
| | | (SEQ ID | NO: 222) | NO: 230) | (G4S) | ECD |
| | | NO: 315) | | | motifs | (SEQ ID |
| | | | | | (SEQ ID | NO: 94) |
| | | | | | NO: 224) | |
| FIG. 14A | 327 | Variant PD1 | GSG4S | Fc | four | Variant |
| | | ECD | (SEQ ID | (SEQ ID | GGGGS | CD86 IgV |
| | | (SEQ ID | NO: 222) | NO: 230) | (G4S) | (SEQ ID |
| | | NO: 315) | | | motifs | NO: 150) |
| | | | | | (SEQ ID | |
| | | | | | NO: 224) | |
| | 327 | Variant PD1 | GSG4S | Fc | four | Variant |
| | | ECD | (SEQ ID | (SEQ ID | GGGGS | CD86 IgV |
| | | (SEQ ID | NO: 222) | NO: 230) | (G4S) | (SEQ ID |
| | | NO: 315) | | | motifs | NO: 150) |
| | | | | | (SEQ ID | |
| | | | | | NO: 224) | |
| FIG. 14B | 329 | Variant PD1 | GSG4S | Fc | — | — |
| | | ECD | linker | (SEQ ID | | |
| | | (SEQ ID | (SEQ ID | NO: 324) | | |
| | | NO: 315) | NO: 222) | | | |
| | 329 | Variant PD1 | GSG4S | Fc | — | — |
| | | ECD | linker | (SEQ ID | | |
| | | (SEQ ID | (SEQ ID | NO: 325) | | |
| | | NO: 315) | NO: 222) | | | |
| FIG. 14C | 328 | Variant PD1 | GSG4S | Fc | — | — |
| | | ECD | linker | (SEQ ID | | |
| | | (SEQ ID | (SEQ ID | NO: 324) | | |
| | | NO: 315) | NO: 222) | | | |
| | 330 | Fc | four | Variant | — | — |
| | | (SEQ ID | GGGGS | CD86 | | |
| | | NO: 325) | (G4S) | IgV | | |
| | | | motifs | (SEQ ID | | |
| | | | (SEQ ID | NO: 150) | | |
| | | | NO: 224) | | | |
| FIG. 14D | 328 | Variant PD1 | GSG4S | Fc | — | — |
| | | ECD | linker | (SEQ ID | | |
| | | (SEQ ID | (SEQ ID | NO: 324) | | |
| | | NO: 315) | NO: 222) | | | |
| | 331 | Variant PD1 | GSG4S | Fc | four | Variant |
| | | ECD | linker | (SEQ ID | GGGGS | CD86 |
| | | (SEQ ID | (SEQ ID | NO: 325) | (G4S) | IgV |
| | | NO: 315) | NO: 222) | | motifs | (SEQ ID |
| | | | | | (SEQ ID | NO: 150) |
| | | | | | NO: 224) | |

### B. Binding

To assess binding activity, the exemplary formatted stack constructs were incubated with Jurkat cells expressing CD28, the cognate binding partner of CD86, and with K562 cells transduced to express PDL1, the cognate binding partner of PD-1. The cells (50,000 cells/well) were incubated on ice for 30 min with the formatted stack constructs set forth in Table E7 at various concentrations (starting concentration 100 nM, 8 serial 1:4 dilutions titrated down). Cells were then washed with FACS buffer and resuspended with 50 μL anti-Fc antibody-PE for 30 min. Binding was assessed by flow cytometry and Mean Fluorescence Intensity (MFI) was determined.

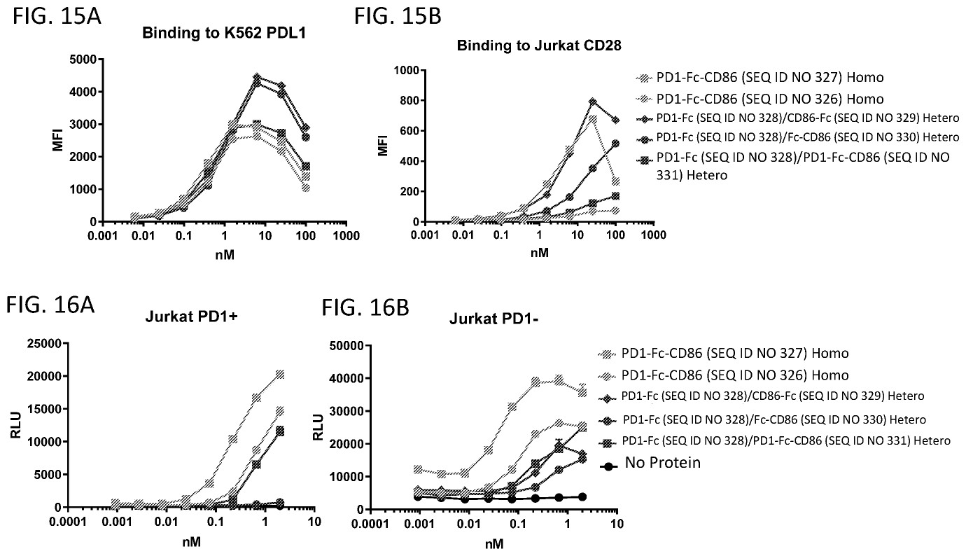

As shown in FIGS. 15A and 15B, exemplary PD-1-CD86 stack construct formats retain binding to PDL1 and CD28 compared to exemplary variant unformatted control stacks. These data indicate that the different stacked molecule formats retain binding activity to cognate binding partners.

### C. T Cell Costimulation

To test whether exemplary stack constructs could drive target-specific costimulation of T cells, a transfected cell system including a T cell reporter line for measuring costimulation was used. Jurkat cells including an IL-2 promoter luciferase reporter and that expressed or did not express PD1 were used to evaluate costimulatory function. To stimulate the Jurkat cells, K562 cells were engineered for use as artificial antigen presenting cells. Specifically, K562 cells were transduced with a lentivirus encoding a single-chain Fv version of the anti-CD3 antibody OKT3 with or without transduction with a separate lentivirus directing PDL1 expression. K562 cells displaying cell surface anti-CD3 single chain Fv (OKT3) with or without surface PDL1 expression were plated in culture media at 2×10⁴ cells/well. Target cells were incubated for 10 min with exemplary PD1-CD86 stack constructs (titrated down from 2 nM in 8 serial, 1:3 dilutions) at 37° C. The Jurkat effector cells expressing an IL-2-luciferase reporter gene (Promega) were added at 1×10⁵ cells/well to bring the final volume/well to 100 μl. Target and Jurkat cells in the presence or absence of exemplary stack constructs were incubated for 5 hours at 37° C. Plates were removed from the incubator and acclimated to room temperature for 15 minutes. 100 μL of cell lysis and luciferase substrate solution (BioGlo luciferase reagent, Promega) was added to each well and the plates were incubated on an orbital shaker for 10 minutes. Luminescence was measured with a 1 second per well integration time using a Cytation 3 imaging reader (BioTek Instruments). Relative luminescence values (RLU) were determined for each test sample and reported.

As shown in FIGS. 16A and 16B, incubation with the exemplary stack constructs provided costimulation to the T cells in the presence of PDL1+K562/OKT3 cells regardless of whether the T cells were PD1+ or PD1−. These results suggest that stack constructs are capable of delivering a T cell costimulatory signal even in the presence of strong checkpoint blockade.

### D. Cytokine Production

To assess the ability of exemplary CD86-PD-1 stack constructs (see Table E7) to facilitate cytokine production in T cells, an HLA-A2+HPV+ target cell line known to express PD-L1 (SCC152 PDL1+) was co-cultured with T cells transduced to express an exemplary recombinant HPV16 E6-specific T cell receptor (TCR) in the presence of various concentrations of the exemplary formatted stacks. SCC152 PDL1+ cells were seeded into a 96-well plate at 40,000 cells/well with culture media and incubated overnight at 37° C. The following day, various concentrations of the exemplary formatted stack constructs (0.666 nM, titrated at 1:3 dilution for 5-point titration) and 40,000 T cells expressing the recombinant HPV16 E6-specific T cell receptor (TCR) were added to each well. For comparison, cells were incubated with a variant CD80 IgV-Fc fusion protein known to bind CD28 and PD-L1, with mock transduced T cells (mock), or incubated in the absence of formatted stack constructs (no protein). Plates were incubated at 37° C., and supernatant was collected after 24 hrs of incubation to test for secreted cytokines, e.g., IFNg, IL-2, TNFa.

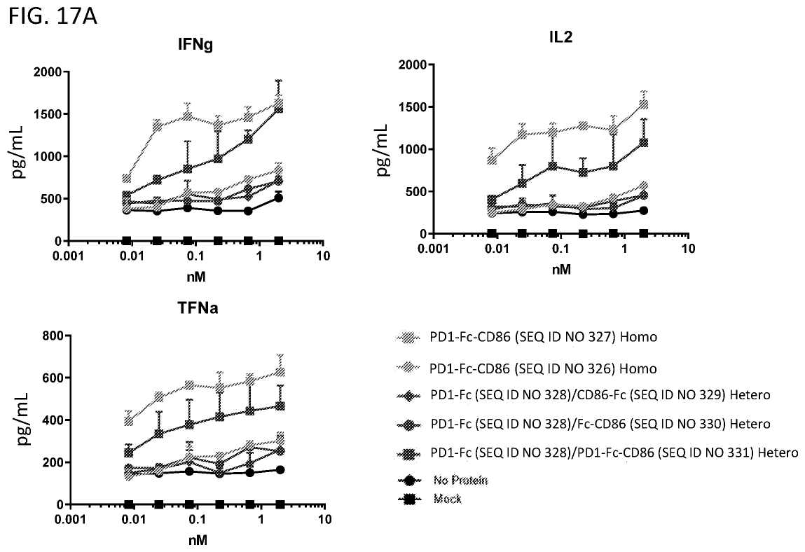

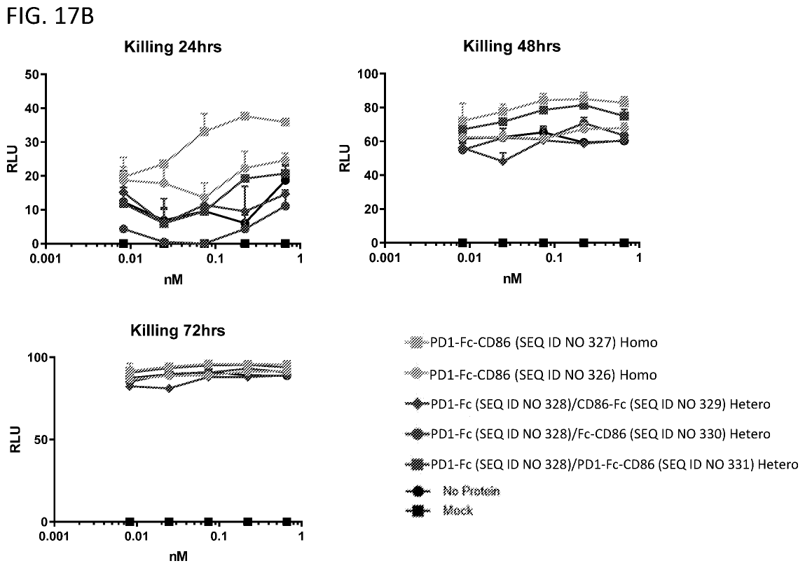

As shown in FIG. 17A, formatted CD86-PD-1 stack constructs induced higher levels of IFNg, IL-2, and TNFa cytokine production at 24 hrs relative to control. These data indicate that formatted CD86-PD-1 stack constructs facilitate costimulation and cytokine production in target specific T cells.

### E. Cytotoxic Activity

Exemplary CD86-PD-1 stack constructs (see Table E7) were assessed for their ability to facilitate T cell cytotoxic activity. HLA-A2+HPV+ target cell line SCC152 PDL1+ were seeded into a 96-well plate at 20,000 cells per well with culture media and incubated overnight at 37° C. The following day, the culture media was discarded, and the cells were incubated for 10 min at 37° C. with 100 μL of luciferin. Various concentrations of the exemplary formatted stack constructs (0.666 nM, titrated at 1:3 dilution for 5-point titration) and 20,000 T cells transduced with an exemplary recombinant HPV16 E6-specific T cell receptor (TCR) were added to each well and incubated for 10 min at 37° C. before being centrifuged (1300 rpm) for 30 sec. For comparison, cells were incubated with a variant CD80 IgV-Fc fusion protein known to bind CD28 and PD-L1, mock transduced T cells (mock), or in the absence of formatted stack constructs (no protein). Plates were incubated at 37° C., and relative luminescence values (RLU) were determined after 24, 48, and 72 hrs of incubation.

FIG. 17B shows cell killing, as measured by RLU, at 24, 48, and 72 hrs at each concentration. CD86-PD-1 stack constructs enhanced T cell cytotoxic activity at 24 hours compared to control. These data support that the CD86-PD-1 stack constructs are capable of costimulating T cells to induce T cell cytotoxic activity.

### Example 11

### Assessment of Binding and Costimulatory Function of Conjugates HER2 and EGFR Targeting Antibody

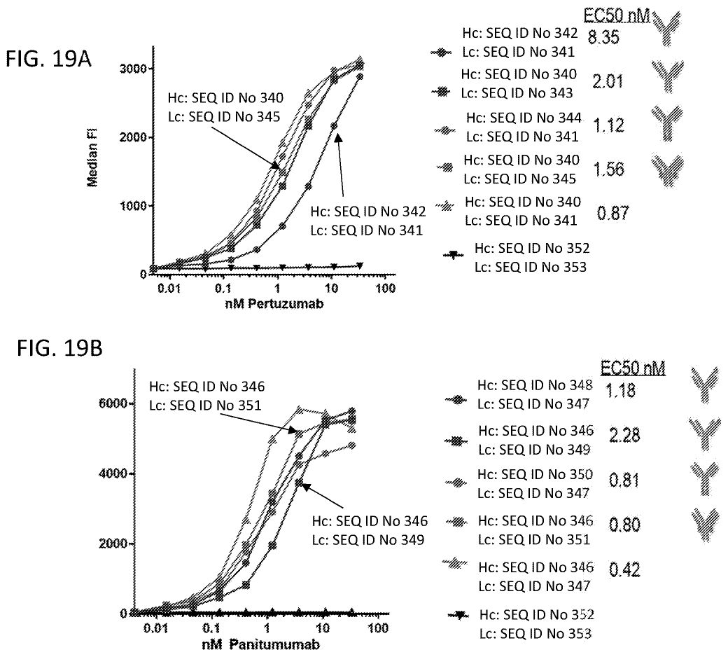

Exemplary variant CD86 IgV molecules were conjugated to HER2 and EGFR targeting antibodies to form conjugates (fusion proteins). The variant CD86 IgV domain set forth by SEQ ID NO: 150 was fused to the amino or carboxyl termini of the light chain or heavy chain of the HER2 targeting antibody pertuzumab or the EGFR targeting antibody panitumumab with intervening G4S linkers (see Table E8). Exemplary configurations of conjugates (fusion proteins) are shown in FIG. 18. DNA encoding each of the constructs diagrammed in FIGS. 19A and 19B was transfected into HEK-293 cells and secreted proteins were purified by Protein A and size exclusion chromatography. The resultant conjugate proteins were next assessed for retention of appropriate binding properties.

TABLE E8
Exemplary CD86-Antibody Conjugates
and Control Antibodies

| | Heavy Chain (HC) | Light Chain (LC) |
|---|---|---|
| Description | SEQ ID NO | SEQ ID NO |
| Pertuzumab | 340 | 341 |
| CD86-HC | 342 | 341 |
| Pertuzumab | | |
| CD86-LC | 340 | 343 |
| Pertuzumab | | |
| HC-CD86 | 344 | 341 |
| Pertuzumab | | |
| LC-CD86 | 340 | 345 |
| Pertuzumab | | |
| Panitumumab | 346 | 347 |
| CD86-HC | 348 | 347 |
| Panitumumab | | |
| CD86-LC | 346 | 349 |
| Panitumumab | | |
| HC-CD86 | 350 | 347 |
| Panitumumab | | |
| LC-CD86 | 346 | 351 |
| Panitumumab | | |

Control Antibody

| | | |
|---|---|---|
| Ramucirumab | 352 | 353 |

### A. Binding

To assess the ability of the exemplary pertuzumab-CD86 conjugates (fusion proteins) to bind HER2, SCC-152 cells were incubated with various concentrations of exemplary conjugates (see Table E8), pertuzumab, or control antibody (ramucirumab). SCC152 cells (50,000 cells/well) were incubated on ice for 30 min with the CD86 antibody conjugates (fusion proteins) set forth in Table E8 at various concentrations (starting concentration 33 nM, 9 serial 1:3 dilutions titrated down). Cells were then washed with FACS buffer and resuspended with 50 μL anti-Fc antibody-PE for 30 min. Binding was assessed by flow cytometry and Median Fluorescence Intensity (MFI) was determined. As shown in FIG. 19A, conjugates retained binding to HER2.

To assess the ability of the exemplary panitumumab-CD86 conjugates to bind EGFR, CHO cells transduced to express EGFR were incubated with various concentrations of exemplary conjugates (see Table E8), panitumumab, or control antibody (ramucirumab). CHO-EGFR cells (50,000 cells/well) were incubated on ice for 30 min with the CD86 antibody fusion conjugate constructs set forth in Table E8 at various concentrations (starting concentration 33 nM, 9 serial 1:3 dilutions titrated down). Cells were then washed with FACS buffer and resuspended with 50 μL anti-Fc antibody-PE for 30 min. Binding was assessed by flow cytometry and Median Fluorescence Intensity (MFI) was determined. As shown in FIG. 19B, conjugates retained binding to EGFR.

B. T cell Costimulation

To test whether exemplary conjugates (fusion proteins) could drive target-specific costimulation of T cells, a transfected cell system including Jurkat cells with an IL-2 promoter luciferase reporter were used to evaluate costimulatory function. To stimulate the Jurkat cells, target SCC-152 cells, which constitutively express HPV viral proteins, were plated in culture media at 2×10⁴ cells/well. Target cells were incubated for 10 min with CD86 antibody fusion conjugates (titrated down from 2 nM in 8 serial, 1:4 dilutions) at 37° C. The Jurkat effector cells expressing an IL-2-luciferase reporter gene (Promega) were added at 1×10⁵ cells/well to bring the final volume/well to 100 μl. Target and Jurkat cells in the presence or absence of exemplary conjugates were incubated for 5 hours at 37° C. Plates were removed from the incubator and acclimated to room temperature for 15 minutes. 100 μL of cell lysis and luciferase substrate solution (BioGlo luciferase reagent, Promega) was added to each well and the plates were incubated on an orbital shaker for 10 minutes. Luminescence was measured with a 1 second per well integration time using a Cytation 3 imaging reader (BioTek Instruments). Relative luminescence values (RLU) were determined for each test sample and reported.

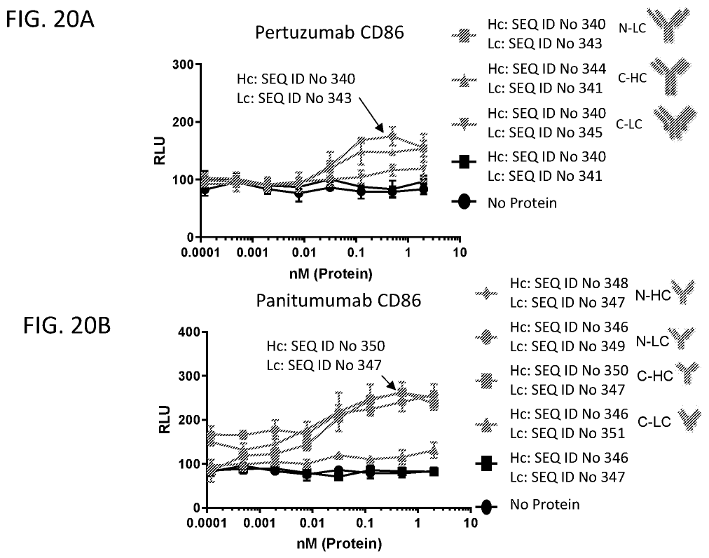

SCC-152 cells were co-cultured with the Jurkat IL2 reporter cell line transduced with an TCR specific for the HPV peptide E6 in the presence or absence of various concentrations of conjugates or control antibody. As shown in FIGS. 20A and 20B, RLU measures revealed that T cells experienced increased costimulation in the presence of conjugates compared to control antibody.

### C. Cytotoxic Activity

Exemplary conjugates were assessed for their ability to facilitate T cell cytotoxic activity. Target cell lines SCC-152 were seeded into a 96-well plate at 20,000 cells per well with culture media and incubated overnight at 37° C. The following day, the culture media was discarded, and the cells were incubated for 10 min at 37° C. with 100 μL of luciferin. 20,000 primary human T cells transduced with E6 TCR were added to each well at various effector to target ratios (E:T) in the presence of 2 nM of an exemplary conjugate and incubated for 10 min at 37° C. before being centrifuged (1300 rpm) for 30 sec. For comparison, cells were incubated with pertuzumab only, panitumumab only, control antibody ramucirumab (mock), or in the absence of protein. Plates were incubated at 37° C., and the percentage of target cell killing was determined after 24 hrs of incubation.

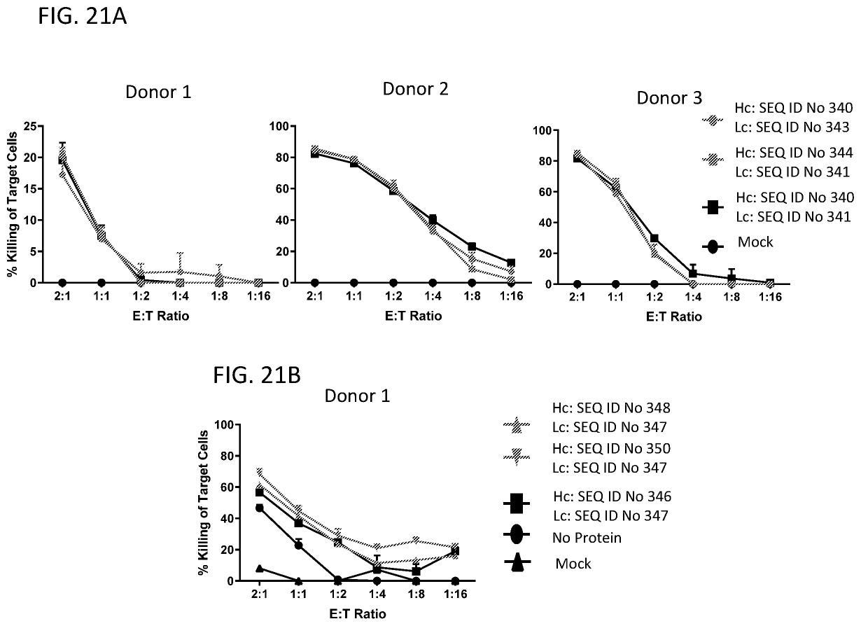

FIGS. 21A and 21B demonstrate minimal increases in percent cell killing after 24 hrs at each E:T in the presence of exemplary conjugates.

### C. Cytokine Production

To assess the ability of exemplary conjugate to facilitate cytokine production in T cells, SCC-152 cells were co-cultured with primary human T cells transduced with E6 TCR (three donors) in the presence of various concentrations of the exemplary conjugates (see Table E8). SCC-152 cells were seeded into 96-well plates at 40,000 cells/well with culture media and incubated overnight at 37° C. The following day, various concentrations of the exemplary conjugates and 40,000 T cells were added to each well. For comparison, cells were incubated with pertuzumab only, panitumumab only, control antibody ramucirumab (mock), or in the absence of protein. Plates were incubated at 37° C., and supernatant was collected after 24 hrs of incubation to test for secreted cytokines, e.g., IFNg, IL-2, TNFa.

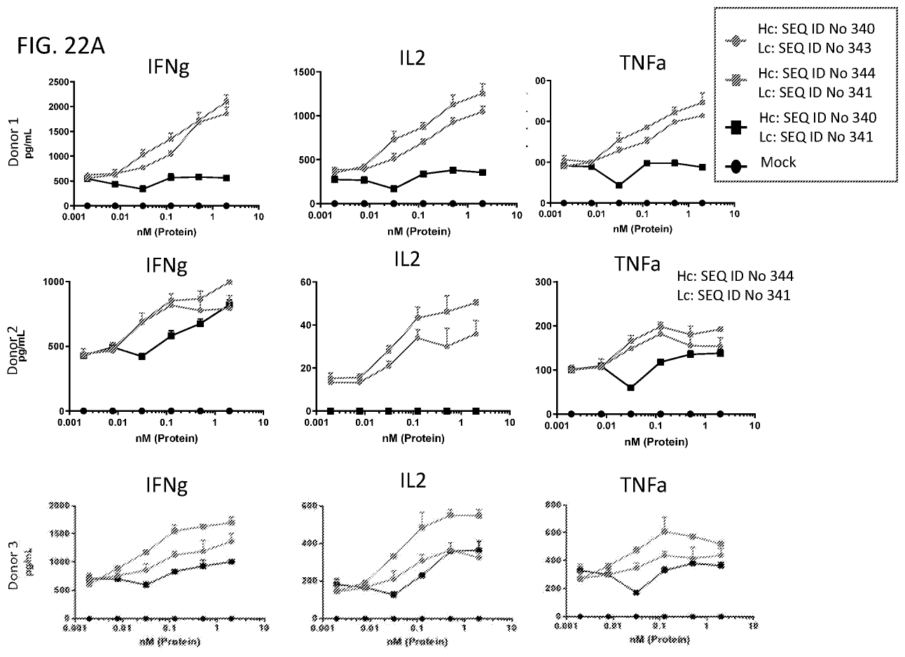

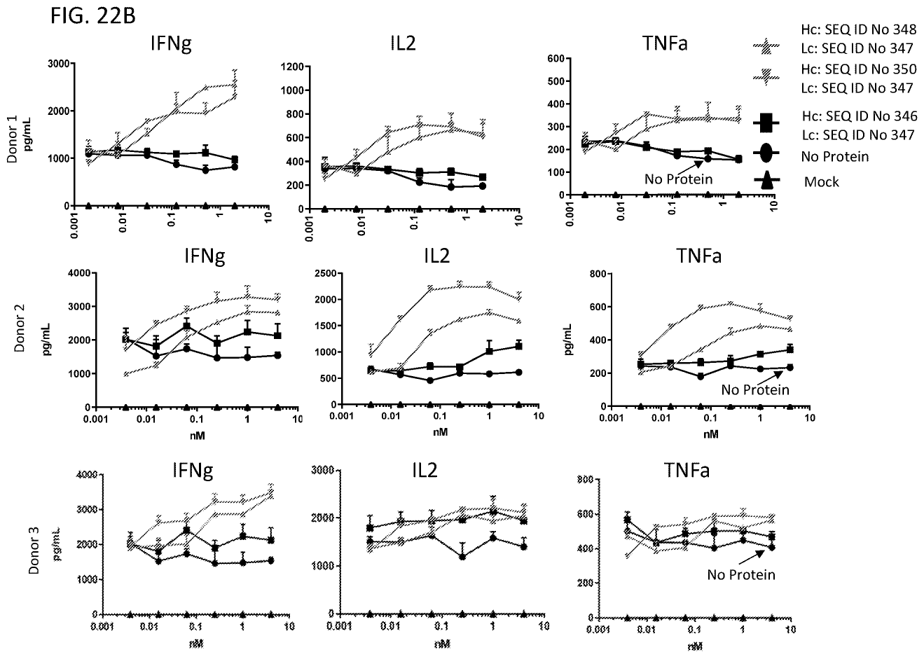

FIGS. 22A and 22B show the concentration of IFNg, IL-2, and TNFa for each donor. As shown, CD86-antibody conjugates resulted in cells exhibiting enhanced cytokine production compared to antibody only controls. These data demonstrate that conjugates are able to facilitate costimulation and cytokine production in T cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A variant CD86 polypeptide, comprising an extracellular domain or an IgV domain or specific binding fragment thereof, wherein the variant CD86 polypeptide comprises an amino acid modification at position 33 with reference to an unmodified CD86 polypeptide, and further comprises one or more amino acid modifications at position(s) selected from 13, 18, 25, 28,38, 39, 40, 43, 45, 52, 53, 60, 68, 71, 77, 79, 80, 82, 86, 88, 89, 90, 92, 93, 97, 102, 104, 113, 114, 123, 128, 129, 132, 133, 137, 141, 143, 144, 148, 153, 154, 158, 170, 172, 175, 178, 180, 181, 183, 185, 192, 193, 196, 197, 198,205,206,207,212,215,216,222,223, or 224, with reference to positions set forth in SEQ ID NO:29.

2.-5. (canceled)

6. The variant CD86 polypeptide of claim 1, wherein the unmodified CD86 polypeptide comprises:
(i) the sequence of amino acids set forth in SEQ ID NO:29,
(ii) a sequence of amino acids that has at least 95% sequence identity to SEQ ID NO:29;
(iii) the sequence of amino acids set forth in SEQ ID NO:123;
(iv) the sequence of amino acids set forth in SEQ ID NO:122; or
(v) a portion of any of (i)-(iv) comprising an IgV domain.

7.-13. (canceled)

14. The variant CD86 polypeptide of claim 1, wherein the variant CD86 polypeptide comprises up to 2, 3, 4, 5, 6, 7, 8, 9, 10, 11, 12, 13, 14, 15, 16, 17, 18, 19 or 20 amino acid modifications.

15. The variant CD86 polypeptide of claim 1, wherein the one or more amino acid modification comprises F33I and further comprises one or more amino acid substitutions selected from A13V, Q18K, Q25L, S28G, E38V, N39D, L40M, L40S, N43K, V45I, F52L, D53G, M60K, D68N, T71A, L77P, I79N, K80E, K80M, K80R, K82T, Q86K, Q86R, I88F, I88T, I89V, H90 L, H90Y, K92I, K93T, M97L, Q102H, N104S, F113S, S114G, N123D, V128A, Y129N, L132M, T133A, I137T, P141A, P143H, K144E, V148D, K153E, K153R, N154D, E158G, V170D, E172G, D175E, I178T, L180S, S181P, S183P, P185S, T192N, I193V, I196V, L197M, E198D, L205S, S206T, S207P, E212V, D215V, P216H, H222T or I223F.

16. The variant CD86 polypeptide of claim 1, comprising one or more amino acid modifications selected from Q25L/F33I/H90Y/V128A/P141A/E158G/S181P, Q18K/Q25L/F33I/L40S/H90L, Q25L/S28G/F33I/F52L/H90L/Q102H/I178T, Q25L/F33I/H90L/K144E/L180S, Q25L/F33I/H90L/K153E/E172G/T192N, Q25L/F33I/Q86R/H90Y/D175E/1196V/E198D, Q25L/F33I/H90L, or Q25L/F33I/Q86R/H90L/K93T.

17.-32. (canceled)

33. The variant CD86 polypeptide of claim 1, wherein the variant CD86 polypeptide comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 29.

34. The variant CD86 polypeptide of claim 1, wherein the variant CD86 polypeptide specifically binds to the ectodomain of CD28 with increased affinity compared to the binding of the unmodified CD86 for the same ectodomain.

35. (canceled)

36. The variant CD86 polypeptide of claim 1, wherein the variant CD86 polypeptide specifically binds to the ectodomain of CTLA-4 with decreased affinity compared to the binding of the unmodified CD86 for the same ectodomain.

37.-39. (canceled)

40. The variant CD86 polypeptide of claim 1, wherein the variant CD86 polypeptide comprises:
(i) the sequence of amino acids set forth in any of SEQ ID NOS: 91, 96, 99-102, 112, or 113, or a specific binding fragment thereof,
(ii) a sequence of amino acids that exhibits at least 95% sequence identity to any of SEQ ID NOS: 91, 96, 99-102, 112, or 113, or a specific binding fragment thereof and that contains the one or more of the amino acid modifications of the respective SEQ ID NO set forth in any of SEQ ID NOS: 91, 96, 99-102, 112, or 113,
(iii) the sequence of amino acids set forth in any of SEQ ID NOs: 147, 152, 155-158, 168, 169, or 129 or a specific binding fragment thereof, or
(iv) a sequence of amino acids that exhibits at least 95% sequence identity to any of SEQ ID NOs:141-177 or a specific binding fragment thereof and that contains the one or more of the amino acid modifications of the respective SEQ ID NO set forth in any of SEQ ID NOs: 147, 152, 155-158, 168, 169, or 129.

41.-45. (canceled)

46. An immunomodulatory protein comprising a variant CD86 polypeptide of claim 1 linked to a multimerization domain.

47. The immunomodulatory protein of claim 46, wherein the multimerization domain is an Fc domain or a variant thereof with reduced effector function.

48.-49. (canceled)

50. The variant CD86 polypeptide of claim 47, wherein the Fc domain comprises the sequence of amino acids set forth in SEQ ID NO: 229 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 229.

51. (canceled)

52. The variant CD86 polypeptide of claim 47, wherein the Fc domain is a variant IgGl Fc domain comprising one or more amino acid modifications selected from among E233P, L234A, L234V, L235A, L235E, G236del, G237A, S267K, N297G, V302C and K447del, each by EU numbering.

53.-58. (canceled)

59. A transmembrane immunomodulatory protein comprising a variant CD86 polypeptide of claim 1 further comprising a transmembrane domain linked, directly or indirectly, to the extracellular domain (ECD) or specific binding fragment thereof of the variant CD86 polypeptide.

60.-65. (canceled)

66. An immunomodulatory protein, comprising a first variant CD86 polypeptide of claim 1 and second variant CD86 polypeptide of claim 1.

67.-70. (canceled)

71. An immunomodulatory protein, comprising the variant CD86 polypeptide of claim 1 linked, directly or indirectly via a linker, to a second polypeptide comprising an immunoglobulin superfamily (IgSF) domain of an IgSF family member.

72.-115. (canceled)

116. A conjugate, comprising the variant CD86 polypeptide of claim 1 linked to a targeting moiety that specifically binds to a molecule on the surface of a cell.

117.-126. (canceled)

127. A nucleic acid molecule(s) encoding the variant CD86 polypeptide of claim 1, an immunomodulatory protein comprising the variant CD86 polypeptide of claim 1, or a conjugate comprising the variant CD86 polypeptide of claim 1.

128. A vector, comprising the nucleic acid molecule of claim 127.

129. A cell, comprising the variant CD86 polypeptide of claim 1.

130. A method of producing a protein comprising a variant CD86 polypeptide, comprising introducing the nucleic acid molecule of claim 127 into a host cell under conditions to express the protein in the cell and further comprising isolating or purifying the protein from the cell.

131.-132. (canceled)

133. An engineered cell, comprising the variant CD86 polypeptide of claim 1, an immunomodulatory protein comprising the variant CD86 polypeptide of claim 1, or a conjugate comprising the variant CD86 polypeptide of claim 1.

134.-140. (canceled)

141. An infectious agent, comprising the variant CD86 polypeptide of claim 1, an immunomodulatory protein comprising the variant CD86 polypeptide of claim 1, or a conjugate comprising the variant CD86 polypeptide of claim 1.

142.-143. (canceled)

144. A pharmaceutical composition, comprising the variant CD86 polypeptide of claim 1, an immunomodulatory protein comprising the variant CD86 polypeptide of claim 1, a conjugate comprising the variant CD86 polypeptide of claim 1, or an engineered cell comprising a variant CD86 polypeptide of claim 1.

145.-146. (canceled)

147. A kit comprising the pharmaceutical composition of claim 144 and instructions for use.

148. A method of modulating an immune response in a subject, the method comprising administering a variant CD86 polypeptide of claim 1 to a subject in need thereof.

149.-152. (canceled)

153. A method of treating a disease or condition in a subject in need thereof, the method comprising administering a variant CD86 polypeptide of claim 1.

154.-172. (canceled)
